SEQUENCE LISTING (595 sequences)

SEQ ID NO: 1 - protein, 205 aa, Artificial Sequence
  Synthetic polypeptide
  MISC_FEATURE (1)..(3): Optional N-terminal residues
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 2 - protein, 157 aa, Artificial Sequence
  Synthetic polypeptide
  MISC_FEATURE (1)..(1): Optional N-terminal residue
MNQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 3 - protein, 114 aa, Artificial Sequence
  Synthetic polypeptide
  MISC_FEATURE (1)..(1): Optional N-terminal residue
MPIFTLNTNIKATDVPSDFLSLTSRLVGLILSKPGSYVAVHINTDQQLSFGGSTNPAAFGTLMSIGGIEPSKNRDHSAVLFDHLNAMLGIPKNRMYIHFVNLNGDDVGWNGTTF

SEQ ID NO: 4 - protein, 157 aa, Artificial Sequence
  Synthetic polypeptide
  MISC_FEATURE (1)..(1): Optional N-terminal residue
MNQHSHKDHETVRIAVVRARWHADIVDACVEAFEIAMAAIGGDRFAVDVFDVPGAYEIPLHARTLAETGRYGAVLGTAFVVNGGIYRHEFVASAVIDGMMNVQLSTGVPVLSAVLTPHRYRDSAEHHRFFAAHFAVKGVEAARACIEILAAREKIAA

SEQ ID NO: 5 - protein, 207 aa, Artificial Sequence
  Synthetic polypeptide
  MISC_FEATURE (1)..(3): Optional N-terminal residues
  MISC_FEATURE (206)..(207): Optional C-terminal residues
MTMEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGS

SEQ ID NO: 6 - protein, 215 aa, Artificial Sequence
  Synthetic polypeptide
  MISC_FEATURE (1)..(3): Optional N-terminal residues
  MISC_FEATURE (206)..(215): Optional C-terminal residues
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTEGSWSHPQFEK

SEQ ID NO: 7 - protein, 215 aa, Artificial Sequence
  Synthetic polypeptide
  MISC_FEATURE (1)..(3): Optional N-terminal residues
  MISC_FEATURE (206)..(215): Optional C-terminal residues
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSWSHPQFEK

SEQ ID NO: 8 - protein, 215 aa, Artificial Sequence
  Synthetic polypeptide
  MISC_FEATURE (1)..(3): Optional N-terminal residues
  MISC_FEATURE (206)..(215): Optional C-terminal residues
MTMEELFKRHTIVAVLRANSVEEAIEKAVAVFAGGVHLIEITFTVPDADTVIKALSVLKEDGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSWSHPQFEK

SEQ ID NO: 9 - protein, 214 aa, Artificial Sequence
  Synthetic polypeptide
  MISC_FEATURE (1)..(11): N-terminal helical linker
  MISC_FEATURE (12)..(12): Optional residue
EKAAKAEEAARMEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTE

SEQ ID NO: 10 - protein, 249 aa, Artificial Sequence
  Synthetic polypeptide
  MISC_FEATURE (1)..(30): N-terminal helical linker
  MISC_FEATURE (233)..(239): Optional residues
  MISC_FEATURE (240)..(249): Optional C-terminal His-tag
GDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSGLVPRGSLEHHHHHH

SEQ ID NO: 11 - protein, 207 aa, Artificial Sequence
  Synthetic polypeptide
  MISC_FEATURE (1)..(3): Optional N-terminal residues
  MISC_FEATURE (206)..(207): Optional C-terminal residues
MKMEELFKKHKIVAVLRANSVEEAIEKAVAVFAGGVHLIEITFTVPDADTVIKALSVLKEKGAIIGAGTVTSVDQARKAVESGAEFIVSPHLDEEISQFAKEKGVFYMPGVMTPTELVKAMKLGHDILKLFPGEVVGPQFVKAMKGPFPNVKFVPTGGVCLDNVAEWFKAGVLAVGVGSALVKGTPDEVREKAKAFVEKIRGATEGS

SEQ ID NO: 12 - protein, 158 aa, Artificial Sequence
  Synthetic polypeptide
NQHSQKDQETVRIAVVRARWHAEIVDAAVSAFEAAMRKIGGERFAVDVFDVPGAYEIPLHARTLAKTGRYGAVLGTAFVVNGGIYRHEFVASAVIDGMMNVQLDTGVPVLSAVLTPHNYDDSDAHTLLFLALFAVKGMEAARAAVEILAAREKIAAGS

SEQ ID NO: 13 - protein, 231 aa, Artificial Sequence
  Synthetic polypeptide
MKMEELFKKHKIVAVLRANSVEEAIEKAVAVFAGGVHLIEITFTVPDADTVIKALSVLKEKGAIIGAGTVTSVDQARKAVESGAEFIVSPHLDEEISQFAKEKGVFYMPGVMTPTELVKAMKLGHDILKLFPGEVVGPQFVKAMKGPFPNVKFVPTGGVCLDNVAEWFKAGVLAVGVGSALVKGTPDEVREKAKAFVEKIRGATEGSGGGCGSGCGSGCGGGCGSGCGGGC

SEQ ID NO: 14 - protein, 213 aa, Artificial Sequence
  Synthetic polypeptide
MEELFKKHKIVAVLRANSVEEAIEKAVAVFAGGVHLIEITFTVPDADTVIKALSVLKEKGAIIGAGTVTSVDQARKAVESGAEFIVSPHLDEEISQFAKEKGVFYMPGVMTPTELVKAMKLGHDILKLFPGEVVGPQFVKAMKGPFPNVKFVPTGGVCLDNVAEWFKAGVLAVGVGSALVKGTPDEVREKAKAFVEKIRGATEGSGGGCGSGC

SEQ ID NO: 15 - protein, 193 aa, Artificial Sequence
  Synthetic polypeptide
  MISC_FEATURE (1)..(31): Optional N-terminal residues
  MISC_FEATURE (188)..(193): Optional C-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGS

SEQ ID NO: 16 - protein, 159 aa, Artificial Sequence
  Synthetic polypeptide
  MISC_FEATURE (1)..(1): Optional N-terminal residue
  MISC_FEATURE (158)..(159): Optional C-terminal residue
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHNYDKSKAHTLLFLALFAVKGMEAARACVEILAAREKIAAGS

SEQ ID NO: 17 - protein, 159 aa, Artificial Sequence
  Synthetic polypeptide
  MISC_FEATURE (1)..(1): Optional N-terminal residue
  MISC_FEATURE (158)..(159): Optional C-terminal residues
MNQHSHKDHETVRIAVVRARWHAFIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHNYDKSNAKTLLFLALFAVKGMEAARACVEILAAREKIAAGS

SEQ ID NO: 18 - protein, 159 aa, Artificial Sequence; identical to SEQ ID NO: 17
  Synthetic polypeptide
  MISC_FEATURE (1)..(1): Optional N-terminal residue
  MISC_FEATURE (158)..(159): Optional N-terminal residue

SEQ ID NO: 19 - protein, 204 aa, Artificial Sequence
  Synthetic polypeptide
  MISC_FEATURE (1)..(31): Optional N-terminal residues
  MISC_FEATURE (188)..(204): Optional C-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGYLDGSGSGS

SEQ ID NO: 20 - protein, 197 aa, Artificial Sequence
  Synthetic polypeptide
  MISC_FEATURE (1)..(31): Optional N-terminal residues
  MISC_FEATURE (188)..(197): Optional C-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGYL

SEQ ID NO: 21 - protein, 159 aa, Artificial Sequence
  Synthetic polypeptide
  MISC_FEATURE (1)..(1): Optional N-terminal residue
  MISC_FEATURE (158)..(159): Optional C-terminal residues
MNQHSQKDQETVRIAVVRARWHAEIVDAAVSAFEAAMRKIGGERFAVDVFDVPGAYEIPLHARTLAKTGRYGAVLGTAFVVNGGIYRHEFVASAVIDGMMNVQLDTGVPVLSAVLTPHNYDDSDAHTLLFLALFAVKGMEAARAAVEILAAREKIAAGS

SEQ ID NO: 22 - protein, 114 aa, Artificial Sequence
  Synthetic polypeptide
  MISC_FEATURE (1)..(1): Optional N-terminal residue
MPIFTLNTNIKADDVPSDFLSLTSRLVGLILSKPGSYVAVHINTDQQLSFGGSTNPAAFGTLMSIGGIEPKKNRDHSAVLFDHLNAMLGIPKNRMYIHFVRLNGKDVGWNGTTF

SEQ ID NO: 23 - protein, 157 aa, Artificial Sequence
  Synthetic polypeptide
  MISC_FEATURE (1)..(1): Optional N-terminal residue
MNQHSHKDHETVRIAVVRARWHADIVDACVEAFEIAMAAIGGDRFAVDVFDVPGAYEIPLHARTLAETGRYGAVLGTAFVVNGGIYRHEFVASAVIDGMMNVQLDTGVPVLSAVLTPHNYDKSKEHHRFFAAHFAVKGVEAARACIEILNAREKIAA

SEQ ID NO: 24 - protein, 90 aa, Artificial Sequence
  Synthetic polypeptide
VSDVPRDLEVVAATPTSLLISWYYPFCAFYYRITYGETGGNSPVQEFTVPRPSDTATISGLKPGVDYTITVYAVTCLGSYSRPISINYRT

SEQ ID NO: 25 - protein, 58 aa, Artificial Sequence
  Synthetic polypeptide
VDNKFNKEMRNAYWEIALLPNLNNQQKRAFIRSLYDDPSQSANLLAEAKKLNDAQAPK

SEQ ID NO: 26 - protein, 124 aa, Artificial Sequence
  Synthetic polypeptide
DLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 27 - protein, 58 aa, Artificial Sequence
  Synthetic polypeptide
VDNKFNKEMWAAWEEIRNLPNLNGWQMTAFIASLVDDPSQSANLLAEAKKLNDAQAPK

SEQ ID NO: 28 - protein, 156 aa, Artificial Sequence
  Synthetic polypeptide
DLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLN

SEQ ID NO: 29 - protein, 118 aa, Artificial Sequence
  Synthetic polypeptide
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGS

SEQ ID NO: 30 - protein, 13 aa, Artificial Sequence
  Synthetic polypeptide
AHIVMVDAYKPTK

SEQ ID NO: 31 - protein, 240 aa, Artificial Sequence
  Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 32 - protein, 241 aa, Artificial Sequence
  Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 33 - protein, 108 aa, Artificial Sequence
  Synthetic polypeptide
GVSDVPRDLEVVAATPTSLLISWDSGRGSYQYYRITYGETGGNSPVQEFTVPGPVHTATISGLKPGVDYTITVYAVTDHKPHADGPHTYHESPISINYRTEIDKGSGC

SEQ ID NO: 34 - protein, 127 aa, Artificial Sequence
  Synthetic polypeptide
MADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTV

SEQ ID NO: 35 - protein, 90 aa, Artificial Sequence
  Synthetic polypeptide
  misc (23)..(29): Xaa can be any other amino acid so as to change the binding properties of the polypeptide.
  misc (51)..(54): Xaa can be any other amino acid so as to change the binding properties of the polypeptide.
  misc (76)..(82): Xaa can be any other amino acid so as to change the binding properties of the polypeptide.
VSDVPRDLEVVAATPTSLLISWXXXXXXXYYRITYGETGGNSPVQEFTVPXXXXTATISGLKPGVDYTITVYAVTXXXXXXXPISINYRT

SEQ ID NO: 36 - protein, 58 aa, Artificial Sequence
  Synthetic polypeptide
  MISC_FEATURE (9)..(11): Xaa can be any other amino acid so as to change the binding properties of the polypeptide.
  MISC_FEATURE (13)..(14): Xaa can be any other amino acid so as to change the binding properties of the polypeptide.
  MISC_FEATURE (17)..(18): Xaa can be any other amino acid so as to change the binding properties of the polypeptide.
  MISC_FEATURE (24)..(25): Xaa can be any other amino acid so as to change the binding properties of the polypeptide.
  MISC_FEATURE (27)..(28): Xaa can be any other amino acid so as to change the binding properties of the polypeptide.
  MISC_FEATURE (31)..(31): Xaa can be any other amino acid so as to change the binding properties of the polypeptide.
  misc_feature (32)..(32): Xaa can be any naturally occurring amino acid
  MISC_FEATURE (34)..(34): Xaa can be any other amino acid so as to change the binding properties of the polypeptide.
  misc_feature (35)..(35): Xaa can be any naturally occurring amino acid
VDNKFNKEXXXAXXEIXXLPNLNXXQXXAFIXSLXDDPSQSANLLAEAKKLNDAQAPK

SEQ ID NO: 37 - protein, 156 aa, Artificial Sequence
  Synthetic polypeptide
  MISC_FEATURE (11)..(11): Xaa can be any other amino acid so as to change the binding properties of the polypeptide.
  misc_feature (12)..(12): Xaa can be any naturally occurring amino acid
  MISC_FEATURE (13)..(13): Xaa can be any other amino acid so as to change the binding properties of the polypeptide.
  misc_feature (14)..(14): Xaa can be any naturally occurring amino acid
  MISC_FEATURE (31)..(31): Xaa can be any other amino acid so as to change the binding properties of the polypeptide.
  MISC_FEATURE (33)..(34): Xaa can be any other amino acid so as to change the binding properties of the polypeptide.
  MISC_FEATURE (36)..(36): Xaa can be any other amino acid so as to change the binding properties of the polypeptide.
  MISC_FEATURE (42)..(45): Xaa can be any other amino acid so as to change the binding properties of the polypeptide.
  misc_feature (46)..(46): Xaa can be any naturally occurring amino acid
  MISC_FEATURE (57)..(57): Xaa can be any other amino acid so as to change the binding properties of the polypeptide.
  MISC_FEATURE (59)..(59): Xaa can be any other amino acid so as to change the binding properties of the polypeptide.
  MISC_FEATURE (64)..(67): Xaa can be any other amino acid so as to change the binding properties of the polypeptide.
  MISC_FEATURE (69)..(69): Xaa can be any other amino acid so as to change the binding properties of the polypeptide.
  MISC_FEATURE (74)..(74): Xaa can be any other amino acid so as to change the binding properties of the polypeptide.
  MISC_FEATURE (77)..(78): Xaa can be any other amino acid so as to change the binding properties of the polypeptide.
  MISC_FEATURE (83)..(84): Xaa can be any other amino acid so as to change the binding properties of the polypeptide.
  MISC_FEATURE (88)..(89): Xaa can be any other amino acid so as to change the binding properties of the polypeptide.
  MISC_FEATURE (96)..(99): Xaa can be any other amino acid so as to change the binding properties of the polypeptide.
  MISC_FEATURE (101)..(101): Xaa can be any other amino acid so as to change the binding properties of the polypeptide.
DLGKKLLEAARXGXDDEVRILMANGADVNAXDXXGXTPLHLAXXXXHLEIVEVLLKXGXDVNAXXXXGXTPLHXAAXXHLEIXXVLLXXGADVNAXXXXGXTPLHLAAHNGHLEIVEVLLKHGADVNAQDKFGKTAFDISIDNGNEDLAEILQKLN

SEQ ID NO: 38 - protein, 239 aa, Artificial Sequence
  Synthetic polypeptide
  MISC_FEATURE (27)..(35): Xaa can be any other amino acid so as to change the binding properties of the polypeptide.
  MISC_FEATURE (50)..(57): Xaa can be any other amino acid so as to change the binding properties of the polypeptide.
  MISC_FEATURE (96)..(107): Xaa can be any other amino acid so as to change the binding properties of the polypeptide.
  MISC_FEATURE (117)..(133): Gly-Ser linker
  MISC_FEATURE (156)..(166): Xaa can be any other amino acid so as to change the binding properties of the polypeptide.
  MISC_FEATURE (178)..(185): Xaa can be any other amino acid so as to change the binding properties of the polypeptide.
  MISC_FEATURE (217)..(229): Xaa can be any other amino acid so as to change the binding properties of the polypeptide.
DIKLQQSGAELARPGASVKMSCKTSGXXXXXXXXXWVKQRPGQGLEWIGXXXXXXXXNYNQKFKDKATLTTDKSSSTAYMQLSSLTSEDSAVYYCXXXXXXXXXXXXWGQGTTLTVSSGGGGSGGGGSGGGGSDIQLTQSPAIMSASPGEKVTMTXXXXXXXXXXXWYQQKSGTSPKXXXXXXXXVASGVPYRFSGSGSGTSYSLTISSMEAEDAAXXXXXXXXXXXXXFGAGTKLELK

SEQ ID NO: 39 - protein, 107 aa, Artificial Sequence
  Synthetic polypeptide
  MISC_FEATURE (23)..(30): Xaa can be any other amino acid so as to change the binding properties of the polypeptide.
  MISC_FEATURE (52)..(55): Xaa can be any other amino acid so as to change the binding properties of the polypeptide.
  MISC_FEATURE (77)..(91): Xaa can be any other amino acid so as to change the binding properties of the polypeptide.
VSDVPRDLEVVAATPTSLLISWXXXXXXXXYYRITYGETGGNSPVQEFTVPXXXXTATISGLKPGVDYTITVYAVTXXXXXXXXXXXXXXXPISINYRTEIDKGSGC

SEQ ID NO: 40 - protein, 127 aa, Artificial Sequence
  Synthetic polypeptide
  MISC_FEATURE (27)..(35): Xaa can be any other amino acid so as to change the binding properties of the polypeptide.
  MISC_FEATURE (54)..(62): Xaa can be any other amino acid so as to change the binding properties of the polypeptide.
  MISC_FEATURE (101)..(118): Xaa can be any other amino acid so as to change the binding properties of the polypeptide.
MADVQLVESGGGLVQAGGSLRLSCAAXXXXXXXXXMSWFRQAPGKEREFVAGIXXXXXXXXXADSVKGRFTISRDNTKNTLYLQMNSLKAEDTAVYYCAVXXXXXXXXXXXXXXXXXXWGQGTQVTV

SEQ ID NO: 41 - protein, 254 aa, Artificial Sequence
  Synthetic polypeptide
AHIVMVDAYKPTKDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 42 - protein, 256 aa, Artificial Sequence
  Synthetic polypeptide
AHIVMVDAYKPTKGSGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 43 - protein, 143 aa, Artificial Sequence
  Synthetic polypeptide
AHIVMVDAYKPTKGSGMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTV

SEQ ID NO: 44 - protein, 2 aa, Artificial Sequence
  Synthetic polypeptide
GS

SEQ ID NO: 45 - protein, 6 aa, Artificial Sequence
  Synthetic polypeptide
GSGSGS

SEQ ID NO: 46 - protein, 9 aa, Artificial Sequence
  Synthetic polypeptide
GGSGGSGGS

SEQ ID NO: 47 - protein, 6 aa, Artificial Sequence
  Synthetic polypeptide
SGSGSG

SEQ ID NO: 48 - protein, 7 aa, Artificial Sequence
  Synthetic polypeptide
SSGSGGS

SEQ ID NO: 49 - protein, 7 aa, Artificial Sequence
  Synthetic polypeptide
PPPPPPP

SEQ ID NO: 50 - protein, 18 aa, Artificial Sequence
  Synthetic polypeptide
STEEGTSESATPESGPGS

SEQ ID NO: 51 - protein, 20 aa, Artificial Sequence
  Synthetic polypeptide
EPATSGSETPGTSESATPES

SEQ ID NO: 52 - protein, 14 aa, Artificial Sequence
  Synthetic polypeptide
SPETSPASTEPEGS

SEQ ID NO: 53 - protein, 17 aa, Artificial Sequence
  Synthetic polypeptide
  MISC_FEATURE (1)..(6): Optional N-terminal residues
  MISC_FEATURE (10)..(17): Optional C-terminal residues
GSLEGSRGYLDGSGSGS

SEQ ID NO: 54 - protein, 16 aa, Artificial Sequence
  Synthetic polypeptide
  MISC_FEATURE (11)..(16): Optional C-terminal residues
YKKSRLGFRVGGSGGS

SEQ ID NO: 55 - protein, 29 aa, Artificial Sequence
  Synthetic polypeptide
AGYFLTYTPKSVTPDGVTLSQKTLTGAVG

SEQ ID NO: 56 - protein, 12 aa, Artificial Sequence
  Synthetic polypeptide
EKAAKAEEAARI

SEQ ID NO: 57 - protein, 18 aa, Artificial Sequence
  Synthetic polypeptide
GDGGRGSRGGDGSGGSSG

SEQ ID NO: 58 - protein, 59 aa, Artificial Sequence
  Synthetic polypeptide
STEEGTSESATPESGPGSEPATSGSETPGTSESATPESGPGTSTEPSEGSAPGTSTEPE

SEQ ID NO: 59 - protein, 59 aa, Artificial Sequence
  Synthetic polypeptide
GGSPGSPAGSPTSTEEGTSESATPESGPGTSTEPSEGSAPGSPAGSPTSTEEGTSTEPE

SEQ ID NO: 60 - protein, 60 aa, Artificial Sequence
  Synthetic polypeptide
PASPASEPASPASEPASPASEPASPASEPASPASEPASPASEPASPASEPASPASEPASP

SEQ ID NO: 61 - protein, 118 aa, Artificial Sequence
  Synthetic polypeptide
STEEGTSESATPESGPGSEPATSGSETPGTSESATPESGPGTSTEPSEGSAPGTSTEPESTEEGTSESATPESGPGSEPATSGSETPGTSESATPESGPGTSTEPSEGSAPGTSTEPE

SEQ ID NO: 62 - protein, 118 aa, Artificial Sequence
  Synthetic polypeptide
STEEGTSESATPESGPGSEPATSGSETPGTSESATPESGPGTSTEPSEGSAPGTSTEPEPASPASEPASPASEPASPASEPASPASEPASPASEPASPASEPASPASEPASPASEPAP

SEQ ID NO: 63 - protein, 59 aa, Artificial Sequence
  Synthetic polypeptide
PETSPASTEPEGSPETSPASTEPEGSPETSPASTEPEGSPETSPASTEPEGSPETSPAS

SEQ ID NO: 64 - protein, 59 aa, Artificial Sequence
  Synthetic polypeptide
PESTGAPGETSPEGSPESTGAPGETSPEGSPESTGAPGETSPEGSPESTGAPGETSPEG

SEQ ID NO: 65 - protein, 59 aa, Artificial Sequence
  Synthetic polypeptide
SEGSAPGTSESATPESGPGTSESATPESGPGTSESATPESGPGSEPATSGSETPGSEPT

SEQ ID NO: 66 - protein, 59 aa, Artificial Sequence
  Synthetic polypeptide
SGSEPEPTSPSETPSPPGGTPGSEATSPTEETGAEGPAGPGPGSEEGSTEGAGTSPEES

SEQ ID NO: 67 - protein, 60 aa, Artificial Sequence
  Synthetic polypeptide
DEADEADEADEADEADEADEADEADEADEADEADEADEADEADEADEADEADEADEADEA

SEQ ID NO: 68 - protein, 60 aa, Artificial Sequence
  Synthetic polypeptide
DEDEADEDEADEDEADEDEADEDEADEDEADEDEADEDEADEDEADEDEADEDEADEDEA

SEQ ID NO: 69 - protein, 60 aa, Artificial Sequence
  Synthetic polypeptide
DEDEDEDEDEADEDEDEDEDEADEDEDEDEDEADEDEDEDEDEADEDEDEDEDEADEDED

SEQ ID NO: 70 - protein, 60 aa, Artificial Sequence
  Synthetic polypeptide
DESDESDESDESDESDESDESDESDESDESDESDESDESDESDESDESDESDESDESDES

SEQ ID NO: 71 - protein, 60 aa, Artificial Sequence
  Synthetic polypeptide
DEDESDEDESDEDESDEDESDEDESDEDESDEDESDEDESDEDESDEDESDEDESDEDES

SEQ ID NO: 72 - protein, 60 aa, Artificial Sequence
  Synthetic polypeptide
DEDEDEDEDESDEDEDEDEDESDEDEDEDEDESDEDEDEDEDESDEDEDEDEDESDEDED

SEQ ID NO: 73 - protein, 60 aa, Artificial Sequence
  Synthetic polypeptide
DETDETDETDETDETDETDETDETDETDETDETDETDETDETDETDETDETDETDETDET

SEQ ID NO: 74 - protein, 60 aa, Artificial Sequence
  Synthetic polypeptide
DEDETDEDETDEDETDEDETDEDETDEDETDEDETDEDETDEDETDEDETDEDETDEDET

SEQ ID NO: 75 - protein, 60 aa, Artificial Sequence
  Synthetic polypeptide
DEDEDEDEDETDEDEDEDEDETDEDEDEDEDETDEDEDEDEDETDEDEDEDEDETDEDED

SEQ ID NO: 76 - protein, 60 aa, Artificial Sequence
  Synthetic polypeptide
DEEDEEDEEDEEDEEDEEDEEDEEDEEDEEDEEDEEDEEDEEDEEDEEDEEDEEDEEDEE

SEQ ID NO: 77 - protein, 60 aa, Artificial Sequence
  Synthetic polypeptide
DEDEEDEDEEDEDEEDEDEEDEDEEDEDEEDEDEEDEDEEDEDEEDEDEEDEDEEDEDEE

SEQ ID NO: 78 - protein, 60 aa, Artificial Sequence
  Synthetic polypeptide
DEDEDEDEDEEDEDEDEDEDEEDEDEDEDEDEEDEDEDEDEDEEDEDEDEDEDEEDEDED

SEQ ID NO: 79 - protein, 60 aa, Artificial Sequence
  Synthetic polypeptide
DEDDEDDEDDEDDEDDEDDEDDEDDEDDEDDEDDEDDEDDEDDEDDEDDEDDEDDEDDED

SEQ ID NO: 80 - protein, 60 aa, Artificial Sequence
  Synthetic polypeptide
DEDEDDEDEDDEDEDDEDEDDEDEDDEDEDDEDEDDEDEDDEDEDDEDEDDEDEDDEDED

SEQ ID NO: 81 - protein, 60 aa, Artificial Sequence
  Synthetic polypeptide
DEDEDEDEDEDDEDEDEDEDEDDEDEDEDEDEDDEDEDEDEDEDDEDEDEDEDEDDEDED

SEQ ID NO: 82 - protein, 60 aa, Artificial Sequence
  Synthetic polypeptide
DEDEQDEDEQDEDEQDEDEQDEDEQDEDEQDEDEQDEDEQDEDEQDEDEQDEDEQDEDEQ

SEQ ID NO: 83 - protein, 60 aa, Artificial Sequence
  Synthetic polypeptide
DEDEDEDEDEQDEDEDEDEDEQDEDEDEDEDEQDEDEDEDEDEQDEDEDEDEDEQDEDED

SEQ ID NO: 84 - protein, 60 aa, Artificial Sequence
  Synthetic polypeptide
DENDENDENDENDENDENDENDENDENDENDENDENDENDENDENDENDENDENDENDEN

SEQ ID NO: 85 - protein, 60 aa, Artificial Sequence
  Synthetic polypeptide
DEDENDEDENDEDENDEDENDEDENDEDENDEDENDEDENDEDENDEDENDEDENDEDEN

SEQ ID NO: 86 - protein, 60 aa, Artificial Sequence
  Synthetic polypeptide
DEDEDEDEDENDEDEDEDEDENDEDEDEDEDENDEDEDEDEDENDEDEDEDEDENDEDED

SEQ ID NO: 87 - protein, 60 aa, Artificial Sequence
  Synthetic polypeptide
DEKDEKDEKDEKDEKDEKDEKDEKDEKDEKDEKDEKDEKDEKDEKDEKDEKDEKDEKDEK

SEQ ID NO: 88 - protein, 60 aa, Artificial Sequence
  Synthetic polypeptide
DEDEKDEDEKDEDEKDEDEKDEDEKDEDEKDEDEKDEDEKDEDEKDEDEKDEDEKDEDEK

SEQ ID NO: 89 - protein, 60 aa, Artificial Sequence
  Synthetic polypeptide
DEDEDEDEDEKDEDEDEDEDEKDEDEDEDEDEKDEDEDEDEDEKDEDEDEDEDEKDEDED

SEQ ID NO: 90 - protein, 60 aa, Artificial Sequence
  Synthetic polypeptide
DERDERDERDERDERDERDERDERDERDERDERDERDERDERDERDERDERDERDERDER

SEQ ID NO: 91 - protein, 60 aa, Artificial Sequence
  Synthetic polypeptide
DEDERDEDERDEDERDEDERDEDERDEDERDEDERDEDERDEDERDEDERDEDERDEDER

SEQ ID NO: 92 - protein, 60 aa, Artificial Sequence
  Synthetic polypeptide
DEDEDEDEDERDEDEDEDEDERDEDEDEDEDERDEDEDEDEDERDEDEDEDEDERDEDED

SEQ ID NO: 93 - protein, 60 aa, Artificial Sequence
  Synthetic polypeptide
DEPDEPDEPDEPDEPDEPDEPDEPDEPDEPDEPDEPDEPDEPDEPDEPDEPDEPDEPDEP

SEQ ID NO: 94 - protein, 60 aa, Artificial Sequence
  Synthetic polypeptide
DEDEPDEDEPDEDEPDEDEPDEDEPDEDEPDEDEPDEDEPDEDEPDEDEPDEDEPDEDEP

SEQ ID NO: 95 - protein, 60 aa, Artificial Sequence
  Synthetic polypeptide
DEDEDEDEDEPDEDEDEDEDEPDEDEDEDEDEPDEDEDEDEDEPDEDEDEDEDEPDEDED

SEQ ID NO: 96 - protein, 60 aa, Artificial Sequence
  Synthetic polypeptide
DEGDEGDEGDEGDEGDEGDEGDEGDEGDEGDEGDEGDEGDEGDEGDEGDEGDEGDEGDEG

SEQ ID NO: 97 - protein, 60 aa, Artificial Sequence
  Synthetic polypeptide
DEDEGDEDEGDEDEGDEDEGDEDEGDEDEGDEDEGDEDEGDEDEGDEDEGDEDEGDEDEG

SEQ ID NO: 98 - protein, 60 aa, Artificial Sequence
  Synthetic polypeptide
DEDEDEDEDEGDEDEDEDEDEGDEDEDEDEDEGDEDEDEDEDEGDEDEDEDEDEGDEDED

SEQ ID NO: 99 - protein, 60 aa, Artificial Sequence
  Synthetic polypeptide
DELDELDELDELDELDELDELDELDELDELDELDELDELDELDELDELDELDELDELDEL

SEQ ID NO: 100 - protein, 60 aa, Artificial Sequence
  Synthetic polypeptide
DEDELDEDELDEDELDEDELDEDELDEDELDEDELDEDELDEDELDEDELDEDELDEDEL

SEQ ID NO: 101 - protein, 60 aa, Artificial Sequence
  Synthetic polypeptide
DEDEDEDEDELDEDEDEDEDELDEDEDEDEDELDEDEDEDEDELDEDEDEDEDELDEDED

SEQ ID NO: 102 - protein, 60 aa, Artificial Sequence
  Synthetic polypeptide
DEIDEIDEIDEIDEIDEIDEIDEIDEIDEIDEIDEIDEIDEIDEIDEIDEIDEIDEIDEI

SEQ ID NO: 103 - protein, 60 aa, Artificial Sequence
  Synthetic polypeptide
DEDEIDEDEIDEDEIDEDEIDEDEIDEDEIDEDEIDEDEIDEDEIDEDEIDEDEIDEDEI

SEQ ID NO: 104 - protein, 60 aa, Artificial Sequence
  Synthetic polypeptide
DEDEDEDEDEIDEDEDEDEDEIDEDEDEDEDEIDEDEDEDEDEIDEDEDEDEDEIDEDED

SEQ ID NO: 105 - protein, 60 aa, Artificial Sequence
  Synthetic polypeptide
RKARKARKARKARKARKARKARKARKARKARKARKARKARKARKARKARKARKARKARKA

SEQ ID NO: 106 - protein, 60 aa, Artificial Sequence
  Synthetic polypeptide
RKRKARKRKARKRKARKRKARKRKARKRKARKRKARKRKARKRKARKRKARKRKARKRKA

SEQ ID NO: 107 - protein, 60 aa, Artificial Sequence
  Synthetic polypeptide
RKSRKSRKSRKSRKSRKSRKSRKSRKSRKSRKSRKSRKSRKSRKSRKSRKSRKSRKSRKS

SEQ ID NO: 108 - protein, 60 aa, Artificial Sequence
  Synthetic polypeptide
RKRKSRKRKSRKRKSRKRKSRKRKSRKRKSRKRKSRKRKSRKRKSRKRKSRKRKSRKRKS

SEQ ID NO: 109 - protein, 60 aa, Artificial Sequence
  Synthetic polypeptide
RKRKRKRKRKSRKRKRKRKRKSRKRKRKRKRKSRKRKRKRKRKSRKRKRKRKRKSRKRKR

SEQ ID NO: 110 - protein, 60 aa, Artificial Sequence
  Synthetic polypeptide
RKTRKTRKTRKTRKTRKTRKTRKTRKTRKTRKTRKTRKTRKTRKTRKTRKTRKTRKTRKT

SEQ ID NO: 111 - protein, 60 aa, Artificial Sequence
  Synthetic polypeptide
RKRKTRKRKTRKRKTRKRKTRKRKTRKRKTRKRKTRKRKTRKRKTRKRKTRKRKTRKRKT

SEQ ID NO: 112 - protein, 60 aa, Artificial Sequence
  Synthetic polypeptide
RKRKRKRKRKTRKRKRKRKRKTRKRKRKRKRKTRKRKRKRKRKTRKRKRKRKRKTRKRKR

SEQ ID NO: 113 - protein, 60 aa, Artificial Sequence
  Synthetic polypeptide
RKERKERKERKERKERKERKERKERKERKERKERKERKERKERKERKERKERKERKERKE

SEQ ID NO: 114 - protein, 60 aa, Artificial Sequence
  Synthetic polypeptide
RKRKERKRKERKRKERKRKERKRKERKRKERKRKERKRKERKRKERKRKERKRKERKRKE

SEQ ID NO: 115 - protein, 60 aa, Artificial Sequence
  Synthetic polypeptide
RKRKRKRKRKERKRKRKRKRKERKRKRKRKRKERKRKRKRKRKERKRKRKRKRKERKRKR

SEQ ID NO: 116 - protein, 60 aa, Artificial Sequence
  Synthetic polypeptide
RKDRKDRKDRKDRKDRKDRKDRKDRKDRKDRKDRKDRKDRKDRKDRKDRKDRKDRKDRKD

SEQ ID NO: 117 - protein, 60 aa, Artificial Sequence
  Synthetic polypeptide
RKRKDRKRKDRKRKDRKRKDRKRKDRKRKDRKRKDRKRKDRKRKDRKRKDRKRKDRKRKD

SEQ ID NO: 118 - protein, 60 aa, Artificial Sequence
  Synthetic polypeptide
RKRKRKRKRKDRKRKRKRKRKDRKRKRKRKRKDRKRKRKRKRKDRKRKRKRKRKDRKRKR

SEQ ID NO: 119 - protein, 60 aa, Artificial Sequence
  Synthetic polypeptide
RKQRKQRKQRKQRKQRKQRKQRKQRKQRKQRKQRKQRKQRKQRKQRKQRKQRKQRKQRKQ

SEQ ID NO: 120 - protein, 60 aa, Artificial Sequence
  Synthetic polypeptide
RKRKQRKRKQRKRKQRKRKQRKRKQRKRKQRKRKQRKRKQRKRKQRKRKQRKRKQRKRKQ

SEQ ID NO: 121 - protein, 60 aa, Artificial Sequence
  Synthetic polypeptide
RKRKRKRKRKQRKRKRKRKRKQRKRKRKRKRKQRKRKRKRKRKQRKRKRKRKRKQRKRKR

SEQ ID NO: 122 - protein, 60 aa, Artificial Sequence
  Synthetic polypeptide
RKNRKNRKNRKNRKNRKNRKNRKNRKNRKNRKNRKNRKNRKNRKNRKNRKNRKNRKNRKN

SEQ ID NO: 123 - protein, 60 aa, Artificial Sequence
  Synthetic polypeptide
RKRKNRKRKNRKRKNRKRKNRKRKNRKRKNRKRKNRKRKNRKRKNRKRKNRKRKNRKRKN

SEQ ID NO: 124 - protein, 60 aa, Artificial Sequence
  Synthetic polypeptide
RKRKRKRKRKNRKRKRKRKRKNRKRKRKRKRKNRKRKRKRKRKNRKRKRKRKRKNRKRKR

SEQ ID NO: 125 - protein, 60 aa, Artificial Sequence
  Synthetic polypeptide
RKKRKKRKKRKKRKKRKKRKKRKKRKKRKKRKKRKKRKKRKKRKKRKKRKKRKKRKKRKK

SEQ ID NO: 126 - protein, 60 aa, Artificial Sequence
  Synthetic polypeptide
RKRKKRKRKKRKRKKRKRKKRKRKKRKRKKRKRKKRKRKKRKRKKRKRKKRKRKKRKRKK

SEQ ID NO: 127 - protein, 60 aa, Artificial Sequence
  Synthetic polypeptide
RKRKRKRKRKKRKRKRKRKRKKRKRKRKRKRKKRKRKRKRKRKKRKRKRKRKRKKRKRKR

SEQ ID NO: 128 - protein, 60 aa, Artificial Sequence
  Synthetic polypeptide
RKRRKRRKRRKRRKRRKRRKRRKRRKRRKRRKRRKRRKRRKRRKRRKRRKRRKRRKRRKR

SEQ ID NO: 129 - protein, 60 aa, Artificial Sequence
  Synthetic polypeptide
RKRKRRKRKRRKRKRRKRKRRKRKRRKRKRRKRKRRKRKRRKRKRRKRKRRKRKRRKRKR

SEQ ID NO: 130 - protein, 60 aa, Artificial Sequence
  Synthetic polypeptide
RKRKRKRKRKRRKRKRKRKRKRRKRKRKRKRKRRKRKRKRKRKRRKRKRKRKRKRRKRKR

SEQ ID NO: 131 - protein, 60 aa, Artificial Sequence
  Synthetic polypeptide
RKPRKPRKPRKPRKPRKPRKPRKPRKPRKPRKPRKPRKPRKPRKPRKPRKPRKPRKPRKP

SEQ ID NO: 132 - protein, 60 aa, Artificial Sequence
  Synthetic polypeptide
RKRKPRKRKPRKRKPRKRKPRKRKPRKRKPRKRKPRKRKPRKRKPRKRKPRKRKPRKRKP

SEQ ID NO: 133 - protein, 60 aa, Artificial Sequence
  Synthetic polypeptide
RKRKRKRKRKPRKRKRKRKRKPRKRKRKRKRKPRKRKRKRKRKPRKRKRKRKRKPRKRKR

SEQ ID NO: 134 - protein, 60 aa, Artificial Sequence
  Synthetic polypeptide
RKGRKGRKGRKGRKGRKGRKGRKGRKGRKGRKGRKGRKGRKGRKGRKGRKGRKGRKGRKG

SEQ ID NO: 135 - protein, 60 aa, Artificial Sequence
  Synthetic polypeptide
RKRKGRKRKGRKRKGRKRKGRKRKGRKRKGRKRKGRKRKGRKRKGRKRKGRKRKGRKRKG

SEQ ID NO: 136 - protein, 60 aa, Artificial Sequence
  Synthetic polypeptide
RKRKRKRKRKGRKRKRKRKRKGRKRKRKRKRKGRKRKRKRKRKGRKRKRKRKRKGRKRKR

SEQ ID NO: 137 - protein, 60 aa, Artificial Sequence
  Synthetic polypeptide
RKLRKLRKLRKLRKLRKLRKLRKLRKLRKLRKLRKLRKLRKLRKLRKLRKLRKLRKLRKL

SEQ ID NO: 138 - protein, 60 aa, Artificial Sequence
  Synthetic polypeptide
RKRKLRKRKLRKRKLRKRKLRKRKLRKRKLRKRKLRKRKLRKRKLRKRKLRKRKLRKRKL

SEQ ID NO: 139 - protein, 60 aa, Artificial Sequence
  Synthetic polypeptide
RKRKRKRKRKLRKRKRKRKRKLRKRKRKRKRKLRKRKRKRKRKLRKRKRKRKRKLRKRKR

SEQ ID NO: 140 - protein, 60 aa, Artificial Sequence
  Synthetic polypeptide
RKIRKIRKIRKIRKIRKIRKIRKIRKIRKIRKIRKIRKIRKIRKIRKIRKIRKIRKIRKI

SEQ ID NO: 141 - protein, 60 aa, Artificial Sequence
  Synthetic polypeptide
RKRKIRKRKIRKRKIRKRKIRKRKIRKRKIRKRKIRKRKIRKRKIRKRKIRKRKIRKRKI

SEQ ID NO: 142 - protein, 60 aa, Artificial Sequence
  Synthetic polypeptide
RKRKRKRKRKIRKRKRKRKRKIRKRKRKRKRKIRKRKRKRKRKIRKRKRKRKRKIRKRKR

SEQ ID NO: 143 - protein, 60 aa, Artificial Sequence
  Synthetic polypeptide
GSAGSAGSAGSAGSAGSAGSAGSAGSAGSAGSAGSAGSAGSAGSAGSAGSAGSAGSAGSA

SEQ ID NO: 144 - protein, 60 aa, Artificial Sequence
  Synthetic polypeptide
GSGSAGSGSAGSGSAGSGSAGSGSAGSGSAGSGSAGSGSAGSGSAGSGSAGSGSAGSGSA

SEQ ID NO: 145 - protein, 60 aa, Artificial Sequence
  Synthetic polypeptide
GSGSGSGSGSAGSGSGSGSGSAGSGSGSGSGSAGSGSGSGSGSAGSGSGSGSGSAGSGSG

SEQ ID NO: 146 - protein, 60 aa, Artificial Sequence
  Synthetic polypeptide
GSSGSSGSSGSSGSSGSSGSSGSSGSSGSSGSSGSSGSSGSSGSSGSSGSSGSSGSSGSS

SEQ ID NO: 147 - protein, 60 aa, Artificial Sequence
  Synthetic polypeptide
GSGSSGSGSSGSGSSGSGSSGSGSSGSGSSGSGSSGSGSSGSGSSGSGSSGSGSSGSGSS

SEQ ID NO: 148 - protein, 60 aa, Artificial Sequence
  Synthetic polypeptide
GSGSGSGSGSSGSGSGSGSGSSGSGSGSGSGSSGSGSGSGSGSSGSGSGSGSGSSGSGSG

SEQ ID NO: 149 - protein, 60 aa, Artificial Sequence
  Synthetic polypeptide
GSTGSTGSTGSTGSTGSTGSTGSTGSTGSTGSTGSTGSTGSTGSTGSTGSTGSTGSTGST

SEQ ID NO: 150 - protein, 60 aa, Artificial Sequence
  Synthetic polypeptide
GSGSTGSGSTGSGSTGSGSTGSGSTGSGSTGSGSTGSGSTGSGSTGSGSTGSGSTGSGST

SEQ ID NO: 151 - protein, 60 aa, Artificial Sequence
  Synthetic polypeptide
GSGSGSGSGSTGSGSGSGSGSTGSGSGSGSGSTGSGSGSGSGSTGSGSGSGSGSTGSGSG

SEQ ID NO: 152 - protein, 60 aa, Artificial Sequence
  Synthetic polypeptide
GSEGSEGSEGSEGSEGSEGSEGSEGSEGSEGSEGSEGSEGSEGSEGSEGSEGSEGSEGSE

SEQ ID NO: 153 - protein, 60 aa, Artificial Sequence
  Synthetic polypeptide
GSGSEGSGSEGSGSEGSGSEGSGSEGSGSEGSGSEGSGSEGSGSEGSGSEGSGSEGSGSE

SEQ ID NO: 154 - protein, 60 aa, Artificial Sequence
  Synthetic polypeptide
GSGSGSGSGSEGSGSGSGSGSEGSGSGSGSGSEGSGSGSGSGSEGSGSGSGSGSEGSGSG

SEQ ID NO: 155 - protein, 60 aa, Artificial Sequence
  Synthetic polypeptide
GSDGSDGSDGSDGSDGSDGSDGSDGSDGSDGSDGSDGSDGSDGSDGSDGSDGSDGSDGSD

SEQ ID NO: 156 - protein, 60 aa, Artificial Sequence
  Synthetic polypeptide
GSGSDGSGSDGSGSDGSGSDGSGSDGSGSDGSGSDGSGSDGSGSDGSGSDGSGSDGSGSD

SEQ ID NO: 157 - protein, 60 aa, Artificial Sequence
  Synthetic polypeptide
GSGSGSGSGSDGSGSGSGSGSDGSGSGSGSGSDGSGSGSGSGSDGSGSGSGSGSDGSGSG

SEQ ID NO: 158 - protein, 60 aa, Artificial Sequence
  Synthetic polypeptide
GSQGSQGSQGSQGSQGSQGSQGSQGSQGSQGSQGSQGSQGSQGSQGSQGSQGSQGSQGSQ

SEQ ID NO: 159 - protein, 60 aa, Artificial Sequence
  Synthetic polypeptide
GSGSQGSGSQGSGSQGSGSQGSGSQGSGSQGSGSQGSGSQGSGSQGSGSQGSGSQGSGSQ

SEQ ID NO: 160 - protein, 60 aa, Artificial Sequence
  Synthetic polypeptide
GSGSGSGSGSQGSGSGSGSGSQGSGSGSGSGSQGSGSGSGSGSQGSGSGSGSGSQGSGSG

SEQ ID NO: 161 - protein, 60 aa, Artificial Sequence
  Synthetic polypeptide
GSNGSNGSNGSNGSNGSNGSNGSNGSNGSNGSNGSNGSNGSNGSNGSNGSNGSNGSNGSN

SEQ ID NO: 162 - protein, 60 aa, Artificial Sequence
  Synthetic polypeptide
GSGSNGSGSNGSGSNGSGSNGSGSNGSGSNGSGSNGSGSNGSGSNGSGSNGSGSNGSGSN

SEQ ID NO: 163 - protein, 60 aa, Artificial Sequence
  Synthetic polypeptide
GSGSGSGSGSNGSGSGSGSGSNGSGSGSGSGSNGSGSGSGSGSNGSGSGSGSGSNGSGSG

SEQ ID NO: 164 - protein, 60 aa, Artificial Sequence
  Synthetic polypeptide
GSKGSKGSKGSKGSKGSKGSKGSKGSKGSKGSKGSKGSKGSKGSKGSKGSKGSKGSKGSK

SEQ ID NO: 165 - protein, 60 aa, Artificial Sequence
  Synthetic polypeptide
GSGSKGSGSKGSGSKGSGSKGSGSKGSGSKGSGSKGSGSKGSGSKGSGSKGSGSKGSGSK

SEQ ID NO: 166 - protein, 60 aa, Artificial Sequence
  Synthetic polypeptide
GSGSGSGSGSKGSGSGSGSGSKGSGSGSGSGSKGSGSGSGSGSKGSGSGSGSGSKGSGSG

SEQ ID NO: 167 - protein, 60 aa, Artificial Sequence
  Synthetic polypeptide
GSRGSRGSRGSRGSRGSRGSRGSRGSRGSRGSRGSRGSRGSRGSRGSRGSRGSRGSRGSR

SEQ ID NO: 168 - protein, 60 aa, Artificial Sequence
  Synthetic polypeptide
GSGSRGSGSRGSGSRGSGSRGSGSRGSGSRGSGSRGSGSRGSGSRGSGSRGSGSRGSGSR

SEQ ID NO: 169 - protein, 60 aa, Artificial Sequence
  Synthetic polypeptide
GSGSGSGSGSRGSGSGSGSGSRGSGSGSGSGSRGSGSGSGSGSRGSGSGSGSGSRGSGSG

SEQ ID NO: 170 - protein, 60 aa, Artificial Sequence
  Synthetic polypeptide
GSPGSPGSPGSPGSPGSPGSPGSPGSPGSPGSPGSPGSPGSPGSPGSPGSPGSPGSPGSP

SEQ ID NO: 171 - protein, 60 aa, Artificial Sequence
  Synthetic polypeptide
GSGSPGSGSPGSGSPGSGSPGSGSPGSGSPGSGSPGSGSPGSGSPGSGSPGSGSPGSGSP

SEQ ID NO: 172 - protein, 60 aa, Artificial Sequence
  Synthetic polypeptide
GSGSGSGSGSPGSGSGSGSGSPGSGSGSGSGSPGSGSGSGSGSPGSGSGSGSGSPGSGSG

SEQ ID NO: 173 - protein, 60 aa, Artificial Sequence
  Synthetic polypeptide
GSGGSGGSGGSGGSGGSGGSGGSGGSGGSGGSGGSGGSGGSGGSGGSGGSGGSGGSGGSG

SEQ ID NO: 174 - protein, 60 aa, Artificial Sequence
  Synthetic polypeptide
GSGSGGSGSGGSGSGGSGSGGSGSGGSGSGGSGSGGSGSGGSGSGGSGSGGSGSGGSGSG

SEQ ID NO: 175 - protein, 60 aa, Artificial Sequence
  Synthetic polypeptide
GSGSGSGSGSGGSGSGSGSGSGGSGSGSGSGSGGSGSGSGSGSGGSGSGSGSGSGGSGSG

SEQ ID NO: 176 - protein, 60 aa, Artificial Sequence
  Synthetic polypeptide
GSLGSLGSLGSLGSLGSLGSLGSLGSLGSLGSLGSLGSLGSLGSLGSLGSLGSLGSLGSL

SEQ ID NO: 177 - protein, 60 aa, Artificial Sequence
  Synthetic polypeptide
GSGSLGSGSLGSGSLGSGSLGSGSLGSGSLGSGSLGSGSLGSGSLGSGSLGSGSLGSGSL

SEQ ID NO: 178 - protein, 60 aa, Artificial Sequence
  Synthetic polypeptide
GSGSGSGSGSLGSGSGSGSGSLGSGSGSGSGSLGSGSGSGSGSLGSGSGSGSGSLGSGSG

SEQ ID NO: 179 - protein, 60 aa, Artificial Sequence
  Synthetic polypeptide
GSIGSIGSIGSIGSIGSIGSIGSIGSIGSIGSIGSIGSIGSIGSIGSIGSIGSIGSIGSI

SEQ ID NO: 180 - protein, 60 aa, Artificial Sequence
  Synthetic polypeptide
GSGSIGSGSIGSGSIGSGSIGSGSIGSGSIGSGSIGSGSIGSGSIGSGSIGSGSIGSGSI

SEQ ID NO: 181 - protein, 60 aa, Artificial Sequence
  Synthetic polypeptide
GSGSGSGSGSIGSGSGSGSGSIGSGSGSGSGSIGSGSGSGSGSIGSGSGSGSGSIGSGSG

SEQ ID NO: 182 - protein, 60 aa, Artificial Sequence
  Synthetic polypeptide
STASTASTASTASTASTASTASTASTASTASTASTASTASTASTASTASTASTASTASTA

SEQ ID NO: 183 - protein, 60 aa, Artificial Sequence
  Synthetic polypeptide
STSTASTSTASTSTASTSTASTSTASTSTASTSTASTSTASTSTASTSTASTSTASTSTA

SEQ ID NO: 184 - protein, 60 aa, Artificial Sequence
  Synthetic polypeptide
STSTSTSTSTASTSTSTSTSTASTSTSTSTSTASTSTSTSTSTASTSTSTSTSTASTSTS

SEQ ID NO: 185 - protein, 60 aa, Artificial Sequence
  Synthetic polypeptide
STSSTSSTSSTSSTSSTSSTSSTSSTSSTSSTSSTSSTSSTSSTSSTSSTSSTSSTSSTS

SEQ ID NO: 186 - protein, 60 aa, Artificial Sequence
  Synthetic polypeptide
STSTSSTSTSSTSTSSTSTSSTSTSSTSTSSTSTSSTSTSSTSTSSTSTSSTSTSSTSTS

SEQ ID NO: 187 - protein, 60 aa, Artificial Sequence
  Synthetic polypeptide
STSTSTSTSTSSTSTSTSTSTSSTSTSTSTSTSSTSTSTSTSTSSTSTSTSTSTSSTSTS

SEQ ID NO: 188 - protein, 60 aa, Artificial Sequence
  Synthetic polypeptide
STTSTTSTTSTTSTTSTTSTTSTTSTTSTTSTTSTTSTTSTTSTTSTTSTTSTTSTTSTT

SEQ ID NO: 189 - protein, 60 aa, Artificial Sequence
  Synthetic polypeptide
STSTTSTSTTSTSTTSTSTTSTSTTSTSTTSTSTTSTSTTSTSTTSTSTTSTSTTSTSTT

SEQ ID NO: 190 - protein, 60 aa, Artificial Sequence
  Synthetic polypeptide
STSTSTSTSTTSTSTSTSTSTTSTSTSTSTSTTSTSTSTSTSTTSTSTSTSTSTTSTSTS

SEQ ID NO: 191 - protein, 60 aa, Artificial Sequence
  Synthetic polypeptide
STESTESTESTESTESTESTESTESTESTESTESTESTESTESTESTESTESTESTESTE

SEQ ID NO: 192 - protein, 60 aa, Artificial Sequence
  Synthetic polypeptide
STSTESTSTESTSTESTSTESTSTESTSTESTSTESTSTESTSTESTSTESTSTESTSTE

SEQ ID NO: 193 - protein, 60 aa, Artificial Sequence
  Synthetic polypeptide
STSTSTSTSTESTSTSTSTSTESTSTSTSTSTESTSTSTSTSTESTSTSTSTSTESTSTS

SEQ ID NO: 194 - protein, 60 aa, Artificial Sequence
  Synthetic polypeptide
STDSTDSTDSTDSTDSTDSTDSTDSTDSTDSTDSTDSTDSTDSTDSTDSTDSTDSTDSTD

SEQ ID NO: 195 - protein, 60 aa, Artificial Sequence
  Synthetic polypeptide
STSTDSTSTDSTSTDSTSTDSTSTDSTSTDSTSTDSTSTDSTSTDSTSTDSTSTDSTSTD

SEQ ID NO: 196 - protein, 60 aa, Artificial Sequence
  Synthetic polypeptide
STSTSTSTSTDSTSTSTSTSTDSTSTSTSTSTDSTSTSTSTSTDSTSTSTSTSTDSTSTS

SEQ ID NO: 197 - protein, 60 aa, Artificial Sequence
  Synthetic polypeptide
STQSTQSTQSTQSTQSTQSTQSTQSTQSTQSTQSTQSTQSTQSTQSTQSTQSTQSTQSTQ

SEQ ID NO: 198 - protein, 60 aa, Artificial Sequence
  Synthetic polypeptide
STSTQSTSTQSTSTQSTSTQSTSTQSTSTQSTSTQSTSTQSTSTQSTSTQSTSTQSTSTQ

SEQ ID NO: 199 - protein, 60 aa, Artificial Sequence
  Synthetic polypeptide
STSTSTSTSTQSTSTSTSTSTQSTSTSTSTSTQSTSTSTSTSTQSTSTSTSTSTQSTSTS

SEQ ID NO: 200 - protein, 60 aa, Artificial Sequence
  Synthetic polypeptide
STNSTNSTNSTNSTNSTNSTNSTNSTNSTNSTNSTNSTNSTNSTNSTNSTNSTNSTNSTN

SEQ ID NO: 201 - protein, 60 aa, Artificial Sequence
  Synthetic polypeptide
STSTNSTSTNSTSTNSTSTNSTSTNSTSTNSTSTNSTSTNSTSTNSTSTNSTSTNSTSTN

SEQ ID NO: 202 - protein, 60 aa, Artificial Sequence
  Synthetic polypeptide
STSTSTSTSTNSTSTSTSTSTNSTSTSTSTSTNSTSTSTSTSTNSTSTSTSTSTNSTSTS

SEQ ID NO: 203 - protein, 60 aa, Artificial Sequence
  Synthetic polypeptide
STKSTKSTKSTKSTKSTKSTKSTKSTKSTKSTKSTKSTKSTKSTKSTKSTKSTKSTKSTK

SEQ ID NO: 204 - protein, 60 aa, Artificial Sequence
  Synthetic polypeptide
STSTKSTSTKSTSTKSTSTKSTSTKSTSTKSTSTKSTSTKSTSTKSTSTKSTSTKSTSTK

SEQ ID NO: 205 - protein, 60 aa, Artificial Sequence
  Synthetic polypeptide
STSTSTSTSTKSTSTSTSTSTKSTSTSTSTSTKSTSTSTSTSTKSTSTSTSTSTKSTSTS

SEQ ID NO: 206 - protein, 60 aa, Artificial Sequence
  Synthetic polypeptide
STRSTRSTRSTRSTRSTRSTRSTRSTRSTRSTRSTRSTRSTRSTRSTRSTRSTRSTRSTR

SEQ ID NO: 207 - protein, 60 aa, Artificial Sequence
  Synthetic polypeptide
STSTRSTSTRSTSTRSTSTRSTSTRSTSTRSTSTRSTSTRSTSTRSTSTRSTSTRSTSTR

SEQ ID NO: 208 - protein, 60 aa, Artificial Sequence
  Synthetic polypeptide
STSTSTSTSTRSTSTSTSTSTRSTSTSTSTSTRSTSTSTSTSTRSTSTSTSTSTRSTSTS

SEQ ID NO: 209 - protein, 60 aa, Artificial Sequence
  Synthetic polypeptide
STPSTPSTPSTPSTPSTPSTPSTPSTPSTPSTPSTPSTPSTPSTPSTPSTPSTPSTPSTP

SEQ ID NO: 210 - protein, 60 aa, Artificial Sequence
  Synthetic polypeptide
STSTPSTSTPSTSTPSTSTPSTSTPSTSTPSTSTPSTSTPSTSTPSTSTPSTSTPSTSTP

SEQ ID NO: 211 - protein, 60 aa, Artificial Sequence
  Synthetic polypeptide
STSTSTSTSTPSTSTSTSTSTPSTSTSTSTSTPSTSTSTSTSTPSTSTSTSTSTPSTSTS

SEQ ID NO: 212 - protein, 60 aa, Artificial Sequence
  Synthetic polypeptide
STGSTGSTGSTGSTGSTGSTGSTGSTGSTGSTGSTGSTGSTGSTGSTGSTGSTGSTGSTG

SEQ ID NO: 213 - protein, 60 aa, Artificial Sequence
  Synthetic polypeptide
STSTGSTSTGSTSTGSTSTGSTSTGSTSTGSTSTGSTSTGSTSTGSTSTGSTSTGSTSTG

SEQ ID NO: 214 - protein, 60 aa, Artificial Sequence
  Synthetic polypeptide
STSTSTSTSTGSTSTSTSTSTGSTSTSTSTSTGSTSTSTSTSTGSTSTSTSTSTGSTSTS

SEQ ID NO: 215 - protein, 60 aa, Artificial Sequence
  Synthetic polypeptide
STLSTLSTLSTLSTLSTLSTLSTLSTLSTLSTLSTLSTLSTLSTLSTLSTLSTLSTLSTL

SEQ ID NO: 216 - protein, 60 aa, Artificial Sequence
  Synthetic polypeptide
STSTLSTSTLSTSTLSTSTLSTSTLSTSTLSTSTLSTSTLSTSTLSTSTLSTSTLSTSTL

SEQ ID NO: 217 - protein, 60 aa, Artificial Sequence
  Synthetic polypeptide
STSTSTSTSTLSTSTSTSTSTLSTSTSTSTSTLSTSTSTSTSTLSTSTSTSTSTLSTSTS

SEQ ID NO: 218 - protein, 60 aa, Artificial Sequence
  Synthetic polypeptide
STISTISTISTISTISTISTISTISTISTISTISTISTISTISTISTISTISTISTISTI

SEQ ID NO: 219 - protein, 60 aa, Artificial Sequence
  Synthetic polypeptide
STSTISTSTISTSTISTSTISTSTISTSTISTSTISTSTISTSTISTSTISTSTISTSTI

SEQ ID NO: 220 - protein, 60 aa, Artificial Sequence
  Synthetic polypeptide
STSTSTSTSTISTSTSTSTSTISTSTSTSTSTISTSTSTSTSTISTSTSTSTSTISTSTS

SEQ ID NO: 221 - protein, 60 aa, Artificial Sequence
  Synthetic polypeptide
QNAQNAQNAQNAQNAQNAQNAQNAQNAQNAQNAQNAQNAQNAQNAQNAQNAQNAQNAQNA

SEQ ID NO: 222 - protein, 60 aa, Artificial Sequence
  Synthetic polypeptide
QNQNAQNQNAQNQNAQNQNAQNQNAQNQNAQNQNAQNQNAQNQNAQNQNAQNQNAQNQNA

SEQ ID NO: 223 - protein, 60 aa, Artificial Sequence
  Synthetic polypeptide
QNQNQNQNQNAQNQNQNQNQNAQNQNQNQNQNAQNQNQNQNQNAQNQNQNQNQNAQNQNQ

SEQ ID NO: 224 - protein, 60 aa, Artificial Sequence
  Synthetic polypeptide
QNSQNSQNSQNSQNSQNSQNSQNSQNSQNSQNSQNSQNSQNSQNSQNSQNSQNSQNSQNS

SEQ ID NO: 225 - protein, 60 aa, Artificial Sequence
  Synthetic polypeptide
QNQNSQNQNSQNQNSQNQNSQNQNSQNQNSQNQNSQNQNSQNQNSQNQNSQNQNSQNQNS

SEQ ID NO: 226 - protein, 60 aa, Artificial Sequence
  Synthetic polypeptide
QNQNQNQNQNSQNQNQNQNQNSQNQNQNQNQNSQNQNQNQNQNSQNQNQNQNQNSQNQNQ

SEQ ID NO: 227 - protein, 60 aa, Artificial Sequence
  Synthetic polypeptide
QNTQNTQNTQNTQNTQNTQNTQNTQNTQNTQNTQNTQNTQNTQNTQNTQNTQNTQNTQNT

SEQ ID NO: 228 - protein, 60 aa, Artificial Sequence
  Synthetic polypeptide
QNQNTQNQNTQNQNTQNQNTQNQNTQNQNTQNQNTQNQNTQNQNTQNQNTQNQNTQNQNT

SEQ ID NO: 229 - protein, 60 aa, Artificial Sequence
  Synthetic polypeptide
QNQNQNQNQNTQNQNQNQNQNTQNQNQNQNQNTQNQNQNQNQNTQNQNQNQNQNTQNQNQ

SEQ ID NO: 230 - protein, 60 aa, Artificial Sequence
  Synthetic polypeptide
QNEQNEQNEQNEQNEQNEQNEQNEQNEQNEQNEQNEQNEQNEQNEQNEQNEQNEQNEQNE

SEQ ID NO: 231 - protein, 60 aa, Artificial Sequence
  Synthetic polypeptide
QNQNEQNQNEQNQNEQNQNEQNQNEQNQNEQNQNEQNQNEQNQNEQNQNEQNQNEQNQNE

SEQ ID NO: 232 - protein, 60 aa, Artificial Sequence
  Synthetic polypeptide
QNQNQNQNQNEQNQNQNQNQNEQNQNQNQNQNEQNQNQNQNQNEQNQNQNQNQNEQNQNQ

SEQ ID NO: 233 - protein, 60 aa, Artificial Sequence
  Synthetic polypeptide
QNDQNDQNDQNDQNDQNDQNDQNDQNDQNDQNDQNDQNDQNDQNDQNDQNDQNDQNDQND

SEQ ID NO: 234 - protein, 60 aa, Artificial Sequence
  Synthetic polypeptide
QNQNDQNQNDQNQNDQNQNDQNQNDQNQNDQNQNDQNQNDQNQNDQNQNDQNQNDQNQND

SEQ ID NO: 235 - protein, 60 aa, Artificial Sequence
  Synthetic polypeptide
QNQNQNQNQNDQNQNQNQNQNDQNQNQNQNQNDQNQNQNQNQNDQNQNQNQNQNDQNQNQ

SEQ ID NO: 236 - protein, 60 aa, Artificial Sequence
  Synthetic polypeptide
QNQQNQQNQQNQQNQQNQQNQQNQQNQQNQQNQQNQQNQQNQQNQQNQQNQQNQQNQQNQ

SEQ ID NO: 237 - protein, 60 aa, Artificial Sequence
  Synthetic polypeptide
QNQNQQNQNQQNQNQQNQNQQNQNQQNQNQQNQNQQNQNQQNQNQQNQNQQNQNQQNQNQ

SEQ ID NO: 238 - protein, 60 aa, Artificial Sequence
  Synthetic polypeptide
QNQNQNQNQNQQNQNQNQNQNQQNQNQNQNQNQQNQNQNQNQNQQNQNQNQNQNQQNQNQ

SEQ ID NO: 239 - protein, 60 aa, Artificial Sequence
  Synthetic polypeptide
QNNQNNQNNQNNQNNQNNQNNQNNQNNQNNQNNQNNQNNQNNQNNQNNQNNQNNQNNQNN

SEQ ID NO: 240 - protein, 60 aa, Artificial Sequence
  Synthetic polypeptide
QNQNNQNQNNQNQNNQNQNNQNQNNQNQNNQNQNNQNQNNQNQNNQNQNNQNQNNQNQNN

SEQ ID NO: 241 - protein, 60 aa, Artificial Sequence
  Synthetic polypeptide
QNQNQNQNQNNQNQNQNQNQNNQNQNQNQNQNNQNQNQNQNQNNQNQNQNQNQNNQNQNQ

SEQ ID NO: 242 - protein, 60 aa, Artificial Sequence
  Synthetic polypeptide
QNKQNKQNKQNKQNKQNKQNKQNKQNKQNKQNKQNKQNKQNKQNKQNKQNKQNKQNKQNK

SEQ ID NO: 243 - protein, 60 aa, Artificial Sequence
  Synthetic polypeptide
QNQNKQNQNKQNQNKQNQNKQNQNKQNQNKQNQNKQNQNKQNQNKQNQNKQNQNKQNQNK

SEQ ID NO: 244 - protein, 60 aa, Artificial Sequence
  Synthetic polypeptide
QNQNQNQNQNKQNQNQNQNQNKQNQNQNQNQNKQNQNQNQNQNKQNQNQNQNQNKQNQNQ

SEQ ID NO: 245 - protein, 60 aa, Artificial Sequence
  Synthetic polypeptide
QNRQNRQNRQNRQNRQNRQNRQNRQNRQNRQNRQNRQNRQNRQNRQNRQNRQNRQNRQNR

SEQ ID NO: 246 - protein, 60 aa, Artificial Sequence
  Synthetic polypeptide
QNQNRQNQNRQNQNRQNQNRQNQNRQNQNRQNQNRQNQNRQNQNRQNQNRQNQNRQNQNR

SEQ ID NO: 247 - protein, 60 aa, Artificial Sequence
  Synthetic polypeptide
QNQNQNQNQNRQNQNQNQNQNRQNQNQNQNQNRQNQNQNQNQNRQNQNQNQNQNRQNQNQ

SEQ ID NO: 248 - protein, 60 aa, Artificial Sequence
  Synthetic polypeptide
QNPQNPQNPQNPQNPQNPQNPQNPQNPQNPQNPQNPQNPQNPQNPQNPQNPQNPQNPQNP

SEQ ID NO: 249 - protein, 60 aa, Artificial Sequence
  Synthetic polypeptide
QNQNPQNQNPQNQNPQNQNPQNQNPQNQNPQNQNPQNQNPQNQNPQNQNPQNQNPQNQNP

SEQ ID NO: 250 - protein, 60 aa, Artificial Sequence
  Synthetic polypeptide
QNQNQNQNQNPQNQNQNQNQNPQNQNQNQNQNPQNQNQNQNQNPQNQNQNQNQNPQNQNQ

SEQ ID NO: 251 - protein, 60 aa, Artificial Sequence
  Synthetic polypeptide
QNGQNGQNGQNGQNGQNGQNGQNGQNGQNGQNGQNGQNGQNGQNGQNGQNGQNGQNGQNG

SEQ ID NO: 252 - protein, 60 aa, Artificial Sequence
  Synthetic polypeptide
QNQNGQNQNGQNQNGQNQNGQNQNGQNQNGQNQNGQNQNGQNQNGQNQNGQNQNGQNQNG

SEQ ID NO: 253 - protein, 60 aa, Artificial Sequence
  Synthetic polypeptide
QNQNQNQNQNGQNQNQNQNQNGQNQNQNQNQNGQNQNQNQNQNGQNQNQNQNQNGQNQNQ

SEQ ID NO: 254 - protein, 60 aa, Artificial Sequence
  Synthetic polypeptide
QNLQNLQNLQNLQNLQNLQNLQNLQNLQNLQNLQNLQNLQNLQNLQNLQNLQNLQNLQNL

SEQ ID NO: 255 - protein, 60 aa, Artificial Sequence
  Synthetic polypeptide
QNQNLQNQNLQNQNLQNQNLQNQNLQNQNLQNQNLQNQNLQNQNLQNQNLQNQNLQNQNL

SEQ ID NO: 256 - protein, 60 aa, Artificial Sequence
  Synthetic polypeptide
QNQNQNQNQNLQNQNQNQNQNLQNQNQNQNQNLQNQNQNQNQNLQNQNQNQNQNLQNQNQ

SEQ ID NO: 257 - protein, 60 aa, Artificial Sequence
  Synthetic polypeptide
QNIQNIQNIQNIQNIQNIQNIQNIQNIQNIQNIQNIQNIQNIQNIQNIQNIQNIQNIQNI

SEQ ID NO: 258 - protein, 60 aa, Artificial Sequence
  Synthetic polypeptide
QNQNIQNQNIQNQNIQNQNIQNQNIQNQNIQNQNIQNQNIQNQNIQNQNIQNQNIQNQNI

SEQ ID NO: 259 - protein, 60 aa, Artificial Sequence
  Synthetic polypeptide
QNQNQNQNQNIQNQNQNQNQNIQNQNQNQNQNIQNQNQNQNQNIQNQNQNQNQNIQNQNQ

SEQ ID NO: 260 - protein, 60 aa, Artificial Sequence
  Synthetic polypeptide
GEAGEAGEAGEAGEAGEAGEAGEAGEAGEAGEAGEAGEAGEAGEAGEAGEAGEAGEAGEA

SEQ ID NO: 261 - protein, 60 aa, Artificial Sequence
  Synthetic polypeptide
GEGEAGEGEAGEGEAGEGEAGEGEAGEGEAGEGEAGEGEAGEGEAGEGEAGEGEAGEGEA

SEQ ID NO: 262 - protein, 60 aa, Artificial Sequence
  Synthetic polypeptide
GEGEGEGEGEAGEGEGEGEGEAGEGEGEGEGEAGEGEGEGEGEAGEGEGEGEGEAGEGEG

SEQ ID NO: 263 - protein, 60 aa, Artificial Sequence
  Synthetic polypeptide
GESGESGESGESGESGESGESGESGESGESGESGESGESGESGESGESGESGESGESGES

SEQ ID NO: 264 - protein, 60 aa, Artificial Sequence
  Synthetic polypeptide
GEGESGEGESGEGESGEGESGEGESGEGESGEGESGEGESGEGESGEGESGEGESGEGES

SEQ ID NO: 265 - protein, 60 aa, Artificial Sequence
  Synthetic polypeptide
GEGEGEGEGESGEGEGEGEGESGEGEGEGEGESGEGEGEGEGESGEGEGEGEGESGEGEG

SEQ ID NO: 266 - protein, 60 aa, Artificial Sequence
  Synthetic polypeptide
GETGETGETGETGETGETGETGETGETGETGETGETGETGETGETGETGETGETGETGET

SEQ ID NO: 267 - protein, 60 aa, Artificial Sequence
  Synthetic polypeptide
GEGETGEGETGEGETGEGETGEGETGEGETGEGETGEGETGEGETGEGETGEGETGEGET

SEQ ID NO: 268 - protein, 60 aa, Artificial Sequence
  Synthetic polypeptide
GEGEGEGEGETGEGEGEGEGETGEGEGEGEGETGEGEGEGEGETGEGEGEGEGETGEGEG

SEQ ID NO: 269 - protein, 60 aa, Artificial Sequence
  Synthetic polypeptide
GEEGEEGEEGEEGEEGEEGEEGEEGEEGEEGEEGEEGEEGEEGEEGEEGEEGEEGEEGEE

SEQ ID NO: 270 - protein, 60 aa, Artificial Sequence
  Synthetic polypeptide
GEGEEGEGEEGEGEEGEGEEGEGEEGEGEEGEGEEGEGEEGEGEEGEGEEGEGEEGEGEE

SEQ ID NO: 271 - protein, 60 aa, Artificial Sequence
  Synthetic polypeptide
GEGEGEGEGEEGEGEGEGEGEEGEGEGEGEGEEGEGEGEGEGEEGEGEGEGEGEEGEGEG

SEQ ID NO: 272 - protein, 60 aa, Artificial Sequence
  Synthetic polypeptide
GEDGEDGEDGEDGEDGEDGEDGEDGEDGEDGEDGEDGEDGEDGEDGEDGEDGEDGEDGED

SEQ ID NO: 273 - protein, 60 aa, Artificial Sequence
  Synthetic polypeptide
GEGEDGEGEDGEGEDGEGEDGEGEDGEGEDGEGEDGEGEDGEGEDGEGEDGEGEDGEGED

SEQ ID NO: 274 - protein, 60 aa, Artificial Sequence
  Synthetic polypeptide
GEGEGEGEGEDGEGEGEGEGEDGEGEGEGEGEDGEGEGEGEGEDGEGEGEGEGEDGEGEG

SEQ ID NO: 275 - protein, 60 aa, Artificial Sequence
  Synthetic polypeptide
GEQGEQGEQGEQGEQGEQGEQGEQGEQGEQGEQGEQGEQGEQGEQGEQGEQGEQGEQGEQ

SEQ ID NO: 276 - protein, 60 aa, Artificial Sequence
  Synthetic polypeptide
GEGEQGEGEQGEGEQGEGEQGEGEQGEGEQGEGEQGEGEQGEGEQGEGEQGEGEQGEGEQ

SEQ ID NO: 277 - protein, 60 aa, Artificial Sequence
  Synthetic polypeptide
GEGEGEGEGEQGEGEGEGEGEQGEGEGEGEGEQGEGEGEGEGEQGEGEGEGEGEQGEGEG

SEQ ID NO: 278 - protein, 60 aa, Artificial Sequence
  Synthetic polypeptide
GENGENGENGENGENGENGENGENGENGENGENGENGENGENGENGENGENGENGENGEN

SEQ ID NO: 279 - protein, 60 aa, Artificial Sequence
  Synthetic polypeptide
GEGENGEGENGEGENGEGENGEGENGEGENGEGENGEGENGEGENGEGENGEGENGEGEN

SEQ ID NO: 280 - protein, 60 aa, Artificial Sequence
  Synthetic polypeptide
GEGEGEGEGENGEGEGEGEGENGEGEGEGEGENGEGEGEGEGENGEGEGEGEGENGEGEG

SEQ ID NO: 281 - protein, 60 aa, Artificial Sequence
  Synthetic polypeptide
GEKGEKGEKGEKGEKGEKGEKGEKGEKGEKGEKGEKGEKGEKGEKGEKGEKGEKGEKGEK

SEQ ID NO: 282 - protein, 60 aa, Artificial Sequence
  Synthetic polypeptide
GEGEKGEGEKGEGEKGEGEKGEGEKGEGEKGEGEKGEGEKGEGEKGEGEKGEGEKGEGEK

SEQ ID NO: 283 - protein, 60 aa, Artificial Sequence
  Synthetic polypeptide
GEGEGEGEGEKGEGEGEGEGEKGEGEGEGEGEKGEGEGEGEGEKGEGEGEGEGEKGEGEG

SEQ ID NO: 284 - protein, 60 aa, Artificial Sequence
  Synthetic polypeptide
GERGERGERGERGERGERGERGERGERGERGERGERGERGERGERGERGERGERGERGER

SEQ ID NO: 285 - protein, 60 aa, Artificial Sequence
  Synthetic polypeptide
GEGERGEGERGEGERGEGERGEGERGEGERGEGERGEGERGEGERGEGERGEGERGEGER

SEQ ID NO: 286 - protein, 60 aa, Artificial Sequence
  Synthetic polypeptide
GEGEGEGEGERGEGEGEGEGERGEGEGEGEGERGEGEGEGEGERGEGEGEGEGERGEGEG

SEQ ID NO: 287 - protein, 60 aa, Artificial Sequence
  Synthetic polypeptide
GEPGEPGEPGEPGEPGEPGEPGEPGEPGEPGEPGEPGEPGEPGEPGEPGEPGEPGEPGEP

SEQ ID NO: 288 - protein, 60 aa, Artificial Sequence
  Synthetic polypeptide
GEGEPGEGEPGEGEPGEGEPGEGEPGEGEPGEGEPGEGEPGEGEPGEGEPGEGEPGEGEP

SEQ ID NO: 289 - protein, 60 aa, Artificial Sequence
  Synthetic polypeptide
GEGEGEGEGEPGEGEGEGEGEPGEGEGEGEGEPGEGEGEGEGEPGEGEGEGEGEPGEGEG

SEQ ID NO: 290 - protein, 60 aa, Artificial Sequence
  Synthetic polypeptide
GEGGEGGEGGEGGEGGEGGEGGEGGEGGEGGEGGEGGEGGEGGEGGEGGEGGEGGEGGEG

SEQ ID NO: 291 - protein, 60 aa, Artificial Sequence
  Synthetic polypeptide
GEGEGGEGEGGEGEGGEGEGGEGEGGEGEGGEGEGGEGEGGEGEGGEGEGGEGEGGEGEG

SEQ ID NO: 292 - protein, 60 aa, Artificial Sequence
  Synthetic polypeptide
GEGEGEGEGEGGEGEGEGEGEGGEGEGEGEGEGGEGEGEGEGEGGEGEGEGEGEGGEGEG

SEQ ID NO: 293 - protein, 60 aa, Artificial Sequence
  Synthetic polypeptide
GELGELGELGELGELGELGELGELGELGELGELGELGELGELGELGELGELGELGELGEL

SEQ ID NO: 294 - protein, 60 aa, Artificial Sequence
  Synthetic polypeptide
GEGELGEGELGEGELGEGELGEGELGEGELGEGELGEGELGEGELGEGELGEGELGEGEL

SEQ ID NO: 295 - protein, 60 aa, Artificial Sequence
  Synthetic polypeptide
GEGEGEGEGELGEGEGEGEGELGEGEGEGEGELGEGEGEGEGELGEGEGEGEGELGEGEG

SEQ ID NO: 296 - protein, 60 aa, Artificial Sequence
  Synthetic polypeptide
GEIGEIGEIGEIGEIGEIGEIGEIGEIGEIGEIGEIGEIGEIGEIGEIGEIGEIGEIGEI

SEQ ID NO: 297 - protein, 60 aa, Artificial Sequence
  Synthetic polypeptide
GEGEIGEGEIGEGEIGEGEIGEGEIGEGEIGEGEIGEGEIGEGEIGEGEIGEGEIGEGEI

SEQ ID NO: 298 - protein, 60 aa, Artificial Sequence
  Synthetic polypeptide
GEGEGEGEGEIGEGEGEGEGEIGEGEGEGEGEIGEGEGEGEGEIGEGEGEGEGEIGEGEG

SEQ ID NO: 299 - protein, 60 aa, Artificial Sequence
  Synthetic polypeptide
EKAEKAEKAEKAEKAEKAEKAEKAEKAEKAEKAEKAEKAEKAEKAEKAEKAEKAEKAEKA

SEQ ID NO: 300 - protein, 60 aa, Artificial Sequence
  Synthetic polypeptide
EKEKAEKEKAEKEKAEKEKAEKEKAEKEKAEKEKAEKEKAEKEKAEKEKAEKEKAEKEKA

SEQ ID NO: 301 - protein, 60 aa, Artificial Sequence
  Synthetic polypeptide
EKEKEKEKEKAEKEKEKEKEKAEKEKEKEKEKAEKEKEKEKEKAEKEKEKEKEKAEKEKE

SEQ ID NO: 302 - protein, 60 aa, Artificial Sequence
  Synthetic polypeptide
EKSEKSEKSEKSEKSEKSEKSEKSEKSEKSEKSEKSEKSEKSEKSEKSEKSEKSEKSEKS

SEQ ID NO: 303 - protein, 60 aa, Artificial Sequence
  Synthetic polypeptide
EKEKSEKEKSEKEKSEKEKSEKEKSEKEKSEKEKSEKEKSEKEKSEKEKSEKEKSEKEKS

SEQ ID NO: 304 - protein, 60 aa, Artificial Sequence
  Synthetic polypeptide
EKEKEKEKEKSEKEKEKEKEKSEKEKEKEKEKSEKEKEKEKEKSEKEKEKEKEKSEKEKE

SEQ ID NO: 305 - protein, 60 aa, Artificial Sequence
  Synthetic polypeptide
EKTEKTEKTEKTEKTEKTEKTEKTEKTEKTEKTEKTEKTEKTEKTEKTEKTEKTEKTEKT

SEQ ID NO: 306 - protein, 60 aa, Artificial Sequence
  Synthetic polypeptide
EKEKTEKEKTEKEKTEKEKTEKEKTEKEKTEKEKTEKEKTEKEKTEKEKTEKEKTEKEKT

SEQ ID NO: 307 - protein, 60 aa, Artificial Sequence
  Synthetic polypeptide
EKEKEKEKEKTEKEKEKEKEKTEKEKEKEKEKTEKEKEKEKEKTEKEKEKEKEKTEKEKE

SEQ ID NO: 308 - protein, 60 aa, Artificial Sequence
  Synthetic polypeptide
EKEEKEEKEEKEEKEEKEEKEEKEEKEEKEEKEEKEEKEEKEEKEEKEEKEEKEEKEEKE

SEQ ID NO: 309 - protein, 60 aa, Artificial Sequence
  Synthetic polypeptide
EKEKEEKEKEEKEKEEKEKEEKEKEEKEKEEKEKEEKEKEEKEKEEKEKEEKEKEEKEKE

SEQ ID NO: 310 - protein, 60 aa, Artificial Sequence
  Synthetic polypeptide
EKEKEKEKEKEEKEKEKEKEKEEKEKEKEKEKEEKEKEKEKEKEEKEKEKEKEKEEKEKE

SEQ ID NO: 311 - protein, 60 aa, Artificial Sequence
  Synthetic polypeptide
EKDEKDEKDEKDEKDEKDEKDEKDEKDEKDEKDEKDEKDEKDEKDEKDEKDEKDEKDEKD

SEQ ID NO: 312 - protein, 60 aa, Artificial Sequence
  Synthetic polypeptide
EKEKDEKEKDEKEKDEKEKDEKEKDEKEKDEKEKDEKEKDEKEKDEKEKDEKEKDEKEKD

SEQ ID NO: 313 - protein, 60 aa, Artificial Sequence
  Synthetic polypeptide
EKEKEKEKEKDEKEKEKEKEKDEKEKEKEKEKDEKEKEKEKEKDEKEKEKEKEKDEKEKE

SEQ ID NO: 314 - protein, 60 aa, Artificial Sequence
  Synthetic polypeptide
EKQEKQEKQEKQEKQEKQEKQEKQEKQEKQEKQEKQEKQEKQEKQEKQEKQEKQEKQEKQ

SEQ ID NO: 315 - protein, 60 aa, Artificial Sequence
  Synthetic polypeptide
EKEKQEKEKQEKEKQEKEKQEKEKQEKEKQEKEKQEKEKQEKEKQEKEKQEKEKQEKEKQ

SEQ ID NO: 316 - protein, 60 aa, Artificial Sequence
  Synthetic polypeptide
EKEKEKEKEKQEKEKEKEKEKQEKEKEKEKEKQEKEKEKEKEKQEKEKEKEKEKQEKEKE

SEQ ID NO: 317 - protein, 60 aa, Artificial Sequence
  Synthetic polypeptide
EKNEKNEKNEKNEKNEKNEKNEKNEKNEKNEKNEKNEKNEKNEKNEKNEKNEKNEKNEKN

SEQ ID NO: 318 - protein, 60 aa, Artificial Sequence
  Synthetic polypeptide
EKEKNEKEKNEKEKNEKEKNEKEKNEKEKNEKEKNEKEKNEKEKNEKEKNEKEKNEKEKN

SEQ ID NO: 319 - protein, 60 aa, Artificial Sequence
  Synthetic polypeptide
EKEKEKEKEKNEKEKEKEKEKNEKEKEKEKEKNEKEKEKEKEKNEKEKEKEKEKNEKEKE

SEQ ID NO: 320 - protein, 60 aa, Artificial Sequence
  Synthetic polypeptide
EKKEKKEKKEKKEKKEKKEKKEKKEKKEKKEKKEKKEKKEKKEKKEKKEKKEKKEKKEKK

SEQ ID NO: 321 - protein, 60 aa, Artificial Sequence
  Synthetic polypeptide
EKEKKEKEKKEKEKKEKEKKEKEKKEKEKKEKEKKEKEKKEKEKKEKEKKEKEKKEKEKK

SEQ ID NO: 322 - protein, 60 aa, Artificial Sequence
  Synthetic polypeptide
EKEKEKEKEKKEKEKEKEKEKKEKEKEKEKEKKEKEKEKEKEKKEKEKEKEKEKKEKEKE

SEQ ID NO: 323 - protein, 60 aa, Artificial Sequence
  Synthetic polypeptide
EKREKREKREKREKREKREKREKREKREKREKREKREKREKREKREKREKREKREKREKR

SEQ ID NO: 324 - protein, 60 aa, Artificial Sequence
  Synthetic polypeptide
EKEKREKEKREKEKREKEKREKEKREKEKREKEKREKEKREKEKREKEKREKEKREKEKR

SEQ ID NO: 325 - protein, 60 aa, Artificial Sequence
  Synthetic polypeptide
EKEKEKEKEKREKEKEKEKEKREKEKEKEKEKREKEKEKEKEKREKEKEKEKEKREKEKE

SEQ ID NO: 326 - protein, 60 aa, Artificial Sequence
  Synthetic polypeptide
EKPEKPEKPEKPEKPEKPEKPEKPEKPEKPEKPEKPEKPEKPEKPEKPEKPEKPEKPEKP

SEQ ID NO: 327 - protein, 60 aa, Artificial Sequence
  Synthetic polypeptide
EKEKPEKEKPEKEKPEKEKPEKEKPEKEKPEKEKPEKEKPEKEKPEKEKPEKEKPEKEKP

SEQ ID NO: 328 - protein, 60 aa, Artificial Sequence
  Synthetic polypeptide
EKEKEKEKEKPEKEKEKEKEKPEKEKEKEKEKPEKEKEKEKEKPEKEKEKEKEKPEKEKE

SEQ ID NO: 329 - protein, 60 aa, Artificial Sequence
  Synthetic polypeptide
EKEKGEKEKGEKEKGEKEKGEKEKGEKEKGEKEKGEKEKGEKEKGEKEKGEKEKGEKEKG

SEQ ID NO: 330 - protein, 60 aa, Artificial Sequence; identical to SEQ ID NO: 329
  Synthetic polypeptide

SEQ ID NO: 331 - protein, 60 aa, Artificial Sequence
  Synthetic polypeptide
EKLEKLEKLEKLEKLEKLEKLEKLEKLEKLEKLEKLEKLEKLEKLEKLEKLEKLEKLEKL

SEQ ID NO: 332 - protein, 60 aa, Artificial Sequence
  Synthetic polypeptide
EKEKLEKEKLEKEKLEKEKLEKEKLEKEKLEKEKLEKEKLEKEKLEKEKLEKEKLEKEKL

SEQ ID NO: 333 - protein, 60 aa, Artificial Sequence
  Synthetic polypeptide
EKEKEKEKEKLEKEKEKEKEKLEKEKEKEKEKLEKEKEKEKEKLEKEKEKEKEKLEKEKE

SEQ ID NO: 334 - protein, 60 aa, Artificial Sequence
  Synthetic polypeptide
EKIEKIEKIEKIEKIEKIEKIEKIEKIEKIEKIEKIEKIEKIEKIEKIEKIEKIEKIEKI

SEQ ID NO: 335 - protein, 60 aa, Artificial Sequence
  Synthetic polypeptide
EKEKIEKEKIEKEKIEKEKIEKEKIEKEKIEKEKIEKEKIEKEKIEKEKIEKEKIEKEKI

SEQ ID NO: 336 - protein, 60 aa, Artificial Sequence
  Synthetic polypeptide
EKEKEKEKEKIEKEKEKEKEKIEKEKEKEKEKIEKEKEKEKEKIEKEKEKEKEKIEKEKE

SEQ ID NO: 337 - protein, 60 aa, Artificial Sequence
  Synthetic polypeptide
ESAESAESAESAESAESAESAESAESAESAESAESAESAESAESAESAESAESAESAESA

SEQ ID NO: 338 - protein, 60 aa, Artificial Sequence
  Synthetic polypeptide
ESESAESESAESESAESESAESESAESESAESESAESESAESESAESESAESESAESESA

SEQ ID NO: 339 - protein, 60 aa, Artificial Sequence
  Synthetic polypeptide
ESESESESESAESESESESESAESESESESESAESESESESESAESESESESESAESESE

SEQ ID NO: 340 - protein, 60 aa, Artificial Sequence
  Synthetic polypeptide
ESSESSESSESSESSESSESSESSESSESSESSESSESSESSESSESSESSESSESSESS

SEQ ID NO: 341 - protein, 60 aa, Artificial Sequence
  Synthetic polypeptide
ESESSESESSESESSESESSESESSESESSESESSESESSESESSESESSESESSESESS

SEQ ID NO: 342 - protein, 60 aa, Artificial Sequence
  Synthetic polypeptide
ESESESESESSESESESESESSESESESESESSESESESESESSESESESESESSESESE

SEQ ID NO: 343 - protein, 60 aa, Artificial Sequence
  Synthetic polypeptide
ESTESTESTESTESTESTESTESTESTESTESTESTESTESTESTESTESTESTESTEST

SEQ ID NO: 344 - protein, 60 aa, Artificial Sequence
  Synthetic polypeptide
ESESTESESTESESTESESTESESTESESTESESTESESTESESTESESTESESTESEST

SEQ ID NO: 345 - protein, 60 aa, Artificial Sequence
  Synthetic polypeptide
ESESESESESTESESESESESTESESESESESTESESESESESTESESESESESTESESE

SEQ ID NO: 346 - protein, 60 aa, Artificial Sequence
  Synthetic polypeptide
ESEESEESEESEESEESEESEESEESEESEESEESEESEESEESEESEESEESEESEESE

SEQ ID NO: 347 - protein, 60 aa, Artificial Sequence
  Synthetic polypeptide
ESESEESESEESESEESESEESESEESESEESESEESESEESESEESESEESESEESESE

SEQ ID NO: 348 - protein, 60 aa, Artificial Sequence
  Synthetic polypeptide
ESESESESESEESESESESESEESESESESESEESESESESESEESESESESESEESESE

SEQ ID NO: 349 - protein, 60 aa, Artificial Sequence
  Synthetic polypeptide
ESDESDESDESDESDESDESDESDESDESDESDESDESDESDESDESDESDESDESDESD

SEQ ID NO: 350 - protein, 60 aa, Artificial Sequence
  Synthetic polypeptide
ESESDESESDESESDESESDESESDESESDESESDESESDESESDESESDESESDESESD

SEQ ID NO: 351 - protein, 60 aa, Artificial Sequence
  Synthetic polypeptide
ESESESESESDESESESESESDESESESESESDESESESESESDESESESESESDESESE

SEQ ID NO: 352 - protein, 60 aa, Artificial Sequence
  Synthetic polypeptide
ESQESQESQESQESQESQESQESQESQESQESQESQESQESQESQESQESQESQESQESQ

SEQ ID NO: 353 - protein, 60 aa, Artificial Sequence
  Synthetic polypeptide
ESESQESESQESESQESESQESESQESESQESESQESESQESESQESESQESESQESESQ

SEQ ID NO: 354 - protein, 60 aa, Artificial Sequence
  Synthetic polypeptide
ESESESESESQESESESESESQESESESESESQESESESESESQESESESESESQESESE

SEQ ID NO: 355 - protein, 60 aa, Artificial Sequence
  Synthetic polypeptide
ESNESNESNESNESNESNESNESNESNESNESNESNESNESNESNESNESNESNESNESN

SEQ ID NO: 356 - protein, 60 aa, Artificial Sequence
  Synthetic polypeptide
ESESNESESNESESNESESNESESNESESNESESNESESNESESNESESNESESNESESN

SEQ ID NO: 357 - protein, 60 aa, Artificial Sequence
  Synthetic polypeptide
ESESESESESNESESESESESNESESESESESNESESESESESNESESESESESNESESE

SEQ ID NO: 358 - protein, 60 aa, Artificial Sequence
  Synthetic polypeptide
ESKESKESKESKESKESKESKESKESKESKESKESKESKESKESKESKESKESKESKESK

SEQ ID NO: 359 - protein, 60 aa, Artificial Sequence
  Synthetic polypeptide
ESESKESESKESESKESESKESESKESESKESESKESESKESESKESESKESESKESESK

SEQ ID NO: 360 - protein, 60 aa, Artificial Sequence
  Synthetic polypeptide
ESESESESESKESESESESESKESESESESESKESESESESESKESESESESESKESESE

SEQ ID NO: 361 - protein, 60 aa, Artificial Sequence
  Synthetic polypeptide
ESRESRESRESRESRESRESRESRESRESRESRESRESRESRESRESRESRESRESRESR

SEQ ID NO: 362 - protein, 60 aa, Artificial Sequence
  Synthetic polypeptide
ESESRESESRESESRESESRESESRESESRESESRESESRESESRESESRESESRESESR

SEQ ID NO: 363 - protein, 60 aa, Artificial Sequence
  Synthetic polypeptide
ESESESESESRESESESESESRESESESESESRESESESESESRESESESESESRESESE

SEQ ID NO: 364 - protein, 60 aa, Artificial Sequence
  Synthetic polypeptide
ESPESPESPESPESPESPESPESPESPESPESPESPESPESPESPESPESPESPESPESP

SEQ ID NO: 365 - protein, 60 aa, Artificial Sequence
  Synthetic polypeptide
ESESPESESPESESPESESPESESPESESPESESPESESPESESPESESPESESPESESP

SEQ ID NO: 366 - protein, 60 aa, Artificial Sequence
  Synthetic polypeptide
ESESESESESPESESESESESPESESESESESPESESESESESPESESESESESPESESE

SEQ ID NO: 367 - protein, 60 aa, Artificial Sequence
  Synthetic polypeptide
ESGESGESGESGESGESGESGESGESGESGESGESGESGESGESGESGESGESGESGESG

SEQ ID NO: 368 - protein, 60 aa, Artificial Sequence
  Synthetic polypeptide
ESESGESESGESESGESESGESESGESESGESESGESESGESESGESESGESESGESESG

SEQ ID NO: 369 - protein, 60 aa, Artificial Sequence
  Synthetic polypeptide
ESESESESESGESESESESESGESESESESESGESESESESESGESESESESESGESESE

SEQ ID NO: 370 - protein, 60 aa, Artificial Sequence
  Synthetic polypeptide
ESLESLESLESLESLESLESLESLESLESLESLESLESLESLESLESLESLESLESLESL

SEQ ID NO: 371 - protein, 60 aa, Artificial Sequence
  Synthetic polypeptide
ESESLESESLESESLESESLESESLESESLESESLESESLESESLESESLESESLESESL

SEQ ID NO: 372 - protein, 60 aa, Artificial Sequence
  Synthetic polypeptide
ESESESESESLESESESESESLESESESESESLESESESESESLESESESESESLESESE

SEQ ID NO: 373 - protein, 60 aa, Artificial Sequence
  Synthetic polypeptide
ESIESIESIESIESIESIESIESIESIESIESIESIESIESIESIESIESIESIESIESI

SEQ ID NO: 374 - protein, 60 aa, Artificial Sequence
  Synthetic polypeptide
ESESIESESIESESIESESIESESIESESIESESIESESIESESIESESIESESIESESI

SEQ ID NO: 375 - protein, 60 aa, Artificial Sequence
  Synthetic polypeptide
ESESESESESIESESESESESIESESESESESIESESESESESIESESESESESIESESE

SEQ ID NO: 376 - protein, 60 aa, Artificial Sequence
  Synthetic polypeptide
EQAEQAEQAEQAEQAEQAEQAEQAEQAEQAEQAEQAEQAEQAEQAEQAEQAEQAEQAEQA

SEQ ID NO: 377 - protein, 60 aa, Artificial Sequence
  Synthetic polypeptide
EQEQAEQEQAEQEQAEQEQAEQEQAEQEQAEQEQAEQEQAEQEQAEQEQAEQEQAEQEQA

SEQ ID NO: 378 - protein, 60 aa, Artificial Sequence
  Synthetic polypeptide
EQEQEQEQEQAEQEQEQEQEQAEQEQEQEQEQAEQEQEQEQEQAEQEQEQEQEQAEQEQE

SEQ ID NO: 379 - protein, 60 aa, Artificial Sequence
  Synthetic polypeptide
EQSEQSEQSEQSEQSEQSEQSEQSEQSEQSEQSEQSEQSEQSEQSEQSEQSEQSEQSEQS

SEQ ID NO: 380 - protein, 60 aa, Artificial Sequence
  Synthetic polypeptide
EQEQSEQEQSEQEQSEQEQSEQEQSEQEQSEQEQSEQEQSEQEQSEQEQSEQEQSEQEQS

SEQ ID NO: 381 - protein, 60 aa, Artificial Sequence
  Synthetic polypeptide
EQEQEQEQEQSEQEQEQEQEQSEQEQEQEQEQSEQEQEQEQEQSEQEQEQEQEQSEQEQE

SEQ ID NO: 382 - protein, 60 aa, Artificial Sequence
  Synthetic polypeptide
EQTEQTEQTEQTEQTEQTEQTEQTEQTEQTEQTEQTEQTEQTEQTEQTEQTEQTEQTEQT

SEQ ID NO: 383 - protein, 60 aa, Artificial Sequence
  Synthetic polypeptide
EQEQTEQEQTEQEQTEQEQTEQEQTEQEQTEQEQTEQEQTEQEQTEQEQTEQEQTEQEQT

SEQ ID NO: 384 - protein, 60 aa, Artificial Sequence
  Synthetic polypeptide
EQEQEQEQEQTEQEQEQEQEQTEQEQEQEQEQTEQEQEQEQEQTEQEQEQEQEQTEQEQE

SEQ ID NO: 385 - protein, 60 aa, Artificial Sequence
  Synthetic polypeptide
EQEEQEEQEEQEEQEEQEEQEEQEEQEEQEEQEEQEEQEEQEEQEEQEEQEEQEEQEEQE

SEQ ID NO: 386 - protein, 60 aa, Artificial Sequence
  Synthetic polypeptide
EQEQEEQEQEEQEQEEQEQEEQEQEEQEQEEQEQEEQEQEEQEQEEQEQEEQEQEEQEQE

SEQ ID NO: 387 - protein, 60 aa, Artificial Sequence
  Synthetic polypeptide
EQEQEQEQEQEEQEQEQEQEQEEQEQEQEQEQEEQEQEQEQEQEEQEQEQEQEQEEQEQE

SEQ ID NO: 388 - protein, 60 aa, Artificial Sequence
  Synthetic polypeptide
EQDEQDEQDEQDEQDEQDEQDEQDEQDEQDEQDEQDEQDEQDEQDEQDEQDEQDEQDEQD

SEQ ID NO: 389 - protein, 60 aa, Artificial Sequence
  Synthetic polypeptide
EQEQDEQEQDEQEQDEQEQDEQEQDEQEQDEQEQDEQEQDEQEQDEQEQDEQEQDEQEQD

SEQ ID NO: 390 - protein, 60 aa, Artificial Sequence
  Synthetic polypeptide
EQEQEQEQEQDEQEQEQEQEQDEQEQEQEQEQDEQEQEQEQEQDEQEQEQEQEQDEQEQE

SEQ ID NO: 391 - protein, 60 aa, Artificial Sequence
  Synthetic polypeptide
EQQEQQEQQEQQEQQEQQEQQEQQEQQEQQEQQEQQEQQEQQEQQEQQEQQEQQEQQEQQ

SEQ ID NO: 392 - protein, 60 aa, Artificial Sequence
  Synthetic polypeptide
EQEQQEQEQQEQEQQEQEQQEQEQQEQEQQEQEQQEQEQQEQEQQEQEQQEQEQQEQEQQ

SEQ ID NO: 393 - protein, 60 aa, Artificial Sequence
  Synthetic polypeptide
EQEQEQEQEQQEQEQEQEQEQQEQEQEQEQEQQEQEQEQEQEQQEQEQEQEQEQQEQEQE

SEQ ID NO: 394 - protein, 60 aa, Artificial Sequence
  Synthetic polypeptide
EQNEQNEQNEQNEQNEQNEQNEQNEQNEQNEQNEQNEQNEQNEQNEQNEQNEQNEQNEQN

SEQ ID NO: 395 - protein, 60 aa, Artificial Sequence
  Synthetic polypeptide
EQEQNEQEQNEQEQNEQEQNEQEQNEQEQNEQEQNEQEQNEQEQNEQEQNEQEQNEQEQN

SEQ ID NO: 396 - protein, 60 aa, Artificial Sequence
  Synthetic polypeptide
EQEQEQEQEQNEQEQEQEQEQNEQEQEQEQEQNEQEQEQEQEQNEQEQEQEQEQNEQEQE

SEQ ID NO: 397 - protein, 60 aa, Artificial Sequence
  Synthetic polypeptide
EQKEQKEQKEQKEQKEQKEQKEQKEQKEQKEQKEQKEQKEQKEQKEQKEQKEQKEQKEQK

SEQ ID NO: 398 - protein, 60 aa, Artificial Sequence
  Synthetic polypeptide
EQEQKEQEQKEQEQKEQEQKEQEQKEQEQKEQEQKEQEQKEQEQKEQEQKEQEQKEQEQK

SEQ ID NO: 399 - protein, 60 aa, Artificial Sequence
  Synthetic polypeptide
EQEQEQEQEQKEQEQEQEQEQKEQEQEQEQEQKEQEQEQEQEQKEQEQEQEQEQKEQEQE

SEQ ID NO: 400 - protein, 60 aa, Artificial Sequence
  Synthetic polypeptide
EQREQREQREQREQREQREQREQREQREQREQREQREQREQREQREQREQREQREQREQR

SEQ ID NO: 401 - protein, 60 aa, Artificial Sequence
  Synthetic polypeptide
EQEQREQEQREQEQREQEQREQEQREQEQREQEQREQEQREQEQREQEQREQEQREQEQR

SEQ ID NO: 402 - protein, 60 aa, Artificial Sequence
  Synthetic polypeptide
EQEQEQEQEQREQEQEQEQEQREQEQEQEQEQREQEQEQEQEQREQEQEQEQEQREQEQE

SEQ ID NO: 403 - protein, 60 aa, Artificial Sequence
  Synthetic polypeptide
EQPEQPEQPEQPEQPEQPEQPEQPEQPEQPEQPEQPEQPEQPEQPEQPEQPEQPEQPEQP

SEQ ID NO: 404 - protein, 60 aa, Artificial Sequence
  Synthetic polypeptide
EQEQPEQEQPEQEQPEQEQPEQEQPEQEQPEQEQPEQEQPEQEQPEQEQPEQEQPEQEQP

SEQ ID NO: 405 - protein, 60 aa, Artificial Sequence
  Synthetic polypeptide
EQEQEQEQEQPEQEQEQEQEQPEQEQEQEQEQPEQEQEQEQEQPEQEQEQEQEQPEQEQE

SEQ ID NO: 406 - protein, 60 aa, Artificial Sequence
  Synthetic polypeptide
EQGEQGEQGEQGEQGEQGEQGEQGEQGEQGEQGEQGEQGEQGEQGEQGEQGEQGEQGEQG

SEQ ID NO: 407 - protein, 60 aa, Artificial Sequence
  Synthetic polypeptide
EQEQGEQEQGEQEQGEQEQGEQEQGEQEQGEQEQGEQEQGEQEQGEQEQGEQEQGEQEQG

SEQ ID NO: 408 - protein, 60 aa, Artificial Sequence
  Synthetic polypeptide
EQEQEQEQEQGEQEQEQEQEQGEQEQEQEQEQGEQEQEQEQEQGEQEQEQEQEQGEQEQE

SEQ ID NO: 409 - protein, 60 aa, Artificial Sequence
  Synthetic polypeptide
EQLEQLEQLEQLEQLEQLEQLEQLEQLEQLEQLEQLEQLEQLEQLEQLEQLEQLEQLEQL

SEQ ID NO: 410 - protein, 60 aa, Artificial Sequence
  Synthetic polypeptide
EQEQLEQEQLEQEQLEQEQLEQEQLEQEQLEQEQLEQEQLEQEQLEQEQLEQEQLEQEQL

SEQ ID NO: 411 - protein, 60 aa, Artificial Sequence
  Synthetic polypeptide
EQEQEQEQEQLEQEQEQEQEQLEQEQEQEQEQLEQEQEQEQEQLEQEQEQEQEQLEQEQE

SEQ ID NO: 412 - protein, 60 aa, Artificial Sequence
  Synthetic polypeptide
EQIEQIEQIEQIEQIEQIEQIEQIEQIEQIEQIEQIEQIEQIEQIEQIEQIEQIEQIEQI

SEQ ID NO: 413 - protein, 60 aa, Artificial Sequence
  Synthetic polypeptide
EQEQIEQEQIEQEQIEQEQIEQEQIEQEQIEQEQIEQEQIEQEQIEQEQIEQEQIEQEQI

SEQ ID NO: 414 - protein, 60 aa, Artificial Sequence
  Synthetic polypeptide
EQEQEQEQEQIEQEQEQEQEQIEQEQEQEQEQIEQEQEQEQEQIEQEQEQEQEQIEQEQE

SEQ ID NO: 415 - protein, 60 aa, Artificial Sequence
  Synthetic polypeptide
EPAEPAEPAEPAEPAEPAEPAEPAEPAEPAEPAEPAEPAEPAEPAEPAEPAEPAEPAEPA

SEQ ID NO: 416 - protein, 60 aa, Artificial Sequence
  Synthetic polypeptide
EPEPAEPEPAEPEPAEPEPAEPEPAEPEPAEPEPAEPEPAEPEPAEPEPAEPEPAEPEPA

SEQ ID NO: 417 - protein, 60 aa, Artificial Sequence
  Synthetic polypeptide
EPEPEPEPEPAEPEPEPEPEPAEPEPEPEPEPAEPEPEPEPEPAEPEPEPEPEPAEPEPE

SEQ ID NO: 418 - protein, 60 aa, Artificial Sequence
  Synthetic polypeptide
EPSEPSEPSEPSEPSEPSEPSEPSEPSEPSEPSEPSEPSEPSEPSEPSEPSEPSEPSEPS

SEQ ID NO: 419 - protein, 60 aa, Artificial Sequence
  Synthetic polypeptide
EPEPSEPEPSEPEPSEPEPSEPEPSEPEPSEPEPSEPEPSEPEPSEPEPSEPEPSEPEPS

SEQ ID NO: 420 - protein, 60 aa, Artificial Sequence
  Synthetic polypeptide
EPEPEPEPEPSEPEPEPEPEPSEPEPEPEPEPSEPEPEPEPEPSEPEPEPEPEPSEPEPE

SEQ ID NO: 421 - protein, 60 aa, Artificial Sequence
  Synthetic polypeptide
EPTEPTEPTEPTEPTEPTEPTEPTEPTEPTEPTEPTEPTEPTEPTEPTEPTEPTEPTEPT

SEQ ID NO: 422 - protein, 60 aa, Artificial Sequence
  Synthetic polypeptide
EPEPTEPEPTEPEPTEPEPTEPEPTEPEPTEPEPTEPEPTEPEPTEPEPTEPEPTEPEPT

SEQ ID NO: 423 - protein, 60 aa, Artificial Sequence
  Synthetic polypeptide
EPEPEPEPEPTEPEPEPEPEPTEPEPEPEPEPTEPEPEPEPEPTEPEPEPEPEPTEPEPE

SEQ ID NO: 424 - protein, 60 aa, Artificial Sequence
  Synthetic polypeptide
EPEEPEEPEEPEEPEEPEEPEEPEEPEEPEEPEEPEEPEEPEEPEEPEEPEEPEEPEEPE

SEQ ID NO: 425 - protein, 60 aa, Artificial Sequence
  Synthetic polypeptide
EPEPEEPEPEEPEPEEPEPEEPEPEEPEPEEPEPEEPEPEEPEPEEPEPEEPEPEEPEPE

SEQ ID NO: 426 - protein, 60 aa, Artificial Sequence
  Synthetic polypeptide
EPEPEPEPEPEEPEPEPEPEPEEPEPEPEPEPEEPEPEPEPEPEEPEPEPEPEPEEPEPE

SEQ ID NO: 427 - protein, 60 aa, Artificial Sequence
  Synthetic polypeptide
EPDEPDEPDEPDEPDEPDEPDEPDEPDEPDEPDEPDEPDEPDEPDEPDEPDEPDEPDEPD

SEQ ID NO: 428 - protein, 60 aa, Artificial Sequence
  Synthetic polypeptide
EPEPDEPEPDEPEPDEPEPDEPEPDEPEPDEPEPDEPEPDEPEPDEPEPDEPEPDEPEPD

SEQ ID NO: 429 - protein, 60 aa, Artificial Sequence
  Synthetic polypeptide
EPEPEPEPEPDEPEPEPEPEPDEPEPEPEPEPDEPEPEPEPEPDEPEPEPEPEPDEPEPE

SEQ ID NO: 430 - protein, 60 aa, Artificial Sequence
  Synthetic polypeptide
EPQEPQEPQEPQEPQEPQEPQEPQEPQEPQEPQEPQEPQEPQEPQEPQEPQEPQEPQEPQ

SEQ ID NO: 431 - protein, 60 aa, Artificial Sequence
  Synthetic polypeptide
EPEPQEPEPQEPEPQEPEPQEPEPQEPEPQEPEPQEPEPQEPEPQEPEPQEPEPQEPEPQ

SEQ ID NO: 432 - protein, 60 aa, Artificial Sequence
  Synthetic polypeptide
EPEPEPEPEPQEPEPEPEPEPQEPEPEPEPEPQEPEPEPEPEPQEPEPEPEPEPQEPEPE

SEQ ID NO: 433 - protein, 60 aa, Artificial Sequence
  Synthetic polypeptide
EPNEPNEPNEPNEPNEPNEPNEPNEPNEPNEPNEPNEPNEPNEPNEPNEPNEPNEPNEPN

SEQ ID NO: 434 - protein, 60 aa, Artificial Sequence
  Synthetic polypeptide
EPEPNEPEPNEPEPNEPEPNEPEPNEPEPNEPEPNEPEPNEPEPNEPEPNEPEPNEPEPN

SEQ ID NO: 435 - protein, 60 aa, Artificial Sequence
  Synthetic polypeptide
EPEPEPEPEPNEPEPEPEPEPNEPEPEPEPEPNEPEPEPEPEPNEPEPEPEPEPNEPEPE

SEQ ID NO: 436 - protein, 60 aa, Artificial Sequence
  Synthetic polypeptide
EPKEPKEPKEPKEPKEPKEPKEPKEPKEPKEPKEPKEPKEPKEPKEPKEPKEPKEPKEPK

SEQ ID NO: 437 - protein, 60 aa, Artificial Sequence
  Synthetic polypeptide
EPEPKEPEPKEPEPKEPEPKEPEPKEPEPKEPEPKEPEPKEPEPKEPEPKEPEPKEPEPK

SEQ ID NO: 438 - protein, 60 aa, Artificial Sequence
  Synthetic polypeptide
EPEPEPEPEPKEPEPEPEPEPKEPEPEPEPEPKEPEPEPEPEPKEPEPEPEPEPKEPEPE

SEQ ID NO: 439 - protein, 60 aa, Artificial Sequence
  Synthetic polypeptide
EPREPREPREPREPREPREPREPREPREPREPREPREPREPREPREPREPREPREPREPR

SEQ ID NO: 440 - protein, 60 aa, Artificial Sequence
  Synthetic polypeptide
EPEPREPEPREPEPREPEPREPEPREPEPREPEPREPEPREPEPREPEPREPEPREPEPR

SEQ ID NO: 441 - protein, 60 aa, Artificial Sequence
  Synthetic polypeptide
EPEPEPEPEPREPEPEPEPEPREPEPEPEPEPREPEPEPEPEPREPEPEPEPEPREPEPE

SEQ ID NO: 442 - protein, 60 aa, Artificial Sequence
  Synthetic polypeptide
EPPEPPEPPEPPEPPEPPEPPEPPEPPEPPEPPEPPEPPEPPEPPEPPEPPEPPEPPEPP

SEQ ID NO: 443 - protein, 60 aa, Artificial Sequence
  Synthetic polypeptide
EPEPPEPEPPEPEPPEPEPPEPEPPEPEPPEPEPPEPEPPEPEPPEPEPPEPEPPEPEPP

SEQ ID NO: 444 - protein, 60 aa, Artificial Sequence
  Synthetic polypeptide
EPEPEPEPEPPEPEPEPEPEPPEPEPEPEPEPPEPEPEPEPEPPEPEPEPEPEPPEPEPE

SEQ ID NO: 445 - protein, 60 aa, Artificial Sequence
  Synthetic polypeptide
EPGEPGEPGEPGEPGEPGEPGEPGEPGEPGEPGEPGEPGEPGEPGEPGEPGEPGEPGEPG

SEQ ID NO: 446 - protein, 60 aa, Artificial Sequence
  Synthetic polypeptide
EPEPGEPEPGEPEPGEPEPGEPEPGEPEPGEPEPGEPEPGEPEPGEPEPGEPEPGEPEPG

SEQ ID NO: 447 - protein, 60 aa, Artificial Sequence
  Synthetic polypeptide
EPEPEPEPEPGEPEPEPEPEPGEPEPEPEPEPGEPEPEPEPEPGEPEPEPEPEPGEPEPE

SEQ ID NO: 448 - protein, 60 aa, Artificial Sequence
  Synthetic polypeptide
EPLEPLEPLEPLEPLEPLEPLEPLEPLEPLEPLEPLEPLEPLEPLEPLEPLEPLEPLEPL

SEQ ID NO: 449 - protein, 60 aa, Artificial Sequence
  Synthetic polypeptide
EPEPLEPEPLEPEPLEPEPLEPEPLEPEPLEPEPLEPEPLEPEPLEPEPLEPEPLEPEPL

SEQ ID NO: 450 - protein, 60 aa, Artificial Sequence
  Synthetic polypeptide
EPEPEPEPEPLEPEPEPEPEPLEPEPEPEPEPLEPEPEPEPEPLEPEPEPEPEPLEPEPE

SEQ ID NO: 451 - protein, 60 aa, Artificial Sequence
  Synthetic polypeptide
EPIEPIEPIEPIEPIEPIEPIEPIEPIEPIEPIEPIEPIEPIEPIEPIEPIEPIEPIEPI

SEQ ID NO: 452 - protein, 60 aa, Artificial Sequence
  Synthetic polypeptide
EPEPIEPEPIEPEPIEPEPIEPEPIEPEPIEPEPIEPEPIEPEPIEPEPIEPEPIEPEPI

SEQ ID NO: 453 - protein, 60 aa, Artificial Sequence
  Synthetic polypeptide
EPEPEPEPEPIEPEPEPEPEPIEPEPEPEPEPIEPEPEPEPEPIEPEPEPEPEPIEPEPE

SEQ ID NO: 454 - protein, 60 aa, Artificial Sequence
  Synthetic polypeptide
PASAPASAPASAPASAPASAPASAPASAPASAPASAPASAPASAPASAPASAPASAPASA

SEQ ID NO: 455 - protein, 60 aa, Artificial Sequence
  Synthetic polypeptide
PASPASAPASPASAPASPASAPASPASAPASPASAPASPASAPASPASAPASPASAPASP

SEQ ID NO: 456 - protein, 60 aa, Artificial Sequence
  Synthetic polypeptide
PASPASPASPASPASAPASPASPASPASPASAPASPASPASPASPASAPASPASPASPAS

SEQ ID NO: 457 - protein, 60 aa, Artificial Sequence
  Synthetic polypeptide
PASSPASSPASSPASSPASSPASSPASSPASSPASSPASSPASSPASSPASSPASSPASS

SEQ ID NO: 458 - protein, 60 aa, Artificial Sequence
  Synthetic polypeptide
PASPASSPASPASSPASPASSPASPASSPASPASSPASPASSPASPASSPASPASSPASP

SEQ ID NO: 459 - protein, 60 aa, Artificial Sequence
  Synthetic polypeptide
PASPASPASPASPASSPASPASPASPASPASSPASPASPASPASPASSPASPASPASPAS

SEQ ID NO: 460 - protein, 60 aa, Artificial Sequence
  Synthetic polypeptide
PASTPASTPASTPASTPASTPASTPASTPASTPASTPASTPASTPASTPASTPASTPAST

SEQ ID NO: 461 - protein, 60 aa, Artificial Sequence
  Synthetic polypeptide
PASPASTPASPASTPASPASTPASPASTPASPASTPASPASTPASPASTPASPASTPASP

SEQ ID NO: 462 - protein, 60 aa, Artificial Sequence
  Synthetic polypeptide
PASPASPASPASPASTPASPASPASPASPASTPASPASPASPASPASTPASPASPASPAS

SEQ ID NO: 463 - protein, 60 aa, Artificial Sequence
  Synthetic polypeptide
PASEPASEPASEPASEPASEPASEPASEPASEPASEPASEPASEPASEPASEPASEPASE

SEQ ID NO: 464 - protein, 60 aa, Artificial Sequence; identical to SEQ ID NO: 60
  Synthetic polypeptide

SEQ ID NO: 465 - protein, 60 aa, Artificial Sequence
  Synthetic polypeptide
PASPASPASPASPASEPASPASPASPASPASEPASPASPASPASPASEPASPASPASPAS

SEQ ID NO: 466 - protein, 60 aa, Artificial Sequence
  Synthetic polypeptide
PASDPASDPASDPASDPASDPASDPASDPASDPASDPASDPASDPASDPASDPASDPASD

SEQ ID NO: 467 - protein, 60 aa, Artificial Sequence
  Synthetic polypeptide
PASPASDPASPASDPASPASDPASPASDPASPASDPASPASDPASPASDPASPASDPASP

SEQ ID NO: 468 - protein, 60 aa, Artificial Sequence
  Synthetic polypeptide
PASPASPASPASPASDPASPASPASPASPASDPASPASPASPASPASDPASPASPASPAS

SEQ ID NO: 469 - protein, 60 aa, Artificial Sequence
  Synthetic polypeptide
PASQPASQPASQPASQPASQPASQPASQPASQPASQPASQPASQPASQPASQPASQPASQ

SEQ ID NO: 470 - protein, 60 aa, Artificial Sequence
  Synthetic polypeptide
PASPASQPASPASQPASPASQPASPASQPASPASQPASPASQPASPASQPASPASQPASP

SEQ ID NO: 471 - protein, 60 aa, Artificial Sequence
  Synthetic polypeptide
PASPASPASPASPASQPASPASPASPASPASQPASPASPASPASPASQPASPASPASPAS

SEQ ID NO: 472 - protein, 60 aa, Artificial Sequence
  Synthetic polypeptide
PASNPASNPASNPASNPASNPASNPASNPASNPASNPASNPASNPASNPASNPASNPASN

SEQ ID NO: 473 - protein, 60 aa, Artificial Sequence
  Synthetic polypeptide
PASPASNPASPASNPASPASNPASPASNPASPASNPASPASNPASPASNPASPASNPASP

SEQ ID NO: 474 - protein, 60 aa, Artificial Sequence
  Synthetic polypeptide
PASPASPASPASPASNPASPASPASPASPASNPASPASPASPASPASNPASPASPASPAS

SEQ ID NO: 475 - protein, 60 aa, Artificial Sequence
  Synthetic polypeptide
PASKPASKPASKPASKPASKPASKPASKPASKPASKPASKPASKPASKPASKPASKPASK

SEQ ID NO: 476 - protein, 60 aa, Artificial Sequence
  Synthetic polypeptide
PASPASKPASPASKPASPASKPASPASKPASPASKPASPASKPASPASKPASPASKPASP

SEQ ID NO: 477 - protein, 60 aa, Artificial Sequence
  Synthetic polypeptide
PASPASPASPASPASKPASPASPASPASPASKPASPASPASPASPASKPASPASPASPAS

SEQ ID NO: 478 - protein, 60 aa, Artificial Sequence
  Synthetic polypeptide
PASRPASRPASRPASRPASRPASRPASRPASRPASRPASRPASRPASRPASRPASRPASR

SEQ ID NO: 479 - protein, 60 aa, Artificial Sequence
  Synthetic polypeptide
PASPASRPASPASRPASPASRPASPASRPASPASRPASPASRPASPASRPASPASRPASP

SEQ ID NO: 480 - protein, 60 aa, Artificial Sequence
  Synthetic polypeptide
PASPASPASPASPASRPASPASPASPASPASRPASPASPASPASPASRPASPASPASPAS

SEQ ID NO: 481 - protein, 60 aa, Artificial Sequence
  Synthetic polypeptide
PASPPASPPASPPASPPASPPASPPASPPASPPASPPASPPASPPASPPASPPASPPASP

SEQ ID NO: 482 - protein, 60 aa, Artificial Sequence
  Synthetic polypeptide
PASPASPPASPASPPASPASPPASPASPPASPASPPASPASPPASPASPPASPASPPASP

SEQ ID NO: 483 - protein, 60 aa, Artificial Sequence
  Synthetic polypeptide
PASPASPASPASPASPPASPASPASPASPASPPASPASPASPASPASPPASPASPASPAS

SEQ ID NO: 484 - protein, 60 aa, Artificial Sequence
  Synthetic polypeptide
PASGPASGPASGPASGPASGPASGPASGPASGPASGPASGPASGPASGPASGPASGPASG

SEQ ID NO: 485 - protein, 60 aa, Artificial Sequence
  Synthetic polypeptide
PASPASGPASPASGPASPASGPASPASGPASPASGPASPASGPASPASGPASPASGPASP

SEQ ID NO: 486 - protein, 60 aa, Artificial Sequence
  Synthetic polypeptide
PASPASPASPASPASGPASPASPASPASPASGPASPASPASPASPASGPASPASPASPAS

SEQ ID NO: 487 - protein, 60 aa, Artificial Sequence
  Synthetic polypeptide
PASLPASLPASLPASLPASLPASLPASLPASLPASLPASLPASLPASLPASLPASLPASL

SEQ ID NO: 488 - protein, 60 aa, Artificial Sequence
  Synthetic polypeptide
PASPASLPASPASLPASPASLPASPASLPASPASLPASPASLPASPASLPASPASLPASP

SEQ ID NO: 489 - protein, 60 aa, Artificial Sequence
  Synthetic polypeptide
PASPASPASPASPASLPASPASPASPASPASLPASPASPASPASPASLPASPASPASPAS

SEQ ID NO: 490 - protein, 60 aa, Artificial Sequence
  Synthetic polypeptide
PASIPASIPASIPASIPASIPASIPASIPASIPASIPASIPASIPASIPASIPASIPASI

SEQ ID NO: 491 - protein, 60 aa, Artificial Sequence
  Synthetic polypeptide
PASPASIPASPASIPASPASIPASPASIPASPASIPASPASIPASPASIPASPASIPASP

SEQ ID NO: 492 - protein, 60 aa, Artificial Sequence
  Synthetic polypeptide
PASPASPASPASPASIPASPASPASPASPASIPASPASPASPASPASIPASPASPASPAS

SEQ ID NO: 493 - protein, 60 aa, Artificial Sequence
  Synthetic polypeptide
GGSPGSPAGSPTSTEEGTSESATPESGPGTSTEPSEGSAPGSPAGSPTSTEEGTSTEPSE

SEQ ID NO: 494 - protein, 60 aa, Artificial Sequence
  Synthetic polypeptide
GSAPGTSTEPSEGSAPGTSESATPESGPGSEPATSGSETPGSEPATSGSETPGSPAGSPT

SEQ ID NO: 495 - protein, 60 aa, Artificial Sequence
  Synthetic polypeptide
STEEGTSESATPESGPGTSTEPSEGSAPGTSTEPSEGSAPGSPAGSPTSTEEGTSTEPSE

SEQ ID NO: 496 - protein, 60 aa, Artificial Sequence
  Synthetic polypeptide
GSAPGTSTEPSEGSAPGTSESATPESGPGTSTEPSEGSAPGTSESATPESGPGSEPATSG

SEQ ID NO: 497 - protein, 60 aa, Artificial Sequence
  Synthetic polypeptide
SETPGTSTEPSEGSAPGTSTEPSEGSAPGTSESATPESGPGTSESATPESGPGSPAGSPT

SEQ ID NO: 498 - protein, 60 aa, Artificial Sequence
  Synthetic polypeptide
STEEGTSESATPESGPGSEPATSGSETPGTSESATPESGPGTSTEPSEGSAPGTSTEPSE

SEQ ID NO: 499 - protein, 60 aa, Artificial Sequence
  Synthetic polypeptide
GSAPGTSTEPSEGSAPGTSTEPSEGSAPGTSTEPSEGSAPGTSTEPSEGSAPGSPAGSPT

SEQ ID NO: 500 - protein, 60 aa, Artificial Sequence
  Synthetic polypeptide
STEEGTSTEPSEGSAPGTSESATPESGPGSEPATSGSETPGTSESATPESGPGSEPATSG

SEQ ID NO: 501 - protein, 60 aa, Artificial Sequence
  Synthetic polypeptide
SETPGTSESATPESGPGTSTEPSEGSAPGTSESATPESGPGSPAGSPTSTEEGSPAGSPT

SEQ ID NO: 502 - protein, 60 aa, Artificial Sequence
  Synthetic polypeptide
STEEGSPAGSPTSTEEGTSESATPESGPGTGTSESATPESGPGSEPATSGSETPGTSESA

SEQ ID NO: 503 - protein, 60 aa, Artificial Sequence
  Synthetic polypeptide
TPESGPGSEPATSGSETPGTSESATPESGPGTSTEPSEGSAPGSPAGSPTSTEEGTSESA

SEQ ID NO: 504 - protein, 60 aa, Artificial Sequence
  Synthetic polypeptide
TPESGPGSEPATSGSETPGTSESATPESGPGSPAGSPTSTEEGSPAGSPTSTEEGTSTEP

SEQ ID NO: 505 - protein, 60 aa, Artificial Sequence
  Synthetic polypeptide
SEGSAPGTSESATPESGPGTSESATPESGPGTSESATPESGPGSEPATSGSETPGSEPAT

SEQ ID NO: 506 - protein, 60 aa, Artificial Sequence
  Synthetic polypeptide
SGSETPGSPAGSPTSTEEGTSTEPSEGSAPGTSTEPSEGSAPGSEPATSGSETPGTSESA

SEQ ID NO: 507 - protein, 60 aa, Artificial Sequence
  Synthetic polypeptide
GTSTEPSEGSAPGTSTEPSEGSAPGSEPATSGSETPGTSESATPESGPGTSTEPSEGSAP

SEQ ID NO: 508 - protein, 60 aa, Artificial Sequence
  Synthetic polypeptide
STEPSEGSAPSTEPSEGSAPSTEPSEGSAPSTEPSEGSAPSTEPSEGSAPSTEPSEGSAP

SEQ ID NO: 509 - protein, 60 aa, Artificial Sequence
  Synthetic polypeptide
GSPAGSPTSTEEGTGSPAGSPTSTEEGTGSPAGSPTSTEEGTGSPAGSPTSTEEGTGSPA

SEQ ID NO: 510 - protein, 60 aa, Artificial Sequence
  Synthetic polypeptide
STEPSEGSAPGSPAGSPTSTEEGTSTEPSEGSAPGSPAGSPTSTEEGTSTEPSEGSAPGS

SEQ ID NO: 511 - protein, 60 aa, Artificial Sequence
  Synthetic polypeptide
PSTADPSTADPSTADPSTADPSTADPSTADPSTADPSTADPSTADPSTADPSTADPSTAD

SEQ ID NO: 512 - protein, 60 aa, Artificial Sequence
  Synthetic polypeptide
PSTADGSTADPSTADGSTADPSTADGSTADPSTADGSTADPSTADGSTADPSTADGSTAD

SEQ ID NO: 513 - protein, 60 aa, Artificial Sequence
  Synthetic polypeptide
PSTAKPSTAKPSTAKPSTAKPSTAKPSTAKPSTAKPSTAKPSTAKPSTAKPSTAKPSTAK

SEQ ID NO: 514 - protein, 118 aa, Artificial Sequence; identical to SEQ ID NO: 61
  Synthetic polypeptide

SEQ ID NO: 515 - protein, 118 aa, Artificial Sequence; identical to SEQ ID NO: 62
  Synthetic polypeptide

SEQ ID NO: 516 - protein, 59 aa, Artificial Sequence; identical to SEQ ID NO: 63
  Synthetic polypeptide

SEQ ID NO: 517 - protein, 59 aa, Artificial Sequence; identical to SEQ ID NO: 64
  Synthetic polypeptide

SEQ ID NO: 518 - protein, 59 aa, Artificial Sequence; identical to SEQ ID NO: 66
  Synthetic polypeptide

SEQ ID NO: 519 - protein, 157 aa, Artificial Sequence
  Synthetic polypeptide
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 520 - protein, 157 aa, Artificial Sequence
  Synthetic polypeptide
MNQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 521 - protein, 157 aa, Artificial Sequence
  Synthetic polypeptide
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 522 - protein, 157 aa, Artificial Sequence
  Synthetic polypeptide
  MISC_FEATURE (9)..(9): Xaa can be Y or H
  MISC_FEATURE (38)..(38): Xaa can be A or R
  MISC_FEATURE (82)..(82): Xaa can be N or D
  MISC_FEATURE (87)..(87): Xaa can be R or D
  MISC_FEATURE (95)..(95): Xaa can be D or N
  misc_feature (97)..(97): Xaa can be any naturally occurring amino acid
  MISC_FEATURE (105)..(105): Xaa can be S or D or N
  MISC_FEATURE (119)..(119): Xaa can be R or E or N
  MISC_FEATURE (121)..(121): Xaa can be R or D or E
  MISC_FEATURE (122)..(122): Xaa can be D or K
  MISC_FEATURE (124)..(124): Xaa can be D or K
  MISC_FEATURE (126)..(126): Xaa can be H or D
MNQHSHKDXETVRIAVVRARWHAEIVDACVSAFEAAMXDIGGDRFAVDVFDVPGAYEIPLHARTLAETGRYGAVLGTAFVVXGGIYXHEFVASAVIXGMMNVQLXTGVPVLSAVLTPHXYXXSXAXTLLFLALFAVKGMEAARACVEILAAREKIAA

SEQ ID NO: 523 - protein, 205 aa, Artificial Sequence
  Synthetic polypeptide
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCEWFKAGVLAVGVGDALVKGDPDEVREKAKKFVEKIRGCTE

SEQ ID NO: 524 - protein, 205 aa, Artificial Sequence
  Synthetic polypeptide
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVEAMKGPFPNVKFVPTGGVDLDDVCEWFDAGVLAVGVGDALVEGDPDEVREDAKEFVEEIRGCTE

SEQ ID NO: 525 - protein, 205 aa, Artificial Sequence
  Synthetic polypeptide
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTE

SEQ ID NO: 526 - protein, 205 aa, Artificial Sequence
  Synthetic polypeptide
  MISC_FEATURE (126)..(126): Xaa can be T or D
  MISC_FEATURE (139)..(139): Xaa can be Q or E
  MISC_FEATURE (142)..(142): Xaa can be K or E
  MISC_FEATURE (160)..(160): Xaa can be N or D
  MISC_FEATURE (163)..(163): Xaa can be N or D
  MISC_FEATURE (166)..(166): Xaa can be E or K
  MISC_FEATURE (169)..(169): Xaa can be K or D
  MISC_FEATURE (179)..(179): Xaa can be S or K or D
  MISC_FEATURE (183)..(183): Xaa can be K or E
  MISC_FEATURE (185)..(185): Xaa can be T or D or K
  MISC_FEATURE (192)..(192): Xaa can be K or D
  MISC_FEATURE (195)..(195): Xaa can be A or E or K
  MISC_FEATURE (198)..(198): Xaa can be E or K
  MISC_FEATURE (199)..(199): Xaa can be K or E
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHXILKLFPGEVVGPXFVXAMKGPFPNVKFVPTGGVXLDXVCXWFXAGVLAVGVGXALVXGXPDEVREXAKXFVXXIRGCTE

SEQ ID NO: 527 - protein, 157 aa, Artificial Sequence
  Synthetic polypeptide
MNQHSHKDHETVRIAVVRARWHADIVDACVEAFEIAMAAIGGDRFAVDVFDVPGAYEIPLHARTLAETGRYGAVLGTAFVVNGGIYRHEFVASAVIDGMMNVQLDTGVPVLSAVLTPHRYRDSDEHHRFFAAHFAVKGVEAARACIEILNAREKIAA

SEQ ID NO: 528 - protein, 157 aa, Artificial Sequence
  Synthetic polypeptide
MNQHSHKDHETVRIAVVRARWHADIVDACVEAFEIAMAAIGGDRFAVDVFDVPGAYEIPLHARTLAETGRYGAVLGTAFVVDGGIYDHEFVASAVIDGMMNVQLDTGVPVLSAVLTPHEYEDSDEDHEFFAAHFAVKGVEAARACIEILNAREKIAA

SEQ ID NO: 529 - protein, 157 aa, Artificial Sequence
  Synthetic polypeptide
  MISC_FEATURE (9)..(9): Xaa can be Y or H
  MISC_FEATURE (82)..(82): Xaa can be N or D
  MISC_FEATURE (87)..(87): Xaa can be R or D
  MISC_FEATURE (105)..(105): Xaa can be S or D
  MISC_FEATURE (119)..(119): Xaa can be R or E
  MISC_FEATURE (121)..(121): Xaa can be R or E
  MISC_FEATURE (124)..(124): Xaa can be A or D
  MISC_FEATURE (126)..(126): Xaa can be H or D
  MISC_FEATURE (128)..(128): Xaa can be R or E
  MISC_FEATURE (150)..(150): Xaa can be A or N
MNQHSHKDXETVRIAVVRARWHADIVDACVEAFEIAMAAIGGDRFAVDVFDVPGAYEIPLHARTLAETGRYGAVLGTAFVVXGGIYXHEFVASAVIDGMMNVQLXTGVPVLSAVLTPHXYXDSXEXHXFFAAHFAVKGVEAARACIEILXAREKIAA

SEQ ID NO: 530 - protein, 114 aa, Artificial Sequence
  Synthetic polypeptide
MPIFTLNTNIKADDVPSDFLSLTSRLVGLILSKPGSYVAVHINTDQQLSFGGSTNPAAFGTLMSIGGIEPDKNRDHSAVLFDHLNAMLGIPKNRMYIHFVNLNGDDVGWNGTTF

SEQ ID NO: 531 - protein, 114 aa, Artificial Sequence
  Synthetic polypeptide
MPIFTLNTNIKADDVPSDFLSLTSRLVGLILSEPGSYVAVHINTDQQLSFGGSTNPAAFGTLMSIGGIEPDKNEDHSAVLFDHLNAMLGIPKNRMYIHFVDLDGDDVGWNGTTF

SEQ ID NO: 532 - protein, 114 aa, Artificial Sequence
  Synthetic polypeptide
  MISC_FEATURE (13)..(13): Xaa can be T or D
  MISC_FEATURE (33)..(33): Xaa can be K or E
  MISC_FEATURE (71)..(71): Xaa can be S or D
  MISC_FEATURE (74)..(74): Xaa can be R or E
  MISC_FEATURE (101)..(101): Xaa can be N or D
  MISC_FEATURE (103)..(103): Xaa can be N or D
MPIFTLNTNIKAXDVPSDFLSLTSRLVGLILSXPGSYVAVHINTDQQLSFGGSTNPAAFGTLMSIGGIEPXKNXDHSAVLFDHLNAMLGIPKNRMYIHFVXLXGDDVGWNGTTF

SEQ ID NO: 533 - DNA, 6 bp, Artificial Sequence
  Synthetic oligonucleotide
  misc_feature (1)..(6): r is A or G
rrrrrr

SEQ ID NO: 534 - DNA, 4 bp, Artificial Sequence
  Synthetic oligonucleotide
cttt

SEQ ID NO: 535 - DNA, 46 bp, Artificial Sequence
  Synthetic oligonucleotide
  misc_feature (1)..(13): Optional 5′ residues
  misc_feature (26)..(46): Optional 3′ residues
ctcgagggttctagggggtatctttgacggctccggttccggttct

SEQ ID NO: 536 - DNA, 29 bp, Artificial Sequence
  Synthetic oligonucleotide
  misc_feature (1)..(3): Optional 5′ residues
  misc_feature (11)..(29): Optional 3′ residues
tacaaaaaagcaggcttggcttccgggta

SEQ ID NO: 537 - DNA, 65 bp, Artificial Sequence
  Synthetic oligonucleotide
  misc_feature (11)..(18): Optional residues
  misc_feature (58)..(65): Optional 3′ residues
accccaaaagcgtaacgcctgacggagtgactttgagccagaaaacgctcacgggtgctgtcggt

SEQ ID NO: 538 - protein, 13 aa, Artificial Sequence
  Synthetic polypeptide
PETSPASTEPEGS

SEQ ID NO: 539 - protein, 15 aa, Artificial Sequence
  Synthetic polypeptide
PESTGAPGETSPEGS

SEQ ID NO: 540 - protein, 5 aa, Artificial Sequence
  Synthetic polypeptide
ESESG

SEQ ID NO: 541 - protein, 294 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGYLDGSGSGSVSDVPRDLEVVAATPTSLLISWYYPFCAFYYRITYGETGGNSPVQEFTVPRPSDTATISGLKPGVDYTITVYAVTCLGSYSRPISINYRT

SEQ ID NO: 542 - protein, 262 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGYLDGSGSGSVDNKFNKEMRNAYWEIALLPNLNNQQKRAFIRSLYDDPSQSANLLAEAKKLNDAQAPK

SEQ ID NO: 543 - protein, 328 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGYLDGSGSGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 544 - protein, 262 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGYLDGSGSGSVDNKFNKEMWAAWEEIRNLPNLNGWQMTAFIASLVDDPSQSANLLAEAKKLNDAQAPK

SEQ ID NO: 545 - protein, 360 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGYLDGSGSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLN

SEQ ID NO: 546 - protein, 312 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGYLDGSGSGSGVSDVPRDLEVVAATPTSLLISWDSGRGSYQYYRITYGETGGNSPVQEFTVPGPVHTATISGLKPGVDYTITVYAVTDHKPHADGPHTYHESPISINYRTEIDKGSGC

SEQ ID NO: 547 - protein, 322 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGYLDGSGSGSGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGS

SEQ ID NO: 548 - protein, 445 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGYLDGSGSGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 549 - protein, 444 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGYLDGSGSGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 550 - protein, 331 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGYLDGSGSGSMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTV

SEQ ID NO: 551 - protein, 262 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGNLDGSGSGSVDNKFNKEMRNAYWEIALLPNLNNQQKRAFIRSLYDDPSQSANLLAEAKKLNDAQAPK

SEQ ID NO: 552 - protein, 328 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGNLDGSGSGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 553 - protein, 262 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGNLDGSGSGSVDNKFNKEMWAAWEEIRNLPNLNGWQMTAFIASLVDDPSQSANLLAEAKKLNDAQAPK

SEQ ID NO: 554 - protein, 360 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGNLDGSGSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLN

SEQ ID NO: 555 - protein, 312 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGNLDGSGSGSGVSDVPRDLEVVAATPTSLLISWDSGRGSYQYYRITYGETGGNSPVQEFTVPGPVHTATISGLKPGVDYTITVYAVTDHKPHADGPHTYHESPISINYRTEIDKGSGC

SEQ ID NO: 556 - protein, 322 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGNLDGSGSGSGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGS

SEQ ID NO: 557 - protein, 444 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGNLDGSGSGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 558 - protein, 445 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGNLDGSGSGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 559 - protein, 331 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGNLDGSGSGSMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTV

SEQ ID NO: 560 - protein, 312 aa, Artificial Sequence; identical to SEQ ID NO: 555
  Synthetic polypeptide
  MISC_FEATURE (1)..(31)

SEQ ID NO: 561 - protein, 294 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAFIVDACVSAFEAAMRKIGGERFAVDVFDVPGAYEIPLHARTLAKTGRYGAVLGTAFVVNGGIYRHEFVASAVIDGMMNVQLDTGVPVLSAVLTPHNYDKSNAKTLLFLALFAVKGMEAARACVEILAAREKIAAGSLEGSRGNLDGSGSGSVSDVPRDLEVVAATPTSLLISWYYPFCAFYYRITYGETGGNSPVQEFTVPRPSDTATISGLKPGVDYTITVYAVTCLGSYSRPISINYRT

SEQ ID NO: 562 - protein, 294 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAEIVDAAVSAFEAAMRKIGGERFAVDVFDVPGAYEIPLHARTLAKTGRYGAVLGTAFVVNGGIYRHEFVASAVIDGMMNVQLDTGVPVLSAVLTPHNYDDSDAHTLLFLALFAVKGMEAARAAVEILAAREKIAAGSLEGSRGYLDGSGSGSVSDVPRDLEVVAATPTSLLISWYYPFCAFYYRITYGETGGNSPVQEFTVPRPSDTATISGLKPGVDYTITVYAVTCLGSYSRPISINYRT

SEQ ID NO: 563 - protein, 262 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAEIVDAAVSAFEAAMRKIGGERFAVDVFDVPGAYEIPLHARTLAKTGRYGAVLGTAFVVNGGIYRHEFVASAVIDGMMNVQLDTGVPVLSAVLTPHNYDDSDAHTLLFLALFAVKGMEAARAAVEILAAREKIAAGSLEGSRGYLDGSGSGSVDNKFNKEMRNAYWEIALLPNLNNQQKRAFIRSLYDDPSQSANLLAEAKKLNDAQAPK

SEQ ID NO: 564 - protein, 328 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAEIVDAAVSAFEAAMRKIGGERFAVDVFDVPGAYEIPLHARTLAKTGRYGAVLGTAFVVNGGIYRHEFVASAVIDGMMNVQLDTGVPVLSAVLTPHNYDDSDAHTLLFLALFAVKGMEAARAAVEILAAREKIAAGSLEGSRGYLDGSGSGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 565 - protein, 262 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAEIVDAAVSAFEAAMRKIGGERFAVDVFDVPGAYEIPLHARTLAKTGRYGAVLGTAFVVNGGIYRHEFVASAVIDGMMNVQLDTGVPVLSAVLTPHNYDDSDAHTLLFLALFAVKGMEAARAAVEILAAREKIAAGSLEGSRGYLDGSGSGSVDNKFNKEMWAAWEEIRNLPNLNGWQMTAFIASLVDDPSQSANLLAEAKKLNDAQAPK

SEQ ID NO: 566 - protein, 360 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAEIVDAAVSAFEAAMRKIGGERFAVDVFDVPGAYEIPLHARTLAKTGRYGAVLGTAFVVNGGIYRHEFVASAVIDGMMNVQLDTGVPVLSAVLTPHNYDDSDAHTLLFLALFAVKGMEAARAAVEILAAREKIAAGSLEGSRGYLDGSGSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLN

SEQ ID NO: 567 - protein, 312 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAEIVDAAVSAFEAAMRKIGGERFAVDVFDVPGAYEIPLHARTLAKTGRYGAVLGTAFVVNGGIYRHEFVASAVIDGMMNVQLDTGVPVLSAVLTPHNYDDSDAHTLLFLALFAVKGMEAARAAVEILAAREKIAAGSLEGSRGYLDGSGSGSGVSDVPRDLEVVAATPTSLLISWDSGRGSYQYYRITYGETGGNSPVQEFTVPGPVHTATISGLKPGVDYTITVYAVTDHKPHADGPHTYHESPISINYRTEIDKGSGC

SEQ ID NO: 568 - protein, 322 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAEIVDAAVSAFEAAMRKIGGERFAVDVFDVPGAYEIPLHARTLAKTGRYGAVLGTAFVVNGGIYRHEFVASAVIDGMMNVQLDTGVPVLSAVLTPHNYDDSDAHTLLFLALFAVKGMEAARAAVEILAAREKIAAGSLEGSRGYLDGSGSGSGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGS

SEQ ID NO: 569 - protein, 445 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAEIVDAAVSAFEAAMRKIGGERFAVDVFDVPGAYEIPLHARTLAKTGRYGAVLGTAFVVNGGIYRHEFVASAVIDGMMNVQLDTGVPVLSAVLTPHNYDDSDAHTLLFLALFAVKGMEAARAAVEILAAREKIAAGSLEGSRGYLDGSGSGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 570 - protein, 444 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAEIVDAAVSAFEAAMRKIGGERFAVDVFDVPGAYEIPLHARTLAKTGRYGAVLGTAFVVNGGIYRHEFVASAVIDGMMNVQLDTGVPVLSAVLTPHNYDDSDAHTLLFLALFAVKGMEAARAAVEILAAREKIAAGSLEGSRGYLDGSGSGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 571 - protein, 331 aa, Artificial Sequence
  Synthetic polypeptide
  MISC_FEATURE (1)..(31): Optional N-terminal residues
MGSSHHHHHHSSGLVPRGSEQKLISEEDLGSNQHSQKDQETVRIAVVRARWHAEIVDAAVSAFEAAMRKIGGERFAVDVFDVPGAYEIPLHARTLAKTGRYGAVLGTAFVVNGGIYRHEFVASAVIDGMMNVQLDTGVPVLSAVLTPHNYDDSDAHTLLFLALFAVKGMEAARAAVEILAAREKIAAGSLEGSRGYLDGSGSGSMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTV

SEQ ID NO: 572 - protein, 339 aa, Artificial Sequence
  Synthetic polypeptide
  MISC_FEATURE (91)..(108): optional residues
  MISC_FEATURE (329)..(339): Optional C-terminal residues
VSDVPRDLEVVAATPTSLLISWYYPFCAFYYRITYGETGGNSPVQEFTVPRPSDTATISGLKPGVDYTITVYAVTCLGSYSRPISINYRTGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSGLVPRGSLEHHHHHH

SEQ ID NO: 573 - protein, 307 aa, Artificial Sequence
  Synthetic polypeptide
  MISC_FEATURE (59)..(77): optional residues
  MISC_FEATURE (296)..(307): Optional C-terminal residues
VDNKFNKEMRNAYWEIALLPNLNNQQKRAFIRSLYDDPSQSANLLAEAKKLNDAQAPKGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSGLVPRGSLEHHHHHH

SEQ ID NO: 574 - protein, 373 aa, Artificial Sequence
  Synthetic polypeptide
  MISC_FEATURE (125)..(142): optional residues
  MISC_FEATURE (363)..(373): Optional C-terminal residues
DLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSGLVPRGSLEHHHHHH

SEQ ID NO: 575 - protein, 307 aa, Artificial Sequence
  Synthetic polypeptide
  MISC_FEATURE (59)..(76): optional residues
  MISC_FEATURE (298)..(307): Optional C-terminal residues
VDNKFNKEMWAAWEEIRNLPNLNGWQMTAFIASLVDDPSQSANLLAEAKKLNDAQAPKGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSGLVPRGSLEHHHHHH

SEQ ID NO: 576 - protein, 405 aa, Artificial Sequence
  Synthetic polypeptide
  MISC_FEATURE (156)..(174): optional residues
  MISC_FEATURE (395)..(405): Optional C-terminal residues
DLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSGLVPRGSLEHHHHHH

SEQ ID NO: 577 - protein, 367 aa, Artificial Sequence
  Synthetic polypeptide
  MISC_FEATURE (119)..(136): optional residues
  MISC_FEATURE (357)..(367): Optional C-terminal residues
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGSGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSGLVPRGSLEHHHHHH

SEQ ID NO: 578 - protein, 262 aa, Artificial Sequence
  Synthetic polypeptide
  MISC_FEATURE (13)..(31): optional residues
  MISC_FEATURE (252)..(262): optional residues
AHIVMVDAYKPTKGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSGLVPRGSLEHHHHHH

SEQ ID NO: 579 - protein, 489 aa, Artificial Sequence
  Synthetic polypeptide
  MISC_FEATURE (241)..(258): optional residues
  MISC_FEATURE (479)..(489): Optional C-terminal residues
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSGLVPRGSLEHHHHHH

SEQ ID NO: 580 - protein, 490 aa, Artificial Sequence
  Synthetic polypeptide
  MISC_FEATURE (242)..(259): optional residues
  MISC_FEATURE (480)..(490): Optional C-terminal residues
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSGLVPRGSLEHHHHHH

SEQ ID NO: 581 - protein, 357 aa, Artificial Sequence
  Synthetic polypeptide
  MISC_FEATURE (108)..(126): optional residues
  MISC_FEATURE (347)..(357): Optional C-terminal residues
GVSDVPRDLEVVAATPTSLLISWDSGRGSYQYYRITYGETGGNSPVQEFTVPGPVHTATISGLKPGVDYTITVYAVTDHKPHADGPHTYHESPISINYRTEIDKGSGCGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSGLVPRGSLEHHHHHH

SEQ ID NO: 582 - protein, 376 aa, Artificial Sequence
  Synthetic polypeptide
  MISC_FEATURE (128)..(145): optional residues
  MISC_FEATURE (366)..(376): Optional C-terminal residues
MADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTEGSGLVPRGSLEHHHHHH

SEQ ID NO: 583 - protein, 472 aa, Artificial Sequence
  Synthetic polypeptide
  MISC_FEATURE (241)..(270): optional linker
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTE

SEQ ID NO: 584 - protein, 473 aa, Artificial Sequence
  Synthetic polypeptide
  MISC_FEATURE (242)..(271): optional linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTE

SEQ ID NO: 585 - protein, 339 aa, Artificial Sequence
  Synthetic polypeptide
  MISC_FEATURE (108)..(137): optional linker
GSGVSDVPRDLEVVAATPTSLLISWDSGRGSYQYYRITYGETGGNSPVQEFTVPGPVHTATISGLKPGVDYTITVYAVTDHKPHADGPHTYHESPISINYRTEIDKGGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTE

SEQ ID NO: 586 - protein, 388 aa, Artificial Sequence
  Synthetic polypeptide
  MISC_FEATURE (157)..(186): optional linker
DLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTE

SEQ ID NO: 587 - protein, 322 aa, Artificial Sequence
  Synthetic polypeptide
  MISC_FEATURE (91)..(120): optional linker
VSDVPRDLEVVAATPTSLLISWYYPFCAFYYRITYGETGGNSPVQEFTVPRPSDTATISGLKPGVDYTITVYAVTCLGSYSRPISINYRTGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTE

SEQ ID NO: 588 - protein, 290 aa, Artificial Sequence
  Synthetic polypeptide
  MISC_FEATURE (59)..(88): optional linker
VDNKFNKEMRNAYWEIALLPNLNNQQKRAFIRSLYDDPSQSANLLAEAKKLNDAQAPKGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTE

SEQ ID NO: 589 - protein, 356 aa, Artificial Sequence
  Synthetic polypeptide
  MISC_FEATURE (125)..(154): optional linker
DLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTE

SEQ ID NO: 590 - protein, 359 aa, Artificial Sequence
  Synthetic polypeptide
  MISC_FEATURE (128)..(157): optional linker
MADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTE

SEQ ID NO: 591 - protein, 349 aa, Artificial Sequence
  Synthetic polypeptide
  MISC_FEATURE (118)..(147): optional linker
EVQLQASGGGFVQPGGSLRLSCAASGFSSSNYAMGWFRQAPGKEREFVSAISRWDNVKAYYADSVKGRFTISRDNSKNTVYLQMNSLRAEDTATYYCAMVDDYWDPGYWGQGTQVTVGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTE

SEQ ID NO: 592 - protein, 350 aa, Artificial Sequence
  Synthetic polypeptide
  MISC_FEATURE (119)..(148): optional linker
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGSGDGGRGSRGGDGSGGSSGEKAAKAEEAARIEELFKRHTIVAVLRANSVEEAIEKAVAVFAGGVHLIEITFTVPDADTVIKALSVLKEDGAIIGAGTVTSVDQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGNALVKGNPDKVREKAKKFVKKIRGCTE

SEQ ID NO: 593 - protein, 60 aa, Artificial Sequence
  Synthetic polypeptide
DEQDEQDEQDEQDEQDEQDEQDEQDEQDEQDEQDEQDEQDEQDEQDEQDEQDEQDEQDEQ

SEQ ID NO: 594 - protein, 60 aa, Artificial Sequence
  Synthetic polypeptide
RKRKRKRKRKARKRKRKRKRKARKRKRKRKRKARKRKRKRKRKARKRKRKRKRKARKRKR

SEQ ID NO: 595 - protein, 60 aa, Artificial Sequence
  Synthetic polypeptide
EKGEKGEKGEKGEKGEKGEKGEKGEKGEKGEKGEKGEKGEKGEKGEKGEKGEKGEKGEKG

(19) United States
(12) Patent Application Publication
(10) Pub. No.: US 2021/0380641 A1
(43) Pub. Date: Dec. 9, 2021

(54) SELF-ASSEMBLING PROTEIN STRUCTURES AND COMPONENTS THEREOF

(71) Applicant: UNIVERSITY OF WASHINGTON, Seattlle, WA (US)

(72) Inventors: Gabriel BUTTERFIELD, Seattle, WA (US); Marc Joseph LAJOIE, Seattle, WA (US); Neil P. KING, Seattle, WA (US); David BAKER, Seattle, WA (US); Daniel ELLIS, Seattle, WA (US)

(21) Appl. No.: 16/762,565

(22) PCT Filed: Nov. 9, 2018
(86) PCT No.: PCT/US2018/059943

§ 371 Date: May 8, 2020

Related U.S. Application Data
(60) Provisional application No. 62/686,576, filed on Jun. 18, 2018.
(60) Provisional application No. 62/583,937, filed on Nov. 9, 2017.

(51) Int. Cl. C07K 14/00; C12N 15/87; B82Y 40/00

(52) U.S. Cl. CPC C07K 14/001; B82Y 40/00; C12N 15/87

(57) ABSTRACT

Synthetic nanostructures, polypeptides that are useful, for example, in making synthetic nanostructures, and methods for using synthetic nanostructures are disclosed herein.

### RELATED APPLICATIONS

This application claims priority to U.S. Provisional Application Ser. No. 62/583,937 filed Nov. 9, 2017 and 62/686,576 filed Jun. 18, 2018, each incorporated by reference herein in their entirety.

### FEDERAL FUNDING STATEMENT

This invention was made with government support under Grant No. 2015184301, awarded by the National Science Foundation and Grant No. W911NF-15-1-0645, awarded by the U.S. Army Research Office. The government has certain rights in the invention.

### BACKGROUND

Molecular self- and co-assembly of proteins into highly ordered, symmetric supramolecular complexes is an elegant and powerful means of patterning matter at the atomic scale. Recent years have seen advances in the development of self-assembling biomaterials, particularly those composed of nucleic acids. DNA has been used to create, for example, nanoscale shapes and patterns, molecular containers, and three-dimensional macroscopic crystals. Methods for designing self-assembling proteins have progressed more slowly, yet the functional and physical properties of proteins make them attractive as building blocks for the development of advanced functional materials and delivery tools.

### SUMMARY OF THE INVENTION

In a first aspect, the disclosure provides isolated polypeptides comprising an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length to the amino acid sequence of SEQ ID NO:1, wherein the polypeptide includes 1, 2, 3, 4, 5, 6, 7, 8, 9, 10, 11, or all 12 amino acid changes from SEQ ID NO:1 selected from the group consisting of K2T, K9R, K11T, K61D, E74D, T126D, E166K, S179K/N, T185K/N, E188K, A195K, and E198K. In one embodiment, the polypeptide includes 1, 2, 3, 4, or all 5 or more of the following amino acid changes from SEQ ID NO:1: C76A, C100A, N160C, C165A, and C203A. In a further embodiment, the polypeptide comprises an amino acid sequence at least 50%, 60%, 70%, 75%, 80%, 85%, 90%, 95%, 96%, 97%, 98%, 99%, or 100% identical to the full length of the amino acid sequence of a polypeptide selected from the group consisting of SEQ ID NOS:5-14.

In a second aspect, the disclosure provides isolated polypeptides comprising an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence of SEQ ID NO:2, wherein the polypeptide includes 1, 2, 3, 4, 5, 6, 7, 8, 9, 10, 11, 12, or all 13 amino acid changes from SEQ ID NO:2 selected from the group consisting of H6Q, Y9H/Q, E24F/M, A38R, D39K, D43E, E67K, S105D, R119N, R121D, D122K, D124K/N, and H126K. In one embodiment, the polypeptide includes 1 or both of the following amino acid changes from SEQ ID NO:2: C29A and C145A. In a further embodiment, the polypeptide comprises an amino acid sequence at least 50%, 60%, 70%, 75%, 80%, 85%, 90%, 95%, 96%, 97%, 98%, 99%, or 100% identical to the full length of the amino acid sequence of a polypeptide selected from the group consisting of SEQ ID NOS:15-21.

In a third aspect, the disclosure provides isolated polypeptides comprising an amino acid sequence that is at least 50% identical to the full length of the amino acid sequence of SEQ ID NO:3, wherein the polypeptide includes 1, 2, 3, or all 4 amino acid changes from SEQ ID NO:3 selected from the group consisting of T13D, S71K, N101R, and D105K. In one embodiment, the polypeptide comprises an amino acid sequence at least 50%, 60%, 70%, 75%, 80%, 85%, 90%, 95%, 96%, 97%, 98%, 99%, or 100% identical to the full length of the amino acid sequence of SEQ ID NO: 22.

In a fourth aspect, the disclosure provides isolated polypeptides comprising an amino acid sequence that is at least 50% identical to the full length of the amino acid sequence of SEQ ID NO:4, wherein the polypeptide includes 1, 2, 3, 4, 5, or all 6 amino acid changes from SEQ ID NO:4 selected from the group consisting of S105D, R119N, R121D, D122K, A124K, and A150N. In one embodiment, the polypeptide comprises an amino acid sequence at least 50%, 60%, 70%, 75%, 80%, 85%, 90%, 95%, 96%, 97%, 98%, 99%, or 100% identical to the full length of the amino acid sequence of SEQ ID NO: 23.

In one embodiment of any aspect of the disclosure, the polypeptide further comprises a targeting domain linked to the polypeptide. In one embodiment, the targeting domain is a polypeptide targeting domain, including but not limited to polypeptides selected from the group consisting of an antibody, an scFv, a nanobody, a DARPin, an affibody, a monobody, adnectin, an alphabody, an albumin-binding domain, an adhiron, an affilin, an affimer, an affitin, an anticalin, an armadillo repeat proteins, a tetranectin, an avimer/maxibody, a centyrin, a fynomer, a kunitz domain, an obody/OB-fold, a PRONECTIN®, a repebody, CD47, an RNA binding domain, and a bovine immunodefficiency virus Tat RNA-binding peptide (Btat). In another embodiment, the polypeptide targeting domain comprises an amino acid sequence at least 50%, 60%, 70%, 80%, 90%, 95%, or 100% identical to the full length of the amino acid sequence selected from the group consisting of SEQ ID Nos. 24-43. In another embodiment, the amino acid sequence of the polypeptides including a targeting domain, and optionally an amino acid linker, is at least 50%, 60%, 70%, 80%, 90%, or 100% identical to the full length of the amino acid sequence selected from the group consisting of SEQ ID Nos. 541-592. In another embodiment, the polypeptides may further comprise a stabilization domain, including but not limited to those selected from the group consisting of SEQ ID NOS: 58-518 and 593-595.

In another aspect, the disclosure provides nanostructures comprising

(a) a plurality of first assemblies, each first assembly comprising a plurality of identical first polypeptides, wherein the first polypeptides comprise the polypeptide of any embodiment of the first aspect of the disclosure; and

(b) a plurality of second assemblies, each second assembly comprising a plurality of identical second polypeptides, wherein the second polypeptides
(i) comprise the polypeptide of any embodiment of the second aspect of the disclosure, or
(ii) are at least 75%, 80%, 85%, 90%, 91%, 92%, 93%, 94%, 95%, 96%, 97%, 98%, or 99% identical over the length of the amino acid sequence selected from the group consisting of SEQ IDS NOS: 2, and 519-522;

wherein the plurality of first assemblies non-covalently interact with the plurality of second assemblies to form a nanostructure.

In another aspect, the disclosure provides nanostructures, comprising:

(a) a plurality of first assemblies, each first assembly comprising a plurality of identical first polypeptides, wherein the first polypeptides
(i) comprise the polypeptide of any embodiment of the first aspect of the disclosure, or
(ii) are at least 75%, 80%, 85%, 90%, 91%, 92%, 93%, 94%, 95%, 96%, 97%, 98%, or 99% identical over the length of the amino acid sequence selected from the group consisting of SEQ IDS NO:1 and 523-526; and

(b) a plurality of second assemblies, each second assembly comprising a plurality of identical second polypeptides, wherein the second polypeptides comprise the polypeptide of any embodiment of the second aspect of the disclosure;

wherein the plurality of first assemblies non-covalently interact with the plurality of second assemblies to form a nanostructure.

In a further aspect, the disclosure provides nanostructures comprising

(a) a plurality of first assemblies, each first assembly comprising a plurality of identical first polypeptides, wherein the first polypeptides comprise the polypeptide of any embodiment of the third aspect of the disclosure; and

(b) a plurality of second assemblies, each second assembly comprising a plurality of identical second polypeptides, wherein the second polypeptides
(i) comprise the polypeptide of any embodiment of the fourth aspect of the disclosure, or
(ii) are at least 75%, 80%, 85%, 90%, 91%, 92%, 93%, 94%, 95%, 96%, 97%, 98%, or 99% identical over the length of the amino acid sequence selected from the group consisting of SEQ IDS NOS: 4 and 527-529;

wherein the plurality of first assemblies non-covalently interact with the plurality of second assemblies to form a nanostructure.

In another aspect, the disclosure provides nanostructures, comprising:

(a) a plurality of first assemblies, each first assembly comprising a plurality of identical first polypeptides, wherein the first polypeptides
(i) comprise the polypeptide of any embodiment of the third aspect of the disclosure, or
(ii) wherein the first polypeptides are at least 75%, 80%, 85%, 90%, 91%, 92%, 93%, 94%, 95%, 96%, 97%, 98%, or 99% identical over the length of the amino acid sequence selected from the group consisting of SEQ IDS NOS: 3 and 530-532; and

(b) a plurality of second assemblies, each second assembly comprising a plurality of identical second polypeptides, wherein the second polypeptides comprise the polypeptide of any embodiment of the fourth aspect of the disclosure;

wherein the plurality of first assemblies non-covalently interact with the plurality of second assemblies to form a nanostructure.

In a further aspect, the disclosure provides polynucleotides encoding the polypeptide of any embodiment and aspect of the disclosure, recombinant expression vectors comprising the polynucleotides of the disclosure operably linked to a control sequence, recombinant host cells comprising the recombinant expression vectors of the disclosure, and nanostructures of any embodiment or aspect of the disclosure comprising the recombinant expression vector packaged within the nanostructure.

In various embodiments the nanostructures of the disclosure may comprise a therapeutic packaged within the nanostructure; in one non-limiting embodiment, the therapeutic comprises a therapeutic nucleic acid, such as an RNA therapeutic.

In another aspect, the disclosure provides uses for the polypeptides of all embodiments and aspects to prepare the nanostructures of the disclosure, and use of the nanostructures of all embodiments and aspects for targeting delivery of a therapeutic in vitro or in vivo.

In another aspect, the disclosure provides compositions comprising a synthetic nucleocapsid composed of a computationally-designed capsid derived from proteins that are of non-viral and/or non-container origin and designed to contact each other, wherein the capsid contacts a nucleic acid encoding its own genetic information.

In another aspect, the disclosure provides methods of generating polypeptides that self-assemble and package nucleic acid that encodes the polypeptides, comprising:

(a) symmetrically docking one or more polypeptides into an icosahedral geometry;

(b) redesigning the interior surfaces of the polypeptides to have a net charge between −200 and +1200, or between +100 and +900;

(c) encoding the polypeptides in a nucleic acid sequence;

(d) optionally introducing sequence variation in the nucleic acid sequence;

(e) introducing the nucleic acid(s) into a cell;

(f) culturing the cell under conditions to cause expression of the nucleic acid to produce the polypeptide in the cell; and

(g) isolating polypeptides that self-assemble and package the nucleic acid that encodes the polypeptide.

In another aspect, the disclosure provides methods of generating the polypeptides or nanostructures of any of the claims herein, wherein the methods comprise any methods disclosed herein.

In a further aspect, the disclosure provides synthetic nucleocapsids comprising:

a plurality of first oligomeric polypeptides, each first oligomeric polypeptide comprising a plurality of identical first synthetic polypeptides;

a plurality of second oligomeric polypeptides, each second oligomeric polypeptide comprising a plurality of identical second synthetic polypeptides;

wherein the plurality of first oligomeric polypeptides and the plurality of second oligomeric polypeptides interact non-covalently and assemble into an icosahedral geometry with an interior cavity (a synthetic capsid) that contacts a nucleic acid encoding the polypeptide components of the synthetic nucleocapsid;

wherein the synthetic nucleocapsid does not require viral proteins or naturally-occurring non-viral container proteins, and the first oligomeric polypeptides and second oligomeric polypeptides are selected to provide a positive net charge on the interior surface.

In various embodiments, the first assemblies and second assemblies may be selected to provide the synthetic nucleocapsid with a net interior charge of between about +100 and about +900, between about +200 and about +800, between about +250 and about +750, between about +250 and about +650, between about +250 and about +500, between about +250 and about +450, between about +300 and about +750, between about +300 and about +650, between about +300 and about +500, or between about +300 and about +450. In other embodiments, the first assemblies and second assemblies may be selected to provide the synthetic nucleocapsid with a circulation half-life in live mice of at least 10 minutes, 1 hour, 2 hours, 3 hours, 4 hours, or 4.5 hours.

In further embodiments, the synthetic nucleocapsid may exhibit improved genome packaging, for example, at least one full-length RNA per 1,000 synthetic nucleocapsids, at least five full-length RNA per 1,000 synthetic nucleocapsids, at least 10 full-length RNA per 1,000 synthetic nucleocapsids, at least 25 full-length RNA per 1,000 synthetic nucleocapsids, at least 50 full-length RNA per 1,000 synthetic nucleocapsids, at least 75 full-length RNA per 1,000 synthetic nucleocapsids, or at least 90 full-length RNA per 1,000 synthetic nucleocapsids.

In other embodiments, the synthetic nucleocapsid may exhibit a half-life of greater than 0.5, 0.75 hours, 1 hour, or 1.5 hours at 37° C. in the presence of RNase A, with the RNase being present at a concentration of 10 μg/mL. In further embodiments, the synthetic nucleocapsid includes a plurality of pores, with each pore having an area of less than about 2000, 1800, 1600, 1000, 600, 300, or 150 angstroms².

In another embodiment, at least one, two, three, or more (such as all) first synthetic polypeptide may comprise a linked targeting domain, and/or at least one, two, three, or more (such as all) second synthetic polypeptide may comprise a linked targeting domain. In one embodiment the targeting domain may be a polypeptide targeting domain, including but not limited to a polypeptide selected from the group consisting of an antibody, an antibody, an scFv, a nanobody, a DARPin, an affibody, a monobody, adnectin, an alphabody, an albumin-binding domain, an adhiron, an affilin, an affimer, an affitin, an anticalin, an armadillo repeat proteins, a tetranectin, an avimer/maxibody, a centyrin, a fynomer, a kunitz domain, an obody/OB-fold, a PRONECTIN®, a repebody, and CD47. In various further embodiments, the polypeptide targeting domain may comprise an amino acid sequence at least 50%, 60%, 70%, 80%, 90%, 95%, or 100% identical to a full length of an amino acid sequence selected from the group consisting of SEQ ID NOs: 24-43. In other embodiments, (i) the at least one first synthetic polypeptide or the at least one second synthetic polypeptide, and (ii) the polypeptide targeting domain may be linked by a non-covalent attachment or a covalent attachment, including but not limited to covalently linked by translational fusion. In further embodiments, the first synthetic polypeptides and/or the second synthetic polypeptides may comprise any embodiment or combination of embodiments of the first and second polypeptides disclosed herein for use in the nanostructures of the disclosure. In further embodiments, each first assembly may comprise 3 copies of the identical first polypeptide, and each second assembly may comprise 5 copies of the identical second polypeptide.

### DESCRIPTION OF THE FIGURES

The foregoing aspects and many of the attendant advantages of this invention will become more readily appreciated as the same become better understood by reference to the following detailed description, when taken in conjunction with the accompanying drawings.

FIG. 1. Biochemical characterization of synthetic nucleocapsids. a. Design model of I53-50-v1. Increasing the net positive interior charge permits RNA encapsulation. b. Synthetic nucleocapsids encapsulate their own mRNA genomes while assembling into icosahedral capsids inside E. coli cells. c. Negative-stain electron micrographs of I53-50-v1 (positively-charged interior) and I53-50-Btat (RNA binding tat peptide from bovine immunodeficiency virus). d,e. Synthetic nucleocapsids were purified, treated with RNase A, and electrophoresed on non-denaturing 1% agarose gels then stained with Coomassie (protein, d) and SYBR gold (nucleic acid, e). Nucleic acids co-migrated with capsid proteins for I53-50-v1 and I53-50-Btat, but not for the original I53-50. f. Full-length synthetic nucleocapsid genomes were recovered from each sample by RT-PCR. White + and − indicate PCR performed on template prepared with and without reverse transcriptase, respectively, confirming that I53-50-v1 and I53-50-Btat package their own full-length RNA genomes.

FIG. 2. Evolution of optimal interior charge for RNA packaging. a. A library of plasmids encoding synthetic nucleocapsid variants is transformed into E. coli. Each cell in the population produces a unique synthetic nucleocapsid variant. Nucleocapsids are purified en masse from cell lysates and challenged (e.g., RNase, heat, blood, mouse circulation). The capsid-protected mRNA is then recovered and amplified using RT-qPCR, re-cloned into a plasmid library, and transformed into E. coli for another generation. b-f. Combinatorial libraries targeting nine residues on the interior surface of I53-50 (Table S1) were used to investigate how interior surface charge affects RNA packaging in the presence or absence of a positively charged RNA binding peptide (Btat). Three rounds of evolution were performed with two independent biological replicates. b. The evolved populations converged toward narrow distributions of interior net charge: Btat-library from 215±114 (mean±standard deviation) to 388±87, Btat+ library from 733±119 to 662±91. The net interior charge of each variant was calculated from its sequence by summing the positive and negative residues on the interior surface. Black lines are without Btat and gray lines are with Btat; dashed lines are naïve populations and solid lines are round 3 selected populations. c. Rank order list of variants observed in both biological replicates; 1170 unique variants outperformed I53-50-v1. I53-50-v2 was created based on the second most highly enriched variant from the Btat-library. d,e. Log enrichment values for each mutation explored in the combinatorial surface charge optimization library. All except two of the lysine residues were beneficial in the absence of the positively charged Btat, whereas most lysine residues were disfavored in the presence of Btat. f. Design model of I53-50-v2. Although the net interior surface charge did not change from I53-50-v1 to I53-50-v2, the spatial configuration of charged residues impacted genome packaging efficiency (see FIG. 4a).

FIG. 3. Size Exclusion Chromatography of nucleocapsids. RNA-packaging capsids show identical size exclusion chromatography (SEC) retention volume as the original published capsid. Three versions of I53-50 and I53-47 were analyzed: v0 is the original published design, v1 has the designed positively charged interior, and Btat has the BIV Tat RNA-binding peptide translationally fused to the C-terminus of the capsid trimer subunit. a. SEC traces of I53-50 capsids were performed on a GE superose 6 increase column. b. SDS-PAGE of samples before and after SEC purification shows both subunits in the expected 1:1 stoichiometry. c, d. SEC traces and SDS-PAGE for I53-47 capsids

FIG. 4. Increased fitness devolved synthetic nucleocapsids, Evolution drastically increases the property under selection without compromising previously evolved properties. a-c. Time courses of full-length RNA genomes per 1000 capsids isolated after challenge: a. 10 μg/mL, RNase A at 37° C. (RNase, n=3), b. Heparinized whole murine blood at 37° C. (Blood, n=3), and c. in vivo circulation in mice (Live mouse, n=5), d. Summary of improved nucleocapsid properties, including total packaged RNA (10 μg/mL RNase A for 10 min at 25° C. to degrade non-encapsulated RNA. n=3). The colored arrows in a-c indicate the 6-hour time point represented in the summary plot. Five synthetic nucleocapsids were tested: I53-50-v0 (original assembly which did not package its full length mRNA), I53-50-v1 (design with positive interior surface for packaging RNA), I53-50-v2 (evolution-optimized interior surface), I53-50-v3 (evolution-optimized residues lining the capsid pore), and I53-50-v4 (evolution-optimized exterior surface for increased circulation in living mice). Evolution resulted in efficient genome encapsulation for I53-50-v2 and its derivatives (approximately 1 RNA genome per 14 icosahedral capsids for I53-50-v2), protection from blood for I53-50-v3 and I53-50-v4 (82% and 71% protection, respectively), and increased circulation half-life for I53-50-v4 (4.5 hours serum half-life), Full-length RNA genomes were quantitated by RT-qPCR, capsid proteins were quantitated by Qubit, and genomes per capsid were calculated based on these values by dividing the number of genomes by the number of capsids, e. Nucleocapsid genomes are enriched and ribosomal RNA is depleted in nucleocapsids. f. Top 13 RNA transcripts encapsulated in I53-50-v4. Nucleocapsid genomes account for more than 74% of the packaged transcripts. g,h. The relative biodistribution of intact I53-50-v3 (g) and I53-50-v4 (h) nucleocapsids was evaluated by RT-qPCR of their full-length genomes recovered from mouse organs harvested 5 minutes or 4 hours after retro-orbital injection. No obvious tissue tropism was observed for either nucleocapsid. At four hours post injection, I53-50-v3 had largely disappeared, while I53-50-v4 remained predominantly in the blood with lower levels in the other tissues. Error bars represent standard error of the mean.

FIG. 5A. Top candidate testing to choose I53-50-v2 with improved genome packaging. New variants were created rationally based on the best sequences from the evolved interior charge optimization (FIG. 2) and interface (fig. S2) libraries. The amount of packaged full-length mRNA was compared for each of these nucleocapsids. Each nucleocapsid was expressed, purified by IMAC, and treated with 10 μg/mL RNAse A at 20° C. for 10 minutes in triplicate. RT-qPCR was used to determine the relative amount of full length mRNA packaged in each variant. Cq values are reported relative to those of I53-50-v1 (Cq_{I53-50-v1}−Cqᵥₐᵣᵢₐₙₜ). The charge-optimized variant with E24F was chosen as I53-50-v2 based on this data. In the absence of a discernable difference in packaging between E24M and E24F, E24F was selected due to the apparent preference for hydrophobic residues at that position (fig. S2). Error bars represent standard error of the mean.

FIG. 5B-C. Complete deep mutational scanning data from FIG. 5A for the pentamer (FIG. 5B) and the trimer (FIG. 5C). Log enrichment values are indicated for every residue at every position in both subunits of I53-50-v2. The first column shows single letter amino acid codes for the mutations, and the first row shows the residue number in each sequence. Residues for which less than 10 counts were observed in the naïve library are denoted Na. Enrichment values are the average of two biological replicates (10 μg/mL RNAse A, 37° C., 1 hour).

FIG. 6. Deleterious lysine residues removed from I53-50-v1 mapped to the icosahedral pore. Retrospectively, we observed that the deleterious lysine residues removed from I53-50-v1 to produce I53-50-v2 (FIG. 2d; trimeric subunit: K179N, pentameric subunit: K124N) are in close proximity to the synthetic nucleocapsid pore. Therefore, the same mechanism that provided the selective pressure to remove the lysines surrounding the pore during the deep mutational scanning experiment may also explain these mutations from the interior charge optimization experiment (FIG. 2).

FIG. 7. Top candidate testing to choose I53-50-v3 with improved nuclease resistance. a. Log enrichment values for each mutation explored in the combinatorial library to remove positively charged residues near the nucleocapsid pore. A single round of selection (10 μg/mL RNAse A, 37° C., 1 hour) was performed. b. Enriched variants selected from the combinatorial library were expressed, purified by IMAC and SEC, and treated with 10 μg/mL RNAse A at 37° C. for 1 hour in duplicate. RT-qPCR was used to determine the relative amount of full length mRNA packaged in each variant. Cq values are reported relative to those of I53-50-v2 (Cq_{I53-50-v2}−Cqᵥₐᵣᵢₐₙₜ). The variant labeled Pore_Mut_4 was chosen as I53-50-v3 based on this data. Data points represent the values of two independent biological replicates, and bars represent the mean of these values.

FIG. 8. RNase protection is assembly dependent. Introduction of charged residues at the hydrophobic interface between subunits (trimeric subunit: V29R; pentameric subunit: A38R) compromises both assembly and RNase protection. a. SDS-PAGE analysis of the soluble fraction of E. coli lysate, IMAC-purified protein, and SEC-purified protein. Both subunits of I53-50-v3-KO express solubly, but only the 6×his-tagged pentamer is observed after IMAC. The lack of untagged trimer suggests that assembly does not occur. b. RT-qPCR of RNase A-treated nucleocapsids show a large increase in the number of PCR cycles required to recover nucleic acid when the icosahedral assembly interface is disrupted.

FIG. 9. Evolution of surface mutations that increase circulation time in living mice. Log enrichment values between the injected pool and RNA recovered from the tail vein 60 minutes later. Values for residues not in the designed combinatorial library left blank. Note the strong enrichment of the E67K mutation and corresponding depletion of the native E67 allele.

FIG. 10. Negative-stain transmission electron microscopy (EM) of nucleocapsids. EM shows that evolved variants of I53-50 and I53-47 maintain the same morphology as the initial computationally designed material.

FIG. 11. Negative-stain transmission electron microscopy class averages. a. Two-dimensional class averages of I53-50-v0 (7979 particles) and I53-50-v4 (7120 particles) datasets showing the percentage of the total particles present in each class. I53-50-v4 nucleocapsids are on average denser than unfilled I53-50-v0 assemblies, especially near the inner surface of the capsid. b. All I53-50-v0 and I53-50-v4 particles from panel a were combined into a single set (15,119 particles), and twenty class averages were made from the combined data. Class averages were grouped into three bins (v0 dominant has ≤25% I53-50-v4, v4 dominant has ≥74% I53-50-v4, and mixed has the rest) and arranged from left to right with increasing fraction of I53-50-v4 particles (shown below each class). The v0 dominant classes appear more similar to the I53-50-v0 class averages in panel a, while the v4 dominant classes appear more similar to the I53-50-v4 class averages. The percentage of the complete I53-50-v4 dataset found in each class is shown above each class average. c. Table presenting the bins into which I53-50-v4 particles were assigned. We found that 64% of I53-50-v4 particles were present in the v4 dominant classes, which also appear to be more filled than the v0-dominant classes. Although TEM cannot determine the nature of the contents, encapsulated RNA is plausible.

FIG. 12. Summary of encapsulated RNA composition analysis. a. Flow chart explaining the relationship between bulk RNA measurements and RT-qPCR quantitation. Bulk RNA measurements also account for cellular RNA and nucleocapsid genome fragments, whereas RT-qPCR only quantitates full-length genomes. Nucleocapsid genome: capsid ratios based on these measurements are reported in parentheses. b. Stacked bar blot describing the fractions of total encapsulated RNA that are full-length or fragmented nucleocapsid genome.

FIG. 13. Design models of synthetic nucleocapsid versions 1 through 4. Trimer subunits are colored green and pentamer subunits are colored cyan. Mutations with respect to the previous version are colored blue (increases positive charge and/or decreases negative charge [e.g., E→N, N→K, E→K]), orange (no change in charge [e.g., E→D, N→T, K→R], or red (decreases positive charge and/or increases negative charge [e.g., N→E, K→N, K→E]).

FIGS. 14. I53-47 nucleocapsids. a. Design model of I53-47 and negative-stain electron micrographs of I53-47-v1 (designed positively charged interior) and I53-47-Btat (BIV Tat RNA-binding peptide translationally fused to the C-terminus of the capsid trimeric subunit). b. Synthetic nucleocapsids were Ni-NTA-purified, RNase-treated, and electrophoresed on non-denaturing 1% agarose gels. The gels were stained with Coomassie (protein; b) and SYBR gold (nucleic acid, c). Nucleic acids co-migrated with capsid proteins for all three versions of I53-47, suggesting that all versions package nucleic acid. d. Full-length synthetic nucleocapsid genomes were recovered from each sample by RT-PCR. White + and − headings indicate PCR performed on template prepared with and without reverse transcriptase, respectively, confirming that all versions package their own full-length RNA genomes.

FIG. 15. SDS PAGE of Synthetic Nucleocapsids genetically fused to targeting domains. Synthetic Nucleocapsids were produced in E. coli Lemo21 and harvested by mechanical lysis as described in the methods. Synthetic Nucleocapsids were purified by Ni-NTA affinity chromatography (Ni) and Size Exclusion Chromatography (SEC), then analyzed by SDS-PAGE. Three bands are observed: trimeric component alone (˜23 kDa), pentameric component alone (˜19 kDa), and pentameric component translationally fused to the targeting domain via a frameshift linker (26-37 kDa). The targeting domains were: A. DARPin targeting EGFR B. DARPin targeting Her2 C. affibody targeting Her2 and D. affibody targeting EGFR. The molecular weight marker is Bio-rad dual extra molecular weight standard.

FIG. 16. SDS-PAGE of Synthetic Nucleocapsids genetically fused to targeting domains before and after thrombin cleavage. Synthetic Nucleocapsids were produced in E. coli Lemo21 and harvested by mechanical lysis as described in the methods. Synthetic Nucleocapsids were purified by Ni-NTA affinity chromatography (Ni) followed by dialysis into PBS, protease cleavage of 6×histidine tag with thrombin, and concentration in a spin concentrator with a 10,000 dalton molecular weight cutoff Three bands are observed: trimeric component alone (˜23 kDa), pentameric component alone (˜19 kDa), and pentameric component translationally fused to the targeting domain via a frameshift linker (26-37 kDa). The targeting domains are: A. no targeting domain B. Spycatcher™ C. affibody targeting Her2 D. darpin targeting Her2 E. affibody targeting EGFR F. darpin targeting EGFR G. adnectin targeting EGFR. The marker is Bio-rad dual extra molecular weight standard.

FIG. 17. Negative-stain transmission electron microscopy. Fully formed synthetic nucleocapsids are observed for all binding domain fusions. Note the similarity to the capsid displaying only a myc tag (A). The targeting domains are: A. V4-myc only B. V4-myc Her2 affibody C. V4-myc Her2 darpin D. V4-myc EGFR Affibody E. V4-myc EGFR Darpin F. V4-myc EGFR adnectin. 6 μl of purified protein at 0.001-0.01 mg/ml, were applied to glow discharged, carbon-coated 300-mesh copper grids, washed with Milli-Q water and stained with 0.75% uranyl formate. Data were collected on a 100 kV Morgagni M268 transmission electron microscope (HI) equipped with an Orius charge-coupled device (CCD) camera (Gatan).

FIG. 18. Targeted synthetic nucleocapsids bind specifically to 293Freestyle cells expressing HER2 or EGFR. 100 nM synthetic nucleocapsids labeled with AlexaFluor568 (I53-50-v4-GSprfB-HER2_DARPin, I53-50-v4-GSprfB-EGFR_affibody, and I53-50-v4-GSprfB-EGFR_DARPin) were diluted into PBSF and incubated with 293Freestyle cell lines that either expressed no additional proteins, HER2-EGFP, or EGFR-iRED. Flow cytometry was performed on an LSRII to analyze AlexaFluor568 binding (y-axis; 561 nm laser, 610/20 detector) versus HER2-EGFP expression (y-axis; 488 nm laser, 530/30 detector) or EGFR-iRED expression (x-axis; 637 nm laser, 670/30 detector). AlexaFluor568 binding correlates with HER2 or EGFR expression level, confirming that the synthetic nucleocapsids bind specifically to the desired targets. A variant of the synthetic nucleocapsid lacking a targeting domain (v4_neg) showed low levels of non-specific binding signal in all three cell lines. PE-conjugated HER2 and EGFR antibodies were used to confirm proper expression of the HER2-EGFP and EGFR-iRED markers. Each plot represents a mixed culture of 293Freestyle, 293Freestyle HER2-EGFP, and 293Freestyle EGFR-iRED cells labeled with the indicated synthetic nucleocapsid. No compensation was performed because AlexaFluor568 labeling requires HER2-EGFP or EGFR-iRED expression.

FIG. 19. Targeted synthetic nucleocapsids bind specifically to RAM cells stably expressing HER2, EGFR, and GFP. Flow cytometry was performed on an LSRII to analyze GFP expression (x-axis; 488 nm laser, 530/30 detector) and AlexaFluor568-labeled nucleocapsid binding (y-axis; 561 nm laser, 610/20 detector). AlexaFluor568 binding correlates with GFP expression for the HER2 DARPin, EGFR affibody, EGFR DARPin, and EGFR adnectin, confirming that binding is dependent on expression of the targeted marker (HER2 or EGFR). The labels indicate the targeting domain displayed on the I53-50-v4 nucleocapsid via a GSprfB linker. No compensation was performed because all cell lines in the experiment express GFP.

FIG. 20. SDS-PAGE analysis of v4_v0_cys and v4_v0_cys_6x_GGGC. Synthetic Nucleocapsids were produced in E. coli Lemo21 and harvested by mechanical lysis as described in the methods. Synthetic Nucleocapsids were purified by Ni-NTA affinity chromatography. Two bands are observed: trimeric component (˜22 kDa (v4_v0_cys_Trimer), ˜24 kDa (v4_v0_cys_Trimer_6x_Cys)), pentameric component alone (˜19 kDa).

FIG. 21. Native agarose gels of Synthetic Nucleocapsids genetically fused to targeting domains shows protection of nucleic acid from RNase degradation. Synthetic nucleocapsids were produced in E. coli Lemo21 and harvested by mechanical lysis as described in the methods. Synthetic nucleocapsids were purified by Ni-NTA affinity chromatography (Ni) then analyzed on Native Agarose gels stained with SYBR gold. The targeting domains were: A. no targeting domain B. DARPin targeting EGFR C. DARPin targeting Her2 D. affibody targeting Her2 and E. affibody targeting EGFR.

FIG. 22. SDS-PAGE of Synthetic Nucleocapsids with targeting domains fused to the amino terminus of the trimer component. Synthetic nucleocapsids were produced in E. coli Lemo21 and harvested by mechanical lysis as described in the methods. Synthetic nucleocapsids were purified by Ni-NTA affinity chromatography. The band corresponding to the weight of the trimeric component with fused binder is emphasized with an arrow (˜35-50 kDa). The pentameric subunit is also observed at ˜19 kDa). Other bands likely represent contaminating E. coli proteins. A. I53-50-v4-aCD3_ntrimer B. I53-50-v4-ad_EGFR_ntrimer C. I53-50-v4-spycatcher_ntrimer

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

As used herein, “about” means+/−5% of the recited parameter.

All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In a first aspect, the disclosure provides isolated non-naturally occurring polypeptides comprising an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length to the amino acid sequence of SEQ ID NO:1, wherein the polypeptide includes one or more amino acid change from SEQ ID NO:1 selected from the group consisting of K2T, K9R, K11T, K61D, E74D, T126D, E166K, S179K/N, T185K/N, E188K, A195K, and E198K.

| | | Conserved interface |
|---|---|---|
| Name | Amino acid sequence | residues |
| I53-50A | (MKM)EELFKKHKIVAVLRANSVEEAIEKAVAVFA | 153-50A: 25, 29, 33, 54 |
| SEQ ID | GGVHLIEITFTVPDADTVIKALSVLKEKGAIIGAGT | 57: Non-conserved |
| NO: 1 | VTSVEQCRKAVESGAEFIVSPHLDEEISQFCKEKG | interface residue |
| TRIMER | VFYMPGVMTPTELVKAMKLGHTILKLFPGEVVGP | |
| | QFVKAMKGPFPNVKFVPTGGVNLDNVCEWFKAG | |
| | VLAVGVGSALVKGTPDEVREKAKAFVEKIRGCTE | |

The polypeptides of this first aspect were designed for their ability to self-assemble in pairs with I53-50 pentamer polypeptides disclosed herein to form significantly improved nanostructures as disclosed herein. The nanostructures of the disclosure are capable of, for example, significant improved packaging of cargo such as RNA, including their own genome and thus serve as designed nucleocapsids, as described in the examples that follow. The polypeptides are also shown to be significantly improved in attaching targeting domains and to significantly improve in vivo circulation time. The synthetic polypeptides and nanostructures described herein comprise non-naturally occurring sequences of protein assemblies encoded by non-naturally occurring sequences of polynucleotides. In an application, the polypeptides and nanoparticles described herein are not derived from naturally occurring viral particles, and can be adapted to targeted delivery of cargo. Unlike most viruses, which are composed of proteins that adopt multiple different conformations during capsid assembly and/or dock in domain-swapped conformations, the nanoparticles of the disclosure comprise highly stable subunits that adopt a single conformation, fold independently, and dock into simple icosahedral symmetry. This allows them to tolerate the attachment of modular cargo packaging domains on the interior as described herein (such as, for example, BIV Tat RNA binding domain, and the like) and/or modular cell targeting domains on the exterior, as described in detail herein.

The polypeptides are non-naturally occurring, as they are synthetic. Table 1 provides the amino acid sequence of the “reference” polypeptide (SEQ ID NO:1), with the polypeptides of this first aspect of the disclosure including one or more amino acid change from SEQ ID NO:1 selected from the group consisting of K2T, K9R, K11T, K61D, E74D, T126D, E166K, S179K/N, T185K/N, E188K, A195K, and E198K. In various embodiments, the polypeptides of this first aspect of the disclosure include 1, 2, 3, 4, 5, 6, 7, 8, 9, 10, 11, or all 12 amino acid changes from SEQ ID NO:1 selected from the group consisting of K2T, K9R, K11T, K61D, E74D, T126D, E166K, S179K/N, T185K/N, E188K, A195K, and E198K.

The right hand column in Table 1 identifies the residue numbers in the reference polypeptide that were identified as conserved residues present at the interface of resulting assembled nanostructures of the disclosure (i.e.: “conserved interface residues”). In various embodiments, the isolated polypeptides of the first aspect of the disclosure have an amino acid sequence identical to the amino acid sequence of SEQ ID NO:1 at least at 1, 2, 3, or all 4 identified interface position selected from the group consisting of residues 25, 29, 33, and 54, and wherein the polypeptide is optionally identical to the amino acid sequence of SEQ ID NO:1 at residue 57 (a non-conserved interface residue).

Deep mutational scanning of the polypeptides of this first aspect and other aspects of the disclosure were carried out as described in the examples that follow, demonstrating the significant variation tolerated by the polypeptides without disrupting subsequent assembly into nanostructures. In one non-limiting embodiment of all the polypeptides of the disclosure, the recited permissible variation from the reference peptide (as opposed to the defined mutations) comprises conservative amino acid substitutions. As used here, “conservative amino acid substitution” means that: hydrophobic amino acids (Ala, Cys, Gly, Pro, Met, See, Sme, Val, Ile, Leu) can only be substituted with other hydrophobic amino acids; hydrophobic amino acids with bulky side chains (Phe, Tyr, Trp) can only be substituted with other hydrophobic amino acids with bulky side chains; amino acids with positively charged side chains (Arg, His, Lys) can only be substituted with other amino acids with positively charged side chains; amino acids with negatively charged side chains (Asp, Glu) can only be substituted with other amino acids with negatively charged side chains; and amino acids with polar uncharged side chains (Ser, Thr, Asn, Gln) can only be substituted with other amino acids with polar uncharged side chains.

In various specific embodiments, the polypeptides of this first aspect include a set of amino acid substitutions relative to SEQ ID NO:1 selected from the group consisting of:

(a) T126D, E166K, S179K, T185K, A195K, and E198K (corresponding to I53-50-v1 disclosed in the examples, which includes amino acid changes resulting in changes in the surface of the folded polypeptide);

(b) T126D, E166K, S179K/N, T185K/N, E188K, A195K, and E198K (corresponding to I53-50-v2 disclosed in the examples, which includes an additional amino acid change in a likely surface residue);

(c) K2T, K9R, K11T, K61D, T126D, E166K, S179K/N, T185K/N, E188K, A195K, and E198K (corresponding to I53-50-v3 disclosed in the examples, which includes changes in amino acid residues near the pore region);

(d) K2T, K9R, K11T, K61D, E74D, T126D, E166K, S179K/N, T185K/N, E188K, A195K, and E198K (corresponding to I53-50-v4 disclosed in the examples, which includes amino acid changes in exterio4 surface residues); and

(e) E74D, C76A, C100A, T126D, C165A, and C203A (including amino acid changes resulting in changes in the interior charge and exterior surface residues).

In one embodiment of any of the polypeptides of this first aspect, the polypeptide may have a N160C change relative to SEQ ID NO:1. In a further embodiment of any of the polypeptides of this first aspect, the polypeptides may include 1, 2, 3, 4, or all 5 or more of the following amino acid changes from SEQ ID NO:1: C76A, C100A, C165A, and C203A. In one specific embodiment, the polypeptides of this first aspect include each of the following amino acid substitutions relative to SEQ ID NO:1: K2T, K9R, K11T, K61D, E74D, T126D, E166K, S179N, T185N, E188K, A195K, and E198K.

In various further embodiments, the polypeptides of this first aspect comprises an amino acid sequence at least 50%, 60%, 70%, 75%, 80%, 85%, 90%, 95%, 96%, 97%, 98%, 99%, or 100% identical to the full length of the amino acid sequence of a polypeptide selected from the group consisting of SEQ ID NOS:5-14:

SEQ ID 05: I53-50-v4 trimeric component (sequences
in parentheses are optional)
(MTM)EELFKRHTIVAVLRANSVEEAIEKAVAVFAGGVHLIEITFTVPDA
DTVIKALSVLKEDGAIIGAGTVTSVDQCRKAVESGAEFIVSPHLDEEISQ
FCKEKGVFYMPGVMTPTELVKAMKLGHDILKLFPGEVVGPQFVKAMKGPF
PNVKFVPTGGVNLDNVCKWFKAGVLAVGVGNALVKGNPDKVREKAKKFVK
KIRGCTE(GS)
SEQ ID 06: I53-50-v1 trimeric component A
(MKM)EELFKKHKIVAVLRANSVEEAIEKAVAVFAGGVHLIEITFTVPDA
DTVIKALSVLKEKGAIIGAGTVTSVEQCRKAVESGAEFIVSPHLDEEISQ
FCKEKGVFYMPGVMTPTELVKAMKLGHDILKLFPGEVVGPQFVKAMKGPF
PNVKFVPTGGVNLDNVCKWFKAGVLAVGVGKALVKGKPDEVREKAKKFVK
KIRGCTE(GSWSHPQFEK)
SEQ ID 07: I53-50-v2 trimeric component A
(MKM)EELFKKHKIVAVLRANSVEEAIEKAVAVFAGGVHLIEITFTVPDA
DTVIKALSVLKEKGAIIGAGTVTSVEQCRKAVESGAEFIVSPHLDEEISQ
FCKEKGVFYMPGVMTPTELVKAMKLGHDILKLFPGEVVGPQFVKAMKGPF
PNVKFVPTGGVNLDNVCKWFKAGVLAVGVGNALVKGNPDKVREKAKKFVK
KIRGCTE(GSWSHPQFEK)
SEQ ID 08: I53-50-v3 trimeric component A
(MTM)EELFKRHTIVAVLRANSVEEAIEKAVAVFAGGVHLIEITFTVPDA
DTVIKALSVLKEDGAIIGAGTVTSVEQCRKAVESGAEFIVSPHLDEEISQ
FCKEKGVFYMPGVMTPTELVKAMKLGHDILKLFPGEVVGPQFVKAMKGPF
PNVKFVPTGGVNLDNVCKWFKAGVLAVGVGNALVKGNPDKVREKAKKFVK
KIRGCTE(GSWSHPQFEK)
SEQ ID 09: I53-50-v4 trimeric component with
helical linker
EKAAKAEEAAR(M)EELFKRHTIVAVLRANSVEEAIEKAVAVFAGGVHLI
EITFTVPDADTVIKALSVLKEDGAIIGAGTVTSVDQCRKAVESGAEFIVS
PHLDEEISQFCKEKGVFYMPGVMTPTELVKAMKLGHDILKLFPGEVVGPQ
FVKAMKGPFPNVKFVPTGGVNLDNVCKWFKAGVLAVGVGNALVKGNPDKV
REKAKKFVKKIRGCTE
SEQ ID 10: I53-50-v4 trimeric component with
helical linker, flexible linker, and 6xhis tag
GDGGRGSRGGDGSGGSSGEKAAKAEEAARIEELFKRHTIVAVLRANSVEE
AIEKAVAVFAGGVHLIEITFTVPDADTVIKALSVLKEDGAIIGAGTVTSV
DQCRKAVESGAEFIVSPHLDEEISQFCKEKGVFYMPGVMTPTELVKAMKL
GHDILKLFPGEVVGPQFVKAMKGPFPNVKFVPTGGVNLDNVCKWFKAGVL
AVGVGNALVKGNPDKVREKAKKFVKKIRGCTE(GSGLVPR)(GSLEHHHH
HH)
SEQ ID 11: v4_v0_cys_Trimer
(MKM)EELFKKHKIVAVLRANSVEEAIEKAVAVFAGGVHLIEITFTVPDA
DTVIKALSVLKEKGAIIGAGTVTSVDQARKAVESGAEFIVSPHLDEEISQ
FAKEKGVFYMPGVMTPTELVKAMKLGHDILKLFPGEVVGPQFVKAMKGPF
PNVKFVPTGGVCLDNVAEWFKAGVLAVGVGSALVKGTPDEVREKAKAFVE
KIRGATE(GS)
SEQ ID 12: v4_v0_cys_Pentamer
NQHSQKDQETVRIAVVRARWHAEIVDAAVSAFEAAMRKIGGERFAVDVFD
VPGAYEIPLHARTLAKTGRYGAVLGTAFVVNGGIYRHEFVASAVIDGMMN
VQLDTGVPVLSAVLTPHNYDDSDAHTLLFLALFAVKGMEAARAAVEILAA
REKIAAGS
SEQ ID 13: v4_v0_cys_Trimer_6x_Cys
MKMEELFKKHKIVAVLRANSVEEAIEKAVAVFAGGVHLIEITFTVPDADT
VIKALSVLKEKGAIIGAGTVTSVDQARKAVESGAEFIVSPHLDEEISQFA
KEKGVFYMPGVMTPTELVKAMKLGHDILKLFPGEVVGPQFVKAMKGPFPN
VKFVPTGGVCLDNVAEWFKAGVLAVGVGSALVKGTPDEVREKAKAFVEKI
RGATEGSGGGCGSGCGSGCGGGCGSGCGGGC
SEQ ID 14: v4_v0_cys_Trimer_2x_Cys_
MEELFKKHKIVAVLRANSVEEAIEKAVAVFAGGVHLIEITFTVPDADTVI
KALSVLKEKGAIIGAGTVTSVDQARKAVESGAEFIVSPHLDEEISQFAKE
KGVFYMPGVMTPTELVKAMKLGHDILKLFPGEVVGPQFVKAMKGPFPNVK
FVPTGGVCLDNVAEWFKAGVLAVGVGSALVKGTPDEVREKAKAFVEKIRG
ATEGSGGGCGSGC

In a second aspect, the disclosure provides isolated non-naturally occurring polypeptides comprising an amino acid sequence that are at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence of SEQ ID NO:2, wherein the polypeptide includes one or more amino acid change from SEQ ID NO:2 selected from the group consisting of H6Q, Y9H/Q, E24F/M, A38R, D39K, D43E, E67K, S105D, R119N, R121D, D122K, D124K/N, and H126K.

| | | Conserved |
|---|---|---|
| | | interface |
| Name | Amino acid sequence | residues |
| I53-50B | (M)NQHSHKDYETVRIAVVRARW | I53-50B: 132 |
| SEQ ID | HAEIVDACVSAFEAAMADIGGDR | Non-conserved |
| NO: 2 | FAVDVFDVPGAYEIPLHARTLAE | interface |
| PENTAMER | TGRYGAVLGTAFVVNGGIYRHEF | residues: |
| | VASAVIDGMMNVQLSTGVPVLSA | 24, 28, 36, 124, |
| | VLTPHRYRDSDAHTLLFLALFAV | 125, 127, 128, |
| | KGMEAARACVEILAAREKIAA | 129, 131, 133, |
| | | 135, 139 |

The polypeptides of this second aspect were designed for their ability to self-assemble in pairs with I53-50 trimer polypeptides disclosed herein to form significantly improved nanostructures disclosed herein. The polypeptides are non-naturally occurring, as they are synthetic. Table 2 provides the amino acid sequence of the “reference” polypeptide (SEQ ID NO:2), with the polypeptides of this first aspect of the disclosure including one or more amino acid change from SEQ ID NO:1 selected from the group consisting of H6Q, Y9H/Q, E24F/M, A38R, D39K, D43E, E67K, S105D, R119N, R121D, D122K, D124K/N, and H126K. In various embodiments, the polypeptides of this first aspect of the disclosure include 1, 2, 3, 4, 5, 6, 7, 8, 9, 10, 11, 12, or all 13 amino acid changes from SEQ ID NO:1 selected from the group consisting of H6Q, Y9H/Q, E24F/M, A38R, D39K, D43E, E67K, S105D, R119N, R121D, D122K, D124K/N, and H126K.

The right hand column in Table 2 identifies the residue numbers in the reference polypeptide that were identified as conserved residues present at the interface of resulting assembled nanostructures of the disclosure (i.e.: “conserved interface residues”). In various embodiments, the polypeptides of the second aspect of the disclosure have an amino acid sequence identical to the amino acid sequence of SEQ ID NO:2 at at residue 132. In various other embodiments, the polypeptides of the second aspect of the disclosure may be identical to SEQ ID NO:2 at 1, 2, 3, 4, 5, 6, 7, 8, 9, 10, 11, or all 12 identified non-conserved interface positions 24, 28, 36, 124, 125, 127, 128, 129, 131, 133, 135, and 139. In one specific embodiment, the amino acid sequence of the polypeptides of this second aspect are identical to the amino acid sequence of SEQ ID NO:2 at least at 1, 2, 3, 4, or all 5 identified interface positions selected from the group consisting of residues 128, 131, 132, 133, and 135.

In various specific embodiments, the polypeptides of this first aspect include a set of amino acid substitutions relative to SEQ ID NO:2 selected from the group consisting of:

(a) Y9H, A38R, S105D, R119N, R121D, D122K, and D124K (corresponding to I53-50-v1 disclosed in the examples, which includes amino acid changes resulting in changes in the surface of the folded polypeptide);

(b) Y9H, E24F/M, A38R, S105D, R119N, R121D, D122K, K124N, and H126K (corresponding to I53-50-v2 disclosed in the examples, which includes an additional amino acid change in a likely surface residue)

(c) H6Q, Y9H/Q, E24F/M, A38R, S105D, R119N, R121D, D122K, K124N, and H126K (corresponding to I53-50-v3 disclosed in the examples, which includes changes in surface amino acid residues); and

(d) H6Q, Y9H/Q, E24F/M, A38R, D39K, D43E, E67K, S105D, R119N, R121D, D122K, K124N, and H126K (corresponding to I53-50-v4 disclosed in the examples, which includes amino acid changes in exterio4 surface residues).

In one specific embodiment, the polypeptide includes each of the following amino acid substitutions relative to SEQ ID NO:2: H6Q, Y9Q, E24F A38R, D39K, D43E, E67K, S105D, R119N, R121D, D122K, K124N, and H126K.

In one embodiment of any polypeptides of the second aspect, the polypeptide may include 1 or both of the following amino acid changes from SEQ ID NO:2: C29A and C145A. In various other embodiments, the polypeptides of the second aspect comprise an amino acid sequence at least 50%, 60%, 70%, 75%, 80%, 85%, 90%, 95%, 96%, 97%, 98%, 99%, or 100% identical to the full length of the amino acid sequence of a polypeptide selected from the group consisting of SEQ ID NOS:15-21:

SEQ ID 15: I53-50-v4 pentameric component
(sequences in parentheses are optional)
(MGSSHHHHHHSSGLVPRGSEQKLISEEDLGS)NQHSQKDQETVRIAVVRA
RWHAFIVDACVSAFEAAMRKIGGERFAVDVFDVPGAYEIPLHARTLAKTGR
YGAVLGTAFVVNGGIYRHEFVASAVIDGMMNVQLDTGVPVLSAVLTPHNYD
KSNAKTLLFLALFAVKGMEAARACVEILAAREKIAA(GSLEGS)
SEQ ID 16: I53-50-v1 pentameric component B
(M)NQHSHKDHETVRIAVVRARWHAEIVDACVSAFEAAMRDIGGDRFAVDV
FDVPGAYEIPLHARTLAETGRYGAVLGTAFVVNGGIYRHEFVASAVIDGMM
NVQLDTGVPVLSAVLTPHNYDKSKAHTLLFLALFAVKGMEAARACVEILAA
REKIAA(GS)
SEQ ID 17: I53-50-v2 pentameric component B
(M)NQHSHKDHETVRIAVVRARWHAFIVDACVSAFEAAMRDIGGDRFAVDV
FDVPGAYEIPLHARTLAETGRYGAVLGTAFVVNGGIYRHEFVASAVIDGMM
NVQLDTGVPVLSAVLTPHNYDKSNAKTLLFLALFAVKGMEAARACVEILAA
REKIAA(GS)
SEQ ID 18: I53-50-v3 pentameric component B
(M)NQHSHKDHETVRIAVVRARWHAFIVDACVSAFEAAMRDIGGDRFAVDV
FDVPGAYEIPLHARTLAETGRYGAVLGTAFVVNGGIYRHEFVASAVIDGMM
NVQLDTGVPVLSAVLTPHNYDKSNAKTLLFLALFAVKGMEAARACVEILAA
REKIAA(GS)
SEQ ID 19: I53-50-v4 pentameric component with
C-terminal prfB linker (frameshifted)
(MGSSHHHHHHSSGLVPRGSEQKLISEEDLGS)NQHSQKDQETVRIAVVRA
RWHAFIVDACVSAFEAAMRKIGGERFAVDVFDVPGAYEIPLHARTLAKTGR
YGAVLGTAFVVNGGIYRHEFVASAVIDGMMNVQLDTGVPVLSAVLTPHNYD
KSNAKTLLFLALFAVKGMEAARACVEILAAREKIAA(GSLEGSRGYLDGSG
SGS)
SEQ ID 20: I53-50-v4 pentameric component with
C-terminal prfB linker (not frameshifted)
(MGSSHHHHHHSSGLVPRGSEQKLISEEDLGS)NQHSQKDQETVRIAVVRA
RWHAFIVDACVSAFEAAMRKIGGERFAVDVFDVPGAYEIPLHARTLAKTGR
YGAVLGTAFVVNGGIYRHEEVASAVIDGMMNVQLDTGVPVLSAVLTPHNYD
KSNAKTLLFLALFAVKGMEAARACVEILAAREKIAA(GSLEGSRGYL)
SEQ ID 21: v4_v0_cys_Pentamer
(M)NQHSQKDQETVRIAVVRARWHAEIVDAAVSAFEAAMRKIGGERFAVDV
FDVPGAYEIPLHARTLAKTGRYGAVLGTAFVVNGGIYRHEFVASAVIDGMM
NVQLDTGVPVLSAVLTPHNYDDSDAHTLLFLALFAVKGMEAARAAVEILAA
REKIAA(GS)

In a third aspect, the disclosure provides isolated non-naturally occurring polypeptides comprising an amino acid sequence that are at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence of SEQ ID NO:3, wherein the polypeptide includes one or more amino acid change from SEQ ID NO:3 selected from the group consisting of T13D, S71K, N101R, and D105K.

| | | Interface |
|---|---|---|
| Name | Amino acid sequence | residues |
| I53-47A | (M)PIFTLNTNIKATDVPSDFLSLTSRLVGL | I53-47A: |
| SEQ ID | ILSKPGSYVAVHINTDQQLSFGGSTNPAAFG | 22, 25, 29, |
| NO: 3 | TLMSIGGIEPSKNRDHSAVLFDHLNAMLGIP | 72, 79, 86, |
| TRIMER | KNRMYIHFVNLNGDDVGWNGTTF | 87 |

The polypeptides of third first aspect were designed for their ability to self-assemble in pairs with I53-47 pentamer polypeptides disclosed herein to form significantly improved nanostructures, including significant improved packaging of cargo such as RNA. The polypeptides are non-naturally occurring, as they are synthetic. Table 3 provides the amino acid sequence of the “reference” polypeptide (SEQ ID NO:3), with the polypeptides of this third aspect of the disclosure including one or more amino acid change from SEQ ID NO:3 selected from the group consisting of T13D, S71K, N101R, and D105K. In various embodiments, the polypeptides of this third aspect of the disclosure include 1, 2, 3, or all 4 amino acid changes from SEQ ID NO:3 selected from the group consisting of T13D, S71K, N101R, and D105K.

The right hand column in Table 3 identifies the residue numbers in the reference polypeptide that were identified as residues present at the interface of resulting assembled nanostructures of the disclosure (i.e.: “conserved interface residues”). In various embodiments, the polypeptides of the third aspect of the disclosure have an amino acid sequence identical to the amino acid sequence of SEQ ID NO:3 at least at 1, 2, 3, 4, 5, 6, or all 7, identified interface position selected from the group consisting of residues 22, 25, 29, 72, 79, 86, and 87. In a further embodiment, the polypeptides of this third aspect comprise an amino acid sequence at least 50%, 60%, 70%, 75%, 80%, 85%, 90%, 95%, 96%, 97%, 98%, 99%, or 100% identical to the full length of the amino acid sequence of SEQ ID NO: 22:

SEQ ID 22: I53-47-v1 trimeric component
(M)PIFTLNTNIKADDVPSDFLSLTSRLVGLILSKPGSYVAVHINTDQQLS
FGGSTNPAAFGTLMSIGGIEPKKNRDHSAVLFDHLNAMLGIPKNRMYIHFV
RLNGKDVGWNGTTF

In a fourth aspect, the disclosure provides isolated non-naturally occurring polypeptides comprising an amino acid sequence that are at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence of SEQ ID NO:4, wherein the polypeptide includes one or more amino acid change from SEQ ID NO:4 selected from the group consisting of S105D, R119N, R121D, D122K, A124K, and A150N.

| | | Interface |
|---|---|---|
| Name | Amino acid sequence | residues |
| I53-47B | (M)NQHSHKDHETVRIAVVRARWHADIV | I53-47B: |
| SEQ ID | DACVEAFEIAMAAIGGDRFAVDVFDVPG | 28, 31, 35, |
| NO: 4 | AYEIPLHARTLAETGRYGAVLGTAFVVN | 36, 39, |
| PENTAMER | GGIYRHEEVASAVIDGMMNVQLSTGVPV | 131, 132, |
| | LSAVLTPHRYRDSAEHHRFFAAHFAVKG | 135, 139, |
| | VEAARACIEILAAREKIAA | 146 |

The polypeptides of this fourth aspect were designed for their ability to self-assemble in pairs with I53-47 trimer polypeptides disclosed herein to form significantly improved nanostructures as disclosed herein. The polypeptides are non-naturally occurring, as they are synthetic. Table 4 provides the amino acid sequence of the “reference” polypeptide (SEQ ID NO:4), with the polypeptides of this fourth aspect of the disclosure including one or more amino acid change from SEQ ID NO:4 selected from the group consisting of S105D, R119N, R121D, D122K, A124K, and A150N. In various embodiments, the polypeptides of this fourth aspect of the disclosure include 1, 2, 3, 4, 5, or all 6 amino acid changes from SEQ ID NO:4 selected from the group consisting of S105D, R119N, R121D, D122K, A124K, and A150N.

The right hand column in Table 4 identifies the residue numbers in the reference polypeptide that were identified as residues present at the interface of resulting assembled nanostructures of the disclosure (i.e.: “interface residues”). In various embodiments, the polypeptides of the fourth aspect of the disclosure have an amino acid sequence identical to the amino acid sequence of SEQ ID NO:4 at least at 1, 2, 3, 4, 5, 6, 7, 8, 9, or all 10 identified interface position selected from the group consisting of residues 28, 31, 35, 36, 39, 131, 132, 135, 139, and 146. In a further embodiment, the polypeptides of this third aspect comprise an amino acid sequence at least 50%, 60%, 70%, 75%, 80%, 85%, 90%, 95%, 96%, 97%, 98%, 99%, or 100% identical to the full length of the amino acid sequence of SEQ ID NO: 23:

SEQ ID 23: I53-47-v1 pentameric component
(M)NQHSHKDHETVRIAVVRARWHADIVDACVEAFEIAMAAIGGDRFAVDV
FDVPGAYEIPLHARTLAETGRYGAVLGTAFVVNGGIYRHEFVASAVIDGMM
NVQLDTGVPVLSAVLTPHNYDKSKEHHRFFAAHFAVKGVEAARACIEILNA
REKIAA

In one embodiment of all four aspects of the polypeptides of the disclosure, the polypeptides may further comprise a targeting domain linked to the polypeptide. As used herein, a “targeting domain” is any moiety that can direct binding of the polypeptides to a target of interest. The inventors have discovered that one or more modular targeting domains can be incorporated (for example, operably linked, chemical conjugation, crosslinking, or the like) with the polypeptides and nanoparticles such that the one or more modular targeting domains are exposed on the exterior of nanoparticles without compromising the ability of the targeting domain to specifically bind to cells expressing its target. In this regard, the target can comprise, for example, a protein target, a small molecule target, a chemical target, an extracellular surface target, etc. The modular nature of the synthetic nanoparticles of the disclosure provides an advantage over existing viral capsids by allowing facile retargeting to alternative cells expressing different targets.

Any targeting domain may be used as suitable for an intended purpose. In one embodiment, the targeting domain may comprise a polypeptide targeting domain. In one such embodiment, the polypeptide targeting domain is a globular protein-binding domain that can fold and function on its own (i.e., the globular protein-binding domain can bind target with or without linkage to the polypeptides of the present disclosure. Such polypeptide binding domains are modular and can be readily swapped with other targeting domains. The targeting domain may be naturally occurring or designed.

In various other embodiments, the polypeptide targeting domain may comprise a polypeptide selected from the group consisting of an antibody, an scFv, a nanobody, a DARPin, an affibody, a monobody, adnectin, an alphabody, an albumin-binding domain, an adhiron, an affilin, an affimer, an affitin, an anticalin, an armadillo repeat proteins, a tetranectin, an avimer/maxibody, a centyrin, a fynomer, a kunitz domain, an obody/OB-fold, a PRONECTIN®, a repebody, CD47, an RNA binding domain, and a bovine immunodefficiency virus Tat RNA-binding peptide (Btat). In various specific embodiments, the polypeptide targeting domain comprises an amino acid sequence at least 50%, 60%, 70%, 80%, 90%, 95%, or 100% identical to the full length of the amino acid sequence selected from the group consisting of SEQ ID Nos. 24-43 (listed as Seq ID Nos. 7-17 or 65-67 in the priority application).

The specific amino acid sequences in the brackets can be changed depending on the desired binding specificity to a particular target.

SEQ ID 24 (Seq ID: Monobody targeting EphA2
VSDVPRDLEVVAATPTSLLISW[YYPFCAF]YYRITYGETGGNSPVQEFTV
P[RPSD]TATISGLKPGVDYTITVYAVT[CLGSYSR]PISINYRT
SEQ ID 25: Affibody targeting Her2
VDNKFNKE[MRN]A[YW]EI[AL]LPNLN[NQ]Q[KR]AFI[R]SL[Y]DD
PSQSANLLAEAKKLNDAQAPK
SEQ ID 26: DARPin targeting Her2
DLGKKLLEAAR[A]G[Q]DDEVRILMANGADVNA[K]D[EY]G[L]TPL
[Y]LA[TAHG]HLEIVEVLLK[N]G[A]DVNA[VDAI]G[F]TPLH[L]AA
[FIG]HLEI[AE]VLL[KH]GADVNA[QDKF]G[K]TAFDISIGNGNEDLA
EILQKLN
SEQ ID 27: Affibody targeting EGFR
VDNKFNKE[MWA]A[WE]EI[RN]LPNLN[GW]Q[MT]AFI[A]SL[V]DD
PSQSANLLAEAKKLNDAQAPK
SEQ ID 28: DARPin targeting EGFR
DLGKKLLEAAR[A]G[Q]DDEVRILMANGADVNA[D]D[TW]G[W]TPLHL
A[AYQG]HLEIVEVLLK[N]G[A]DVNA[YDYI]G[W]TPLH[L]AA[DG]
HLEI[VE]VLL[KN]GADVNA[SDYI]G[D]TPLHLAAHNGHLEIVEVLLK
HGADVNAQDKFGKTAFDISIDNGNEDLAEILQKLN
SEQ ID 29: spycatcher
GAMVDTLSGLSSEQGQSGDMTIEEDSATHIKFSKRDEDGKELAGATMELRD
SSGKTISTWISDGQVKDFYLYPGKYTFVETAAPDGYEVATAITFTVNEQGQ
VTVNGKATKGDAHIGS
SEQ ID 30: spytag
AHIVMVDAYKPTK
SEQ ID 31: scFv targeting CD3
DIKLQQSGAELARPGASVKMSCKTSG[YTFTRYTMH]WVKQRPGQGLEWIG
[YINPSRGYT]NYNQKFKDKATLTTDKSSSTAYMQLSSLTSEDSAVYYC[A
RYYDDHYCLDY]WGQGTTLTVSSGGGGSGGGGSGGGGSDIQLTQSPAIMSA
SPGEKVTMT[CRASSSVSYMN]WYQQKSGTSPK[RWIYDTSK]VASGVPYR
FSGSGSGTSYSLTISSMEAEDAA[TYYCQQWSSNPLT]FGAGTKLELK
SEQ ID 32: scFv targeting CD19
DIQMTQTTSSLSASLGDRVTIS[CRASQDISKYLN]WYQQKPDGTVK[LLI
YHTSR]LHSGVPSRFSGSGSGTDYSLTISNLEQEDIA[TYFCQQGNTLPY
T]FGGGTKLEITGGGGSGGGGSGGGGSEVKLQESGPGLVAPSQSLSVTCTV
SG[VSLPDYGVS]WIRQPPRKGLEWLG[VIWGSETT]YYNSALKSRLTIIK
DNSKSQVFLKMNSLQTDDTAIYYC[AKHYYYGGSYAMDY]WGQGTSVTVS
SEQ ID 33: Adnectin targeting EGER
GVSDVPRDLEVVAATPTELLISW[DSGRGSYQ]YYRITYGETGGNSPVQEF
TVP[GPVH]TATISGLIKPGVDYTITVYAVT[DHKPHADGPHTYHES]PIS
INYRTEIDKGSGC
SEQ ID 34: LaG17 nanobody targeting EGFP
MADVQLVESGGGLVQAGGSLRLSCAA[SGRTISMAA]MSWFRQAPGKEREF
VAGI[SRSAGSAVH]ADSVKGRFTISRDNTKNTLYLQMNSLKAEDTAVYYC
AV[RTSGFFGSIPRTGTAFDY]WGQGTQVTV

The listed amino acid positions (denoted with the letter “X”) for each class of binding domain can be mutated to other amino acids so as to change the binding properties of the protein. These mutations can include added or removed residues in addition to changes in amino acid identity:

SEQ ID 35: Monobody
23-29, 51-54, 76-82
VSDVPRDLEVVAATPTSLLISW[XXXXXXX]YYRITYGETGGNSPVQEFTV
P[XXXX]TATISGLKPGVDYTITVYAVT[XXXXXXX]PISINYRT
SEQ ID 36: Affibody
9-11, 13-14, 17-18, 24-25, 27-28, 32, 35
VDNKFNKE[XXX]A[XX]EI[XX]LPNLN[XX]Q[XX]AFI[X]SL[X]DD
PSQSANLLAEAKKLNDAQAPK
SEQ ID 37: Darpin
12, 14, 31, 33-34, 36, 40, 43-46, 57, 59, 64-67,
69, 74, 77-78, 83-84, 88-89, 96-99, 101
DLGKKLLEAAR[X]G[X]DDEVRILMANGADVNA[X]D[XX]G[X]TPLHL
A[XXXX]HLEIVEVLLK[X]G[X]DVNA[XXXX]G[X]TPLH[X]AA[XX]
HLEI[XX]VLL[XX]GADVNA[XXXX]G[X]TPLHLAAHNGHLEIVEVLLK
HGADVNAQDKFGKTAFDISIDNGNEDLAEILQKLN
SEQ ID 38: scFv (alternative linkers between the
heavy and light chains can substitute for the
(GGGGS)x3 linker indicated in parentheses.)
27-35, 50-58, 97-108, 157-167, 179-186, 218-230
DIKLQQSGAELARPGASVKMSCKTSG[XXXXXXXXX]WVKQRPGQGLEWIG
[XXXXXXXX]NYNQKFKDKATLTTDKSSSTAYMQLSSLTSEDSAVYYC[XX
XXXXXXXXXX]WGQGTTLTV(SSGGGGSGGGGSGGGGS)DIQLTQSPAIMS
ASPGEKVTMT[XXXXXXXXXXX]WYQQKSGTSPK[XXXXXXXX]VASGVPY
RFSGSGSGTSYSLTISSMEAEDAA[XXXXXXXXXXXXX]FGAGTKLELK
SEQ ID 39: adnectin
23-30, 52-55, 77-91
VSDVPRDLEVVAATPTSLLISW[XXXXXXXX]YYRITYGETGGNSPVQEFT
VP[XXXX]TATISGLKPGVDYTITVYAVT[XXXXXXXXXXXXXXX]PISIN
YRTEIDKGSGC
SEQ ID 40: nanobody
27-35, 54-62, 101-118
MADVQLVESGGGLVQAGGSLRLSCAA[XXXXXXXXX]MSWFRQAPGKEREF
VAGI[XXXXXXXXX]ADSVKGRFTISRDNTKNTLYLQMNSLKAEDTAVYYC
AV[XXXXXXXXXXXXXXXXXX]WGQGTQVTV
SEQ ID 41: spytag_CD19_scFv
AHIVMVDAYKPTKDIQMTQTTSSLSASLGDRVTISCRASQDISKYLNWYQQ
KPDGTVKLLIYHTSRLHSGVPSRFSGSGSGTDYSLTISNLEQEDIATYFCQ
QGNTLPYTFGGGTKLEITGGGGSGGGGSGGGGSEVKLQESGPGLVAPSQSL
SVTCTVSGVSLPDYGVSWIRQPPRKGLEWLGVIWGSETTYYNSALKSRLTI
IKDNSKSQVFLKMNSLQTDDTAIYYCAKHYYYGGSYAMDYWGQGTSVTVS
SEQ ID 42: spytag_CD3_scFv
AHIVMVDAYKPTKGSGDIKLQQSGAELARPGASVKMSCKTSGYTFTRYTMH
WVKQRPGQGLEWIGYINPSRGYTNYNQKFKDKATLTTDKSSSTAYMQLSSL
TSEDSAVYYCARYYDDHYCLDYWGQGTTLTVSSGGGGSGGGGSGGGGSDIQ
LTQSPAIMSASPGEKVTMTCRASSSVSYMNWYQQKSGTSPKRWIYDTSKVA
SGVPYRFSGSGSGTSYSLTISSMEAEDAATYYCQQWSSNPLTFGAGTKLEL
K
SEQ ID 43: spytag_LaG17_nanobody
AHIVMVDAYKPTKGSGMADVQLVESGGGLVQAGGSLRLSCAASGRTISMAA
MSWFRQAPGKEREFVAGISRSAGSAVHADSVKGRFTISRDNTKNTLYLQMN
SLKAEDTAVYYCAVRTSGFFGSIPRTGTAFDYWGQGTQVTV

In one embodiment, the polypeptide and the targeting domain may be linked by a non-covalent attachment. Any suitable non-covalent attachment may be used (ex: biotin-streptavidin linkers, etc.) In a further embodiment, the polypeptide and the targeting domain may be linked by a covalent attachment. Any suitable covalent attachment may be used, including but not limited to translational fusion (when the targeting domain is a polypeptide), and post-translational linkages, such as linkage through an amino acid side chain and a functional group (including but not limited to linkage between a cysteine side chain and a maleimide functional group or between a lysine die chain and NHS-ester functional group, or various post-translational enzymatic reactions including but not limited to sortase, split intein, SPYTAG®/SPYCATCHER®, etc.).

The targeting domain may be linked to the polypeptide of any of the four aspects of the disclosure at the N-terminus, the C-terminus, or both. In one embodiment, the polypeptides may comprise a peptide linker positioned between the polypeptide and the polypeptide targeting domain expressed as a translational fusion. Any linker may be used as suitable for an intended purpose; there is no specific amino acid residue or length requirement, as folded protein domains may be linked by a vast number of different polypeptide sequences while still retaining the same functional properties. In one embodiment, the peptide linker may comprise a frameshift sequence (i.e.: a linker that causes the ribosome to make a mistake and start translating in a different frame). This embodiment is useful for controlling valency of the targeting domain on the resulting nanostructures of the disclosure. In other specific embodiments, the peptide linker may comprise a peptide at least 50%, 60%, 70%, 80%, 90%, or 100% identical to the full length of the amino acid sequence selected from the group consisting of SEQ ID Nos. 44-57 (listed as Seq ID nos. 18-32 in the priority application):

(a) Glycine serine linkers may be of any length and are defined by high content of glycine and serine residues:

SEQ ID NO: 44:
GS
SEQ ID NO: 45:
GSGSGS
SEQ ID NO: 46:
GGSGGSGGS
SEQ ID NO: 47:
SGSGSG
SEQ ID NO: 48:
SSGSGGS

(b) Polyproline linkers are more rigid than glycine serine linkers: SEQ ID NO:49: PPPPPPP

(c) XTEN-like linkers are composed of mainly hydrophilic amino acids:

SEQ ID NO: 50:
STEEGTSESATPESGPGS
SEQ ID NO: 51:
EPATSGSETPGTSESATPES
SEQ ID NO: 52:
SPETSPASTEPEGS

(d) Polypeptide linker sequences capable of inducing frameshifting (post-frameshifting sequence is shown; All sequences in parentheses are optional)

SEQ ID NO: 53:
GSprfB (GSLEGS)RGYL(DGSGSGS)
SEQ ID NO: 54:
AtAOS-encoded amino acids YKKSRLGFRV(GGSGGS)
SEQ ID NO: 55:
Additional frameshift DNA sequence
AGYFLTYTPKSVTPDGVTLSQKTLTGAVG
(e) Helical Linker Sequence
EKAAKAEEAARI (SEQ ID NO: 56)
(f) Additional Linker Sequence
GDGGRGSRGGDGSGGSSG (SEQ ID NO: 57).

Thus, in various embodiments, the polypeptides may comprise a polypeptide that is at least 50%, 60%, 70%, 80%, 90%, or 100% identical to the full length of the amino acid sequence comprising (a) a polypeptide having the sequence of any one of SEQ ID NOS:5-23; (b) a targeting domain of any one of SEQ ID NOS:24-43; and (c) an optional linker according to any of SEQ ID NOS:44-57.

In various non-limiting embodiments, the polypeptides linked to targeting domains may comprise a polypeptide that is at least 50%, 60%, 70%, 80%, 90%, or 100 identical to the full length of the amino acid sequence selected from the group consisting of SEQ ID Nos.: 541-592:

Sequences of Binding Domains Translationally Fused to the C-Terminus of the Pentameric Subunit Via prfB Frameshift Linker
Underlined sequences are optional purification tags;
Bold sequences are optional myc tags;
Italics sequences are linkers;
All sequences in parentheses are optional;
Targeting domain sequences can have the same variable residues indicated in SEQ ID NOS:24-43

SEQ ID 541: I53-50-v4 pentamer_prfB_denovo_EphA2_monobody
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGYLDGSGSGSVSDVPRDLEVVAATPTSLLISWYYPECAFYYRITYGETGGNS
PVQEFTVPRPSDTATISGLKPGVDYTITVYAVTCLGSYSRPISINYRT
SEQ ID 542: I53-50-v4 pentamer_prfB_Her2_affibody
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGYLDGSGSGSVDNKFNKEMRNAYWEIALLPNLNNQQKRAFIRSLYDDPSQSA
NLLAEAKKLNDAQAPK
SEQ ID 543: I53-50-v4 pentamer_prfB_Her2_DARPin
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGYLDGSGSGSDLGKKLLEAARAGQDDEVRILMANGADVNAKDEYGLTPLYLA
TAHGHLEIVEVLLKNGADVNAVDAIGFTPLHLAAFIGHLEIAEVLLKHGADVNAQDKFGKTA
FDISIGNGNEDLAEILQKLN
SEQ ID 544: I53-50-v4 pentamer_prfB_EGFR_affibcdy
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGYLDGSGSGSVDNKFNKEMWAAWEEIRNLPNLNGWQMTAFIASLVDDPSQSA
NLLAEAKKLNDAQAPK
SEQ ID 545: I53-50-v4 pentamer_prfB_EGFR_DARPin
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGYLDGSGSGSDLGKKLLEAARAGQDDEVRILMANGADVNADDTWGWTPLHLA
AYQGHLEIVEVLLKNGADVNAYDYIGWTPLHLAADGHLEIVEVLLKNGADVNASDYIGDTPL
HLAAHNGHLEIVEVLLKHGADVNAQDKFGKTAFDISIDNGNEDLAEILQKLN
SEQ ID 546: I53-50-v4 pentamer_prfB_EGFR_adnectin
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGYLDGSGSGSGVSDVPRDLEVVAATPTSLLISWDSGRGSYQYYRITYGETGG
NSPVQEFTVPGPVHTATISGLKPGVDYTITVYAVTDHKPHADGPHTYHESPISINYRTEIDK
GSGC
SEQ ID 547: I53-50-v4 pentamer_prfB_spycatcher
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGYLDGSGSGSGAMVDTLSGLSSEQGQSGDMTIEEDSATHIKFSKRDEDGKEL
AGATMELRDSSGKTISTWISDGQVKDFYLYPGKYTFVETAAPDGYEVATAITFTVNEQGQVT
VNGKATKGDAHIGS
SEQ ID 548: I53-50-v4 pentamer_prfB_scFv_CD19
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGYLDGSGSGSDIQMTQTTSSLSASLGDRVTISCRASQDISKYLNWYQQKPDG
TVKLLIYHTSRLHSGVPSRFSGSGSGTDYSLTISNLEQEDIATYFCQQGNILPYTFGGGIKL
EITGGGGSGGGGSGGGGSEVKLQESGPGLVAPSQSLSVTCTVSGVSLPDYGVSWIRQPPRKG
LEWLGVIWGSETTYYNSALKSRLTIIKDNSKSQVFLKMNSLQTDDTAIYYCAKHYYYGGSYA
MDYWGQGTSVTVS
SEQ ID 549: I53-50-v4 pentamer_prfB_scFv_CD3
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGYLDGSGSGSDIKLQQSGAELARPGASVKMSCKTSGYTFTRYTMHWVKQRPG
QGLEWIGYINPSRGYTNYNQKFKDKATLTTDKSSSTAYMQLSSLTSEDSAVYYCARYYDDHY
CLDYWGQGTTLTVSSGGGGSGGGGSGGGGSDIQLTQSPAIMSASPGEKVTMTCRASSSVSYM
NWYQQKSGTSPKRWIYDTSKVASGVPYRFSGSGSGTSYSLTISSMEAEDAATYYCQQWSSNP
LTFGAGTKLELK
SEQ ID 550: I53-50-v4 pentamer_prfB_LaG17_FS_prfB
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGYLDGSGSGSMADVQLVESGGGLVQAGGSLRLSCAASGRTISMAAMSWFRQA
PGKEREFVAGISRSAGSAVHADSVKGRFTISRDNTKNTLYLQMNSLKAEDTAVYYCAVRTSG
FFGSIPRTGTAFDYWGQGTQVTV

Full valency binder sequences
(Underlined sequences are optional purification tags)
(Bold sequences are optional myc tags)
(Italics sequences are linkers)
(All sequences in parentheses are optional)
[binding domain sequences can have the same variable residues indicated in the “Polypeptide sequences of targeting domains” section]

SEQ ID 551: I53-50-v4 pentamer_prfB_Her2_affibody_fullvalency
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGNLDGSGSGSVDNKFNKEMRNAYWEIALLPNLNNQQKRAFIRSLYDDPSQSA
NLLAEAKKLNDAQAPK
SEQ ID 552: I53-50-v4 pentamer_prfB_Her2_DARPin_fullvalency
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGNLDGSGSGSDLGKKLLEAARAGQDDEVRILMANGADVNAKDEYGLTPLYLA
TAHGHLEIVEVLLKNGADVNAVDAIGFTPLHLAAFIGHLEIAEVLLKHGADVNAQDKFGKTA
FDISIGNGNEDLAEILQKLN
SEQ ID 553: I53-50-v4 pentamer_prfB_EGFR_affibody_fullvalency
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGNLDGSGSGSVDNKFNKEMWAAWEEIRNLPNLNGWQMTAFIASLVDDPSQSA
NLLAEAKKLNDAQAPK
SEQ ID 554: I53-50-v4 pentamer_prfB_EGFR_DARPin_fullvalency
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGNLDGSGSGSDLGKKLLEAARAGQDDEVRILMANGADVNADDTWGWTPLHLA
AYQGHLEIVEVLLKNGADVNAYDYIGWTPLHLAADGHLEIVEVLLKNGADVNASDYIGDTPL
HLAAHNGHLEIVEVLLKHGADVNAQDKFGKTAFDISIDNGNEDLAEILQKLN
SEQ ID 555: I53-50-v4 pentamer_prfB_EGFR_adnectin_fullvalency
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGNLDGSGSGSGVSDVPRDLEVVAATPTSLLISWDSGRGSYQYYRITYGETGG
NSPVQEFTVPGPVHTATISGLKPGVDYTITVYAVTDHKPHADGPHTYHESPISINYRTEIDK
GSGC
SEQ ID 556: I53-50-v4 pentamer_prfB_spycatcher_fullvalency
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGNLDGSGSGSGAMVDTLSGLSSEQGQSGDMTIEEDSATHIKFSKRDEDGKEL
AGATMELRDSSGKTISTWISDGQVKDFYLYPGKYTFVETAAPDGYEVATAITFTVNEQGQVT
VNGKATKGDAHIGS
SEQ ID 557: I53-50-v4 pentamer_prfB_CD3_scFv_fullvalency
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGNLDGSGSGSDIKLQQSGAELARPGASVKMSCKTSGYTFTRYTMHWVKQRPG
QGLEWIGYINPSRGYTNYNQKFKDKATLTTDKSSSTAYMQLSSLTSEDSAVYYCARYYDDHY
CLDYWGQGTTLTVSSGGGGSGGGGSGGGGSDIQLTQSPAIMSASPGEKVTMTCRASSSVSYM
NWYQQKSGTSPKRWIYDTSKVASGVPYRFSGSGSGTSYSLTISSMEAEDAATYYCQQWSSNP
LTFGAGTKLELK
SEQ ID 558: I53-50-v4 pentamer_prfB_CD19_scFv_fullvalency
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGNLDGSGSGSDIQMTQTTSSLSASLGDRVTISCRASQDISKYLNWYQQKPDG
TVKLLIYHTSRLHSGVPSRFSGSGSGTDYSLTISNLEQEDIATYFCQQGNTLPYTFGGGTKL
EITGGGGSGGGGSGGGGSEVKLQESGPGLVAPSQSLSVTCTVSGVSLPDYGVSWIRQPPRKG
LEWLGVIWGSETTYYNSALKSRLTIIKDNSKSQVFLKMNSLQTDDTAIYYCAKHYYYGGSYA
MDYWGQGTSVTVS
SEQ ID 559: I53-50-v4 pentamer_prfB_LaG17_nanobody_fullvalency
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGNLDGSGSGSMADVQLVESGGGLVQAGGSLRLSCAASGRTISMAAMSWFRQA
PGKEREFVAGISRSAGSAVHADSVKGRFTISRDNTKNTLYLQMNSLKAEDTAVYYCAVRTSG
FFGSIPRTGTAFDYWGQGTQVTV
SEQ ID 560: I53-50-v4 pentamer_prfB EGFR_Adnectin_fullvalency
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGNLDGSGSGSGVSDVPRDLEVVAATPTSLLISWDSGRGSYQYYRITYGETGG
NSPVQEFTVPGPVHTATISGLKPGVDYTITVYAVTDHKPHADGPHTYHESPISINYRTEIDK
GSGC
SEQ ID 561: I53-50-v4 pentamer_prfB_EphA2_Monobody_fullvalency
(MGSSHHHHHHSSGLVPRGSEQKLISEEDLGS)NQHSQKDQETVRIAVVRARWHAFIVDACV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDKSNAKTLLFLALFAVKGMEAARACVEILAAREK
IAAGSLEGSRGNLDGSGSGSVSDVPRDLEVVAATPTSLLISWYYPFCAFYYRITYGETGGNS
PVQEFTVPRPSDTATISGLKPGVDYTITVYAVTCLGSYSRPISINYRT
Pentamer_v4_v0_cys Fusion to Binding Domains
SEQ ID 562: I53-50-v4_v0 pentamer_prfB_EphA2_monobody
(MGSSHHHHHHSSGLVPRGSEQKLISEEDLGS)NQHSQKDQETVRIAVVRARWHAEIVDAAV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDDSDAHTLLFLALFAVKGMEAARAAVEILAAREK
IAAGSLEGSRGYLDGSGSGSVSDVPRDLEVVAATPTSLLISWYYPFCAFYYRITYGETGGNS
PVQEFTVPRPSDTATISGLKPGVDYTITVYAVTCLGSYSRPISINYRT
SEQ ID 563: I53-50-v4_v0 pentamer_prfB_Her2_affibody
(MGSSHHHHHHSSGLVPRGSEQKLISEEDLGS)NQHSQKDQETVRIAVVRARWHAEIVDAAV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDDSDAHTLLFLALFAVKGMEAARAAVEILAAREK
IAAGSLEGSRGYLDGSGSGSVDNKFNKEMRNAYWEIALLPNLNNQQKRAFIRSLYDDPSQSA
NLLAEAKKLNDAQAPK
SEQ ID 564: I53-50-v4_v0 pentamer_prfB_Her2_DARPin
(MGSSHHHHHHSSGLVPRGSEQKLISEEDLGS)NQHSQKDQETVRIAVVRARWHAEIVDAAV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDDSDAHTLLFLALFAVKGMEAARAAVEILAAREK
IAAGSLEGSRGYLDGSGSGSDLGKKLLEAARAGQDDEVRILMANGADVNAKDEYGLTPLYLA
TAHGHLEIVEVLLKNGADVNAVDAIGFTPLHLAAFIGHLEIAEVLLKHGADVNAQDKFGKTA
FDISIGNGNEDLAEILQKLN
SEQ ID 565: I53-50-v4_v0 pentamer_prfB_EGFR_affibody
(MGSSHHHHHHSSGLVPRGSEQKLISEEDLGS)NQHSQKDQETVRIAVVRARWHAEIVDAAV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDDSDAHTLLFLALFAVKGMEAARAAVEILAAREK
IAAGSLEGSRGYLDGSGSGSVDNKFNKEMWAAWEEIRNLPNLNGWQMTAFIASLVDDPSQSA
NLLAEAKKLNDAQAPK
SEQ ID 566: I53-50-v4_v0 pentamer_prfB_EGFR_DARPin
(MGSSHHHHHHSSGLVPRGSEQKLISEEDLGS)NQHSQKDQETVRIAVVRARWHAEIVDAAV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDDSDAHTLLFLALFAVKGMEAARAAVEILAAREK
IAAGSLEGSRGYLDGSGSGSDLGKKLLEAARAGQDDEVRILMANGADVNADDTWGWTPLHLA
AYQGHLEIVEVLLKNGADVNAYDYIGWTPLHLAADGHLEIVEVLLKNGADVNASDYIGDTPL
HLAAHNGHLEIVEVLLKHGADVNAQDKFGKTAFDISIDNGNEDLAEILQKLN
SEQ ID 567: I53-50-v4_v0 pentamer_prfB_EGFR_adnectin
(MGSSHHHHHHSSGLVPRGSEQKLISEEDLGS)NQHSQKDQETVRIAVVRARWHAEIVDAAV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDDSDAHTLLFLALFAVKGMEAARAAVEILAAREK
IAAGSLEGSRGYLDGSGSGSGVSDVPRDLEVVAATPTSLLISWDSGRGSYQYYRITYGETGG
NSPVQEFTVPGPVHTATISGLKPGVDYTITVYAVTDHKPHADGPHTYHESPISINYRTEIDK
GSGC
SEQ ID 568: I53-50-v4_v0 pentamer_prfB_spycatcher
(MGSSHHHHHHSSGLVPRGSEQKLISEEDLGS)NQHSQKDQETVRIAVVRARWHAEIVDAAV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDDSDAHTLLFLALFAVKGMEAARAAVEILAAREK
IAAGSLEGSRGYLDGSGSGSGAMVDTLSGLSSEQGQSGDMTIEEDSATHIKFSKRDEDGKEL
AGATMELRDSSGKTISTWISDGQVKDFYLYPGKYTFVETAAPDGYEVATAITFTVNEQGQVT
VNGKATKGDAHIGS
SEQ ID 569: I53-50-v4_v0 pentamer_prfB_scFv_CD19
(MGSSHHHHHHSSGLVPRGSEQKLISEEDLGS)NQHSQKDQETVRIAVVRARWHAEIVDAAV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDDSDAHTLLFLALFAVKGMEAARAAVEILAAREK
IAAGSLEGSRGYLDGSGSGSDIQMTQTTSSLSASLGDRVTISCRASQDISKYLNWYQQKPDG
TVKLLIYHTSRLHSGVPSRFSGSGSGTDYSLTISNLEQEDIATYFCQQGNTLPYTFGGGTKL
EITGGGGSGGGGSGGGGSEVKLQESGPGLVAPSQSLSVTCTVSGVSLPDYGVSWIRQPPRKG
LEWLGVIWGSETTYYNSALKSRLTIIKDNSKSQVFLKMNSLQTDDTAIYYCAKHYYYGGSYA
MDYWGQGTSVTVS
SEQ ID 570: I53-50-v4_v0 pentamer_prfB_scFv_CD3
(MGSSHHHHHHSSGLVPRGSEQKLISEEDLGS)NQHSQKDQETVRIAVVRARWHAEIVDAAV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDDSDAHTLLFLALFAVKGMEAARAAVEILAAREK
IAAGSLEGSRGYLDGSGSGSDIKLQQSGAELARPGASVKMSCKTSGYTFTRYTMHWVKQRPG
QGLEWIGYINPSRGYTNYNQKFKDKATLTTDKSSSTAYMQLSSLTSEDSAVYYCARYYDDHY
CLDYWGQGTTLTVSSGGGGSGGGGSGGGGSDIQLTQSPAIMSASPGEKVTMTCRASSSVSYM
NWYQQKSGTSPKRWIYDTSKVASGVPYRFSGSGSGTSYSLTISSMEAEDAATYYCQQWSSNP
LTFGAGTKLELK
SEQ ID 571: I53-50-v4_v0 pentamer_prfB_LaG17_FS_prfB
(MGSSHHHHHHSSGLVPRGSEQKLISEEDLGS)NQHSQKDQETVRIAVVRARWHAEIVDAAV
SAFEAAMRKIGGERFAVDVFDVPGAYEIPLHARTLAKTGRYGAVLGTAFVVNGGIYRHEFVA
SAVIDGMMNVQLDTGVPVLSAVLTPHNYDDSDAHTLLFLALFAVKGMEAARAAVEILAAREK
IAAGSLEGSRGYLDGSGSGSMADVQLVESGGGLVQAGGSLRLSCAASGRTISMAAMSWFRQA
PGKEREFVAGISRSAGSAVHADSVKGRFTISRDNTKNTLYLQMNSLKAEDTAVYYCAVRTSG
FFGSIPRTGTAFDYWGQGTQVTV
Trimer Fusions to binding domains
SEQ ID 572: I53-50-v4 trimeric component with Monobody
targeting EphA2
VSDVPRDLEVVAATPTSLLISWYYPFCAFYYRITYGETGGNSPVQEFTVPRPSDTATISGLK
PGVDYTITVYAVTCLGSYSRPISINYRT(GDGGRGSRGGDGSGGSSG)EKAAKAEEAARIEE
LFKRHTIVAVLRANSVEEAIEKAVAVFAGGVHLIEITFTVPDADTVIKALSVLKEDGAIIGA
GTVTSVDQCRKAVESGAEFIVSPHLDEEISQFCKEKGVFYMPGVMTPTELVKAMKLGHDILK
LFPGEVVGPQFVKAMKGPFPNVKFVPTGGVNLDNVCKWFKAGVLAVGVGNALVKGNPDKVRE
KAKKFVKKIRGCTEGSGLVPR(GSLEHHHHHH)
SEQ ID 573: I53-50-v4 trimeric component with Affibody
targeting Her2
VDNKFNKEMRNAYWEIALLPNLNNQQKRAFIRSLYDDPSQSANLLAEAKKLNDAQAPK(GDG
GRGSRGGDGSGGSSG)EKAAKAEEAARIEELFKRHTIVAVLRANSVEEAIEKAVAVFAGGVH
LIEITFTVPDADTVIKALSVLKEDGAIIGAGTVTSVDQCRKAVESGAEFIVSPHLDEEISQF
CKEKGVFYMPGVMTPTELVKAMKLGHDILKLFPGEVVGPQFVKAMKGPFPNVKFVPTGGVNL
DNVCKWFKAGVLAVGVGNALVKGNPDKVREKAKKFVKKIRGCTEGSGLVPR(GSLEHHHHHH)
SEQ ID 574: I53-50-v4 trimeric component with DARPin targeting
Her2
DLGKKLLEAARAGQDDEVRILMANGADVNAKDEYGLTPLYLATAHGHLEIVEVLLKNGADVN
AVDAIGFTPLHLAAFIGHLEIAEVLLKHGADVNAQDKFGKTAFDISIGNGNEDLAEILQKLN
(GDGGRGSRGGDGSGGSSG)EKAAKAEEAARIEELFKRHTIVAVLRANSVEEAIEKAVAVFA
GGVHLIEITFTVPDADTVIKALSVLKEDGAIIGAGTVTSVDQCRKAVESGAEFIVSPHLDEE
ISQFCKEKGVFYMPGVMTPTELVKAMKLGHDILKLFPGEVVGPQFVKAMKGPFPNVKFVPTG
GVNLDNVCKWFKAGVLAVGVGNALVKGNPDKVREKAKKFVKKIRGCTEGSGLVPR(GSLEHH
HHHH)
SEQ ID 575: I53-50-v4 trimeric component with Affibody
targeting EGFR
VDNKFNKEMWAAWEEIRNLPNLNGWQMTAFIASLVDDPSQSANLLAEAKKLNDAQAPK(GDG
GRGSRGGDGSGGSSG)EKAAKAEEAARIEELFKRHTIVAVLRANSVEEAIEKAVAVFAGGVH
LIEITFTVPDADTVIKALSVLKEDGAIIGAGTVTSVDQCRKAVESGAEFIVSPHLDEEISQF
CKEKGVFYMPGVMTPTELVKAMKLGHDILKLFPGEVVGPQFVKAMKGPFPNVKFVPTGGVNL
DNVCKWFKAGVLAVGVGNALVKGNPDKVREKAKKFVKKIRGCTEGSGLVPR(GSLEHHHHHH)
SEQ ID 576: I53-50-v4 trimeric component with DARPin targeting
EGFR
DLGKKLLEAARAGQDDEVRILMANGADVNADDTWGWTPLHLAAYQGHLEIVEVLLKNGADVN
AYDYIGWTPLHLAADGHLEIVEVLLKNGADVNASDYIGDTPLHLAAHNGHLEIVEVLLKHGA
DVNAQDKFGKTAFDISIDNGNEDLAEILQKLN(GDGGRGSRGGDGSGGSSG)EKAAKAEEAA
RIEELFKRHTIVAVLRANSVEEAIEKAVAVFAGGVHLIEITFTVPDADTVIKALSVLKEDGA
IIGAGTVTSVDQCRKAVESGAEFIVSPHLDEEISQFCKEKGVFYMPGVMTPTELVKAMKLGH
DILKLFPGEVVGPQFVKAMKGPFPNVKFVPTGGVNLDNVCKWFKAGVLAVGVGNALVKGNPD
KVREKAKKFVKKIRGCTEGSGLVPR(GSLEHHHHHH)
SEQ ID 577: I53-50-v4 trimeric component with spycatcher
GAMVDTLSGLSSEQGQSGDMTIEEDSATHIKFSKRDEDGKELAGATMELRDSSGKTISTWIS
DGQVKDFYLYPGKYTFVETAAPDGYEVATAITFTVNEQGQVTVNGKATKGDAHIGS(GDGGR
GSRGGDGSGGSSG)EKAAKAEEAARIEELFKRHTIVAVLRANSVEEAIEKAVAVFAGGVHLI
EITFTVPDADTVIKALSVLKEDGAIIGAGTVTSVDQCRKAVESGAEFIVSPHLDEEISQFCK
EKGVFYMPGVMTPTELVKAMKLGHDILKLFPGEVVGPQFVKAMKGPFPNVKFVPTGGVNLDN
VCKWFKAGVLAVGVGNALVKGNPDKVREKAKKFVKKIRGCTEGSGLVPR(GSLEHHHHHH)
SEQ ID 578: I53-50-v4 trimeric component with spytag
AHIVMVDAYKPTK(GDGGRGSRGGDGSGGSSG)EKAAKAEEAARIEELFKRHTIVAVLRANS
VEEAIEKAVAVFAGGVHLIEITFTVPDADTVIKALSVLKEDGAIIGAGTVTSVDQCRKAVES
GAEFIVSPHLDEEISQFCKEKGVFYMPGVMTPTELVKAMKLGHDILKLFPGEVVGPQFVKAM
KGPFPNVKFVPTGGVNLDNVCKWFKAGVLAVGVGNALVKGNPDKVREKAKKFVKKIRGCTEG
SGLVPR(GSLEHHHHHH)
SEQ ID 579: I53-50-v4 trimeric component with scFv targeting
CD3
DIKLQQSGAELARPGASVKMSCKTSGYTFTRYTMHWVKQRPGQGLEWIGYINPSRGYTNYNQ
KFKDKATLTTDKSSSTAYMQLSSLTSEDSAVYYCARYYDDHYCLDYWGQGTTLTVSSGGGGS
GGGGSGGGGSDIQLTQSPAIMSASPGEKVTMTCRASSSVSYMNWYQQKSGTSPKRWIYDTSK
VASGVPYRFSGSGSGTSYSLTISSMEAEDAATYYCQQWSSNPLTFGAGTKLELK(GDGGRGS
RGGDGSGGSSG)EKAAKAEEAARIEELFKRHTIVAVLRANSVEEAIEKAVAVFAGGVHLIEI
TFTVPDADTVIKALSVLKEDGAIIGAGTVTSVDQCRKAVESGAEFIVSPHLDEEISQFCKEK
GVFYMPGVMTPTELVKAMKLGHDILKLFPGEVVGPQFVKAMKGPFPNVKFVPTGGVNLDNVC
KWFKAGVLAVGVGNALVKGNPDKVREKAKKFVKKIRGCTEGSGLVPR(GSLEHHHHHH)
SEQ ID 580: I53-50-v4 trimeric component with scFv targeting
CD19
DIQMTQTTSSLSASLGDRVTISCRASQDISKYLNWYQQKPDGTVKLLIYHTSRLHSGVPSRF
SGSGSGTDYSLTISNLEQEDIATYFCQQGNTLPYTFGGGTKLEITGGGGSGGGGSGGGGSEV
KLQESGPGLVAPSQSLSVTCTVSGVSLPDYGVSWIRQPPRKGLEWLGVIWGSETTYYNSALK
SRLTIIKDNSKSQVFLKMNSLQTDDTAIYYCAKHYYYGGSYAMDYWGQGTSVTVS(GDGGRG
SRGGDGSGGSSG)EKAAKAEEAARIEELFKRHTIVAVLRANSVEEAIEKAVAVFAGGVHLIE
ITFTVPDADTVIKALSVLKEDGAIIGAGTVTSVDQCRKAVESGAEFIVSPHLDEEISQFCKE
KGVFYMPGVMTPTELVKAMKLGHDILKLFPGEVVGPQFVKAMKGPFPNVKFVPTGGVNLDNV
CKWFKAGVLAVGVGNALVKGNPDKVREKAKKFVKKIRGCTEGSGLVPR(GSLEHHHHHH)
SEQ ID 581: I53-50-v4 trimeric component with Adnectin
targeting EGFR
GVSDVPRDLEVVAATPTSLLISWDSGRGSYQYYRITYGETGGNSPVQEFTVPGPVHTATISG
LKPGVDYTITVYAVTDHKPHADGPHTYHESPISINYRTEIDKGSGC(GDGGRGSRGGDGSGG
SSG)EKAAKAEEAARIEELFKRHTIVAVLRANSVEEAIEKAVAVFAGGVHLIEITFTVPDAD
TVIKALSVLKEDGAIIGAGTVTSVDQCRKAVESGAEFIVSPHLDEEISQFCKEKGVFYMPGV
MTPTELVKAMKLGHDILKLFPGEVVGPQFVKAMKGPFPNVKFVPTGGVNLDNVCKWFKAGVL
AVGVGNALVKGNPDKVREKAKKFVKKIRGCTEGSGLVPR(GSLEHHHHHH)
SEQ ID 582: I53-50-v4 trimeric component with LaG17 nanobody
targeting EGFP
MADVQLVESGGGLVQAGGSLRLSCAASGRTISMAAMSWFRQAPGKEREFVAGISRSAGSAVH
ADSVKGRFTISRDNTKNTLYLQMNSLKAEDTAVYYCAVRTSGFFGSIPRTGTAFDYWGQGTQ
VTV(GDGGRGSRGGDGSGGSSG)EKAAKAEEAARIEELFKRHTIVAVLRANSVEEAIEKAVA
VFAGGVHLIEITFTVPDADTVIKALSVLKEDGAIIGAGTVTSVDQCRKAVESGAEFIVSPHL
DEEISQFCKEKGVFYMPGVMTPTELVKAMKLGHDILKLFPGEVVGPQFVKAMKGPFPNVKFV
PTGGVNLDNVCKWFKAGVLAVGVGNALVKGNPDKVREKAKKFVKKIRGCTEGSGLVPR(GSL
EHHHHHH)

Fusions of binding domains to N-terminus of trimer. Targeting domains are linked using a linker containing both an unstructured section and a helical section. As with other fusions, these linkers could be swapped out for many other linker types.

SEQ ID 583: I53-50-v4-ntrimer_scFv_CD3
DIKLQQSGAELARPGASVKMSCKTSGYTFTRYTMHWVKQRPGQGLEWIGYI
NPSRGYTNYNQKFKDKATLTTDKSSSTAYMQLSSLTSEDSAVYYCARYYDD
HYCLDYWGQGTTLTVSSGGGGSGGGGSGGGGSDIQLTQSPAIMSASPGEKV
TMTCRASSSVSYMNWYQQKSGTSPKRWIYDTSKVASGVPYRFSGSGSGTSY
SLTISSMEAEDAATYYCQQWSSNPLTFGAGTKLELK(GDGGRGSRGGDGSG
GSSGEKAAKAEEAARI)EELFKRHTIVAVLRANSVEEAIEKAVAVFAGGVH
LIEITFTVPDADTVIKALSVLKEDGAIIGAGTVTSVDQCRKAVESGAEFIV
SPHLDEEISQFCKEKGVFYMPGVMTPTELVKAMKLGHDILKLFPGEVVGPQ
FVKAMKGPFPNVKFVPTGGVNLDNVCKWFKAGVLAVGVGNALVGNPDKVRE
KAKKFVKKIRGCTE
SEQ ID 584: I53-50-v4-ntrimer_scFv_CD19
DIQMTQTTSSLSASLGDRVTISCRASQDISKYLNWYQQKPDGTVKLLIYHT
SRLHSGVPSRFSGSGSGTDYSLTISNLEQEDIATYFCQQGNTLPYTFGGGT
KLEITGGGGSGGGGSGGGGSEVKLQESGPGLVAPSQSLSVTCTVSGVSLPD
YGVSWIRQPPRKGLEWLGVIWGSETTYYNSALKSRLTIIKDNSKSQVFLKM
NSLQTDDTAIYYCAKHYYYGGSYAMDYWGQGTSVTVS(GDGGRGSRGGDGS
GGSSGEKAAKAEEAARI)EELFKRHTIVAVLRANSVEEAIEKAVAVFAGGV
HLIEITFTVPDADTVIKALSVLKEDGAIIGAGTVTSVDQCRKAVESGAEFI
VSPHLDEEISQFCKEKGVFYMPGVMTPTELVKAMKLGHDILKLFPGEVVGP
QFVKAMKGPFPNVKFVPTGGVNLDNVCKWFKAGVLAVGVGNALVKGNPDKV
REKAKKFVKKIRGCTE
SEQ ID 585: I53-50-v4-ntrimer_adnectin_EGFR
GSGVSDVPRDLEVVAATPTSLLISWDSGRGSYQYYRITYGETGGNSPVQEF
TVPGPVHTATISGLKPGVDYTITVYAVTDHKPHADGPHTYHESPISINYRT
EIDKG(GDGGRGSRGGDGSGGSSGEKAAKAEEAARI)EELFKRHTIVAVLR
ANSVEEAIEKAVAVFAGGVHLIEITFTVPDADTVIKALSVLKEDGAIIGAG
TVTSVDQCRKAVESGAEFIVSPHLDEEISQFCKEKGVFYMPGVMTPTELVK
AMKLGHDILKLFPGEVVGPQFVKAMKGPFPNVKFVPTGGVNLDNVCKWFKA
GVLAVGVGNALVKGNPDKVREKAKKFVKKIRGCTE
SEQ ID 586: I53-50-v4-ntrimer_darpin_EGFR
DLGKKLLEAARAGQDDEVRILMANGADVNADDTWGWTPLHLAAYQGHLEIV
EVLLKNGADVNAYDYIGWTPLHLAADGHLEIVEVLLKNGADVNASDYIGDT
PLHLAAHNGHLEIVEVLLKHGADVNAQDKFGKTAFDISIDNGNEDLAEILQ
KLN(GDGGRGSRGGDGSGGSSGEKAAKAEEAARI)EELFKRHTIVAVLRAN
SVEEAIEKAVAVFAGGVHLIEITFTVPDADTVIKALSVLKEDGAIIGAGTV
TSVDQCRKAVESGAEFIVSPHLDEEISQFCKEKGVFYMPGVMTPTELVKAM
KLGHDILKLFPGEVVGPQFVKAMKGPFPNVKFVPTGGVNLDNVCKWFKAGV
LAVGVGNALVKGNPDKVREKAKKFVKKIRGCTE
SEQ ID 587: I53-50-v4-ntrimer_monobody_EphAs
VSDVPRDLEVVAATPTSLLISWYYPFCAFYYRITYGETGGNSPVQEFTVPR
PSDTATISGLKPGVDYTITVYAVTCLGSYSRPISINYRT(GDGGRGSRGGD
GSGGSSGEKAAKAEEAARI)EELFKRHTIVAVLRANSVEEAIEKAVAVFAG
GVHLIEITFTVPDADTVIKALSVLKEDGAIIGAGTVTSVDQCRKAVESGAE
FIVSPHLDEEISQFCKEKGVFYMPGVMTPTELVKAMKLGHDILKLFPGEVV
GPQFVKAMKGPFPNVKFVPTGGVNLDNVCKWFKAGVLAVGVGNALVKGNPD
KVREKAKKFVKKIRGCTE
SEQ ID 588: I53-50-v4-ntrimer_affibody_Her2
VDNKFNKEMRNAYWEIALLPNLNNQQKRAFIRSLYDDPSQSANLLAEAKKL
NDAQAPK(GDGGRGSRGGDGSGGSSGEKAAKAEEAARI)EELFKRHTIVAV
LRANSVEEAIEKAVAVFAGGVHLIEITFTVPDADTVIKALSVLKEDGAIIG
AGTVTSVDQCRKAVESGAEFIVSPHLDEEISQFCKEKGVEYMPGVMTPTEL
VKAMKLGHDILKLFPGEVVGPQFVKAMKGPFPNVKFVPIGGVNLDNVCKWF
KAGVLAVGVGNALVKGNPDKVREKAKKFVKKIRGCTE
SEQ ID 589: I53-50-v4-ntrimer_darpin_Her2
DLGKKLLEAARAGQDDEVRILMANGADVNAKDEYGLTPLYLATAHGHLEIV
EVLLKNGADVNAVDAIGFTPLHLAAFIGHLEIAEVLLKHGADVNAQDKFGK
TAFDISIGNGNEDLAEILQKLN(GDGGRGSRGGDGSGGSSGEKAAKAEEAA
RI)EELFKRHTIVAVLRANSVEEAIEKAVAVFAGGVHLIEITFTVPDADTV
IKALSVLKEDGAIIGAGTVTSVDQCRKAVESGAEFIVSPHLDEEISQFCKE
KGVFYMPGVMTPTELVKAMKLGHDILKLFPGEVVGPQFVKAMKGPFPNVKF
VPTGGVNLDNVCKWFKAGVLAVGVGNALVKGNPDKVREKAKKFVKKIRGCT
E
SEQ ID 590: I53-50-v4-ntrimer_Nanobody_Lag17
MADVQLVESGGGLVQAGGSLRLSCAASGRTISMAAMSWFRQAPGKEREFVA
GISRSAGSAVHADSVKGRFTISRDNTKNTLYLQMNSLKAEDTAVYYCAVRT
SGFFGSIPRTGTAFDYWGQGTQVTV(GDGGRGSRGGDGSGGSSGEKAAKAE
EAARI)EELFKRHTIVAVLRANSVEEAIEKAVAVFAGGVHLIEITFTVPDA
DTVIKALSVLKEDGAIIGAGTVTSVDQCRKAVESGAEFIVSPHLDEEISQF
CKEKGVFYMPGVMTPTELVKAMKLGHDILKLFPGEVVGPQFVKAMKGPFPN
VKFVPTGGVNLDNVCKWFKAGVLAVGVGNALVKGNPDKVREKAKKFVKKIR
GCTE
SEQ ID 591: I53-50-v4-ntrimer_sGP7
EVQLQASGGGFVQPGGSLRLSCAASGFSSSNYAMGWFRQAPGKEREFVSAI
SRWDNVKAYYADSVKGRFTISRDNSKNTVYLQMNSLRAEDTATYYCAMVDD
YWDPGYWGQGTQVTV(GDGGRGSRGGDGSGGSSGEKAAKAEEAARI)EELF
KRHTIVAVLRANSVEEAIEKAVAVFAGGVHLIEITFTVPDADTVIKALSVL
KEDGAIIGAGTVTSVDQCRKAVESGAEFIVSPHLDEEISQFCKEKGVFYMP
GVMTPTELVKAMKLGHDILKLFPGEVVGPQFVKAMKGPFPNVKFVPTGGVN
LDNVCKWFKAGVLAVGVGNALVKGNPDKVREKAKKFVKKIRGCTE
SEQ ID 592: I53-50-v4-ntrimer_Spycatcher
GAMVDTLSGLSSEQGQSGDMTIEEDSATHIKFSKRDEDGKELAGATMELRD
SSGKTISTWISDGQVKDFYLYPGKYTFVETAAPDGYEVATAITFTVNEQGQ
VTVNGKATKGDAHIGS(GDGGRGSRGGDGSGGSSGEKAAKAEEAARI)EEL
FKRHTIVAVLRANSVEEAIEKAVAVFAGGVHLIEITFTVPDADTVIKALSV
LKEDGAIIGAGTVTSVDQCRKAVESGAEFIVSPHLDEEISQFCKEKGVFYM
PGVMTPTELVKAMKLGHDILKLFPGEVVGPQFVKAMKGPFPNVKFVPTGGV
NLDNVCKWFKAGVLAVGVGNALVKGNPDKVREKAKKFVKKIRGCTE

In another embodiment, the polypeptides of any aspect of the disclosure may further comprise a stabilization domain to limit/prevent unwanted interactions in vivo that induce clearance from circulation of nanostructures formed from the polypeptides. Any suitable stabilization domain may be used including but not limited to polyethylene glycol. In one embodiment, the stabilization domain comprises a polypeptide stabilization domain; such a polypeptide stabilization domain may be translationally fused to the polypeptide. In various exemplary embodiments, the polypeptide stabilization domain may comprise a peptide selected from the group consisting of SEQ ID NOS:58-518 and 593-595:

SEQ ID 58:
STEEGTSESATPESGPGSEPATSGSETPGTSESATPESGPGTSTEPSEGSAPGTSTEPE
SEQ ID 59:
GGSPGSPAGSPTSTEEGTSESATPESGPGTSTEPSEGSAPGSPAGSPTSTEEGTSTEPE
SEQ ID 60:
PASPASEPASPASEPASPASEPASPASEPASPASEPASPASEPASPASEPASPASEPASP
SEQ ID 61:
STEEGTSESATPESGPGSEPATSGSETPGTSESATPESGPGTSTEPSEGSAPGTSTEPESTE
EGTSESATPESGPGSEPATSGSETPGTSESATPESGPGTSTEPSEGSAPGTSTEPE
SEQ ID 62:
STEEGTSESATPESGPGSEPATSGSETPGTSESATPESGPGTSTEPSEGSAPGTSTEPEPAS
PASEPASPASEPASPASEPASPASEPASPASEPASPASEPASPASEPASPASEPAP
SEQ ID 63:
PETSPASTEPEGSPETSPASTEPEGSPETSPASTEPEGSPETSPASTEPEGSPETSPAS
SEQ ID 64:
PESTGAPGETSPEGSPESTGAPGETSPEGSPESTGAPGETSPEGSPESTGAPGETSPEG
SEQ ID 65:
SEGSAPGTSESATPESGPGTSESATPESGPGTSESATPESGPGSEPATSGSETPGSEPT
SEQ ID 66:
SGSEPEPTSPSETPSPPGGTPGSEATSPTEETGAEGPAGPGPGSEEGSTEGAGTSPEES
SEQ ID NO: 67:
DEADEADEADEADEADEADEADEADEADEADEADEADEADEADEADEADEADEADEADEA
SEQ ID NO: 68:
DEDEADEDEADEDEADEDEADEDEADEDEADEDEADEDEADEDEADEDEADEDEADEDEA
SEQ ID NO: 69:
DEDEDEDEDEADEDEDEDEDEADEDEDEDEDEADEDEDEDEDEADEDEDEDEDEADEDED
SEQ ID NO: 70:
DESDESDESDESDESDESDESDESDESDESDESDESDESDESDESDESDESDESDESDES
SEQ ID NO: 71:
DEDESDEDESDEDESDEDESDEDESDEDESDEDESDEDESDEDESDEDESDEDESDEDES
SEQ ID NO: 72:
DEDEDEDEDESDEDEDEDEDESDEDEDEDEDESDEDEDEDEDESDEDEDEDEDESDEDED
SEQ ID NO: 73:
DETDETDETDETDETDETDETDETDETDETDETDETDETDETDETDETDETDETDETDET
SEQ ID NO: 74:
DEDETDEDETDEDETDEDETDEDETDEDETDEDETDEDETDEDETDEDETDEDETDEDET
SEQ ID NO: 75:
DEDEDEDEDETDEDEDEDEDETDEDEDEDEDETDEDEDEDEDETDEDEDEDEDETDEDED
SEQ ID NO: 76:
DEEDEEDEEDEEDEEDEEDEEDEEDEEDEEDEEDEEDEEDEEDEEDEEDEEDEEDEEDEE
SEQ ID NO: 77:
DEDEEDEDEEDEDEEDEDEEDEDEEDEDEEDEDEEDEDEEDEDEEDEDEEDEDEEDEDEE
SEQ ID NO: 78:
DEDEDEDEDEEDEDEDEDEDEEDEDEDEDEDEEDEDEDEDEDEEDEDEDEDEDEEDEDED
SEQ ID NO: 79:
DEDDEDDEDDEDDEDDEDDEDDEDDEDDEDDEDDEDDEDDEDDEDDEDDEDDEDDEDDED
SEQ ID NO: 80:
DEDEDDEDEDDEDEDDEDEDDEDEDDEDEDDEDEDDEDEDDEDEDDEDEDDEDEDDEDED
SEQ ID NO: 81:
DEDEDEDEDEDDEDEDEDEDEDDEDEDEDEDEDDEDEDEDEDEDDEDEDEDEDEDDEDED
SEQ ID NO: 593:
DEQDEQDEQDEQDEQDEQDEQDEQDEQDEQDEQDEQDEQDEQDEQDEQDEQDEQDEQDEQ
SEQ ID NO: 82:
DEDEQDEDEQDEDEQDEDEQDEDEQDEDEQDEDEQDEDEQDEDEQDEDEQDEDEQDEDEQ
SEQ ID NO: 83:
DEDEDEDEDEQDEDEDEDEDEQDEDEDEDEDEQDEDEDEDEDEQDEDEDEDEDEQDEDED
SEQ ID NO: 84:
DENDENDENDENDENDENDENDENDENDENDENDENDENDENDENDENDENDENDENDEN
SEQ ID NO: 85:
DEDENDEDENDEDENDEDENDEDENDEDENDEDENDEDENDEDENDEDENDEDENDEDEN
SEQ ID NO: 86:
DEDEDEDEDENDEDEDEDEDENDEDEDEDEDENDEDEDEDEDENDEDEDEDEDENDEDED
SEQ ID NO: 87:
DEKDEKDEKDEKDEKDEKDEKDEKDEKDEKDEKDEKDEKDEKDEKDEKDEKDEKDEKDEK
SEQ ID NO: 88:
DEDEKDEDEKDEDEKDEDEKDEDEKDEDEKDEDEKDEDEKDEDEKDEDEKDEDEKDEDEK
SEQ ID NO: 89:
DEDEDEDEDEKDEDEDEDEDEKDEDEDEDEDEKDEDEDEDEDEKDEDEDEDEDEKDEDED
SEQ ID NO: 90:
DERDERDERDERDERDERDERDERDERDERDERDERDERDERDERDERDERDERDERDER
SEQ ID NO: 91:
DEDERDEDERDEDERDEDERDEDERDEDERDEDERDEDERDEDERDEDERDEDERDEDER
SEQ ID NO: 92:
DEDEDEDEDERDEDEDEDEDERDEDEDEDEDERDEDEDEDEDERDEDEDEDEDERDEDED
SEQ ID NO: 93:
DEPDEPDEPDEPDEPDEPDEPDEPDEPDEPDEPDEPDEPDEPDEPDEPDEPDEPDEPDEP
SEQ ID NO: 94:
DEDEPDEDEPDEDEPDEDEPDEDEPDEDEPDEDEPDEDEPDEDEPDEDEPDEDEPDEDEP
SEQ ID NO: 95:
DEDEDEDEDEPDEDEDEDEDEPDEDEDEDEDEPDEDEDEDEDEPDEDEDEDEDEPDEDED
SEQ ID NO: 96:
DEGDEGDEGDEGDEGDEGDEGDEGDEGDEGDEGDEGDEGDEGDEGDEGDEGDEGDEGDEG
SEQ ID NO: 97:
DEDEGDEDEGDEDEGDEDEGDEDEGDEDEGDEDEGDEDEGDEDEGDEDEGDEDEGDEDEG
SEQ ID NO: 98:
DEDEDEDEDEGDEDEDEDEDEGDEDEDEDEDEGDEDEDEDEDEGDEDEDEDEDEGDEDED
SEQ ID NO: 99:
DELDELDELDELDELDELDELDELDELDELDELDELDELDELDELDELDELDELDELDEL
SEQ ID NO: 100:
DEDELDEDELDEDELDEDELDEDELDEDELDEDELDEDELDEDELDEDELDEDELDEDEL
SEQ ID NO: 101:
DEDEDEDEDELDEDEDEDEDELDEDEDEDEDELDEDEDEDEDELDEDEDEDEDELDEDED
SEQ ID NO: 102:
DEIDEIDEIDEIDEIDEIDEIDEIDEIDEIDEIDEIDEIDEIDEIDEIDEIDEIDEIDEI
SEQ ID NO: 103:
DEDEIDEDEIDEDEIDEDEIDEDEIDEDEIDEDEIDEDEIDEDEIDEDEIDEDEIDEDEI
SEQ ID NO: 104:
DEDEDEDEDEIDEDEDEDEDEIDEDEDEDEDEIDEDEDEDEDEIDEDEDEDEDEIDEDED
SEQ ID NO: 105:
RKARKARKARKARKARKARKARKARKARKARKARKARKARKARKARKARKARKARKARKA
SEQ ID NO: 106:
RKRKARKRKARKRKARKRKARKRKARKRKARKRKARKRKARKRKARKRKARKRKARKRKA
SEQ ID NO: 594:
RKRKRKRKRKARKRKRKRKRKARKRKRKRKRKARKRKRKRKRKARKRKRKRKRKARKRKR
SEQ ID NO: 107:
RKSRKSRKSRKSRKSRKSRKSRKSRKSRKSRKSRKSRKSRKSRKSRKSRKSRKSRKSRKS
SEQ ID NO: 108:
RKRKSRKRKSRKRKSRKRKSRKRKSRKRKSRKRKSRKRKSRKRKSRKRKSRKRKSRKRKS
SEQ ID NO: 109:
RKRKRKRKRKSRKRKRKRKRKSRKRKRKRKRKSRKRKRKRKRKSRKRKRKRKRKSRKRKR
SEQ ID NO: 110:
RKTRKTRKTRKTRKTRKTRKTRKTRKTRKTRKTRKTRKTRKTRKTRKTRKTRKTRKTRKT
SEQ ID NO: 111:
RKRKTRKRKTRKRKTRKRKTRKRKTRKRKTRKRKTRKRKTRKRKTRKRKTRKRKTRKRKT
SEQ ID NO: 112:
RKRKRKRKRKTRKRKRKRKRKTRKRKRKRKRKTRKRKRKRKRKTRKRKRKRKRKTRKRKR
SEQ ID NO: 113:
RKERKERKERKERKERKERKERKERKERKERKERKERKERKERKERKERKERKERKERKE
SEQ ID NO: 114:
RKRKERKRKERKRKERKRKERKRKERKRKERKRKERKRKERKRKERKRKERKRKERKRKE
SEQ ID NO: 115:
RKRKRKRKRKERKRKRKRKRKERKRKRKRKRKERKRKRKRKRKERKRKRKRKRKERKRKR
SEQ ID NO: 116:
RKDRKDRKDRKDRKDRKDRKDRKDRKDRKDRKDRKDRKDRKDRKDRKDRKDRKDRKDRKD
SEQ ID NO: 117:
RKRKDRKRKDRKRKDRKRKDRKRKDRKRKDRKRKDRKRKDRKRKDRKRKDRKRKDRKRKD
SEQ ID NO: 118:
RKRKRKRKRKDRKRKRKRKRKDRKRKRKRKRKDRKRKRKRKRKDRKRKRKRKRKDRKRKR
SEQ ID NO: 119:
RKQRKQRKQRKQRKQRKQRKQRKQRKQRKQRKQRKQRKQRKQRKQRKQRKQRKQRKQRKQ
SEQ ID NO: 120:
RKRKQRKRKQRKRKQRKRKQRKRKQRKRKQRKRKQRKRKQRKRKQRKRKQRKRKQRKRKQ
SEQ ID NO: 121:
RKRKRKRKRKQRKRKRKRKRKQRKRKRKRKRKQRKRKRKRKRKQRKRKRKRKRKQRKRKR
SEQ ID NO: 122:
RKNRKNRKNRKNRKNRKNRKNRKNRKNRKNRKNRKNRKNRKNRKNRKNRKNRKNRKNRKN
SEQ ID NO: 123:
RKRKNRKRKNRKRKNRKRKNRKRKNRKRKNRKRKNRKRKNRKRKNRKRKNRKRKNRKRKN
SEQ ID NO: 124:
RKRKRKRKRKNRKRKRKRKRKNRKRKRKRKRKNRKRKRKRKRKNRKRKRKRKRKNRKRKR
SEQ ID NO: 125:
RKKRKKRKKRKKRKKRKKRKKRKKRKKRKKRKKRKKRKKRKKRKKRKKRKKRKKRKKRKK
SEQ ID NO: 126:
RKRKKRKRKKRKRKKRKRKKRKRKKRKRKKRKRKKRKRKKRKRKKRKRKKRKRKKRKRKK
SEQ ID NO: 127:
RKRKRKRKRKKRKRKRKRKRKKRKRKRKRKRKKRKRKRKRKRKKRKRKRKRKRKKRKRKR
SEQ ID NO: 128:
RKRRKRRKRRKRRKRRKRRKRRKRRKRRKRRKRRKRRKRRKRRKRRKRRKRRKRRKRRKR
SEQ ID NO: 129:
RKRKRRKRKRRKRKRRKRKRRKRKRRKRKRRKRKRRKRKRRKRKRRKRKRRKRKRRKRKR
SEQ ID NO: 130:
RKRKRKRKRKRRKRKRKRKRKRRKRKRKRKRKRRKRKRKRKRKRRKRKRKRKRKRRKRKR
SEQ ID NO: 131:
RKPRKPRKPRKPRKPRKPRKPRKPRKPRKPRKPRKPRKPRKPRKPRKPRKPRKPRKPRKP
SEQ ID NO: 132:
RKRKPRKRKPRKRKPRKRKPRKRKPRKRKPRKRKPRKRKPRKRKPRKRKPRKRKPRKRKP
SEQ ID NO: 133:
RKRKRKRKRKPRKRKRKRKRKPRKRKRKRKRKPRKRKRKRKRKPRKRKRKRKRKPRKRKR
SEQ ID NO: 134:
RKGRKGRKGRKGRKGRKGRKGRKGRKGRKGRKGRKGRKGRKGRKGRKGRKGRKGRKGRKG
SEQ ID NO: 135:
RKRKGRKRKGRKRKGRKRKGRKRKGRKRKGRKRKGRKRKGRKRKGRKRKGRKRKGRKRKG
SEQ ID NO: 136:
RKRKRKRKRKGRKRKRKRKRKGRKRKRKRKRKGRKRKRKRKRKGRKRKRKRKRKGRKRKR
SEQ ID NO: 137:
RKLRKLRKLRKLRKLRKLRKLRKLRKLRKLRKLRKLRKLRKLRKLRKLRKLRKLRKLRKL
SEQ ID NO: 138:
RKRKLRKRKLRKRKLRKRKLRKRKLRKRKLRKRKLRKRKLRKRKLRKRKLRKRKLRKRKL
SEQ ID NO: 139:
RKRKRKRKRKLRKRKRKRKRKLRKRKRKRKRKLRKRKRKRKRKLRKRKRKRKRKLRKRKR
SEQ ID NO: 140:
RKIRKIRKIRKIRKIRKIRKIRKIRKIRKIRKIRKIRKIRKIRKIRKIRKIRKIRKIRKI
SEQ ID NO: 141:
RKRKIRKRKIRKRKIRKRKIRKRKIRKRKIRKRKIRKRKIRKRKIRKRKIRKRKIRKRKI
SEQ ID NO: 142:
RKRKRKRKRKIRKRKRKRKRKIRKRKRKRKRKIRKRKRKRKRKIRKRKRKRKRKIRKRKR
SEQ ID NO: 143:
GSAGSAGSAGSAGSAGSAGSAGSAGSAGSAGSAGSAGSAGSAGSAGSAGSAGSAGSAGSA
SEQ ID NO: 144:
GSGSAGSGSAGSGSAGSGSAGSGSAGSGSAGSGSAGSGSAGSGSAGSGSAGSGSAGSGSA
SEQ ID NO: 145:
GSGSGSGSGSAGSGSGSGSGSAGSGSGSGSGSAGSGSGSGSGSAGSGSGSGSGSAGSGSG
SEQ ID NO: 146:
GSSGSSGSSGSSGSSGSSGSSGSSGSSGSSGSSGSSGSSGSSGSSGSSGSSGSSGSSGSS
SEQ ID NO: 147:
GSGSSGSGSSGSGSSGSGSSGSGSSGSGSSGSGSSGSGSSGSGSSGSGSSGSGSSGSGSS
SEQ ID NO: 148:
GSGSGSGSGSSGSGSGSGSGSSGSGSGSGSGSSGSGSGSGSGSSGSGSGSGSGSSGSGSG
SEQ ID NO: 149:
GSTGSTGSTGSTGSTGSTGSTGSTGSTGSTGSTGSTGSTGSTGSTGSTGSTGSTGSTGST
SEQ ID NO: 150:
GSGSTGSGSTGSGSTGSGSTGSGSTGSGSTGSGSTGSGSTGSGSTGSGSTGSGSTGSGST
SEQ ID NO: 151:
GSGSGSGSGSTGSGSGSGSGSTGSGSGSGSGSTGSGSGSGSGSTGSGSGSGSGSTGSGSG
SEQ ID NO: 152:
GSEGSEGSEGSEGSEGSEGSEGSEGSEGSEGSEGSEGSEGSEGSEGSEGSEGSEGSEGSE
SEQ ID NO: 153:
GSGSEGSGSEGSGSEGSGSEGSGSEGSGSEGSGSEGSGSEGSGSEGSGSEGSGSEGSGSE
SEQ ID NO: 154:
GSGSGSGSGSEGSGSGSGSGSEGSGSGSGSGSEGSGSGSGSGSEGSGSGSGSGSEGSGSG
SEQ ID NO: 155:
GSDGSDGSDGSDGSDGSDGSDGSDGSDGSDGSDGSDGSDGSDGSDGSDGSDGSDGSDGSD
SEQ ID NO: 156:
GSGSDGSGSDGSGSDGSGSDGSGSDGSGSDGSGSDGSGSDGSGSDGSGSDGSGSDGSGSD
SEQ ID NO: 157:
GSGSGSGSGSDGSGSGSGSGSDGSGSGSGSGSDGSGSGSGSGSDGSGSGSGSGSDGSGSG
SEQ ID NO: 158:
GSQGSQGSQGSQGSQGSQGSQGSQGSQGSQGSQGSQGSQGSQGSQGSQGSQGSQGSQGSQ
SEQ ID NO: 159:
GSGSQGSGSQGSGSQGSGSQGSGSQGSGSQGSGSQGSGSQGSGSQGSGSQGSGSQGSGSQ
SEQ ID NO: 160:
GSGSGSGSGSQGSGSGSGSGSQGSGSGSGSGSQGSGSGSGSGSQGSGSGSGSGSQGSGSG
SEQ ID NO: 161:
GSNGSNGSNGSNGSNGSNGSNGSNGSNGSNGSNGSNGSNGSNGSNGSNGSNGSNGSNGSN
SEQ ID NO: 162:
GSGSNGSGSNGSGSNGSGSNGSGSNGSGSNGSGSNGSGSNGSGSNGSGSNGSGSNGSGSN
SEQ ID NO: 163:
GSGSGSGSGSNGSGSGSGSGSNGSGSGSGSGSNGSGSGSGSGSNGSGSGSGSGSNGSGSG
SEQ ID NO: 164:
GSKGSKGSKGSKGSKGSKGSKGSKGSKGSKGSKGSKGSKGSKGSKGSKGSKGSKGSKGSK
SEQ ID NO: 165:
GSGSKGSGSKGSGSKGSGSKGSGSKGSGSKGSGSKGSGSKGSGSKGSGSKGSGSKGSGSK
SEQ ID NO: 166:
GSGSGSGSGSKGSGSGSGSGSKGSGSGSGSGSKGSGSGSGSGSKGSGSGSGSGSKGSGSG
SEQ ID NO: 167:
GSRGSRGSRGSRGSRGSRGSRGSRGSRGSRGSRGSRGSRGSRGSRGSRGSRGSRGSRGSR
SEQ ID NO: 168:
GSGSRGSGSRGSGSRGSGSRGSGSRGSGSRGSGSRGSGSRGSGSRGSGSRGSGSRGSGSR
SEQ ID NO: 169:
GSGSGSGSGSRGSGSGSGSGSRGSGSGSGSGSRGSGSGSGSGSRGSGSGSGSGSRGSGSG
SEQ ID NO: 170:
GSPGSPGSPGSPGSPGSPGSPGSPGSPGSPGSPGSPGSPGSPGSPGSPGSPGSPGSPGSP
SEQ ID NO: 171:
GSGSPGSGSPGSGSPGSGSPGSGSPGSGSPGSGSPGSGSPGSGSPGSGSPGSGSPGSGSP
SEQ ID NO: 172:
GSGSGSGSGSPGSGSGSGSGSPGSGSGSGSGSPGSGSGSGSGSPGSGSGSGSGSPGSGSG
SEQ ID NO: 173:
GSGGSGGSGGSGGSGGSGGSGGSGGSGGSGGSGGSGGSGGSGGSGGSGGSGGSGGSGGSG
SEQ ID NO: 174:
GSGSGGSGSGGSGSGGSGSGGSGSGGSGSGGSGSGGSGSGGSGSGGSGSGGSGSGGSGSG
SEQ ID NO: 175:
GSGSGSGSGSGGSGSGSGSGSGGSGSGSGSGSGGSGSGSGSGSGGSGSGSGSGSGGSGSG
SEQ ID NO: 176:
GSLGSLGSLGSLGSLGSLGSLGSLGSLGSLGSLGSLGSLGSLGSLGSLGSLGSLGSLGSL
SEQ ID NO: 177:
GSGSLGSGSLGSGSLGSGSLGSGSLGSGSLGSGSLGSGSLGSGSLGSGSLGSGSLGSGSL
SEQ ID NO: 178:
GSGSGSGSGSLGSGSGSGSGSLGSGSGSGSGSLGSGSGSGSGSLGSGSGSGSGSLGSGSG
SEQ ID NO: 179:
GSIGSIGSIGSIGSIGSIGSIGSIGSIGSIGSIGSIGSIGSIGSIGSIGSIGSIGSIGSI
SEQ ID NO: 180:
GSGSIGSGSIGSGSIGSGSIGSGSIGSGSIGSGSIGSGSIGSGSIGSGSIGSGSIGSGSI
SEQ ID NO: 181:
GSGSGSGSGSIGSGSGSGSGSIGSGSGSGSGSIGSGSGSGSGSIGSGSGSGSGSIGSGSG
SEQ ID NO: 182:
STASTASTASTASTASTASTASTASTASTASTASTASTASTASTASTASTASTASTASTA
SEQ ID NO: 183:
STSTASTSTASTSTASTSTASTSTASTSTASTSTASTSTASTSTASTSTASTSTASTSTA
SEQ ID NO: 184:
STSTSTSTSTASTSTSTSTSTASTSTSTSTSTASTSTSTSTSTASTSTSTSTSTASTSTS
SEQ ID NO: 185:
STSSTSSTSSTSSTSSTSSTSSTSSTSSTSSTSSTSSTSSTSSTSSTSSTSSTSSTSSTS
SEQ ID NO: 186:
STSTSSTSTSSTSTSSTSTSSTSTSSTSTSSTSTSSTSTSSTSTSSTSTSSTSTSSTSTS
SEQ ID NO: 187:
STSTSTSTSTSSTSTSTSTSTSSTSTSTSTSTSSTSTSTSTSTSSTSTSTSTSTSSTSTS
SEQ ID NO: 188:
STTSTTSTTSTTSTTSTTSTTSTTSTTSTTSTTSTTSTTSTTSTTSTTSTTSTTSTTSTT
SEQ ID NO: 189:
STSTTSTSTTSTSTTSTSTTSTSTTSTSTTSTSTTSTSTTSTSTTSTSTTSTSTTSTSTT
SEQ ID NO: 190:
STSTSTSTSTTSTSTSTSTSTTSTSTSTSTSTTSTSTSTSTSTTSTSTSTSTSTTSTSTS
SEQ ID NO: 191:
STESTESTESTESTESTESTESTESTESTESTESTESTESTESTESTESTESTESTESTE
SEQ ID NO: 192:
STSTESTSTESTSTESTSTESTSTESTSTESTSTESTSTESTSTESTSTESTSTESTSTE
SEQ ID NO: 193:
STSTSTSTSTESTSTSTSTSTESTSTSTSTSTESTSTSTSTSTESTSTSTSTSTESTSTS
SEQ ID NO: 194:
STDSTDSTDSTDSTDSTDSTDSTDSTDSTDSTDSTDSTDSTDSTDSTDSTDSTDSTDSTD
SEQ ID NO: 195:
STSTDSTSTDSTSTDSTSTDSTSTDSTSTDSTSTDSTSTDSTSTDSTSTDSTSTDSTSTD
SEQ ID NO: 196:
STSTSTSTSTDSTSTSTSTSTDSTSTSTSTSTDSTSTSTSTSTDSTSTSTSTSTDSTSTS
SEQ ID NO: 197:
STQSTQSTQSTQSTQSTQSTQSTQSTQSTQSTQSTQSTQSTQSTQSTQSTQSTQSTQSTQ
SEQ ID NO: 198:
STSTQSTSTQSTSTQSTSTQSTSTQSTSTQSTSTQSTSTQSTSTQSTSTQSTSTQSTSTQ
SEQ ID NO: 199:
STSTSTSTSTQSTSTSTSTSTQSTSTSTSTSTQSTSTSTSTSTQSTSTSTSTSTQSTSTS
SEQ ID NO: 200:
STNSTNSTNSTNSTNSTNSTNSTNSTNSTNSTNSTNSTNSTNSTNSTNSTNSTNSTNSTN
SEQ ID NO: 201:
STSTNSTSTNSTSTNSTSTNSTSTNSTSTNSTSTNSTSTNSTSTNSTSTNSTSTNSTSTN
SEQ ID NO: 202:
STSTSTSTSTNSTSTSTSTSTNSTSTSTSTSTNSTSTSTSTSTNSTSTSTSTSTNSTSTS
SEQ ID NO: 203:
STKSTKSTKSTKSTKSTKSTKSTKSTKSTKSTKSTKSTKSTKSTKSTKSTKSTKSTKSTK
SEQ ID NO: 204:
STSTKSTSTKSTSTKSTSTKSTSTKSTSTKSTSTKSTSTKSTSTKSTSTKSTSTKSTSTK
SEQ ID NO: 205:
STSTSTSTSTKSTSTSTSTSTKSTSTSTSTSTKSTSTSTSTSTKSTSTSTSTSTKSTSTS
SEQ ID NO: 206:
STRSTRSTRSTRSTRSTRSTRSTRSTRSTRSTRSTRSTRSTRSTRSTRSTRSTRSTRSTR
SEQ ID NO: 207:
STSTRSTSTRSTSTRSTSTRSTSTRSTSTRSTSTRSTSTRSTSTRSTSTRSTSTRSTSTR
SEQ ID NO: 208:
STSTSTSTSTRSTSTSTSTSTRSTSTSTSTSTRSTSTSTSTSTRSTSTSTSTSTRSTSTS
SEQ ID NO: 209:
STPSTPSTPSTPSTPSTPSTPSTPSTPSTPSTPSTPSTPSTPSTPSTPSTPSTPSTPSTP
SEQ ID NO: 210:
STSTPSTSTPSTSTPSTSTPSTSTPSTSTPSTSTPSTSTPSTSTPSTSTPSTSTPSTSTP
SEQ ID NO: 211:
STSTSTSTSTPSTSTSTSTSTPSTSTSTSTSTPSTSTSTSTSTPSTSTSTSTSTPSTSTS
SEQ ID NO: 212:
STGSTGSTGSTGSTGSTGSTGSTGSTGSTGSTGSTGSTGSTGSTGSTGSTGSTGSTGSTG
SEQ ID NO: 213:
STSTGSTSTGSTSTGSTSTGSTSTGSTSTGSTSTGSTSTGSTSTGSTSTGSTSTGSTSTG
SEQ ID NO: 214:
STSTSTSTSTGSTSTSTSTSTGSTSTSTSTSTGSTSTSTSTSTGSTSTSTSTSTGSTSTS
SEQ ID NO: 215:
STLSTLSTLSTLSTLSTLSTLSTLSTLSTLSTLSTLSTLSTLSTLSTLSTLSTLSTLSTL
SEQ ID NO: 216:
STSTLSTSTLSTSTLSTSTLSTSTLSTSTLSTSTLSTSTLSTSTLSTSTLSTSTLSTSTL
SEQ ID NO: 217:
STSTSTSTSTLSTSTSTSTSTLSTSTSTSTSTLSTSTSTSTSTLSTSTSTSTSTLSTSTS
SEQ ID NO: 218:
STISTISTISTISTISTISTISTISTISTISTISTISTISTISTISTISTISTISTISTI
SEQ ID NO: 219:
STSTISTSTISTSTISTSTISTSTISTSTISTSTISTSTISTSTISTSTISTSTISTSTI
SEQ ID NO: 220:
STSTSTSTSTISTSTSTSTSTISTSTSTSTSTISTSTSTSTSTISTSTSTSTSTISTSTS
SEQ ID NO: 221:
QNAQNAQNAQNAQNAQNAQNAQNAQNAQNAQNAQNAQNAQNAQNAQNAQNAQNAQNAQNA
SEQ ID NO: 222:
QNQNAQNQNAQNQNAQNQNAQNQNAQNQNAQNQNAQNQNAQNQNAQNQNAQNQNAQNQNA
SEQ ID NO: 223:
QNQNQNQNQNAQNQNQNQNQNAQNQNQNQNQNAQNQNQNQNQNAQNQNQNQNQNAQNQNQ
SEQ ID NO: 224:
QNSQNSQNSQNSQNSQNSQNSQNSQNSQNSQNSQNSQNSQNSQNSQNSQNSQNSQNSQNS
SEQ ID NO: 225:
QNQNSQNQNSQNQNSQNQNSQNQNSQNQNSQNQNSQNQNSQNQNSQNQNSQNQNSQNQNS
SEQ ID NO: 226:
QNQNQNQNQNSQNQNQNQNQNSQNQNQNQNQNSQNQNQNQNQNSQNQNQNQNQNSQNQNQ
SEQ ID NO: 227:
QNTQNTQNTQNTQNTQNTQNTQNTQNTQNTQNTQNTQNTQNTQNTQNTQNTQNTQNTQNT
SEQ ID NO: 228:
QNQNTQNQNTQNQNTQNQNTQNQNTQNQNTQNQNTQNQNTQNQNTQNQNTQNQNTQNQNT
SEQ ID NO: 229:
QNQNQNQNQNTQNQNQNQNQNTQNQNQNQNQNTQNQNQNQNQNTQNQNQNQNQNTQNQNQ
SEQ ID NO: 230:
QNEQNEQNEQNEQNEQNEQNEQNEQNEQNEQNEQNEQNEQNEQNEQNEQNEQNEQNEQNE
SEQ ID NO: 231:
QNQNEQNQNEQNQNEQNQNEQNQNEQNQNEQNQNEQNQNEQNQNEQNQNEQNQNEQNQNE
SEQ ID NO: 232:
QNQNQNQNQNEQNQNQNQNQNEQNQNQNQNQNEQNQNQNQNQNEQNQNQNQNQNEQNQNQ
SEQ ID NO: 233:
QNDQNDQNDQNDQNDQNDQNDQNDQNDQNDQNDQNDQNDQNDQNDQNDQNDQNDQNDQND
SEQ ID NO: 234:
QNQNDQNQNDQNQNDQNQNDQNQNDQNQNDQNQNDQNQNDQNQNDQNQNDQNQNDQNQND
SEQ ID NO: 235:
QNQNQNQNQNDQNQNQNQNQNDQNQNQNQNQNDQNQNQNQNQNDQNQNQNQNQNDQNQNQ
SEQ ID NO: 236:
QNQQNQQNQQNQQNQQNQQNQQNQQNQQNQQNQQNQQNQQNQQNQQNQQNQQNQQNQQNQ
SEQ ID NO: 237:
QNQNQQNQNQQNQNQQNQNQQNQNQQNQNQQNQNQQNQNQQNQNQQNQNQQNQNQQNQNQ
SEQ ID NO: 238:
QNQNQNQNQNQQNQNQNQNQNQQNQNQNQNQNQQNQNQNQNQNQQNQNQNQNQNQQNQNQ
SEQ ID NO: 239:
QNNQNNQNNQNNQNNQNNQNNQNNQNNQNNQNNQNNQNNQNNQNNQNNQNNQNNQNNQNN
SEQ ID NO: 240:
QNQNNQNQNNQNQNNQNQNNQNQNNQNQNNQNQNNQNQNNQNQNNQNQNNQNQNNQNQNN
SEQ ID NO: 241:
QNQNQNQNQNNQNQNQNQNQNNQNQNQNQNQNNQNQNQNQNQNNQNQNQNQNQNNQNQNQ
SEQ ID NO: 242:
QNKQNKQNKQNKQNKQNKQNKQNKQNKQNKQNKQNKQNKQNKQNKQNKQNKQNKQNKQNK
SEQ ID NO: 243:
QNQNKQNQNKQNQNKQNQNKQNQNKQNQNKQNQNKQNQNKQNQNKQNQNKQNQNKQNQNK
SEQ ID NO: 244:
QNQNQNQNQNKQNQNQNQNQNKQNQNQNQNQNKQNQNQNQNQNKQNQNQNQNQNKQNQNQ
SEQ ID NO: 245:
QNRQNRQNRQNRQNRQNRQNRQNRQNRQNRQNRQNRQNRQNRQNRQNRQNRQNRQNRQNR
SEQ ID NO: 246:
QNQNRQNQNRQNQNRQNQNRQNQNRQNQNRQNQNRQNQNRQNQNRQNQNRQNQNRQNQNR
SEQ ID NO: 247:
QNQNQNQNQNRQNQNQNQNQNRQNQNQNQNQNRQNQNQNQNQNRQNQNQNQNQNRQNQNQ
SEQ ID NO: 248:
QNPQNPQNPQNPQNPQNPQNPQNPQNPQNPQNPQNPQNPQNPQNPQNPQNPQNPQNPQNP
SEQ ID NO: 249:
QNQNPQNQNPQNQNPQNQNPQNQNPQNQNPQNQNPQNQNPQNQNPQNQNPQNQNPQNQNP
SEQ ID NO: 250:
QNQNQNQNQNPQNQNQNQNQNPQNQNQNQNQNPQNQNQNQNQNPQNQNQNQNQNPQNQNQ
SEQ ID NO: 251:
QNGQNGQNGQNGQNGQNGQNGQNGQNGQNGQNGQNGQNGQNGQNGQNGQNGQNGQNGQNG
SEQ ID NO: 252:
QNQNGQNQNGQNQNGQNQNGQNQNGQNQNGQNQNGQNQNGQNQNGQNQNGQNQNGQNQNG
SEQ ID NO: 253:
QNQNQNQNQNGQNQNQNQNQNGQNQNQNQNQNGQNQNQNQNQNGQNQNQNQNQNGQNQNQ
SEQ ID NO: 254:
QNLQNLQNLQNLQNLQNLQNLQNLQNLQNLQNLQNLQNLQNLQNLQNLQNLQNLQNLQNL
SEQ ID NO: 255:
QNQNLQNQNLQNQNLQNQNLQNQNLQNQNLQNQNLQNQNLQNQNLQNQNLQNQNLQNQNL
SEQ ID NO: 256:
QNQNQNQNQNLQNQNQNQNQNLQNQNQNQNQNLQNQNQNQNQNLQNQNQNQNQNLQNQNQ
SEQ ID NO: 257:
QNIQNIQNIQNIQNIQNIQNIQNIQNIQNIQNIQNIQNIQNIQNIQNIQNIQNIQNIQNI
SEQ ID NO: 258:
QNQNIQNQNIQNQNIQNQNIQNQNIQNQNIQNQNIQNQNIQNQNIQNQNIQNQNIQNQNI
SEQ ID NO: 259:
QNQNQNQNQNIQNQNQNQNQNIQNQNQNQNQNIQNQNQNQNQNIQNQNQNQNQNIQNQNQ
SEQ ID NO: 260:
GEAGEAGEAGEAGEAGEAGEAGEAGEAGEAGEAGEAGEAGEAGEAGEAGEAGEAGEAGEA
SEQ ID NO: 261:
GEGEAGEGEAGEGEAGEGEAGEGEAGEGEAGEGEAGEGEAGEGEAGEGEAGEGEAGEGEA
SEQ ID NO: 262:
GEGEGEGEGEAGEGEGEGEGEAGEGEGEGEGEAGEGEGEGEGEAGEGEGEGEGEAGEGEG
SEQ ID NO: 263:
GESGESGESGESGESGESGESGESGESGESGESGESGESGESGESGESGESGESGESGES
SEQ ID NO: 264:
GEGESGEGESGEGESGEGESGEGESGEGESGEGESGEGESGEGESGEGESGEGESGEGES
SEQ ID NO: 265:
GEGEGEGEGESGEGEGEGEGESGEGEGEGEGESGEGEGEGEGESGEGEGEGEGESGEGEG
SEQ ID NO: 266:
GETGETGETGETGETGETGETGETGETGETGETGETGETGETGETGETGETGETGETGET
SEQ ID NO: 267:
GEGETGEGETGEGETGEGETGEGETGEGETGEGETGEGETGEGETGEGETGEGETGEGET
SEQ ID NO: 268:
GEGEGEGEGETGEGEGEGEGETGEGEGEGEGETGEGEGEGEGETGEGEGEGEGETGEGEG
SEQ ID NO: 269:
GEEGEEGEEGEEGEEGEEGEEGEEGEEGEEGEEGEEGEEGEEGEEGEEGEEGEEGEEGEE
SEQ ID NO: 270:
GEGEEGEGEEGEGEEGEGEEGEGEEGEGEEGEGEEGEGEEGEGEEGEGEEGEGEEGEGEE
SEQ ID NO: 271:
GEGEGEGEGEEGEGEGEGEGEEGEGEGEGEGEEGEGEGEGEGEEGEGEGEGEGEEGEGEG
SEQ ID NO: 272:
GEDGEDGEDGEDGEDGEDGEDGEDGEDGEDGEDGEDGEDGEDGEDGEDGEDGEDGEDGED
SEQ ID NO: 273:
GEGEDGEGEDGEGEDGEGEDGEGEDGEGEDGEGEDGEGEDGEGEDGEGEDGEGEDGEGED
SEQ ID NO: 274:
GEGEGEGEGEDGEGEGEGEGEDGEGEGEGEGEDGEGEGEGEGEDGEGEGEGEGEDGEGEG
SEQ ID NO: 275:
GEQGEQGEQGEQGEQGEQGEQGEQGEQGEQGEQGEQGEQGEQGEQGEQGEQGEQGEQGEQ
SEQ ID NO: 276:
GEGEQGEGEQGEGEQGEGEQGEGEQGEGEQGEGEQGEGEQGEGEQGEGEQGEGEQGEGEQ
SEQ ID NO: 277:
GEGEGEGEGEQGEGEGEGEGEQGEGEGEGEGEQGEGEGEGEGEQGEGEGEGEGEQGEGEG
SEQ ID NO: 278:
GENGENGENGENGENGENGENGENGENGENGENGENGENGENGENGENGENGENGENGEN
SEQ ID NO: 279:
GEGENGEGENGEGENGEGENGEGENGEGENGEGENGEGENGEGENGEGENGEGENGEGEN
SEQ ID NO: 280:
GEGEGEGEGENGEGEGEGEGENGEGEGEGEGENGEGEGEGEGENGEGEGEGEGENGEGEG
SEQ ID NO: 281:
GEKGEKGEKGEKGEKGEKGEKGEKGEKGEKGEKGEKGEKGEKGEKGEKGEKGEKGEKGEK
SEQ ID NO: 282:
GEGEKGEGEKGEGEKGEGEKGEGEKGEGEKGEGEKGEGEKGEGEKGEGEKGEGEKGEGEK
SEQ ID NO: 283:
GEGEGEGEGEKGEGEGEGEGEKGEGEGEGEGEKGEGEGEGEGEKGEGEGEGEGEKGEGEG
SEQ ID NO: 284:
GERGERGERGERGERGERGERGERGERGERGERGERGERGERGERGERGERGERGERGER
SEQ ID NO: 285:
GEGERGEGERGEGERGEGERGEGERGEGERGEGERGEGERGEGERGEGERGEGERGEGER
SEQ ID NO: 286:
GEGEGEGEGERGEGEGEGEGERGEGEGEGEGERGEGEGEGEGERGEGEGEGEGERGEGEG
SEQ ID NO: 287:
GEPGEPGEPGEPGEPGEPGEPGEPGEPGEPGEPGEPGEPGEPGEPGEPGEPGEPGEPGEP
SEQ ID NO: 288:
GEGEPGEGEPGEGEPGEGEPGEGEPGEGEPGEGEPGEGEPGEGEPGEGEPGEGEPGEGEP
SEQ ID NO: 289:
GEGEGEGEGEPGEGEGEGEGEPGEGEGEGEGEPGEGEGEGEGEPGEGEGEGEGEPGEGEG
SEQ ID NO: 290:
GEGGEGGEGGEGGEGGEGGEGGEGGEGGEGGEGGEGGEGGEGGEGGEGGEGGEGGEGGEG
SEQ ID NO: 291:
GEGEGGEGEGGEGEGGEGEGGEGEGGEGEGGEGEGGEGEGGEGEGGEGEGGEGEGGEGEG
SEQ ID NO: 292:
GEGEGEGEGEGGEGEGEGEGEGGEGEGEGEGEGGEGEGEGEGEGGEGEGEGEGEGGEGEG
SEQ ID NO: 293:
GELGELGELGELGELGELGELGELGELGELGELGELGELGELGELGELGELGELGELGEL
SEQ ID NO: 294:
GEGELGEGELGEGELGEGELGEGELGEGELGEGELGEGELGEGELGEGELGEGELGEGEL
SEQ ID NO: 295:
GEGEGEGEGELGEGEGEGEGELGEGEGEGEGELGEGEGEGEGELGEGEGEGEGELGEGEG
SEQ ID NO: 296:
GEIGEIGEIGEIGEIGEIGEIGEIGEIGEIGEIGEIGEIGEIGEIGEIGEIGEIGEIGEI
SEQ ID NO: 297:
GEGEIGEGEIGEGEIGEGEIGEGEIGEGEIGEGEIGEGEIGEGEIGEGEIGEGEIGEGEI
SEQ ID NO: 298:
GEGEGEGEGEIGEGEGEGEGEIGEGEGEGEGEIGEGEGEGEGEIGEGEGEGEGEIGEGEG
SEQ ID NO: 299:
EKAEKAEKAEKAEKAEKAEKAEKAEKAEKAEKAEKAEKAEKAEKAEKAEKAEKAEKAEKA
SEQ ID NO: 300:
EKEKAEKEKAEKEKAEKEKAEKEKAEKEKAEKEKAEKEKAEKEKAEKEKAEKEKAEKEKA
SEQ ID NO: 301:
EKEKEKEKEKAEKEKEKEKEKAEKEKEKEKEKAEKEKEKEKEKAEKEKEKEKEKAEKEKE
SEQ ID NO: 302:
EKSEKSEKSEKSEKSEKSEKSEKSEKSEKSEKSEKSEKSEKSEKSEKSEKSEKSEKSEKS
SEQ ID NO: 303:
EKEKSEKEKSEKEKSEKEKSEKEKSEKEKSEKEKSEKEKSEKEKSEKEKSEKEKSEKEKS
SEQ ID NO: 304:
EKEKEKEKEKSEKEKEKEKEKSEKEKEKEKEKSEKEKEKEKEKSEKEKEKEKEKSEKEKE
SEQ ID NO: 305:
EKTEKTEKTEKTEKTEKTEKTEKTEKTEKTEKTEKTEKTEKTEKTEKTEKTEKTEKTEKT
SEQ ID NO: 306:
EKEKTEKEKTEKEKTEKEKTEKEKTEKEKTEKEKTEKEKTEKEKTEKEKTEKEKTEKEKT
SEQ ID NO: 307:
EKEKEKEKEKTEKEKEKEKEKTEKEKEKEKEKTEKEKEKEKEKTEKEKEKEKEKTEKEKE
SEQ ID NO: 308:
EKEEKEEKEEKEEKEEKEEKEEKEEKEEKEEKEEKEEKEEKEEKEEKEEKEEKEEKEEKE
SEQ ID NO: 309:
EKEKEEKEKEEKEKEEKEKEEKEKEEKEKEEKEKEEKEKEEKEKEEKEKEEKEKEEKEKE
SEQ ID NO: 310:
EKEKEKEKEKEEKEKEKEKEKEEKEKEKEKEKEEKEKEKEKEKEEKEKEKEKEKEEKEKE
SEQ ID NO: 311:
EKDEKDEKDEKDEKDEKDEKDEKDEKDEKDEKDEKDEKDEKDEKDEKDEKDEKDEKDEKD
SEQ ID NO: 312:
EKEKDEKEKDEKEKDEKEKDEKEKDEKEKDEKEKDEKEKDEKEKDEKEKDEKEKDEKEKD
SEQ ID NO: 313:
EKEKEKEKEKDEKEKEKEKEKDEKEKEKEKEKDEKEKEKEKEKDEKEKEKEKEKDEKEKE
SEQ ID NO: 314:
EKQEKQEKQEKQEKQEKQEKQEKQEKQEKQEKQEKQEKQEKQEKQEKQEKQEKQEKQEKQ
SEQ ID NO: 315:
EKEKQEKEKQEKEKQEKEKQEKEKQEKEKQEKEKQEKEKQEKEKQEKEKQEKEKQEKEKQ
SEQ ID NO: 316:
EKEKEKEKEKQEKEKEKEKEKQEKEKEKEKEKQEKEKEKEKEKQEKEKEKEKEKQEKEKE
SEQ ID NO: 317:
EKNEKNEKNEKNEKNEKNEKNEKNEKNEKNEKNEKNEKNEKNEKNEKNEKNEKNEKNEKN
SEQ ID NO: 318:
EKEKNEKEKNEKEKNEKEKNEKEKNEKEKNEKEKNEKEKNEKEKNEKEKNEKEKNEKEKN
SEQ ID NO: 319:
EKEKEKEKEKNEKEKEKEKEKNEKEKEKEKEKNEKEKEKEKEKNEKEKEKEKEKNEKEKE
SEQ ID NO: 320:
EKKEKKEKKEKKEKKEKKEKKEKKEKKEKKEKKEKKEKKEKKEKKEKKEKKEKKEKKEKK
SEQ ID NO: 321:
EKEKKEKEKKEKEKKEKEKKEKEKKEKEKKEKEKKEKEKKEKEKKEKEKKEKEKKEKEKK
SEQ ID NO: 322:
EKEKEKEKEKKEKEKEKEKEKKEKEKEKEKEKKEKEKEKEKEKKEKEKEKEKEKKEKEKE
SEQ ID NO: 323:
EKREKREKREKREKREKREKREKREKREKREKREKREKREKREKREKREKREKREKREKR
SEQ ID NO: 324:
EKEKREKEKREKEKREKEKREKEKREKEKREKEKREKEKREKEKREKEKREKEKREKEKR
SEQ ID NO: 325:
EKEKEKEKEKREKEKEKEKEKREKEKEKEKEKREKEKEKEKEKREKEKEKEKEKREKEKE
SEQ ID NO: 326:
EKPEKPEKPEKPEKPEKPEKPEKPEKPEKPEKPEKPEKPEKPEKPEKPEKPEKPEKPEKP
SEQ ID NO: 327:
EKEKPEKEKPEKEKPEKEKPEKEKPEKEKPEKEKPEKEKPEKEKPEKEKPEKEKPEKEKP
SEQ ID NO: 328:
EKEKEKEKEKPEKEKEKEKEKPEKEKEKEKEKPEKEKEKEKEKPEKEKEKEKEKPEKEKE
SEQ ID NO: 595:
EKGEKGEKGEKGEKGEKGEKGEKGEKGEKGEKGEKGEKGEKGEKGEKGEKGEKGEKGEKG
SEQ ID NO: 329:
EKEKGEKEKGEKEKGEKEKGEKEKGEKEKGEKEKGEKEKGEKEKGEKEKGEKEKGEKEKG
SEQ ID NO: 330:
EKEKEKEKEKGEKEKEKEKEKGEKEKEKEKEKGEKEKEKEKEKGEKEKEKEKEKGEKEKE
SEQ ID NO: 331:
EKLEKLEKLEKLEKLEKLEKLEKLEKLEKLEKLEKLEKLEKLEKLEKLEKLEKLEKLEKL
SEQ ID NO: 332:
EKEKLEKEKLEKEKLEKEKLEKEKLEKEKLEKEKLEKEKLEKEKLEKEKLEKEKLEKEKL
SEQ ID NO: 333:
EKEKEKEKEKLEKEKEKEKEKLEKEKEKEKEKLEKEKEKEKEKLEKEKEKEKEKLEKEKE
SEQ ID NO: 334:
EKIEKIEKIEKIEKIEKIEKIEKIEKIEKIEKIEKIEKIEKIEKIEKIEKIEKIEKIEKI
SEQ ID NO: 335:
EKEKIEKEKIEKEKIEKEKIEKEKIEKEKIEKEKIEKEKIEKEKIEKEKIEKEKIEKEKI
SEQ ID NO: 336:
EKEKEKEKEKIEKEKEKEKEKIEKEKEKEKEKIEKEKEKEKEKIEKEKEKEKEKIEKEKE
SEQ ID NO: 337:
ESAESAESAESAESAESAESAESAESAESAESAESAESAESAESAESAESAESAESAESA
SEQ ID NO: 338:
ESESAESESAESESAESESAESESAESESAESESAESESAESESAESESAESESAESESA
SEQ ID NO: 339:
ESESESESESAESESESESESAESESESESESAESESESESESAESESESESESAESESE
SEQ ID NO: 340:
ESSESSESSESSESSESSESSESSESSESSESSESSESSESSESSESSESSESSESSESS
SEQ ID NO: 341:
ESESSESESSESESSESESSESESSESESSESESSESESSESESSESESSESESSESESS
SEQ ID NO: 342:
ESESESESESSESESESESESSESESESESESSESESESESESSESESESESESSESESE
SEQ ID NO: 343:
ESTESTESTESTESTESTESTESTESTESTESTESTESTESTESTESTESTESTESTEST
SEQ ID NO: 344:
ESESTESESTESESTESESTESESTESESTESESTESESTESESTESESTESESTESEST
SEQ ID NO: 345:
ESESESESESTESESESESESTESESESESESTESESESESESTESESESESESTESESE
SEQ ID NO: 346:
ESEESEESEESEESEESEESEESEESEESEESEESEESEESEESEESEESEESEESEESE
SEQ ID NO: 347:
ESESEESESEESESEESESEESESEESESEESESEESESEESESEESESEESESEESESE
SEQ ID NO: 348:
ESESESESESEESESESESESEESESESESESEESESESESESEESESESESESEESESE
SEQ ID NO: 349:
ESDESDESDESDESDESDESDESDESDESDESDESDESDESDESDESDESDESDESDESD
SEQ ID NO: 350:
ESESDESESDESESDESESDESESDESESDESESDESESDESESDESESDESESDESESD
SEQ ID NO: 351:
ESESESESESDESESESESESDESESESESESDESESESESESDESESESESESDESESE
SEQ ID NO: 352:
ESQESQESQESQESQESQESQESQESQESQESQESQESQESQESQESQESQESQESQESQ
SEQ ID NO: 353:
ESESQESESQESESQESESQESESQESESQESESQESESQESESQESESQESESQESESQ
SEQ ID NO: 354:
ESESESESESQESESESESESQESESESESESQESESESESESQESESESESESQESESE
SEQ ID NO: 355:
ESNESNESNESNESNESNESNESNESNESNESNESNESNESNESNESNESNESNESNESN
SEQ ID NO: 356:
ESESNESESNESESNESESNESESNESESNESESNESESNESESNESESNESESNESESN
SEQ ID NO: 357:
ESESESESESNESESESESESNESESESESESNESESESESESNESESESESESNESESE
SEQ ID NO: 358:
ESKESKESKESKESKESKESKESKESKESKESKESKESKESKESKESKESKESKESKESK
SEQ ID NO: 359:
ESESKESESKESESKESESKESESKESESKESESKESESKESESKESESKESESKESESK
SEQ ID NO: 360:
ESESESESESKESESESESESKESESESESESKESESESESESKESESESESESKESESE
SEQ ID NO: 361:
ESRESRESRESRESRESRESRESRESRESRESRESRESRESRESRESRESRESRESRESR
SEQ ID NO: 362:
ESESRESESRESESRESESRESESRESESRESESRESESRESESRESESRESESRESESR
SEQ ID NO: 363:
ESESESESESRESESESESESRESESESESESRESESESESESRESESESESESRESESE
SEQ ID NO: 364:
ESPESPESPESPESPESPESPESPESPESPESPESPESPESPESPESPESPESPESPESP
SEQ ID NO: 365:
ESESPESESPESESPESESPESESPESESPESESPESESPESESPESESPESESPESESP
SEQ ID NO: 366:
ESESESESESPESESESESESPESESESESESPESESESESESPESESESESESPESESE
SEQ ID NO: 367:
ESGESGESGESGESGESGESGESGESGESGESGESGESGESGESGESGESGESGESGESG
SEQ ID NO: 368:
ESESGESESGESESGESESGESESGESESGESESGESESGESESGESESGESESGESESG
SEQ ID NO: 369:
ESESESESESGESESESESESGESESESESESGESESESESESGESESESESESGESESE
SEQ ID NO: 370:
ESLESLESLESLESLESLESLESLESLESLESLESLESLESLESLESLESLESLESLESL
SEQ ID NO: 371:
ESESLESESLESESLESESLESESLESESLESESLESESLESESLESESLESESLESESL
SEQ ID NO: 372:
ESESESESESLESESESESESLESESESESESLESESESESESLESESESESESLESESE
SEQ ID NO: 373:
ESIESIESIESIESIESIESIESIESIESIESIESIESIESIESIESIESIESIESIESI
SEQ ID NO: 374:
ESESIESESIESESIESESIESESIESESIESESIESESIESESIESESIESESIESESI
SEQ ID NO: 375:
ESESESESESIESESESESESIESESESESESIESESESESESIESESESESESIESESE
SEQ ID NO: 376:
EQAEQAEQAEQAEQAEQAEQAEQAEQAEQAEQAEQAEQAEQAEQAEQAEQAEQAEQAEQA
SEQ ID NO: 377:
EQEQAEQEQAEQEQAEQEQAEQEQAEQEQAEQEQAEQEQAEQEQAEQEQAEQEQAEQEQA
SEQ ID NO: 378:
EQEQEQEQEQAEQEQEQEQEQAEQEQEQEQEQAEQEQEQEQEQAEQEQEQEQEQAEQEQE
SEQ ID NO: 379:
EQSEQSEQSEQSEQSEQSEQSEQSEQSEQSEQSEQSEQSEQSEQSEQSEQSEQSEQSEQS
SEQ ID NO: 380:
EQEQSEQEQSEQEQSEQEQSEQEQSEQEQSEQEQSEQEQSEQEQSEQEQSEQEQSEQEQS
SEQ ID NO: 381:
EQEQEQEQEQSEQEQEQEQEQSEQEQEQEQEQSEQEQEQEQEQSEQEQEQEQEQSEQEQE
SEQ ID NO: 382:
EQTEQTEQTEQTEQTEQTEQTEQTEQTEQTEQTEQTEQTEQTEQTEQTEQTEQTEQTEQT
SEQ ID NO: 383:
EQEQTEQEQTEQEQTEQEQTEQEQTEQEQTEQEQTEQEQTEQEQTEQEQTEQEQTEQEQT
SEQ ID NO: 384:
EQEQEQEQEQTEQEQEQEQEQTEQEQEQEQEQTEQEQEQEQEQTEQEQEQEQEQTEQEQE
SEQ ID NO: 385:
EQEEQEEQEEQEEQEEQEEQEEQEEQEEQEEQEEQEEQEEQEEQEEQEEQEEQEEQEEQE
SEQ ID NO: 386:
EQEQEEQEQEEQEQEEQEQEEQEQEEQEQEEQEQEEQEQEEQEQEEQEQEEQEQEEQEQE
SEQ ID NO: 387:
EQEQEQEQEQEEQEQEQEQEQEEQEQEQEQEQEEQEQEQEQEQEEQEQEQEQEQEEQEQE
SEQ ID NO: 388:
EQDEQDEQDEQDEQDEQDEQDEQDEQDEQDEQDEQDEQDEQDEQDEQDEQDEQDEQDEQD
SEQ ID NO: 389:
EQEQDEQEQDEQEQDEQEQDEQEQDEQEQDEQEQDEQEQDEQEQDEQEQDEQEQDEQEQD
SEQ ID NO: 390:
EQEQEQEQEQDEQEQEQEQEQDEQEQEQEQEQDEQEQEQEQEQDEQEQEQEQEQDEQEQE
SEQ ID NO: 391:
EQQEQQEQQEQQEQQEQQEQQEQQEQQEQQEQQEQQEQQEQQEQQEQQEQQEQQEQQEQQ
SEQ ID NO: 392:
EQEQQEQEQQEQEQQEQEQQEQEQQEQEQQEQEQQEQEQQEQEQQEQEQQEQEQQEQEQQ
SEQ ID NO: 393:
EQEQEQEQEQQEQEQEQEQEQQEQEQEQEQEQQEQEQEQEQEQQEQEQEQEQEQQEQEQE
SEQ ID NO: 394:
EQNEQNEQNEQNEQNEQNEQNEQNEQNEQNEQNEQNEQNEQNEQNEQNEQNEQNEQNEQN
SEQ ID NO: 395:
EQEQNEQEQNEQEQNEQEQNEQEQNEQEQNEQEQNEQEQNEQEQNEQEQNEQEQNEQEQN
SEQ ID NO: 396:
EQEQEQEQEQNEQEQEQEQEQNEQEQEQEQEQNEQEQEQEQEQNEQEQEQEQEQNEQEQE
SEQ ID NO: 397:
EQKEQKEQKEQKEQKEQKEQKEQKEQKEQKEQKEQKEQKEQKEQKEQKEQKEQKEQKEQK
SEQ ID NO: 398:
EQEQKEQEQKEQEQKEQEQKEQEQKEQEQKEQEQKEQEQKEQEQKEQEQKEQEQKEQEQK
SEQ ID NO: 399:
EQEQEQEQEQKEQEQEQEQEQKEQEQEQEQEQKEQEQEQEQEQKEQEQEQEQEQKEQEQE
SEQ ID NO: 400:
EQREQREQREQREQREQREQREQREQREQREQREQREQREQREQREQREQREQREQREQR
SEQ ID NO: 401:
EQEQREQEQREQEQREQEQREQEQREQEQREQEQREQEQREQEQREQEQREQEQREQEQR
SEQ ID NO: 402:
EQEQEQEQEQREQEQEQEQEQREQEQEQEQEQREQEQEQEQEQREQEQEQEQEQREQEQE
SEQ ID NO: 403:
EQPEQPEQPEQPEQPEQPEQPEQPEQPEQPEQPEQPEQPEQPEQPEQPEQPEQPEQPEQP
SEQ ID NO: 404:
EQEQPEQEQPEQEQPEQEQPEQEQPEQEQPEQEQPEQEQPEQEQPEQEQPEQEQPEQEQP
SEQ ID NO: 405:
EQEQEQEQEQPEQEQEQEQEQPEQEQEQEQEQPEQEQEQEQEQPEQEQEQEQEQPEQEQE
SEQ ID NO: 406:
EQGEQGEQGEQGEQGEQGEQGEQGEQGEQGEQGEQGEQGEQGEQGEQGEQGEQGEQGEQG
SEQ ID NO: 407:
EQEQGEQEQGEQEQGEQEQGEQEQGEQEQGEQEQGEQEQGEQEQGEQEQGEQEQGEQEQG
SEQ ID NO: 408:
EQEQEQEQEQGEQEQEQEQEQGEQEQEQEQEQGEQEQEQEQEQGEQEQEQEQEQGEQEQE
SEQ ID NO: 409:
EQLEQLEQLEQLEQLEQLEQLEQLEQLEQLEQLEQLEQLEQLEQLEQLEQLEQLEQLEQL
SEQ ID NO: 410:
EQEQLEQEQLEQEQLEQEQLEQEQLEQEQLEQEQLEQEQLEQEQLEQEQLEQEQLEQEQL
SEQ ID NO: 411:
EQEQEQEQEQLEQEQEQEQEQLEQEQEQEQEQLEQEQEQEQEQLEQEQEQEQEQLEQEQE
SEQ ID NO: 412:
EQIEQIEQIEQIEQIEQIEQIEQIEQIEQIEQIEQIEQIEQIEQIEQIEQIEQIEQIEQI
SEQ ID NO: 413:
EQEQIEQEQIEQEQIEQEQIEQEQIEQEQIEQEQIEQEQIEQEQIEQEQIEQEQIEQEQI
SEQ ID NO: 414:
EQEQEQEQEQIEQEQEQEQEQIEQEQEQEQEQIEQEQEQEQEQIEQEQEQEQEQIEQEQE
SEQ ID NO: 415:
EPAEPAEPAEPAEPAEPAEPAEPAEPAEPAEPAEPAEPAEPAEPAEPAEPAEPAEPAEPA
SEQ ID NO: 416:
EPEPAEPEPAEPEPAEPEPAEPEPAEPEPAEPEPAEPEPAEPEPAEPEPAEPEPAEPEPA
SEQ ID NO: 417:
EPEPEPEPEPAEPEPEPEPEPAEPEPEPEPEPAEPEPEPEPEPAEPEPEPEPEPAEPEPE
SEQ ID NO: 418:
EPSEPSEPSEPSEPSEPSEPSEPSEPSEPSEPSEPSEPSEPSEPSEPSEPSEPSEPSEPS
SEQ ID NO: 419:
EPEPSEPEPSEPEPSEPEPSEPEPSEPEPSEPEPSEPEPSEPEPSEPEPSEPEPSEPEPS
SEQ ID NO: 420:
EPEPEPEPEPSEPEPEPEPEPSEPEPEPEPEPSEPEPEPEPEPSEPEPEPEPEPSEPEPE
SEQ ID NO: 421:
EPTEPTEPTEPTEPTEPTEPTEPTEPTEPTEPTEPTEPTEPTEPTEPTEPTEPTEPTEPT
SEQ ID NO: 422:
EPEPTEPEPTEPEPTEPEPTEPEPTEPEPTEPEPTEPEPTEPEPTEPEPTEPEPTEPEPT
SEQ ID NO: 423:
EPEPEPEPEPTEPEPEPEPEPTEPEPEPEPEPTEPEPEPEPEPTEPEPEPEPEPTEPEPE
SEQ ID NO: 424:
EPEEPEEPEEPEEPEEPEEPEEPEEPEEPEEPEEPEEPEEPEEPEEPEEPEEPEEPEEPE
SEQ ID NO: 425:
EPEPEEPEPEEPEPEEPEPEEPEPEEPEPEEPEPEEPEPEEPEPEEPEPEEPEPEEPEPE
SEQ ID NO: 426:
EPEPEPEPEPEEPEPEPEPEPEEPEPEPEPEPEEPEPEPEPEPEEPEPEPEPEPEEPEPE
SEQ ID NO: 427:
EPDEPDEPDEPDEPDEPDEPDEPDEPDEPDEPDEPDEPDEPDEPDEPDEPDEPDEPDEPD
SEQ ID NO: 428:
EPEPDEPEPDEPEPDEPEPDEPEPDEPEPDEPEPDEPEPDEPEPDEPEPDEPEPDEPEPD
SEQ ID NO: 429:
EPEPEPEPEPDEPEPEPEPEPDEPEPEPEPEPDEPEPEPEPEPDEPEPEPEPEPDEPEPE
SEQ ID NO: 430:
EPQEPQEPQEPQEPQEPQEPQEPQEPQEPQEPQEPQEPQEPQEPQEPQEPQEPQEPQEPQ
SEQ ID NO: 431:
EPEPQEPEPQEPEPQEPEPQEPEPQEPEPQEPEPQEPEPQEPEPQEPEPQEPEPQEPEPQ
SEQ ID NO: 432:
EPEPEPEPEPQEPEPEPEPEPQEPEPEPEPEPQEPEPEPEPEPQEPEPEPEPEPQEPEPE
SEQ ID NO: 433:
EPNEPNEPNEPNEPNEPNEPNEPNEPNEPNEPNEPNEPNEPNEPNEPNEPNEPNEPNEPN
SEQ ID NO: 434:
EPEPNEPEPNEPEPNEPEPNEPEPNEPEPNEPEPNEPEPNEPEPNEPEPNEPEPNEPEPN
SEQ ID NO: 435:
EPEPEPEPEPNEPEPEPEPEPNEPEPEPEPEPNEPEPEPEPEPNEPEPEPEPEPNEPEPE
SEQ ID NO: 436:
EPKEPKEPKEPKEPKEPKEPKEPKEPKEPKEPKEPKEPKEPKEPKEPKEPKEPKEPKEPK
SEQ ID NO: 437:
EPEPKEPEPKEPEPKEPEPKEPEPKEPEPKEPEPKEPEPKEPEPKEPEPKEPEPKEPEPK
SEQ ID NO: 438:
EPEPEPEPEPKEPEPEPEPEPKEPEPEPEPEPKEPEPEPEPEPKEPEPEPEPEPKEPEPE
SEQ ID NO: 439:
EPREPREPREPREPREPREPREPREPREPREPREPREPREPREPREPREPREPREPREPR
SEQ ID NO: 440:
EPEPREPEPREPEPREPEPREPEPREPEPREPEPREPEPREPEPREPEPREPEPREPEPR
SEQ ID NO: 441:
EPEPEPEPEPREPEPEPEPEPREPEPEPEPEPREPEPEPEPEPREPEPEPEPEPREPEPE
SEQ ID NO: 442:
EPPEPPEPPEPPEPPEPPEPPEPPEPPEPPEPPEPPEPPEPPEPPEPPEPPEPPEPPEPP
SEQ ID NO: 443:
EPEPPEPEPPEPEPPEPEPPEPEPPEPEPPEPEPPEPEPPEPEPPEPEPPEPEPPEPEPP
SEQ ID NO: 444:
EPEPEPEPEPPEPEPEPEPEPPEPEPEPEPEPPEPEPEPEPEPPEPEPEPEPEPPEPEPE
SEQ ID NO: 445:
EPGEPGEPGEPGEPGEPGEPGEPGEPGEPGEPGEPGEPGEPGEPGEPGEPGEPGEPGEPG
SEQ ID NO: 446:
EPEPGEPEPGEPEPGEPEPGEPEPGEPEPGEPEPGEPEPGEPEPGEPEPGEPEPGEPEPG
SEQ ID NO: 447:
EPEPEPEPEPGEPEPEPEPEPGEPEPEPEPEPGEPEPEPEPEPGEPEPEPEPEPGEPEPE
SEQ ID NO: 448:
EPLEPLEPLEPLEPLEPLEPLEPLEPLEPLEPLEPLEPLEPLEPLEPLEPLEPLEPLEPL
SEQ ID NO: 449:
EPEPLEPEPLEPEPLEPEPLEPEPLEPEPLEPEPLEPEPLEPEPLEPEPLEPEPLEPEPL
SEQ ID NO: 450:
EPEPEPEPEPLEPEPEPEPEPLEPEPEPEPEPLEPEPEPEPEPLEPEPEPEPEPLEPEPE
SEQ ID NO: 451:
EPIEPIEPIEPIEPIEPIEPIEPIEPIEPIEPIEPIEPIEPIEPIEPIEPIEPIEPIEPI
SEQ ID NO: 452:
EPEPIEPEPIEPEPIEPEPIEPEPIEPEPIEPEPIEPEPIEPEPIEPEPIEPEPIEPEPI
SEQ ID NO: 453:
EPEPEPEPEPIEPEPEPEPEPIEPEPEPEPEPIEPEPEPEPEPIEPEPEPEPEPIEPEPE
SEQ ID NO: 454:
PASAPASAPASAPASAPASAPASAPASAPASAPASAPASAPASAPASAPASAPASAPASA
SEQ ID NO: 455:
PASPASAPASPASAPASPASAPASPASAPASPASAPASPASAPASPASAPASPASAPASP
SEQ ID NO: 456:
PASPASPASPASPASAPASPASPASPASPASAPASPASPASPASPASAPASPASPASPAS
SEQ ID NO: 457:
PASSPASSPASSPASSPASSPASSPASSPASSPASSPASSPASSPASSPASSPASSPASS
SEQ ID NO: 458:
PASPASSPASPASSPASPASSPASPASSPASPASSPASPASSPASPASSPASPASSPASP
SEQ ID NO: 459:
PASPASPASPASPASSPASPASPASPASPASSPASPASPASPASPASSPASPASPASPAS
SEQ ID NO: 460:
PASTPASTPASTPASTPASTPASTPASTPASTPASTPASTPASTPASTPASTPASTPAST
SEQ ID NO: 461:
PASPASTPASPASTPASPASTPASPASTPASPASTPASPASTPASPASTPASPASTPASP
SEQ ID NO: 462:
PASPASPASPASPASTPASPASPASPASPASTPASPASPASPASPASTPASPASPASPAS
SEQ ID NO: 463:
PASEPASEPASEPASEPASEPASEPASEPASEPASEPASEPASEPASEPASEPASEPASE
SEQ ID NO: 464:
PASPASEPASPASEPASPASEPASPASEPASPASEPASPASEPASPASEPASPASEPASP
SEQ ID NO: 465:
PASPASPASPASPASEPASPASPASPASPASEPASPASPASPASPASEPASPASPASPAS
SEQ ID NO: 466:
PASDPASDPASDPASDPASDPASDPASDPASDPASDPASDPASDPASDPASDPASDPASD
SEQ ID NO: 467:
PASPASDPASPASDPASPASDPASPASDPASPASDPASPASDPASPASDPASPASDPASP
SEQ ID NO: 468:
PASPASPASPASPASDPASPASPASPASPASDPASPASPASPASPASDPASPASPASPAS
SEQ ID NO: 469:
PASQPASQPASQPASQPASQPASQPASQPASQPASQPASQPASQPASQPASQPASQPASQ
SEQ ID NO: 470:
PASPASQPASPASQPASPASQPASPASQPASPASQPASPASQPASPASQPASPASQPASP
SEQ ID NO: 471:
PASPASPASPASPASQPASPASPASPASPASQPASPASPASPASPASQPASPASPASPAS
SEQ ID NO: 472:
PASNPASNPASNPASNPASNPASNPASNPASNPASNPASNPASNPASNPASNPASNPASN
SEQ ID NO: 473:
PASPASNPASPASNPASPASNPASPASNPASPASNPASPASNPASPASNPASPASNPASP
SEQ ID NO: 474:
PASPASPASPASPASNPASPASPASPASPASNPASPASPASPASPASNPASPASPASPAS
SEQ ID NO: 475:
PASKPASKPASKPASKPASKPASKPASKPASKPASKPASKPASKPASKPASKPASKPASK
SEQ ID NO: 476:
PASPASKPASPASKPASPASKPASPASKPASPASKPASPASKPASPASKPASPASKPASP
SEQ ID NO: 477:
PASPASPASPASPASKPASPASPASPASPASKPASPASPASPASPASKPASPASPASPAS
SEQ ID NO: 478:
PASRPASRPASRPASRPASRPASRPASRPASRPASRPASRPASRPASRPASRPASRPASR
SEQ ID NO: 479:
PASPASRPASPASRPASPASRPASPASRPASPASRPASPASRPASPASRPASPASRPASP
SEQ ID NO: 480:
PASPASPASPASPASRPASPASPASPASPASRPASPASPASPASPASRPASPASPASPAS
SEQ ID NO: 481:
PASPPASPPASPPASPPASPPASPPASPPASPPASPPASPPASPPASPPASPPASPPASP
SEQ ID NO: 482:
PASPASPPASPASPPASPASPPASPASPPASPASPPASPASPPASPASPPASPASPPASP
SEQ ID NO: 483:
PASPASPASPASPASPPASPASPASPASPASPPASPASPASPASPASPPASPASPASPAS
SEQ ID NO: 484:
PASGPASGPASGPASGPASGPASGPASGPASGPASGPASGPASGPASGPASGPASGPASG
SEQ ID NO: 485:
PASPASGPASPASGPASPASGPASPASGPASPASGPASPASGPASPASGPASPASGPASP
SEQ ID NO: 486:
PASPASPASPASPASGPASPASPASPASPASGPASPASPASPASPASGPASPASPASPAS
SEQ ID NO: 487:
PASLPASLPASLPASLPASLPASLPASLPASLPASLPASLPASLPASLPASLPASLPASL
SEQ ID NO: 488:
PASPASLPASPASLPASPASLPASPASLPASPASLPASPASLPASPASLPASPASLPASP
SEQ ID NO: 489:
PASPASPASPASPASLPASPASPASPASPASLPASPASPASPASPASLPASPASPASPAS
SEQ ID NO: 490:
PASIPASIPASIPASIPASIPASIPASIPASIPASIPASIPASIPASIPASIPASIPASI
SEQ ID NO: 491:
PASPASIPASPASIPASPASIPASPASIPASPASIPASPASIPASPASIPASPASIPASP
SEQ ID NO: 492:
PASPASPASPASPASIPASPASPASPASPASIPASPASPASPASPASIPASPASPASPAS
SEQ ID NO: 493:
GGSPGSPAGSPTSTEEGTSESATPESGPGTSTEPSEGSAPGSPAGSPTSTEEGTSTEPSE
SEQ ID NO: 494:
GSAPGTSTEPSEGSAPGTSESATPESGPGSEPATSGSETPGSEPATSGSETPGSPAGSPT
SEQ ID NO: 495:
STEEGTSESATPESGPGTSTEPSEGSAPGTSTEPSEGSAPGSPAGSPTSTEEGTSTEPSE
SEQ ID NO: 496:
GSAPGTSTEPSEGSAPGTSESATPESGPGTSTEPSEGSAPGTSESATPESGPGSEPATSG
SEQ ID NO: 497:
SETPGTSTEPSEGSAPGTSTEPSEGSAPGTSESATPESGPGTSESATPESGPGSPAGSPT
SEQ ID NO: 498:
STEEGTSESATPESGPGSEPATSGSETPGTSESATPESGPGTSTEPSEGSAPGTSTEPSE
SEQ ID NO: 499:
GSAPGTSTEPSEGSAPGTSTEPSEGSAPGTSTEPSEGSAPGTSTEPSEGSAPGSPAGSPT
SEQ ID NO: 500:
STEEGTSTEPSEGSAPGTSESATPESGPGSEPATSGSETPGTSESATPESGPGSEPATSG
SEQ ID NO: 501:
SETPGTSESATPESGPGTSTEPSEGSAPGTSESATPESGPGSPAGSPTSTEEGSPAGSPT
SEQ ID NO: 502:
STEEGSPAGSPTSTEEGTSESATPESGPGTGTSESATPESGPGSEPATSGSETPGTSESA
SEQ ID NO: 503:
TPESGPGSEPATSGSETPGTSESATPESGPGTSTEPSEGSAPGSPAGSPTSTEEGTSESA
SEQ ID NO: 504:
TPESGPGSEPATSGSETPGTSESATPESGPGSPAGSPTSTEEGSPAGSPTSTEEGTSTEP
SEQ ID NO: 505:
SEGSAPGTSESATPESGPGTSESATPESGPGTSESATPESGPGSEPATSGSETPGSEPAT
SEQ ID NO: 506:
SGSETPGSPAGSPTSTEEGTSTEPSEGSAPGTSTEPSEGSAPGSEPATSGSETPGTSESA
SEQ ID NO: 507:
GTSTEPSEGSAPGTSTEPSEGSAPGSEPATSGSETPGTSESATPESGPGTSTEPSEGSAP
SEQ ID NO: 508:
STEPSEGSAPSTEPSEGSAPSTEPSEGSAPSTEPSEGSAPSTEPSEGSAPSTEPSEGSAP
SEQ ID NO: 509:
GSPAGSPTSTEEGTGSPAGSPTSTEEGTGSPAGSPTSTEEGTGSPAGSPTSTEEGTGSPA
SEQ ID NO: 510:
STEPSEGSAPGSPAGSPTSTEEGTSTEPSEGSAPGSPAGSPTSTEEGTSTEPSEGSAPGS
SEQ ID NO: 511:
PSTADPSTADPSTADPSTADPSTADPSTADPSTADPSTADPSTADPSTADPSTADPSTAD
SEQ ID NO: 512:
PSTADGSTADPSTADGSTADPSTADGSTADPSTADGSTADPSTADGSTADPSTADGSTAD
SEQ ID NO: 513:
PSTAKPSTAKPSTAKPSTAKPSTAKPSTAKPSTAKPSTAKPSTAKPSTAKPSTAKPSTAK
SEQ ID NO: 514:
STEEGTSESATPESGPGSEPATSGSETPGTSESATPESGPGTSTEPSEGSAPGTSTEPES
TEEGTSESATPESGPGSEPATSGSETPGTSESATPESGPGTSTEPSEGSAPGTSTEPE
SEQ ID NO: 515:
STEEGTSESATPESGPGSEPATSGSETPGTSESATPESGPGTSTEPSEGSAPGTSTEPEP
ASPASEPASPASEPASPASEPASPASEPASPASEPASPASEPASPASEPASPASEPAP
SEQ ID NO: 516:
PETSPASTEPEGSPETSPASTEPEGSPETSPASTEPEGSPETSPASTEPEGSPETSPAS
SEQ ID NO: 517:
PESTGAPGETSPEGSPESTGAPGETSPEGSPESTGAPGETSPEGSPESTGAPGETSPEG
SEQ ID NO: 518:
SGSEPEPTSPSETPSPPGGTPGSEATSPTEETGAEGPAGPGPGSEEGSTEGAGTSPEES

The isolated polypeptides of the disclosure may be produced recombinantly or synthetically, using standard techniques in the art. The isolated polypeptides of the disclosure can be modified in a number of ways, including but not limited to the ways described above, either before or after assembly of the nanostructures of the invention. As used throughout the present application, the term “polypeptide” is used in its broadest sense to refer to a sequence of subunit amino acids. The polypeptides of the invention may comprise L-amino acids and glycine, D-amino acids (which are resistant to L-amino acid-specific proteases in vivo) and glycine, or a combination of D- and L-amino acids and glycine.

In a fifth aspect, the disclosure provides nanostructures wherein at least one of the plurality of assemblies in the nanostructure is made up of polypeptides of one of the first four aspects of the disclosure. Thus, in one embodiment the nanostructures comprise

(a) a plurality of first assemblies, each first assembly comprising a plurality of identical first polypeptides, wherein the first polypeptides comprise the polypeptide of any embodiment or combination of embodiments of the first aspect of the disclosure (i.e.: I53-50 trimer modified proteins); and

(b) a plurality of second assemblies, each second assembly comprising a plurality of identical second polypeptides, wherein the second polypeptides:
(i) comprise the polypeptide of any embodiment or combination of embodiments of the second aspect of the disclosure; or
(ii) are at least 75%, 80%, 85%, 90%, 91%, 92%, 93%, 94%, 95%, 96%, 97%, 98%, or 99% identical over the length of the amino acid sequence selected from the group consisting of SEQ IDS NO: 2 and 519-522;

wherein the plurality of first assemblies non-covalently interact with the plurality of second assemblies to form a nanostructure.

| 153-50B.1 | MNQHSHKDHETVRIAVVRARWHAEIVDACVSAFEAAMR | Identified interface |
|---|---|---|
| SEQ ID | DIGGDRFAVDVFDVPGAYEIPLHARTLAETGRYGAVLG | positions: 153-50B: |
| NO: 519 | TAFVVNGGIYRHEFVASAVIDGMMNVQLDTGVPVLSAV | 24, 28, 36, 124, 125, 127, |
| | LTPHRYRDSDAHTLLFLALFAVKGMEAARACVEILAAR | 128, 129, 131, 132, 133, |
| | EKIAA | 135, 139 |
| 153-50B.1NegT2 | MNQHSHKDHETVRIAVVRARWHAEIVDACVSAFEAAMR | Identified interface |
| SEQ ID | DIGGDRFAVDVFDVPGAYEIPLHARTLAETGRYGAVLG | positions: 153-50B: |
| NO: 520 | TAFVVDGGIYDHEFVASAVIDGMMNVQLDTGVPVLSAV | 24, 28, 36, 124, 125, 127, |
| | LTPHEYEDSDADTLLFLALFAVKGMEAARACVEILAAR | 128, 129, 131, 132, 133, |
| | EKIAA | 135, 139 |
| 153-50B.4PosT1 | MNQHSHKDHETVRIAVVRARWHAEIVDACVSAFEAAMR | Identified interface |
| SEQ ID | DIGGDRFAVDVFDVPGAYEIPLHARTLAETGRYGAVLG | positions: 153-50B: |
| NO: 521 | TAFVVNGGIYRHEFVASAVINGMMNVQLNTGVPVLSAV | 24, 28, 36, 124, 125, 127, |
| | LTPHNYDKSKAHTLLFLALFAVKGMEAARACVEILAAR | 128, 129, 131, 132, 133, |
| | EKIAA | 135, 139 |

I53-50B genus
(SEQ ID NO: 522)
MNQHSHKD(Y/H)ETVRIAVVRARWHAEIVDACVSAFEAAM(A/R)DIG
GDRFAVDVFDVPGAYEIPLHARTLAETGRYGAVLGTAFVV(N/D)GGIY
(R/D)HEFVASAVI(D/N)GMMNVQL(S/D/N) TGVPVLSAVLTPH
(R/E/N)Y(R/D/E)(D/K)S(D/K)A(H/D)TLLFLALFAVKGMEA
ARACVEILAAREKIAA

The second polypeptides of SEQ ID NO: 2 and 519-522 are polypeptides disclosed in U.S. Pat. No. 9,630,994 (incorporated by reference herein in its entirety) that form homo-pentamers that can non-covalently interact with the polypeptides of the first aspect of the disclosure to generate the nanostructures. The second polypeptides of the second aspect of the disclosure are improved homo-pentamer forming polypeptides as described herein.

In one embodiment, wherein the second polypeptides are at least 75%, 80%, 85%, 90%, 91%, 92%, 93%, 94%, 95%, 96%, 97%, 98%, or 99% identical over the length of the amino acid sequence selected from the group consisting of SEQ IDS NO:2 or 519-522, the second polypeptides may be identical at least at 1, 2, 3, 4, 5, 6, 7, 8, 9, 10, 11, 12, or 13 identified interface positions of the amino acid sequence selected from the group consisting of SEQ IDS NO:2 or 519-522.

In another embodiment the nanostructures comprise

(a) a plurality of first assemblies, each first assembly comprising a plurality of identical first polypeptides, wherein the first polypeptides:
(i) comprise the polypeptide of any embodiment or combination of embodiments of the first aspect of the disclosure (i.e.: I53-50 trimer modified proteins); or
(ii) are at least 75%, 80%, 85%, 90%, 91%, 92%, 93%, 94%, 95%, 96%, 97%, 98%, or 99% identical over the length of the amino acid sequence selected from the group consisting of SEQ IDS NO:1 and 523-526; and

(b) a plurality of second assemblies, each second assembly comprising a plurality of identical second polypeptides, wherein the second polypeptides comprise the polypeptide of any embodiment or combination of embodiments of the second aspect of the disclosure (i.e.: I53-50 pentamer modified proteins);

wherein the plurality of first assemblies non-covalently interact with the plurality of second assemblies to form a nanostructure.

| | | |
|---|---|---|
| 153-50A.1 | MKMEELFKKHKIVAVLRANSVEEAIEKAVAVFAGGVHL | Identified interface |
| SEQ ID | IEITFTVPDADTVIKALSVLKEKGAIIGAGTVTSVEQC | positions: I53-50A: |
| NO: 523 | RKAVESGAEFIVSPHLDEEISQFCKEKGVFYMPGVMTP | 25, 29, 33, 54, 57 |
| | TELVKAMKLGHDILKLFPGEVVGPQFVKAMKGPFPNVK | |
| | FVPTGGVNLDNVCEWFKAGVLAVGVGDALVKGDPDEVR | |
| | EKAKKFVEKIRGCTE | |
| 153-50A.1NegT2 | MKMEELFKKHKIVAVLRANSVEEAIEKAVAVFAGGVHL | Identified interface |
| SEQ ID | IEITFTVPDADTVIKALSVLKEKGAIIGAGTVTSVEQC | positions: I53-50A: |
| NO: 524 | RKAVESGAEFIVSPHLDEEISQFCKEKGVFYMPGVMTP | 25, 29, 33, 54, 57 |
| | TELVKAMKLGHDILKLFPGEVVGPEFVEAMKGPFPNVK | |
| | FVPTGGVDLDDVCEWFDAGVLAVGVGDALVEGDPDEVR | |
| | EDAKEFVEEIRGCTE | |
| 153-50A.11PosT1 | MKMEELFKKHKIVAVLRANSVEEAIEKAVAVFAGGVHL | Identified interface |
| SEQ ID | IEITFTVPDADTVIKALSVLKEKGAIIGAGTVTSVEQC | positions: I53-50A: |
| NO: 525 | RKAVESGAEFIVSPHLDEEISQFCKEKGVFYMPGVMTP | 25, 29, 33, 54, 57 |
| | TELVKAMKLGHDILKLFPGEVVGPQFVKAMKGPFPNVK | |
| | FVPTGGVNLDNVCKWFKAGVLAVGVGKALVKGKPDEVR | |
| | EKAKKFVKKIRGCTE | |

I53-50A genus
(SEQ ID NO: 526)
MKMEELFKKHKIVAVLRANSVEEAIEKAVAVFAGGVHLIEITFTVPDADT
VIKALSVLKEKGAIIGAGTVTSVEQCRKAVESGAEFIVSPHLDEEISQFC
KEKGVFYMPGVMTPTELVKAMKLGH(T/D)ILKLFPGEVVGP(Q/E)FV
(K/E)AMKGPFPNVKFVPTGGV(N/D)LD(N/D)VC(E/K)WF(K/D)A
GVLAVGVG(S/K/D)ALV(K/E)G(T/D/K)PDEVRE(K/D)AK(A/E/K)
FV(E/K)(K/E)IRGCTE

The first polypeptides of SEQ ID NOS: 1 and 523-526 are polypeptides disclosed in U.S. Pat. No. 9,630,994 (incorporated by reference herein in its entirety) that form homo-trimers that can non-covalently interact with the polypeptides of the second aspect of the disclosure to generate the nanostructures. The first polypeptides of the first aspect of the disclosure are improved homo-trimer-forming polypeptides as described herein.

In one embodiment, wherein the first polypeptides are at least 75%, 80%, 85%, 90%, 91%, 92%, 93%, 94%, 95%, 96%, 97%, 98%, or 99% identical over the length of the amino acid sequence selected from the group consisting of SEQ ID NOS: 1 and 523-526, the first polypeptides may be identical at least at 1, 2, 3, 4, 5, 6, 7, 8, 9, 10, 11, 12, or 13 identified interface positions of the amino acid sequence selected from the group consisting of SEQ ID NOS: 1 and 523-526.

In one specific embodiment, the nanostructures may comprise:

(a) a plurality of first assemblies, each first assembly comprising a plurality of identical first polypeptides, wherein the first polypeptides comprise the polypeptide of any embodiment or combination of embodiments of the first aspect of the disclosure; and

(b) a plurality of second assemblies, each second assembly comprising a plurality of identical second polypeptides, wherein the second polypeptides comprise the polypeptide of any embodiment or combination of embodiments of the second aspect of the disclosure;

wherein the plurality of first assemblies non-covalently interact with the plurality of second assemblies to form a nanostructure.

In various further specific embodiments:

(a) the first polypeptides comprises polypeptides having a set of amino acid substitutions relative to SEQ ID NO:1 selected from the group consisting of:
(i) T126D, E166K, S179K, T185K, A195K, and E198K;
(ii) T126D, E166K, S179K/N, T185K/N, E188K, A195K, and E198K;
(iii) K2T, K9R, K11T, K61D, T126D, E166K, S179K/N, T185K/N, E188K, A195K, and E198K;
(iv) K2T, K9R, K11T, K61D, E74D, T126D, E166K, S179K/N, T185K/N, E188K, A195K, and E198K; and
(v) E74D, C76A, C100A, T126D, C165A, C203A.

In other specific embodiments:

(b) the second polypeptides comprise polypeptides having a set of amino acid substitutions relative to SEQ ID NO:2 selected from the group consisting of:
(i) Y9H, A38R, S105D, R119N, R121D, D122K, and D124K;
(ii) Y9H, E24F/M, A38R, S105D, R119N, R121D, D122K, K124N, and H126K;
(iii) H6Q, Y9H/Q, E24F/M, A38R, S105D, R119N, R121D, D122K, K124N, and H126K; and
(iv) H6Q, Y9H/Q, E24F/M, A38R, D39K, D43E, E67K, S105D, R119N, R121D, D122K, K124N, and H126K.

In another embodiment, the nanostructures may comprise

(a) a plurality of first assemblies, each first assembly comprising a plurality of identical first polypeptides, wherein the first polypeptides comprise the polypeptide of any embodiment or combination of embodiments of the third aspect of the disclosure; and

(b) a plurality of second assemblies, each second assembly comprising a plurality of identical second polypeptides, wherein the second polypeptides:
(i) comprise the polypeptide of any embodiment or combination of embodiments of the fourth aspect of the disclosure, or
(ii) are at least 75%, 80%, 85%, 90%, 91%, 92%, 93%, 94%, 95%, 96%, 97%, 98%, or 99% identical over the length of the amino acid sequence selected from the group consisting of SEQ IDS NOS:4 and 527-529;

wherein the plurality of first assemblies non-covalently interact with the plurality of second assemblies to form a nanostructure.

| | | |
|---|---|---|
| 153-47B.1 | MNQHSHKDHETVRIAVVRARWHADIVDACVEAFEIAMA | I53-47B: |
| SEQ ID | AIGGDRFAVDVFDVPGAYEIPLHARTLAETGRYGAVLG | 28, 31, 35, 36, 39, 131, 132, |
| NO: 527 | TAFVVNGGIYRHEFVASAVIDGMMNVQLDTGVPVLSAV | 135, 139, 146 |
| | LTPHRYRDSDEHHRFFAAHFAVKGVEAARACIEILNAR | |
| | EKIAA | |
| 153-47B.1NegT2 | MNQHSHKDHETVRIAVVRARWHADIVDACVEAFEIAMA | I53-47B: |
| SEQ ID | AIGGDRFAVDVFDVPGAYEIPLHARTLAETGRYGAVLG | 28, 31, 35, 36, 39, 131, 132, |
| NO: 528 | TAFVVDGGIYDHEFVASAVIDGMMNVQLDTGVPVLSAV | 135, 139, 146 |
| | LTPHEYEDSDEDHEFFAAHFAVKGVEAARACIEILNAR | |
| | EKIAA | |

I53-47B genus
(SEQ ID NO: 529)
MNQHSHKD(Y/H)ETVRIAVVRARWHADIVDACVEAFEIAMAAIGGDRFA
VDVFDVPGAYEIPLHARTLAETGRYGAVLGTAFVV(N/D)GGIY(R/D)H
EFVASAVIDGMMNVQL(S/D)TGVPVLSAVLTPH(R/E)Y(R/E)DS(A/
D)E(H/D)H(R/E)FFAAHFAVKGVEAARACIEIL(A/N)AREKIAA

The second polypeptides of SEQ ID NOS:4 and 527-529 are polypeptides disclosed in U.S. Pat. No. 9,630,994 (incorporated by reference herein in its entirety) that form homo-pentamers that can non-covalently interact with the polypeptides of the third aspect of the disclosure to generate the nanostructures. The second polypeptides of the fourth aspect of the disclosure are improved homo-pentamer forming polypeptides as described herein.

In one embodiment, wherein the second polypeptides are at least 75%, 80%, 85%, 90%, 91%, 92%, 93%, 94%, 95%, 96%, 97%, 98%, or 99% identical over the length of the amino acid sequence selected from the group consisting of SEQ ID NOS:4 and 527-529, the polypeptides are also identical at least at 1, 2, 3, 4, 5, 6, 7, 8, 9, or 10 identified interface positions of the amino acid sequence selected from the group consisting of SEQ ID NOS:4 and 527-529.

In a further embodiment, the nanostructures comprise

(a) a plurality of first assemblies, each first assembly comprising a plurality of identical first polypeptides, wherein the first polypeptides
(i) comprise the polypeptide of any embodiment or combination of embodiments of the third aspect of the disclosure, or
(ii) wherein the first polypeptides are at least 75%, 80%, 85%, 90%, 91%, 92%, 93%, 94%, 95%, 96%, 97%, 98%, or 99% identical over the length of the amino acid sequence selected from the group consisting of SEQ IDS NO:3 and 530-532; and

(b) a plurality of second assemblies, each second assembly comprising a plurality of identical second polypeptides, wherein the second polypeptides comprise the polypeptide of any embodiment or combination of embodiments of the third aspect of the disclosure;

wherein the plurality of first assemblies non-covalently interact with the plurality of second assemblies to form a nanostructure.

| | | |
|---|---|---|
| I53-47A | (M)PIFTLNTNIKATDVPSDFLSLTSRLVGLILSKPGS | I53-47A: |
| SEQ ID | YVAVHINTDQQLSFGGSTNPAAFGTLMSIGGIEPSKNR | 22, 25, 29, 72, 79, 86, 87 |
| NO: 03 | DHSAVLFDHLNAMLGIPKNRMYIHFVNLNGDDVGWNGT | |
| | TF | |
| 153-47A.I | MPIFTLNTNIKADDVPSDFLSLTSRLVGLILSKPGSYV | I53-47A: |
| SEQ ID | AVHINTDQQLSFGGSTNPAAFGTLMSIGGIEPDKNRDH | 22, 25, 29, 72, 79, 86, 87 |
| NO: 530 | SAVLFDHLNAMLGIPKNRMYIHFVNLNGDDVGWNGTTF | |
| 153-47A.1NegT2 | MPIFTLNTNIKADDVPSDFLSLTSRLVGLILSEPGSYV | I53-47A: |
| SEQ ID | AVHINTDQQLSFGGSTNPAAFGTLMSIGGIEPDKNEDH | 22, 25, 29, 72, 79, 86, 87 |
| NO: 531 | SAVLFDHLNAMLGIPKNRMYIHFVDLDGDDVGWNGTTF | |

I53-47A genus
(SEQ ID NO: 532)
MPIFTLNTNIKA(T/D)DVPSDFLSLTSRLVGLILS(K/E)PGSYVAVHI
NTDQQLSFGGSTNPAAFGTLMSIGGIEP(S/D)KN(R/E)DHSAVLFDHL
NAMLGIPKNRMYIHFV(N/D)L(N/D)GDDVGWNGTTF

The first polypeptides of SEQ IDS NO:3 and 530-532 are polypeptides disclosed in U.S. Pat. No. 9,630,994 (incorporated by reference herein in its entirety) that form homo-trimers that can non-covalently interact with the polypeptides of the second aspect of the disclosure to generate the nanostructures. The first polypeptides of the third aspect of the disclosure are improved homo-trimer-forming polypeptides as described herein.

In one embodiment, wherein the second polypeptides are at least 75%, 80%, 85%, 90%, 91%, 92%, 93%, 94%, 95%, 96%, 97%, 98%, or 99% identical over the length of the amino acid sequence selected from the group consisting of SEQ IDS NO:3 and 530-532, the polypeptides are also identical at least at 1, 2, 3, 4, 5, 6, 7, 8, 9, or 10 identified interface positions of the amino acid sequence selected from the group consisting of SEQ IDS NO:3 and 530-532.

In one specific embodiment, the nanostructures may comprise

(a) a plurality of first assemblies, each first assembly comprising a plurality of identical first polypeptides, wherein the first polypeptides comprise the polypeptide of any embodiment or combination of embodiments of the third aspect of the disclosure; and

(b) a plurality of second assemblies, each second assembly comprising a plurality of identical second polypeptides, wherein the second polypeptides comprise the polypeptide of any embodiment or combination of embodiments of the fourth aspect of the disclosure; wherein the plurality of first assemblies non-covalently interact with the plurality of second assemblies to form a nanostructure.

In another specific embodiment,

(a) the first polypeptides comprises the amino acid sequence of SEQ ID NO:22; and

(b) the second polypeptides comprises the amino acid sequence of SEQ ID NO:23: I53-47-v1 pentameric component.

The nanostructures of any embodiment or combination of embodiments of the disclosure may comprise at least one first polypeptide that comprises a linked targeting domain, and/or at least one second polypeptide that comprises a linked targeting domain. Any suitable targeting domain may be linked to at least one of the first and/or second polypeptides in the nanostructure. Exemplary targeting domains and linkage types (i.e.: covalent or non-covalent) are described in detail herein, and any such targeting domains or combinations thereof may be present in the nanostructures of the disclosure. The targeting domains may be linked to the first and/or second polypeptides in any valency suitable for an intended purpose. In various embodiments, at least two first polypeptides each comprise a linked targeting domain, and/or at least two second polypeptides each comprise a linked targeting domain, up to each of the first polypeptides and/or each of the second polypeptides comprise a linked targeting domain. The targeting domains linked to the first and/or second polypeptides in any nanostructure may identical, or they may bind the same target but not be identical.

In another embodiment, the nanostructure of any embodiment or combination of embodiments of the disclosure may comprise a nucleic acid capable of expressing the at least one first polypeptide and/or the at least one second polypeptide packaged within the nanostructure. In this embodiment, a genome encoding the nanostructure may be packaged within the nanostructure. As described in the examples that follow, the nanostructures of the disclosure have been evolved to result in drastically improved genome packaging (>133-fold), stability in whole murine blood (from less than 3.7% to 71% of packaged RNA protected after 6 hours of treatment), and in vivo circulation time (from less than 5 minutes to 4.5 hours), with some embodiments able to package one full-length RNA genome for every 11 nanostructures. Further, these nanostructures can be modularly retargeted in vitro and in vivo.

The nanostructures have a dimension in the nanometer scale (i.e.: 1 nm to 999 nm). In one embodiment, the nanostructures have a diameter in the nanometer scale. In various other embodiments, each first assembly comprises 3 copies of the identical first polypeptide, and each second assembly comprises 5 copies of the identical second polypeptide.

The nanostructures of the disclosure can be used for any suitable purpose, including but not limited to delivery vehicles, as the nanostructures can encapsulate molecules of interest and/or the first and/or second proteins can be modified to bind to molecules of interest (diagnostics, therapeutics, detectable molecules for imaging and other applications, etc.). The nanostructures of the invention are well suited for several applications, including vaccine design, targeted delivery of therapeutics, and bioenergy. In one embodiment, the nanostructure further comprises a cargo within the nanostructure. As used herein, a “cargo” is any compound or material that can be incorporated on and/or within the nanostructure. For example, polypeptide pairs suitable for nanostructure self-assembly can be expressed/purified independently; they can then be mixed in vitro in the presence of a cargo of interest to produce the nanostructure comprising a cargo. This feature, combined with the protein nanostructures' large lumens and relatively small pore sizes, makes them well suited for the encapsulation of a broad range of cargo including, but not limited to, small molecules, nucleic acids, polymers, and other proteins. In turn, the protein nanostructures of the present invention could be used for many applications in medicine and biotechnology, including targeted drug delivery and vaccine design. For targeted drug delivery, targeting moieties could be fused or conjugated to the protein nanostructure exterior to mediate binding and entry into specific cell populations and drug molecules could be encapsulated in the cage interior for release upon entry to the target cell or sub-cellular compartment. For vaccine design, antigenic epitopes from pathogens could be fused or conjugated to the cage exterior to stimulate development of adaptive immune responses to the displayed epitopes, with adjuvants and other immunomodulatory compounds attached to the exterior and/or encapsulated in the cage interior to help tailor the type of immune response generated for each pathogen. The polypeptide components may be modified as noted above. In one non-limiting example, the polypeptides can be modified, such as by introduction of various cysteine residues at defined positions to facilitate linkage to one or more antigens of interest as cargo, and the nanostructure could act as a scaffold to provide a large number of antigens for delivery as a vaccine to generate an improved immune response. Other modifications of the polypeptides as discussed above may also be useful for incorporating cargo into the nanostructure.

In a sixth aspect, the disclosure provides polynucleotides encoding the polypeptide of any embodiment or combination of embodiments of the first, second, third, or fourth aspects of the disclosure. The polynucleotides may comprise RNA or DNA. Such polynucleotides may comprise additional sequences useful for promoting expression and/or purification of the encoded polypeptides, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure. In one embodiment, the polynucleotides, or expression vectors thereof, may be loaded as cargo into the nanostructures of the disclosure, such that the nanostructures package their own genome as demonstrated in the examples that follow.

In one embodiment, the polynucleotides comprise a peptide linker encoding sequence, wherein the peptide linker encoding sequence is encoded by a DNA sequence that contains a Ribosome Binding Site (RBS)-like motif [RRRRRR (SEQ ID NO:533), where R is A or G], and/or an RNA secondary structure (e.g., hairpin structure), and/or a slippery sequence [e.g., CTTT (SEQ ID NO:534)]. In another embodiment, the DNA sequence has one or more mutations in the RBS-like motif and/or slippery sequence. These embodiments are particularly useful for polynucleotides that encode polypeptides that are translational fusions with polypeptide targeting domains, to control valency of the expressed targeting domain via frameshifting. Exemplary such DNA sequences include, but are not limited to:

(RBS-like motif is bold underlined and can be mutated to control frameshifting frequency)
(Slippery sequence is bold italicized and can be mutated to control frameshifting frequency)
(All sequences in parentheses are optional)

SEQ ID NO: 535: GSprfB
(CTCGAGGGTTCT)AGGGGGTATCTTT(GACGGCTCCGGTTCCGGTTCT)
SEQ ID NO: 536: AtAOS DNA sequence
(TAC)AAAAAAG(CAGGCTTGGCTTCCGGGTA)
SEQ ID NO: 537: Additional frameshift DNA sequence
ACCCCAAAA(GCGTAACGC)CTGACGGAGTGACTTTGAGCCAGAAAACGC
TCACGGGTG(CTGTCGGT)

In another aspect, the present invention provides recombinant expression vectors comprising the polynucleotide of any embodiment or combination of embodiments of the disclosure operatively linked to a suitable control sequence. “Recombinant expression vector” includes vectors that operatively link a nucleic acid coding region or gene to any control sequences capable of effecting expression of the gene product. “Control sequences” operably linked to the polynucleotides of the disclosure are nucleic acid sequences capable of effecting the expression of the polynucleotides. The control sequences need not be contiguous with the polynucleotides, so long as they function to direct the expression thereof. Thus, for example, intervening untranslated yet transcribed sequences can be present between a promoter sequence and the polynucleotides and the promoter sequence can still be considered “operably linked” to the polynucleotides. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive).

In another aspect, the present invention provides host cells that have been transfected with the recombinant expression vectors disclosed herein, wherein the host cells can be either prokaryotic or eukaryotic. The cells can be transiently or stably transfected. A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide.

In a further aspect are provided methods of using the nanostructures of the present invention. The nanostructures of the present disclosure can be used for many applications in medicine and biotechnology, including targeted drug delivery and vaccine design. For targeted drug delivery, targeting moieties could be fused or conjugated to the nanostructure exterior to mediate binding and entry into specific cell populations and drug molecules could be encapsulated in the cage interior for release upon entry to the target cell or sub-cellular compartment. For vaccine design, antigenic epitopes from pathogens could be fused or conjugated to the nanostructure exterior to stimulate development of adaptive immune responses to the displayed epitopes, with adjuvants and other immunomodulatory compounds attached to the exterior and/or encapsulated in the cage interior to help tailor the type of immune response generated for each pathogen. Other uses will be clear to those of skill in the art based on the disclosure relating to polypeptide modifications, nanostructure design, and cargo incorporation.

We report the invention of synthetic nucleocapsids, which are computationally-designed protein containers (capsids) that can encapsulate nucleic acids. In some embodiments, the capsid is composed of proteins that are of non-viral origin and/or non-container origin. In some embodiments, the capsid is derived from a computationally designed polyhedral assembly (e.g., icosahedral, tetrahedral, octahedral). In some embodiments, nucleic acids are encapsulated via simple charge complementarity. In some embodiments, nucleic acids are encapsulated via specific binding interactions with one or more RNA binding domains. The attached manuscript demonstrates a general method for evolving synthetic nucleocapsids. This method should be applicable to any type of non-viral protein container and is here demonstrated for two such containers (I53-50 and I53-47).

### Deep Mutational Scanning:

Deep sequencing of the various libraries of synthetic nucleocapsids enabled evaluation of the sequence-function relationship of large numbers of variants. Each variant represents a non-limiting example of the invention and underscores the generality of the approaches described. For capsids with increased nucleic acid packaging, nuclease protection, or in vivo circulation time, the composition claimed refers not only to the amino acid sequences reported in Supplementary table S3, but also to a family of related sequences found to have positive log enrichment scores in the deep mutational scanning data for each independent property selected. These properties include nucleic acid packaging, nuclease resistance, protease resistance (including proteases in whole murine blood), and in vivo circulation time.

### Independence of Mutations:

Capsids incorporating subsets of the mutations in the reported variants are likely to retain the improved properties, and thus each mutation ought to be protected independently. For example, capsids incorporating only the mutations found to increase circulation time (exterior surface amino acid composition from I53-50-v4) could be implemented without a positively-charged interior (interior surface amino acid composition from I53-50-v0) so as to generate a long-lived capsid without encapsulated nucleic acid. This could be useful for packaging other cargo such as small molecules, proteins, or other polymers.

Embodiments of the invention include a general solution, comprising a nucleocapsid which packages its own RNA and is derived from non-viral proteins. Embodiments may exclude natural, non-viral containers, specifically including but not limited to lumazine synthase, ferritin, and encapsulin. Similar packaging has not been disclosed or suggested in these systems, such that the present disclosure covers these systems in a novel and non-obvious manner.

Example claimed embodiments include:
A composition: comprising a synthetic nucleocapsid composed of a computationally-designed capsid derived from proteins that are of non-viral and/or non-container origin and designed to contact each other, wherein the capsid contacts a nucleic acid encoding its own genetic information.
Any one of the above, wherein that synthetic nucleocapsid is derivatized and subjected to selection to isolate variants with improved function.
Any one of the above, wherein that function is one or more of genome packaging, nuclease resistance, protease resistance, degradative enzyme resistance, increased circulation time in vivo, cell-specific targeting, protein scaffolding, or display of vaccine epitopes.
Any one of the above, wherein the net interior charge is between −200 and +1200.
Any one of the above, wherein a RNA-binding peptide is appended to a terminus of one of the capsid proteins.
Any one of the above, wherein the nucleocapsid pores are <6000 angstrom\hat{ }2.
Any one of the above, wherein the amino acids within 10 angstroms of the nucleocapsid pores comprise one of a net negative charge or a neutral charge.
Any one of the above, wherein a hydrophilic polypeptide is appended to the capsid proteins.
Any one of the above, wherein the hydrophilic polypeptide is one of the sequences in table S3.
A composition, comprising I53-50-v0 sequence (described in the manuscript and disclosed in U.S. Pat. No. 9,630,994 B2) modified with one or more of the following mutations:
   Trimer: T126D, E166K, S179K, T185K, A195K, E198K, S179N, T185N, E188K, K9R, K11T, K61D, E74D; and/or Pentamer: Y9H, A38R, S105D, D122K, D124K, E24F, D124N, H126K, H6Q, H9Q, D39K, D43E, E67K.
A composition, comprising a I53-47 sequence modified with one or more of the following mutations: Trimer: T13D, S71K, N101R, D105K; and/or Pentamer: D122K, D124K.
Any one of the above, wherein a natural and/or functional polypeptide domain is appended to the capsid proteins.
Any one of the above, wherein the natural and/or functional polypeptide domain is CD47.
Any one of the above, wherein the natural and/or functional polypeptide domain is an RNA binding domain.
Any one of the above, wherein the RNA binding domain is the Bovine Immunodefficiency Virus Tat RNA-binding peptide (Btat).
Any one of the above, wherein a natural and/or functional polypeptide is appended to the capsid proteins.
Any one of the above, wherein the natural and/or functional polypeptide is derived from CD47.
Any one of the above, wherein an intact protein domain is appended to the capsid proteins.
A system comprising one or more components as described and/or illustrated herein.
A device comprising one or more elements as described and/or illustrated herein.
A method comprising one or more steps as described and/or illustrated herein.
A non-transitory computer readable medium having computer executable instructions stored thereon that, if executed by one or more processors of a computing device, cause the computing device to perform one or more steps as described and/or illustrated herein.

The synthetic nucleocapsids and synthetic capsids described herein comprise non-naturally occurring sequences of protein assemblies encoded by non-naturally occurring sequences of polynucleotides. In an application, the synthetic capsids described herein are not derived from naturally occurring viral particles, and can be adapted to targeted delivery of cargo. Unlike most viruses, which are composed of proteins that adopt multiple different conformations during capsid assembly and/or dock in domain-swapped conformations, the protein assemblies of the synthetic nucleocapsids and synthetic capsids comprise highly stable subunits that adopt a single conformation, fold independently, and dock into simple icosahedral symmetry. This allows them to tolerate the attachment of modular cargo packaging domains on the interior (such as, for example, BIV Tat RNA binding domain, and the like) and/or modular cell targeting domains on the exterior (such as, for example, scFv, nanobody, DARPin, affibody, monobody, etc.).

Targeted delivery of encapsulated therapeutic cargos (e.g., RNA, DNA, small molecules, peptides, proteins, non-biological polymers) remains a major challenge in medicine. The use of synthetic capsids to deliver therapeutic cargos can avoid problems associated with viral delivery systems (e.g., safety concerns, pre-existing immunity to the viral capsid proteins, inability to package non-nucleic acid cargos, difficulty to formulate) and with nanoparticle delivery systems (e.g., poor targeting to cells other than liver or immune cells, toxicity, immunogenicity, lack of atomic-level control, lack of ability to evolve new tropisms).

The inventors have discovered that one or more modular targeting domains can be incorporated (for example, operably linked, chemical conjugation, crosslinking, or the like) with the synthetic nucleocapsids or synthetic capsids such that the one or more modular targeting domains are exposed on the exterior of synthetic nucleocapsids without compromising the ability of (1) the synthetic nucleocapsids to assemble and package their genome or (2) the targeting domain to specifically bind to cells expressing its target. In this regard, the target can comprise, for example, a protein target, a small molecule target, a chemical target, an extracellular surface target, etc. The modular nature of synthetic nucleocapsids provides an advantage over existing viral capsids by allowing facile retargeting to alternative cells expressing different targets. For example, MS2 bacteriophage and AAV only have a small number of amino acids that can be changed without compromising capsid assembly. Furthermore, they do not tolerate insertion of large protein domains such as DARPins, affibodies, etc.

As used herein, “synthetic” means non-naturally occurring. When referring to synthetic nucleocapsids, “synthetic” includes polypeptide sequences comprising naturally occurring amino acids, but the amino acid sequence of which was non-naturally occurring or not derived from nature and includes polynucleotide sequences comprising naturally occurring nucleic acids, but the polynucleotide sequence of which was non-naturally occurring or not derived from nature. Additional non-natural amino acids and nucleic acids can be substituted for the naturally occurring amino acids or nucleic acids, provided that these substitutions do not alter the ability to adopt a single conformation, to fold independently, and to dock into an assembly with the simple, designed icosahedral symmetry.

In an aspect, the invention comprises compositions comprising, a) a synthetic capsid comprising protein assemblies of non-naturally occurring proteins. In an application the protein assemblies form highly stable subunits that adopt a single conformation, fold independently, and dock into simple icosahedral symmetry. In a further application the synthetic capsid comprises one or more modular targeting domains. In an example, the synthetic nucleocapsid protein assembly can be derived from a nucleocapsid capable of packaging its own genome and evolving complex properties, which has been modified and/or purified in such a manner so as to no longer package its own genome. In another example, the synthetic nucleocapsid protein assembly can be produced without its genome and used to electrostatically package negatively-charged polymers, including but not limited to nucleic acids such as but not limited to single stranded DNA, double stranded DNA, mRNA, siRNA, and artificial nucleic acids, such as peptide nucleic acids (PNA), Morpholino and locked nucleic acids (LNA), glycol nucleic acids (GNA) and threose nucleic acids (TNA). In another example, the interior surface of the protein assembly may be modified with cargo recruitment moieties instead of electrostatically packaging negatively charged polymers. Examples of cargo recruitment moieties include chemically reactive groups (e.g., cysteines for crosslinking with maleimide-functionalized molecules or non-canonical amino acids such as p-acetylphenylalanine that can undergo bioorthogonal bond formation) and polypeptides (e.g., nucleic acid binding domains for recruitment of specific RNA or DNA sequences).

In an example, the synthetic nucleocapsid protein assembly may be a non-natural nucleocapsid protein assembly as described in the U.S. Pat. No. 9,630,994 B2 (Bale, et al.) or the nucleocapsids described in Exhibit A, herein.

In another example, the synthetic nucleocapsid protein assembly may comprise a protein having at least 50%, 60%, 70%, 80%, 90%, 95%, 96%, 97%, 98%, 99%, or 100% amino acid sequence identity to one or more of the amino acid sequences selected from SEQ ID Nos.:01-02 (referred to as SEQ ID NOS: 68-69 in the priority application) herein, or the I53-50-v0 sequence described in U.S. Pat. No. 9,630,994 B2,

(SEQ ID NO: 1; Trimer)
(MKM)EELFKKHKIVAVLRANSVEEAIEKAVAVFAGGVHLIEITFTVPDA
DTVIKALSVLKEKGAIIGAGTVTSVEQCRKAVESGAEFIVSPHLDEEISQ
FCKEKGVFYMPGVMTPTELVKAMKLGHTILKLFPGEVVGPQFVKAMKGPF
PNVKEVPTGGVNLDNVCEWFKAGVLAVGVGSALVKGTPDEVREKAKAFVE
KIRGCTE
(SEQ ID NO: 2 Pentamer)
(M)NQHSHKDYETVRIAVVRARWHAEIVDACVSAFEAAMADIGGDRFAVD
VFDVPGAYEIPLHARTLAETGRYGAVLGTAFVVNGGIYRHEFVASAVIDG
MMNVQLSTGVPVLSAVLTPHRYRDSDAHTLLFLALFAVKGMEAARACVEI
LAAREKIAA

as modified with one or more of the following amino acid changes: (Trimer: T126D, E166K, S179K, T185K, A195K, E198K, S179N, T185N, E188K, K9R, K11T, K61D, E74D; Pentamer Y9H, A38R, S105D, D122K, D124K, E24F, D124N, H126K, H6Q, H9Q, D39K, D43E, E67K, R119N, R121D). Similarly, the protein assembly may comprise a protein having at least 50%, 60%, 70%, 80%, 90%, 95%, 96%, 97%, 98%, 99%, or 100% amino acid sequence identity to a protein selected from one or more of the amino acid sequences of SEQ ID Nos.:03-04 (referred to as SEQ ID NOS: 70-71 in the priority application) herein or to the I53-47 sequence described in U.S. Pat. No. 9,630,994 B2,

(SEQ ID NO: 3 Trimer)
(M)PIFTLNTNIKATDVPSDFLSLTSRLVGLILSKPGSYVAVHINTDQQL
SFGGSTNPAAFGTLMSIGGIEPSKNRDHSAVLFDHLNAMLGIPKNRMYIH
FVNLNGDDVGWNGTTF
(SEQ ID NO: 4 Pentamer)
(M)NQHSHKDHETVRIAWRARWHADIVDACVEAFEIAMAAIGGDRFAVDV
FDVPGAYEIPLHARTLAETGRYGAVLGTAFVVNGGIYRHEFVASAVIDGM
MNVQLSTGVPVLSAVLTPHRYRDSAEHHRFFAAHFAVKGVEAARACIEIL
AAREKIAA

as modified with one or more of the following amino acid changes (Pentamer: S105D, R119N, R121D, D122K, A124K, A150N; Trimer: T13D, 571K, N101R, D105K. In another example, the synthetic nucleocapsid protein assembly may comprise a protein having at least 50%, 60%, 70%, 80%, 90%, 95%, 96%, 97%, 98%, 99%, or 100% amino acid sequence identity to one or more of the icosahedral assemblies described in U.S. Pat. No. 9,630,994 B2, incorporated herein by reference for the amino acid sequences thereof.

In another example, the synthetic nucleocapsid protein assembly comprises a protein selected from one or more of SEQ ID Nos.:01-02 described herein or the I53-50-v0 sequence described in U.S. Pat. No. 9,630,994 B2, as modified with one or more of the following amino acid changes: (Trimer: T126D, E166K, S179K, T185K, A195K, E198K, 5179N, T185N, E188K, K9R, K11T, K61D, E74D; Pentamer Y9H, A38R, S105D, D122K, D124K, E24F, D124N, H126K, H6Q, H9Q, D39K, D43E, E67K, R119N, R121D). Similarly, the synthetic nucleocapsid protein assembly comprises a protein selected from one or more of the amino acid sequence of one or more of SEQ ID Nos.:03-04, herein or to the I53-47 sequence described in U.S. Pat. No. 9,630,994 B2, as modified with one or more of the following amino acid changes: (Pentamer: 5105D, R119N, R121D, D122K, A124K, A150N; Trimer: T13D, S71K, N101R, D105K).

In another embodiment, the synthetic nucleocapsid protein assembly comprises a protein having at least 50%, 60%, 70%, 80%, 90%, 95%, 96%, 97%, 98%, 99%, or 100% amino acid sequence identity to one or more of the amino acid sequences selected from SEQ ID Nos. 5, 15, 19, 20, 9, and 10 (referred to as SEQ ID NOS:1-6 in the priority application), herein, or to the I53-50-v0 sequence described in U.S. Pat. No. 9,630,994 B2. In another example, the synthetic nucleocapsid protein assembly comprises an amino acid sequence selected from one or more of the amino acid sequences of SEQ ID Nos. 5, 15, 19, 20, 9, and 10, herein, I53-50-v0 sequence described in U.S. Pat. No. 9,630,994 B2.

In another example, the targeting domain is a polypeptide. In an embodiment, the targeting domain is a globular protein-binding domain. In a further embodiment, the targeting domain can be, for example, an antibody, scFv, nanobody, DARPin, affibody, monobody, adnectin, alphabody, Albumin-binding domain, Adhiron, Affilin, Affimer, Affitin/Nanofitin, Anticalin, Armadillo repeat proteins, Atrimer/Tetranectin, Avimer/Maxibody, Centyrin, Fynomer, Kunitz domain, Obody/OB-fold, Pronectin, Repebody, or a computationally designed protein.

In an example, the targeting domains described herein can have at least 50%, 60%, 70%, 80%, 90%, 95%, 96%, 97%, 98%, 99%, or 100% sequence identity to one or more sequences selected from SEQ ID Nos 24-43 (referred to as SEQ ID NOS: 7-17 or 65-67 in the priority application), herein. In an embodiment, the targeting domain comprises or consists of one or more amino acid sequences selected from SEQ ID Nos 24-43, herein.

In an example, the amino acid sequence of any the targeting domains can include any amino acid at the positions specified in brackets within the binder sequences and listed in the “Commonly mutated positions in binding domains” portion, herein.

In an example, the synthetic nucleocapsid protein assembly and targeting domain of any combination thereof are linked by a non-covalent attachment [e.g., biotin-streptavidin, protein-protein interaction]. In an example, the synthetic nucleocapsid protein assembly and targeting domain are of any combination thereof linked by a covalent attachment. In an embodiment, the covalent attachment is post-translational [spycatcher-spytag; split intein; click chemistry, etc.]. In another embodiment, the covalent attachment is accomplished via translational fusion. In another embodiment, the translation fusion can be to any terminus or loop in the synthetic nucleocapsid protein assembly. In another embodiment, the translation fusion is to the N-term or C-term of a trimer. In another embodiment, the translation fusion is to the N-term or C-term of a pentamer. In another embodiment, the translation fusion comprises a synthetic nucleocapsid protein assembly, a polypeptide linker, and a targeting domain. In a further embodiment, the polypeptide linker comprises a flexible amino acid sequence that results in display of the targeting domain on every monomer to which it is translationally fused. In a further embodiment, the polypeptide linker comprises a frameshift sequence that results in at least one monomer that does not display the targeting domain. In another embodiment, the polypeptide linker comprises an internal ribosome binding site motif and alternative start site that results in at least one monomer that does not display the targeting domain. In another embodiment, a multicistronic operon comprises both an assembly subunit without a targeting domain and an assembly subunit with a targeting domain that results in at least one monomer that does not display the targeting domain. In a further embodiment, the polypeptide linker has at least 50%, 60%, 70%, 80%, 90%, 95%, 96%, 97%, 98%, 99%, or 100% sequence identity to one or more sequences selected from SEQ ID Nos 44-57 (referred to as SEQ ID NOS:18-32 in the priority application), herein. In an embodiment, the polypeptide linker is selected from SEQ ID Nos 44-57.

In another example, the invention provides a DNA sequence encoding a polypeptide linker that contains a Ribosome Binding Site (RBS)-like motif [RRRRRR (SEQ ID NO:533), where R is A or G], and/or an RNA secondary structure, and/or a slippery sequence [e.g., CTTT (SEQ ID NO:534)]. In an embodiment, one or more mutations in the DNA sequence of the RBS-like motif and/or slippery sequence tune the copy number of the targeting domain.

In an example, the invention comprises compositions comprising, a) a synthetic nucleocapsid protein assembly and b) a targeting domain, wherein the composition comprises a protein with 50%, 60%, 70%, 80%, 90%, 95%, 96%, 97%, 98%, 99%, or 100% sequence identity to one or more sequences selected from one of SEQ ID Nos. 541-561 and 572-582.referred to as SEQ ID NOS:33-64 in the priority application) In an example, the invention comprises compositions comprising, a) a synthetic nucleocapsid protein assembly, and b) a targeting domain, wherein the composition comprises a protein selected from one of SEQ ID Nos. 541-561 and 572-582.

### Example Embodiments

A polypeptide comprising: a) a synthetic capsid protein assembly, and b) a targeting domain.
The polypeptide of claim 1, wherein the synthetic capsid protein assembly comprises an amino acid sequence having at least 50%, 60%, 70%, 80%, or 90% sequence identity to the amino acid sequence selected from SEQ ID Nos. 01-02 or to the I53-50-v0 sequence as disclosed in U.S. Pat. No. 9,630,994 B2 ([[SEQ ID NO:1 Trimer; SEQ ID NO:2 Pentamer]] as modified with one or more of the following amino acid changes: (Trimer: T126D, E166K, S179K, T185K, A195K, E198K, S179N, T185N, E188K, K9R, K11T, K61D, E74D; Pentamer Y9H, A38R, S105D, D122K, D124K, E24F, D124N, H126K, H6Q, H9Q, D39K, D43E, E67K, R119N, R121D) or to the amino acid sequence selected from SEQ ID Nos. 70-71 or to the I53-47 sequence as disclosed in 059630994 B2 as modified with one or more of the following amino acid changes (Pentamer: S105D, R119N, R121D, D122K, A124K, A150N; Trimer: T13D, S71K, N101R, D105K.
The polypeptide of claim 1, wherein the synthetic capsid protein assembly comprises an amino acid sequence selected from SEQ ID Nos 01-02 or to the I53-50-v0 sequence as disclosed in U.S. Pat. No. 9,630,994 B2 as modified with one or more of the following amino acid changes: (Trimer: T126D, E166K, S179K, T185K, A195K, E198K, S179N, T185N, E188K, K9R, K11T, K61D, E74D; Pentamer Y9H, A38R, S105D, D122K, D124K, E24F, D124N, H126K, H6Q, H9Q, D39K, D43E, E67K, R119N, R121D) or the amino acid sequence selected from SEQ ID Nos. SEQ ID 70-71 or to the I53-47 sequence as disclosed in U.S. Pat. No. 9,630,994 B2 as modified with one or more of the following amino acid changes: (Pentamer: S105D, R119N, R121D, D122K, A124K, A150N; Trimer: T13D, S71K, N101R, D105K).
The polypeptide of claim 1, wherein the synthetic capsid protein assembly comprises an amino acid sequence having at least 50%, 60%, 70%, 80%, or 90% sequence identity to an amino acid sequence selected from SEQ ID Nos. 5, 15, 19, 20, 9, and 10 or to the I53-50-v4 sequence described herein.
The polypeptide of claim 1, wherein the synthetic capsid protein assembly comprises an amino acid sequence selected from SEQ ID Nos. 5, 15, 19, 20, 9, and 10 or to the I53-50-v0 sequence described in U.S. Pat. No. 9,630,994 B2.
The polypeptide of any previous claim, wherein the targeting domain is a polypeptide.
The polypeptide of claim 6, wherein the targeting domain is a globular protein-binding domain.
The polypeptide of claim 7, wherein the targeting domain is an antibody, scFv, nanobody, DARPin, affibody, monobody, adnectin, alphabody, Albumin-binding domain, Adhiron, Affilin, Affimer, Affitin/Nanofitin, Anticalin, Armadillo repeat proteins, Atrimer/Tetranectin, Avimer/Maxibody, Centyrin, Fynomer, Kunitz domain, Obody/OB-fold, Pronectin, Repebody, or computationally designed protein.
The polypeptide of any previous claim, wherein the targeting domain has at least 50%, 60%, 70%, 80%, or 90% sequence identity to one or more sequences selected from SEQ ID Nos. 24-43.
The polypeptide of claim 9, wherein the targeting domain comprises an amino acid sequence selected from SEQ ID No. 24-43.
The polypeptide of any previous claim, wherein the amino acid sequence can include any amino acid at the positions specified in brackets within the binder sequences and listed in the “Commonly mutated positions in binding domains” portion of the disclosure.
The polypeptide of any previous claim, wherein the synthetic nucleocapsid protein assembly and targeting domain are linked by a non-covalent attachment [e.g., biotin-streptavidin].
The polypeptide of any of claims 1-11, wherein the synthetic nucleocapsid protein assembly and targeting domain are linked by a covalent attachment.
The polypeptide of claim 13, wherein the covalent attachment is post-translational [spycatcher-spytag; split intein; click chemistry, etc.]
The polypeptide of claim 14, wherein the covalent attachment is accomplished via translational fusion.
The polypeptide of claim 15, wherein the translation fusion can be to any terminus or loop in the protein assembly of claim 1.
The polypeptide of claim 16, wherein the translation fusion is to the N-term or C-term of the trimer.
The polypeptide of claim 17, wherein the translation fusion is to the N-term or C-term of the pentamer.
The polypeptide of any previous claim, comprising a polypeptide linker.
The polypeptide of claim 19, wherein the polypeptide linker comprises a flexible amino acid sequence that results in display of the protein-binding domain on every monomer to which it is translationally fused.
The polypeptide of claim 19, wherein the polypeptide linker comprises a frameshift sequence that results in at least one monomer that does not display the targeting domain.
The polypeptide of any of claims 19-21, wherein the polypeptide linker has at least 50%, 60%, 70%, 80%, or 90% sequence identity to one or more sequences selected from one of SEQ ID Nos. 44-57.
The polypeptide of claim 22, wherein the polypeptide linker is selected from one of SEQ ID Nos. 44-57.
The polypeptide of claim 22, wherein the polypeptide linker is encoded by a DNA sequence that contains a Ribosome Binding Site (RBS)-like motif [RRRRRR (SEQ ID NO:533), where R is A or G], and/or an RNA secondary structure, and/or a slippery sequence [e.g., CTTT (SEQ ID NO:534)].
The polypeptide of claim 24, wherein the DNA sequence has one or more mutations in the RBS-like motif and/or slippery sequence to control the copy number of the targeting domain.
The polypeptide of any previous claim, wherein the amino acid sequence of the polypeptide has at least 50%, 60%, 70%, 80%, or 90% sequence identity to one or more sequences selected from SEQ ID Nos. 541-561 and 572-582 or 583-592, and 11-13.
The polypeptide of any previous claim, wherein the amino acid sequence of the polypeptide comprises an amino acid sequence selected from SEQ ID Nos. 541-561 and 572-582 or 583-592, and 11-13.
A synthetic nucleocapsid comprising the polypeptide of any previous claim.
A synthetic nucleocapsid comprising: a) a synthetic capsid protein assembly, and b) a synthetic genome.
A polynucleotide encoding the polypeptide of any previous claim
A composition comprises the polypeptide of any of claims 1-29 or the polynucleotide of claim 30.
Other polypeptides and polynucleotides described herein.
Use of the polypeptides and polynucleotides described and claimed herein for targeting delivery of encapsulated therapeutics in vitro or in vivo.
Use of the polypeptides and polynucleotides described and claimed herein for targeting delivery of encapsulated therapeutics in treatment of disease.
Other compositions and methods described herein.

The disclosure also provides compositions comprising a synthetic nucleocapsid composed of a computationally-designed capsid derived from proteins that are of non-viral and/or non-container origin and designed to contact each other, wherein the capsid contacts a nucleic acid encoding its own genetic information. In one embodiment, the synthetic nucleocapsid is derivatized and subjected to selection to isolate variants with improved function. In another embodiment, the improved function is one or more of genome packaging, nuclease resistance, protease resistance, degradative enzyme resistance, increased circulation time in vivo, cell-specific targeting, protein scaffolding, or display of vaccine epitopes. In a further embodiment, the net interior charge is between −200 and +1200. In another embodiment, the net interior charge is between +100 and +900. In one embodiment, a RNA-binding peptide is appended to a terminus of one of the capsid proteins. In another embodiment, the nucleocapsid pores are <6000 angstrom\hat{ }2. In a further embodiment, the amino acids within 10 angstroms of the nucleocapsid pores comprise one of a net negative charge or a neutral charge. In one embodiment, a hydrophilic polypeptide is appended to the capsid proteins. In a further embodiment, a targeting moiety is appended to the capsid proteins, including but not limited to a polypeptide targeting moiety (ex: an antibody, an scFv, a nanobody, a DARPin, an affibody, a monobody, adnectin, an alphabody, an albumin-binding domain, an adhiron, an affilin, an affimer, an affitin, an anticalin, an armadillo repeat proteins, a tetranectin, an avimer/maxibody, a centyrin, a fynomer, a kunitz domain, an obody/OB-fold, a PRONECTIN®, or a repebody)

In another aspect, methods of generating polypeptides that self-assemble and package nucleic acid that encodes the polypeptides are provided, comprising:

(a) symmetrically docking one or more polypeptides into an icosahedral geometry;

(b) redesigning the interior surfaces of the polypeptides to have a net charge between −200 and +1200, or between +100 and +900;

(c) encoding the polypeptides in a nucleic acid sequence;

(d) optionally introducing sequence variation in the nucleic acid sequence;

(e) introducing the nucleic acid(s) into a cell;

(f) culturing the cell under conditions to cause expression of the nucleic acid to produce the polypeptide in the cell; and

(g) isolating polypeptides that self-assemble and package the nucleic acid that encodes the polypeptide.

In one embodiment, isolating the polypeptide comprises:

(i) disrupting the cell membrane;

(ii) purifying polypeptide assemblies;

(iii) challenging the polypeptide assembly (e.g., degradative enzyme, blood, circulation, target binding); and

(iv) recovering the nucleic acids encapsulated by the polypeptide assembly.

In another embodiment, the methods further comprise identifying the polypeptides by sequencing. In a further embodiment, the methods further comprise performing one or more rounds of evolution by introducing the recovered nucleic acids into a new cell and repeating steps (e-g) and optionally repeating steps (i-iv).

In another aspect, the disclosure provides methods of generating the polypeptides or nanostructures of any of embodiment or combination of embodiments of the disclosure, wherein the methods comprise any methods disclosed herein, such as those described in the examples that follow.

In a further aspect, the disclosure provides synthetic nucleocapsids comprising: In a further aspect, the disclosure provides synthetic nucleocapsids comprising:

a plurality of first oligomeric polypeptides, each first oligomeric polypeptide comprising a plurality of identical first synthetic polypeptides;

a plurality of second oligomeric polypeptides, each second oligomeric polypeptide comprising a plurality of identical second synthetic polypeptides;

wherein the plurality of first oligomeric polypeptides and the plurality of second oligomeric polypeptides interact non-covalently and assemble into an icosahedral geometry with an interior cavity (a synthetic capsid) that contacts a nucleic acid encoding the polypeptide components of the synthetic nucleocapsid:

wherein the synthetic nucleocapsid does not require viral proteins or naturally-occurring non-viral container proteins, and the first oligomeric polypeptides and second oligomeric polypeptides are selected to provide a positive net charge on the interior surface.

In various embodiments, the first assemblies and second assemblies may be selected to provide the synthetic nucleocapsid with a net interior charge of between about +100 and about +900, between about +200 and about +800, between about +250 and about +750, between about +250 and about +650, between about +250 and about +500, between about +250 and about +450, between about +300 and about +750, between about +300 and about +650, between about +300 and about +500, or between about +300 and about +450. The net interior charge is measured using the methods disclosed in the examples that follow.

In other embodiments, the first assemblies and second assemblies may be selected to provide the synthetic nucleocapsid with a circulation half-life in live mice of at least 10 minutes, 1 hour, 2 hours, 3 hours, 4 hours, or 4.5 hours.

In further embodiments, the synthetic nucleocapsid may exhibit improved genome packaging, for example, at least one full-length RNA per 1,000 synthetic nucleocapsids, at least five full-length RNA per 1,000 synthetic nucleocapsids, at least 10 full-length RNA per 1,000 synthetic nucleocapsids, at least 25 full-length RNA per 1,000 synthetic nucleocapsids, at least 50 full-length RNA per 1,000 synthetic nucleocapsids, at least 75 full-length RNA per 1,000 synthetic nucleocapsids, or at least 90 full-length RNA per 1,000 synthetic nucleocapsids. Within the work described here, a full length RNA is defined as the mRNA molecule encoding the polypeptide components of the nanostructure. However, in some embodiments, an RNA fragment encoding only a subset of the nanostructure, or an RNA payload unrelated to the nanostructure, is used in a particular application, the minimal RNA sequence capable of carrying out the intended function should be quantified for purposes of determining packaging efficiency. The packaging efficiency is defined as the number of moles of full length RNA or (by RT-qPCR) per molar equivalent of intact nanomaterial protein as measured by qubit assay. Further assay details are described in the methods section under In vitro synthetic nucleocapsid selection conditions.

In other embodiments, the synthetic nucleocapsid may exhibit a half-life of greater than 0.5, 0.75 hours, 1 hour, or 1.5 hours at 37° C. in the presence of RNase A, with the RNase being present at a concentration of 10 μg/mL. The half-life is measured using the methods disclosed in the examples that follow, such as described in methods section under In vitro synthetic nucleocapsid selection conditions. In one embodiment, mutations that confer increased half-life include the trimer E67K mutation. In other embodiments, mutations that confer increased resistance to nuclease include 1, 2, 3, or all 4 of K2T, K9R, K11T, K61D.

In further embodiments, the synthetic nucleocapsid includes a plurality of pores, with each pore having an area of less than about 2000, 1800, 1600, 1000, 600, 300, or 150 angstroms². Pore area is determined by measuring the longest dimension at the widest point in the perpendicular dimension.

In another embodiment, at least one, two, three, or more (such as all) first synthetic polypeptide may comprise a linked targeting domain, and/or at least one, two, three, or more (such as all) second synthetic polypeptide may comprise a linked targeting domain. In one embodiment the targeting domain may be a polypeptide targeting domain, including but not limited to a polypeptide selected from the group consisting of an antibody, an antibody, an scFv, a nanobody, a DARPin, an affibody, a monobody, adnectin, an alphabody, an albumin-binding domain, an adhiron, an affilin, an affimer, an affitin, an anticalin, an armadillo repeat proteins, a tetranectin, an avimer/maxibody, a centyrin, a fynomer, a kunitz domain, an obody/OB-fold, a PRONECTIN®, a repebody, and CD47. In various further embodiments, the polypeptide targeting domain may comprise an amino acid sequence at least 50%, 60%, 70%, 80%, 90%, 95%, or 100% identical to a full length of an amino acid sequence selected from the group consisting of SEQ ID NOs: 24-43. In other embodiments, (i) the at least one first synthetic polypeptide or the at least one second synthetic polypeptide, and (ii) the polypeptide targeting domain may be linked by a non-covalent attachment or a covalent attachment, including but not limited to covalently linked by translational fusion. In further embodiments, the first synthetic polypeptides and/or the second synthetic polypeptides may comprise any embodiment or combination of embodiments of the first and second polypeptides disclosed herein for use in the nanostructures of the disclosure. In further embodiments, each first assembly may comprise 3 copies of the identical first polypeptide, and each second assembly may comprise 5 copies of the identical second polypeptide.

### Example 1

### Abstract

Billions of years of evolution have favored efficiency at the expense of modularity, making viral capsids difficult to engineer. Synthetic systems composed of non-viral proteins could provide a “blank slate” to evolve desired properties for drug delivery and other biomedical applications, while avoiding the safety risks and engineering challenges associated with viruses. Here we create synthetic nucleocapsids—computationally designed icosahedral protein assemblies with positively charged inner surfaces capable of packaging their own full-length mRNA genomes—and explore their ability to evolve virus-like properties by generating diversified populations using Escherichia coli as an expression host. Several generations of evolution resulted in drastically improved genome packaging (>133-fold), stability in whole murine blood (from less than 3.7% to 71% of packaged RNA protected after 6 hours of treatment), and in vivo circulation time (from less than 5 minutes to 4.5 hours). The resulting synthetic nucleocapsids package one full-length RNA genome for every 11 icosahedral assemblies. Our results show that there are simple evolutionary paths through which protein assemblies can acquire virus-like genome packaging and protection. The ability to computationally design synthetic nanomaterials and to optimize them through evolution now enables a complementary “bottom-up” approach with considerable advantages in programmability and control.

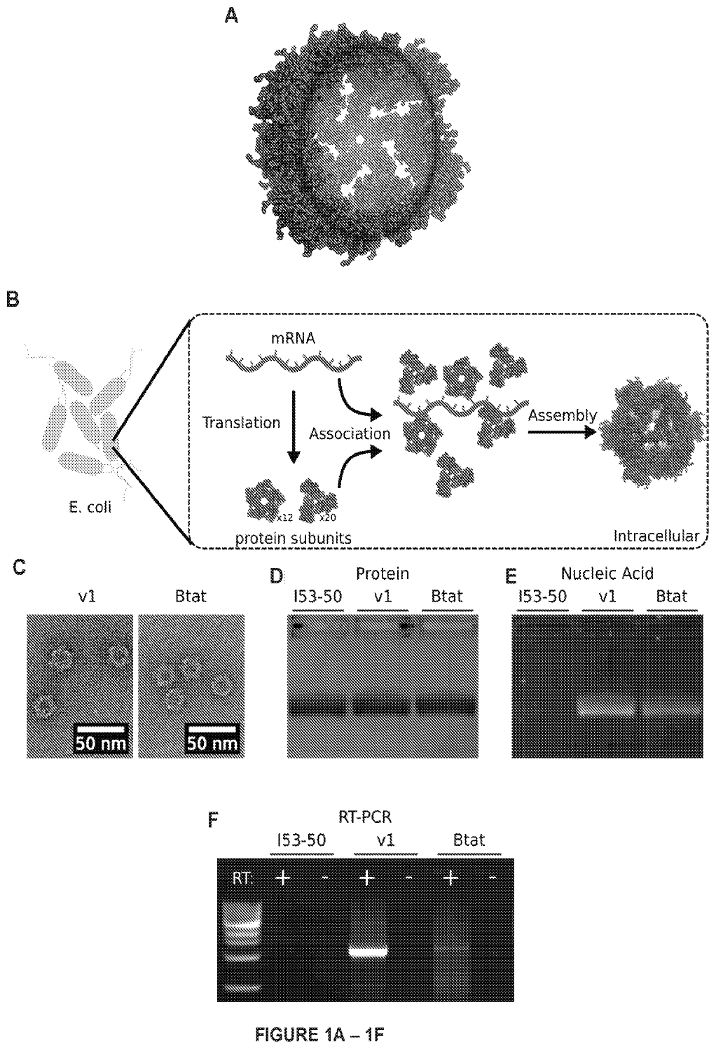

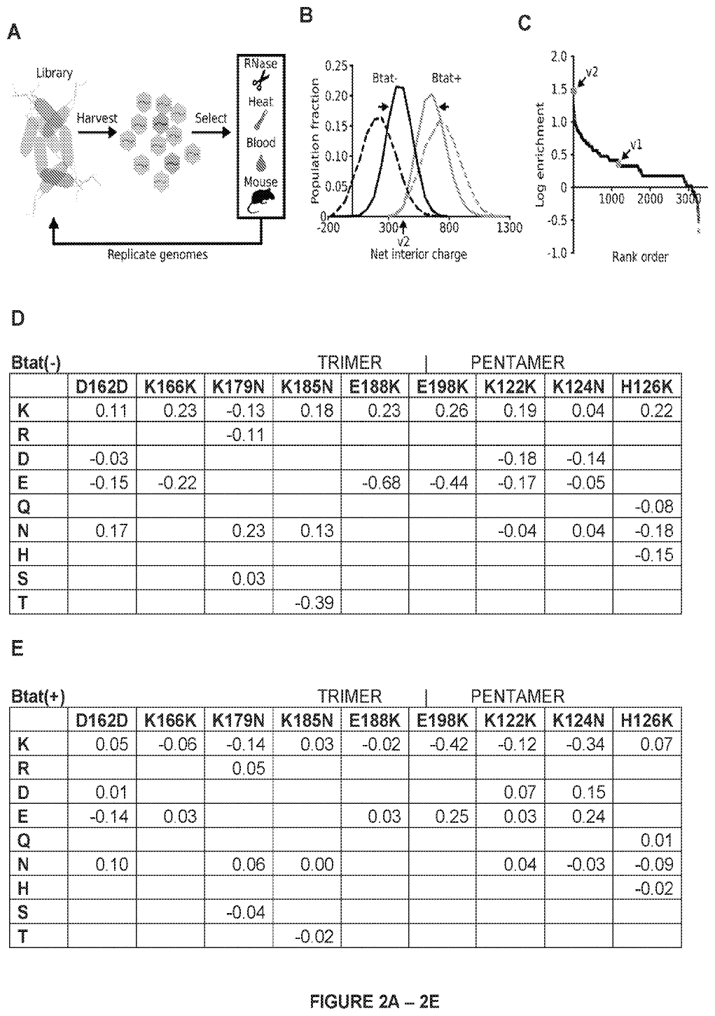

Highly stable and engineerable assemblies in principle could be redesigned to package their own genomes: bicistronic mRNAs encoding the two protein subunits. We investigated this possibility by modifying two assemblies with accessible protein termini and no large pores, I53-47 and I53-50, either by introducing positively charged residues on their interior surfaces (I53-47-v1 and I53-50-v1; FIG. 1a; Table 1) or by genetically fusing the Tat RNA-binding peptide from Bovine Immunodeficiency Virus¹⁵ to the interior-facing C-terminus of one subunit (I53-50-Btat and I53-47-Btat).

TABLE 1
All amino acid substitutions made for each
version relative to the previous version

| | Changes in trimer with | Changes in pentamer with |
|---|---|---|
| Version | respect to previous version | respect to previous version |
| 153-50-v1 | T126D, E166K, S179K, | Y9H, A38R, S105D, |
| | T185K, A195K, E198K | D122K, D124K |
| 153-50-v2 | K179N, K185N, E188K | E24F, K124N, H126K |
| 153-50-v3 | K9R, K11T, K61D | H6Q, H9Q |
| 153-50-v4 | E74D | D39K, D43E, E67K |

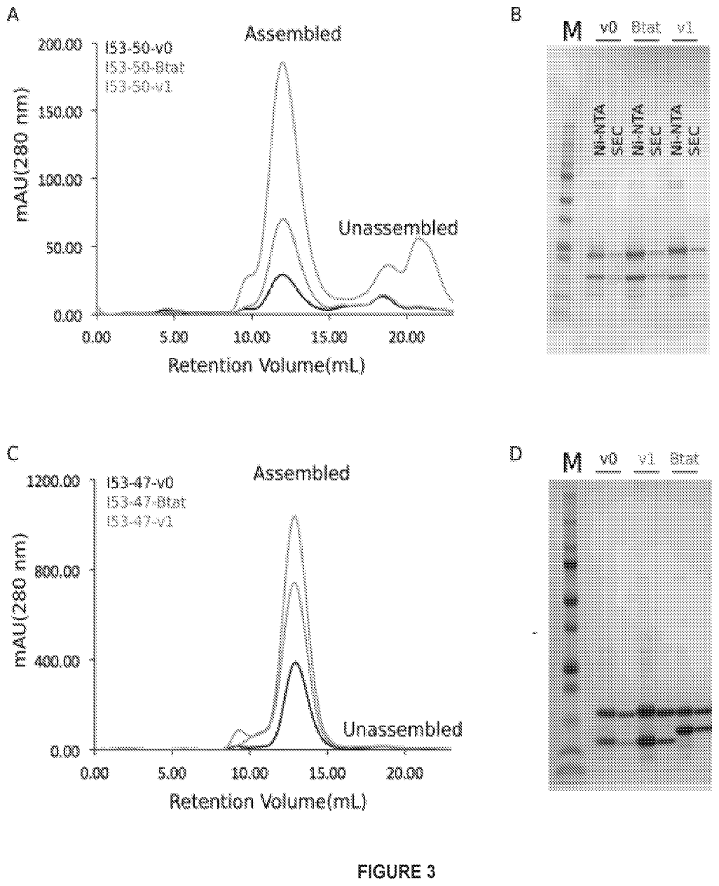

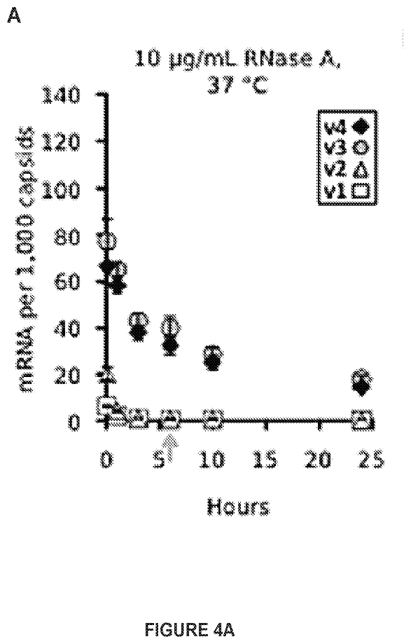

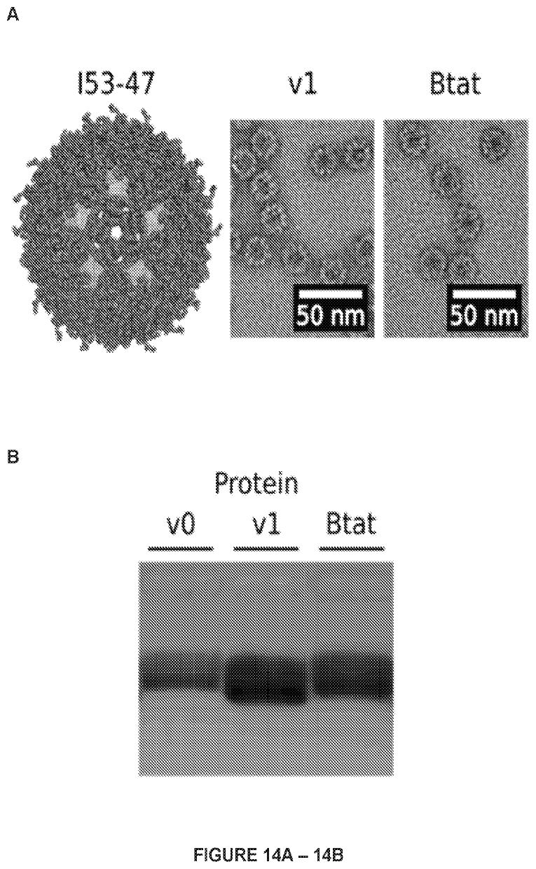

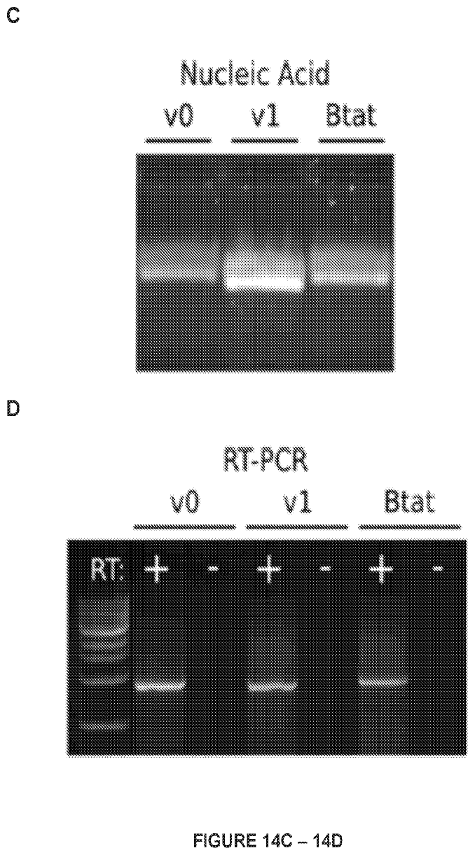

After expression and intracellular assembly in E. coli (FIG. 1b), intact protein assemblies were purified from cell lysates using immobilized metal affinity chromatography (IMAC) and size exclusion chromatography (SEC). The assemblies eluted as a single peak at the same retention volume as the original design (FIG. 3), and intact particles were observed by negative-stain transmission electron microscopy (FIG. 1c). After purification, the assemblies were incubated with RNase A for 10 minutes at 25° C. to degrade any RNA not protected inside the synthetic capsid-like proteins. Nucleic acid and protein co-migrated on native agarose gels (FIG. 1d,e), suggesting the remaining nucleic acid was encapsulated in the protein assembly. Nucleic acid extraction followed by reverse transcription quantitative PCR (RT-qPCR) and Sanger sequencing confirmed that full-length RNA genomes were packaged and protected from RNase by I53-50-v1 and I53-50-Btat but not the original I53-50 design (FIG. 1f); all versions of I53-47 could package their genomes (FIG. 14). In all cases, RT-PCR products were only obtained upon addition of reverse transcriptase, indicating that the protected nucleic acids were RNA and not DNA. We refer to these designed RNA-protein complexes as synthetic nucleocapsids.

To investigate whether synthetic nucleocapsids can evolve, we generated combinatorial libraries of synthetic nucleocapsid variants and selected for improved genome packaging and fitness against nuclease challenge. Nine positions on the interior surfaces of I53-50-v1 and I53-50-Btat were mutated to positive, negative, or uncharged polar amino acids (Table 2) to produce variants with a wide range of interior charge distributions.

TABLE 2

| | | | Starting | Starting | Considered | Selected |
|---|---|---|---|---|---|---|
| Evolution library | Component | Position | variant | aa | aa | aa |
| Interior charge design | Trimer | 126 | I53-50-v0 | T | D | D |
| (packaging) | | | | | | |
| Interior charge design | Trimer | 166 | I53-50-v0 | E | K | K |
| (packaging) | | | | | | |
| Interior charge design | Trimer | 179 | I53-50-v0 | S | K | K |
| (packaging) | | | | | | |
| Interior charge design | Trimer | 185 | I53-50-v0 | T | K | K |
| (packaging) | | | | | | |
| Interior charge design | Trimer | 195 | I53-50-v0 | A | K | K |
| (packaging) | | | | | | |
| Interior charge design | Trimer | 198 | I53-50-v0 | E | K | K |
| (packaging) | | | | | | |
| Interior charge design | Pentamer | 9 | I53-50-v0 | Y | H | H |
| (packaging) | | | | | | |
| Interior charge design | Pentamer | 38 | I53-50-v0 | A | R | R |
| (packaging) | | | | | | |
| Interior charge design | Pentamer | 105 | I53-50-v0 | S | D | D |
| (packaging) | | | | | | |
| Interior charge design | Pentamer | 122 | I53-50-v0 | D | K | K |
| (packaging) | | | | | | |
| Interior charge design | Pentamer | 124 | I53-50-v0 | D | K | K |
| (packaging) | | | | | | |
| Interior charge optimization | Trimer | 162 | I53-50-v1 | D | D, E, K, N | D |
| (packaging) | | | | | | |
| Interior charge optimization | Trimer | 166 | I53-50-v1 | K | E, K | K |
| (packaging) | | | | | | |
| Interior charge optimization | Trimer | 179 | I53-50-v1 | K | S, R, K, N | N |
| (packaging) | | | | | | |
| Interior charge optimization | Trimer | 185 | I53-50-v1 | K | T, T, K, N | N |
| (packaging) | | | | | | |
| Interior charge optimization | Trimer | 188 | I53-50-v1 | E | E, K | K |
| (packaging) | | | | | | |
| Interior charge optimization | Trimer | 198 | I53-50-v1 | K | E, K | K |
| (packaging) | | | | | | |
| Interior charge optimization | Pentamer | 122 | I53-50-v1 | K | D, E, K, N | K |
| (packaging) | | | | | | |
| Interior charge optimization | Pentamer | 124 | I53-50-v1 | K | D, E, K, N | N |
| (packaging) | | | | | | |
| Interior charge optimization | Pentamer | 126 | I53-50-v1 | H | H, Q, K, N | K |
| (packaging) | | | | | | |
| Interface pairwise SSM | Trimer | 21 | I53-50-v1 | V | all 20 aa | V |
| (packaging) | | | | | | |
| Interface pairwise SSM | Trimer | 22 | I53-50-v1 | E | all 20 aa | E |
| (packaging) | | | | | | |
| Interface pairwise SSM | Trimer | 25 | I53-50-v1 | I | all 20 aa | I |
| (packaging) | | | | | | |
| Interface pairwise SSM | Trimer | 26 | I53-50-v1 | E | all 20 aa | E |
| (packaging) | | | | | | |
| Interface pairwise SSM | Trimer | 29 | I53-50-v1 | V | all 20 aa | V |
| (packaging) | | | | | | |
| Interface pairwise SSM | Trimer | 32 | I53-50-v1 | F | all 20 aa | F |
| (packaging) | | | | | | |
| Interface pairwise SSM | Trimer | 33 | I53-50-v1 | A | all 20 aa | A |
| (packaging) | | | | | | |
| Interface pairwise SSM | Trimer | 50 | I53-50-v1 | T | all 20 aa | T |
| (packaging) | | | | | | |
| Interface pairwise SSM | Trimer | 53 | I53-50-v1 | K | all 20 aa | K |
| (packaging) | | | | | | |
| Interface pairwise SSM | Trimer | 54 | I53-50-v1 | A | all 20 aa | A |
| (packaging) | | | | | | |
| Interface pairwise SSM | Trimer | 56 | I53-50-v1 | S | all 20 aa | S |
| (packaging) | | | | | | |
| Interface pairwise SSM | Trimer | 57 | I53-50-v1 | V | all 20 aa | V |
| (packaging) | | | | | | |
| Interface pairwise SSM | Trimer | 58 | I53-50-v1 | L | all 20 aa | L |
| (packaging) | | | | | | |
| Interface pairwise SSM | Trimer | 60 | I53-50-v1 | E | all 20 aa | E |
| (packaging) | | | | | | |
| Interface pairwise SSM | Trimer | 61 | I53-50-v1 | K | all 20 aa | K |
| (packaging) | | | | | | |
| Interface pairwise SSM | Pentamer | 24 | I53-50-v1 | E | all 20 aa | F |
| (packaging) | | | | | | |
| Interface pairwise SSM | Pentamer | 28 | I53-50-v1 | A | all 20 aa | A |
| (packaging) | | | | | | |
| Interface pairwise SSM | Pentamer | 31 | I53-50-v1 | S | all 20 aa | S |
| (packaging) | | | | | | |
| Interface pairwise SSM | Pentamer | 35 | I53-50-v1 | A | all 20 aa | A |
| (packaging) | | | | | | |
| Interface pairwise SSM | Pentamer | 36 | I53-50-v1 | A | all 20 aa | A |
| (packaging) | | | | | | |
| RNaseA/Blood SSM | Trimer | All | I53-50-v2 | — | all 20 aa | — |
| (protection) | | residues | | | | |
| RNaseA/Blood SSM | Pentamer | All | I53-50-v2 | — | all 20 aa | — |
| (protection) | | residues | | | | |
| RNaseA/Blood combinatorial | Trimer | 2 | I53-50-v2 | K | K, N, T, E, | T |
| (protection) | | | | | D, A | |
| RNaseA/Blood combinatorial | Trimer | 8 | I53-50-v2 | K | K, N, T, E, | K |
| (protection) | | | | | D, A | |
| RNaseA/Blood combinatorial | Trimer | 9 | I53-50-v2 | K | K, N, S, R, | R |
| (protection) | | | | | E, D | |
| RNaseA/Blood combinatorial | Trimer | 11 | I53-50-v2 | K | K, N, T, E, | T |
| (protection) | | | | | D, A | |
| RNaseA/Blood combinatorial | Trimer | 61 | I53-50-v2 | K | K, N, T, E, | D |
| (protection) | | | | | D, A | |
| Exterior surface optimization | Trimer | 77 | I53-50-v3 | R | R, E, Q, G | R |
| Lib A (mouse circulation) | | | | | | |
| Exterior surface optimization | Trimer | 98 | I53-50-v3 | Q | K, E, Q | Q |
| Lib A (mouse circulation) | | | | | | |
| Exterior surface optimization | Trimer | 101 | I53-50-v3 | K | K, E, Q | K |
| Lib A (mouse circulation) | | | | | | |
| Exterior surface optimization | Trimer | 103 | I53-50-v3 | K | K, E, Q | K |
| Lib A (mouse circulation) | | | | | | |
| Exterior surface optimization | Pentamer | 6 | I53-50-v3 | H | Q | Q |
| Lib A (mouse circulation) | | | | | | |
| Exterior surface optimization | Pentamer | 9 | I53-50-v3 | H | Q | Q |
| Lib A (mouse circulation) | | | | | | |
| Exterior surface optimization | Pentamer | 20 | I53-50-v3 | R | R, E, Q, G | R |
| Lib A (mouse circulation) | | | | | | |
| Exterior surface optimization | Pentamer | 44 | I53-50-v3 | R | R, E, Q, G | R |
| Lib A (mouse circulation) | | | | | | |
| Exterior surface optimization | Pentamer | 70 | I53-50-v3 | R | R, E, Q, G | R |
| Lib A (mouse circulation) | | | | | | |
| Exterior surface optimization | Trimer | 74 | I53-50-v3 | E | E, D, K, N | D |
| Lib B (mouse circulation) | | | | | | |
| Exterior surface optimization | Trimer | 81 | I53-50-v3 | E | E, D, K, N | E |
| Lib B (mouse circulation) | | | | | | |
| Exterior surface optimization | Trimer | 94 | I53-50-v3 | E | E, D, K, N | E |
| Lib B (mouse circulation) | | | | | | |
| Exterior surface optimization | Trimer | 95 | I53-50-v3 | E | E, D, K, N | E |
| Lib B (mouse circulation) | | | | | | |
| Exterior surface optimization | Trimer | 102 | I53-50-v3 | E | E, D, K, N | E |
| Lib B (mouse circulation) | | | | | | |
| Exterior surface optimization | Pentamer | 6 | I53-50-v3 | H | Q | Q |
| Lib B (mouse circulation) | | | | | | |
| Exterior surface optimization | Pentamer | 9 | I53-50-v3 | H | Q | Q |
| Lib B (mouse circulation) | | | | | | |
| Exterior surface optimization | Pentamer | 34 | I53-50-v3 | E | E, D, K, N | E |
| Lib B (mouse circulation) | | | | | | |
| Exterior surface optimization | Pentamer | 39 | I53-50-v3 | D | E, D, K, N | K |
| Lib B (mouse circulation) | | | | | | |
| Exterior surface optimization | Pentamer | 43 | I53-50-v3 | D | E, D, K, N | E |
| Lib B (mouse circulation) | | | | | | |
| Exterior surface optimization | Pentamer | 67 | I53-50-v3 | E | E, D, K, N | K |
| Lib B (mouse circulation) | | | | | | |
| Exterior surface optimization | Trimer | 74 | I53-50-v3 | E | E, D, K, N | D |
| Lib C (mouse circulation) | | | | | | |
| Exterior surface optimization | Trimer | 77 | I53-50-v3 | R | R, E, Q, G | R |
| Lib C (mouse circulation) | | | | | | |
| Exterior surface optimization | Trimer | 81 | I53-50-v3 | E | E, D, K, N | E |
| Lib C (mouse circulation) | | | | | | |
| Exterior surface optimization | Trimer | 94 | I53-50-v3 | E | E, D, K, N | E |
| Lib C (mouse circulation) | | | | | | |
| Exterior surface optimization | Trimer | 95 | I53-50-v3 | E | E, D, K, N | E |
| Lib C (mouse circulation) | | | | | | |
| Exterior surface optimization | Trimer | 98 | I53-50-v3 | Q | K, E, Q | Q |
| Lib C (mouse circulation) | | | | | | |
| Exterior surface optimization | Trimer | 101 | I53-50-v3 | K | K, E, Q | K |
| Lib C (mouse circulation) | | | | | | |
| Exterior surface optimization | Trimer | 102 | I53-50-v3 | E | E, D, K, N | E |
| Lib C (mouse circulation) | | | | | | |
| Exterior surface optimization | Trimer | 103 | I53-50-v3 | K | K, E, Q | K |
| Lib C (mouse circulation) | | | | | | |
| Exterior surface optimization | Pentamer | 6 | I53-50-v3 | H | Q | Q |
| Lib C (mouse circulation) | | | | | | |
| Exterior surface optimization | Pentamer | 9 | I53-50-v3 | H | Q | Q |
| Lib C (mouse circulation) | | | | | | |
| Exterior surface optimization | Pentamer | 20 | I53-50-v3 | R | R, E, Q, G | R |
| Lib C (mouse circulation) | | | | | | |
| Exterior surface optimization | Pentamer | 34 | I53-50-v3 | E | E, D, K, N | E |
| Lib C (mouse circulation) | | | | | | |
| Exterior surface optimization | Pentamer | 39 | I53-50-v3 | D | E, D, K, N | K |
| Lib C (mouse circulation) | | | | | | |
| Exterior surface optimization | Pentamer | 43 | I53-50-v3 | D | E, D, K, N | E |
| Lib C (mouse circulation) | | | | | | |
| Exterior surface optimization | Pentamer | 44 | I53-50-v3 | R | R, E, Q, G | R |
| Lib C (mouse circulation) | | | | | | |
| Exterior surface optimization | Pentamer | 67 | I53-50-v3 | E | E, D, K, N | K |
| Lib C (mouse circulation) | | | | | | |
| Exterior surface optimization | Pentamer | 70 | I53-50-v3 | R | R, E, Q, G | R |
| Lib C (mouse circulation) | | | | | | |
| I53-50-v3 hydrophilic tails | Pentamer | C-term | I53-50-v3 | — | — | — |
| library (mouse circulation) | | | | | | |

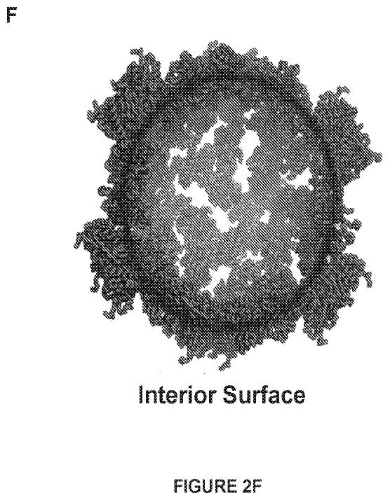

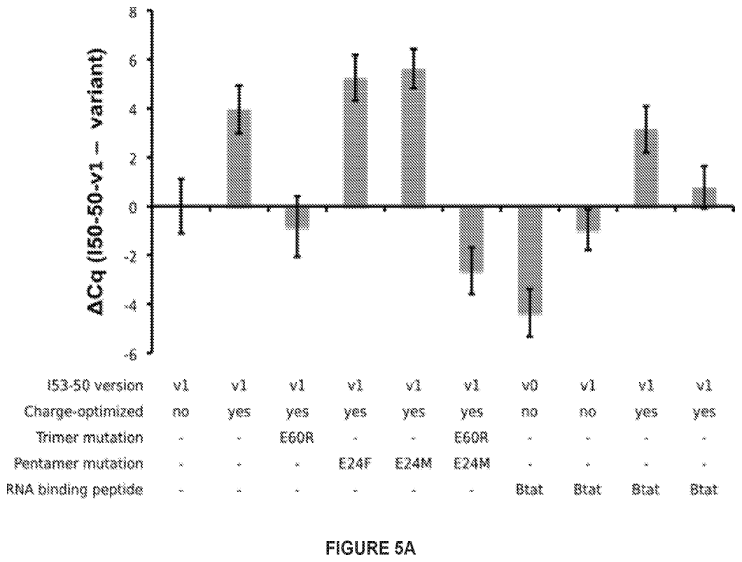

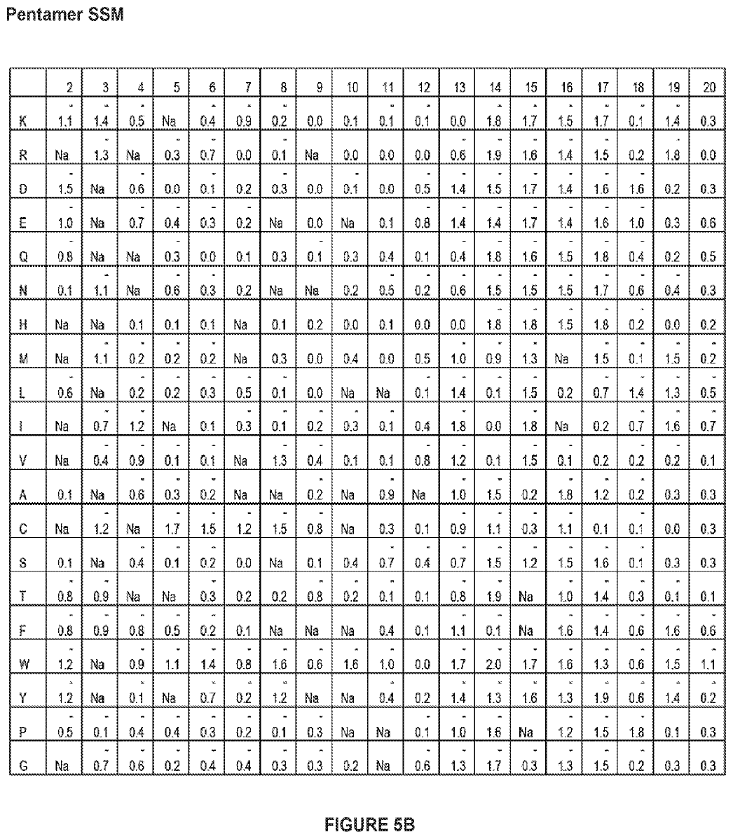

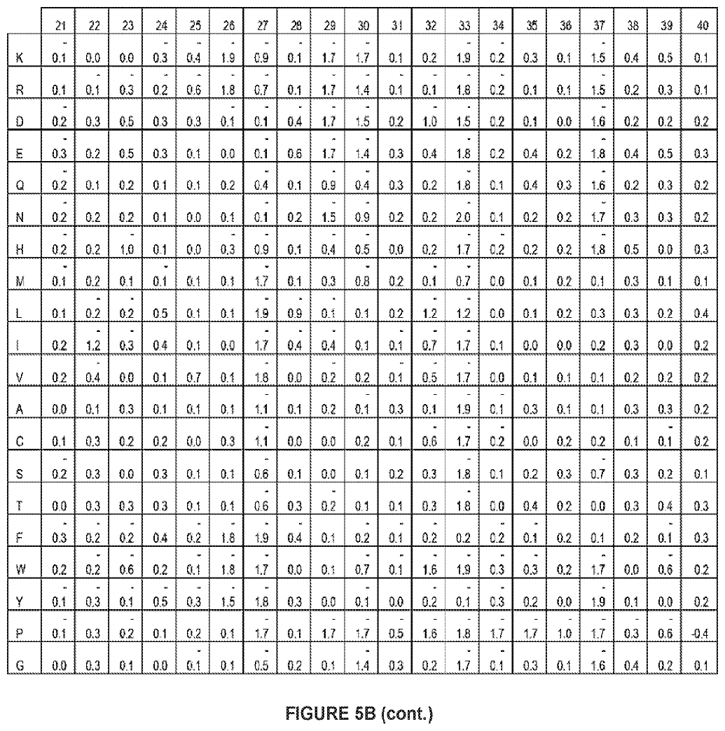

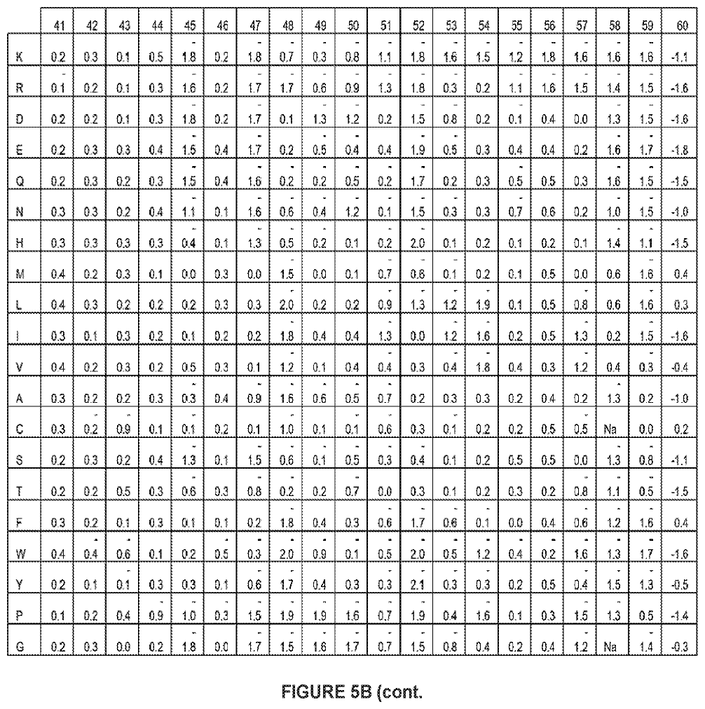

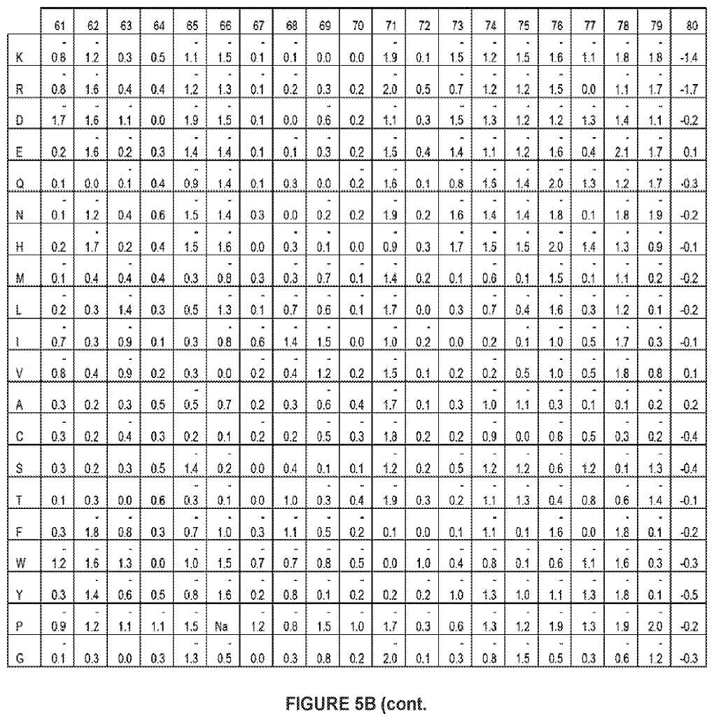

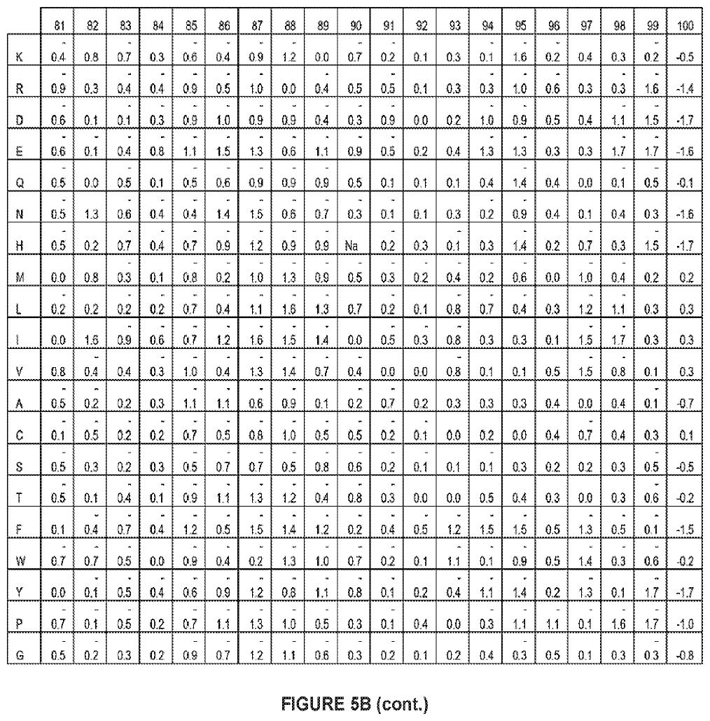

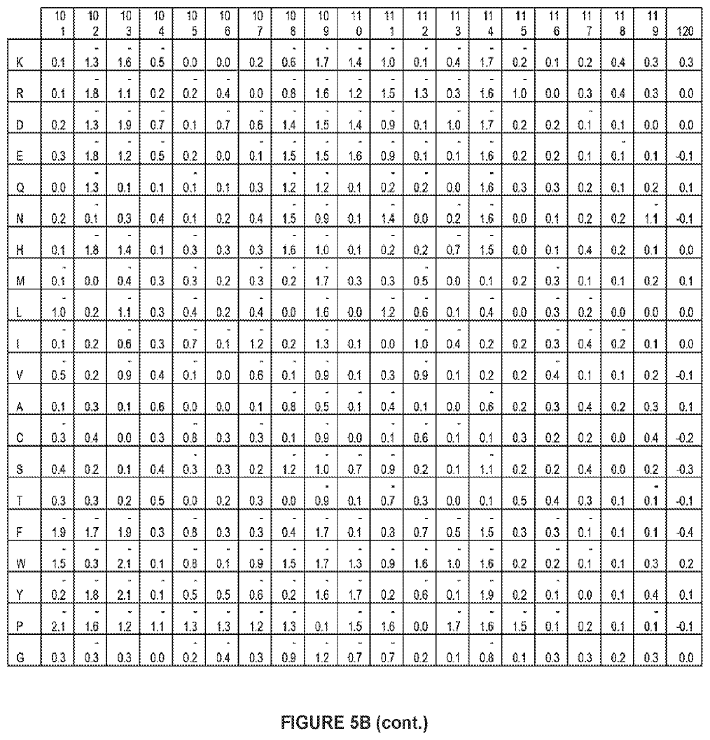

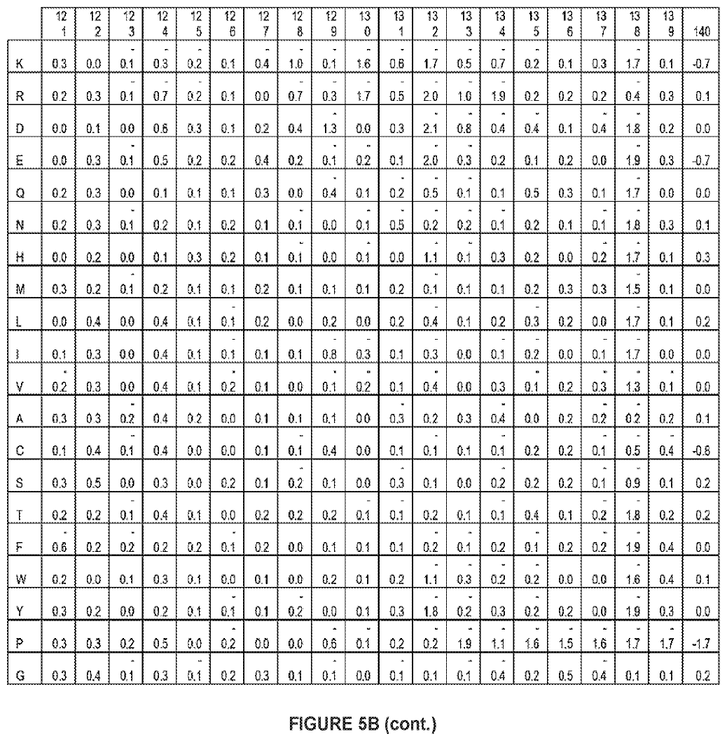

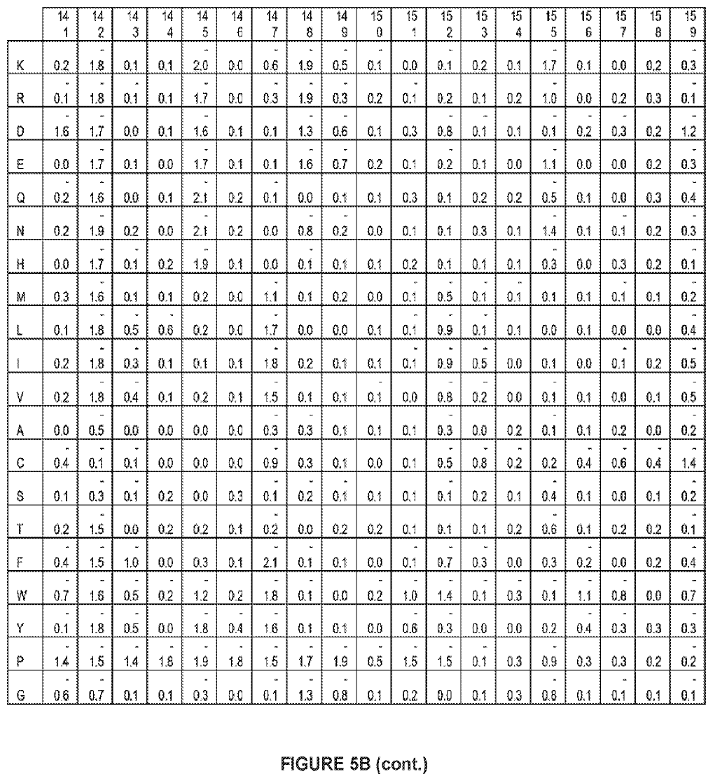

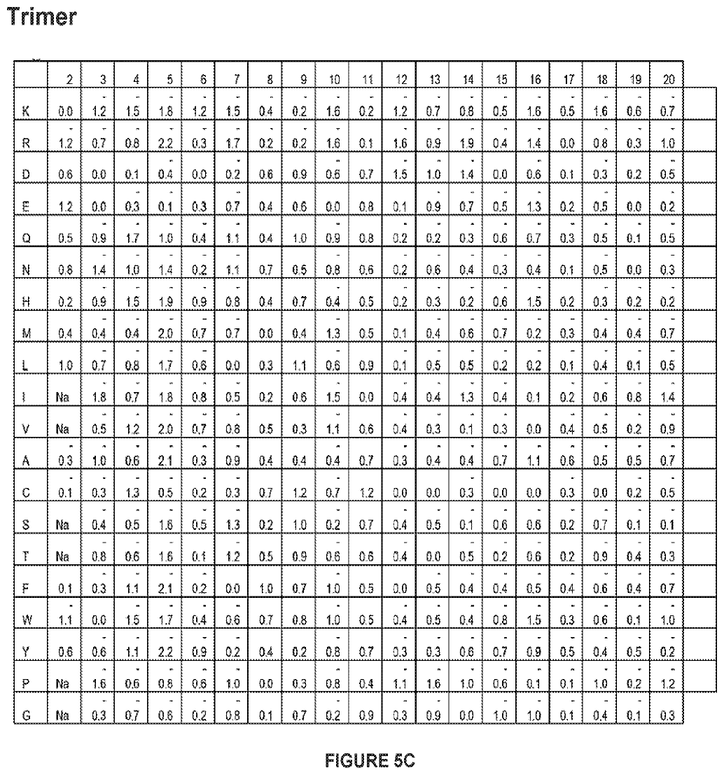

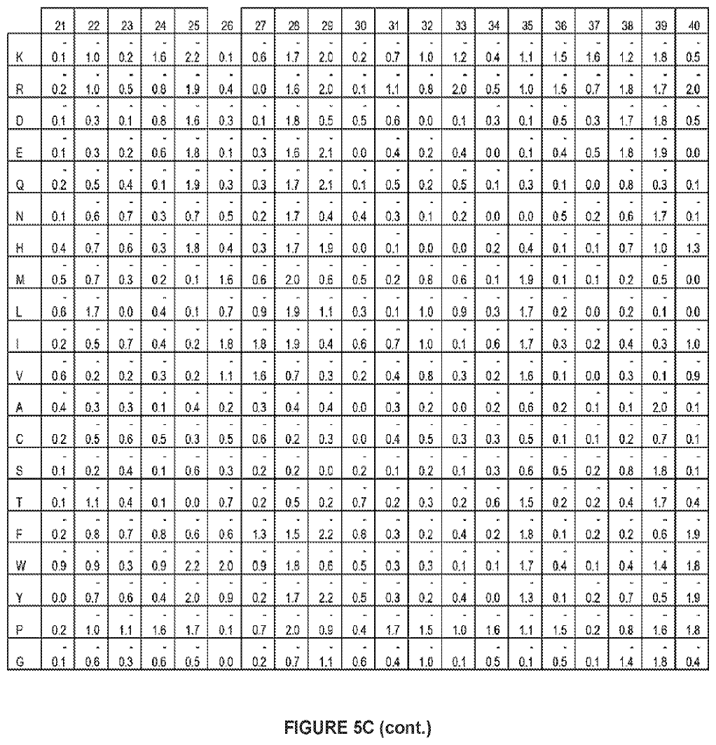

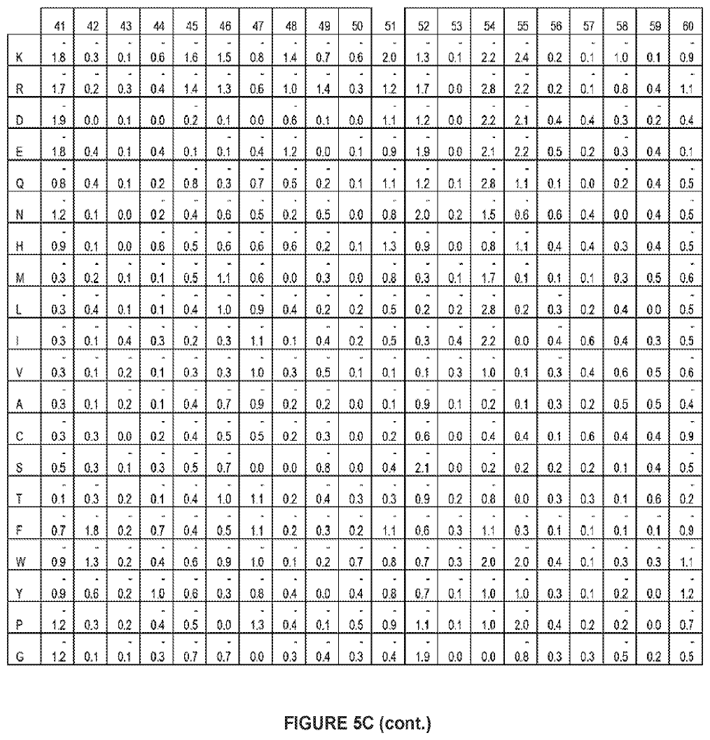

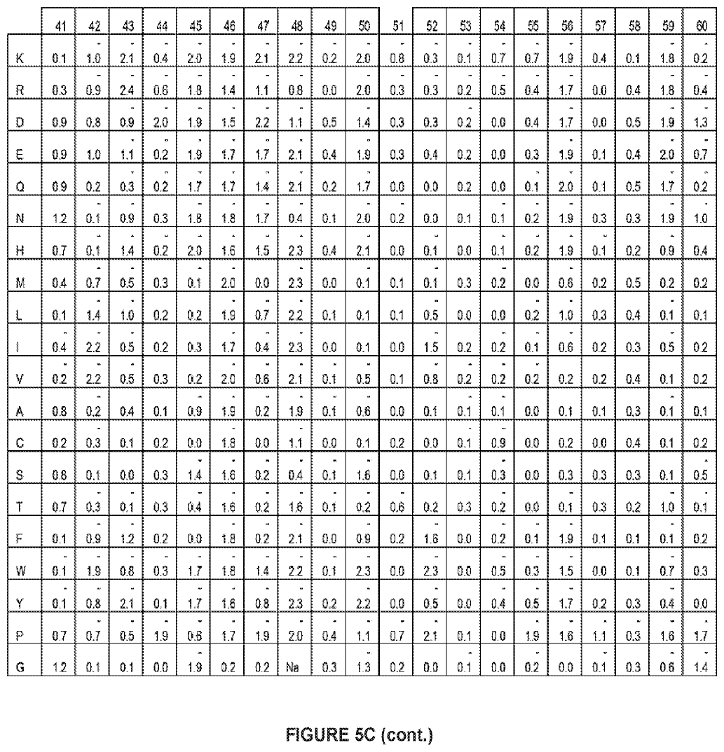

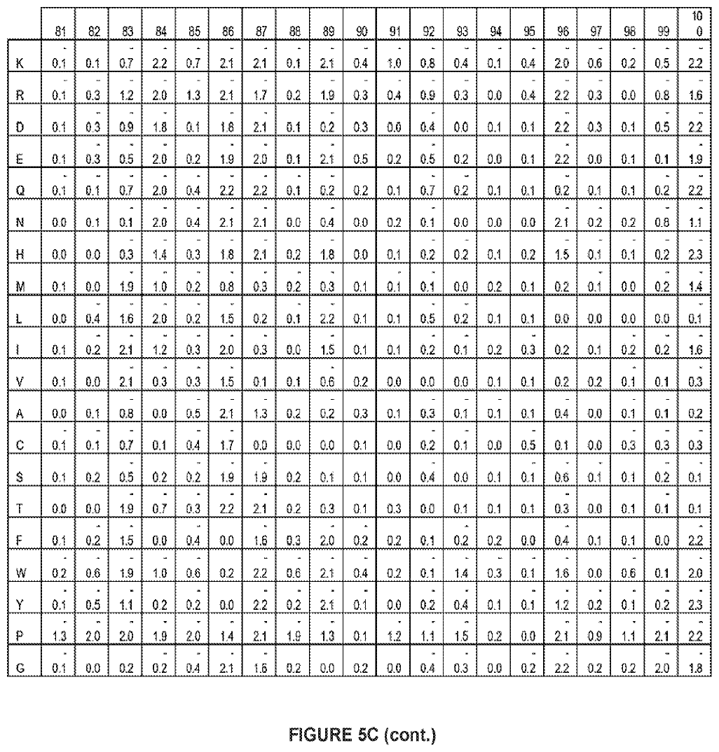

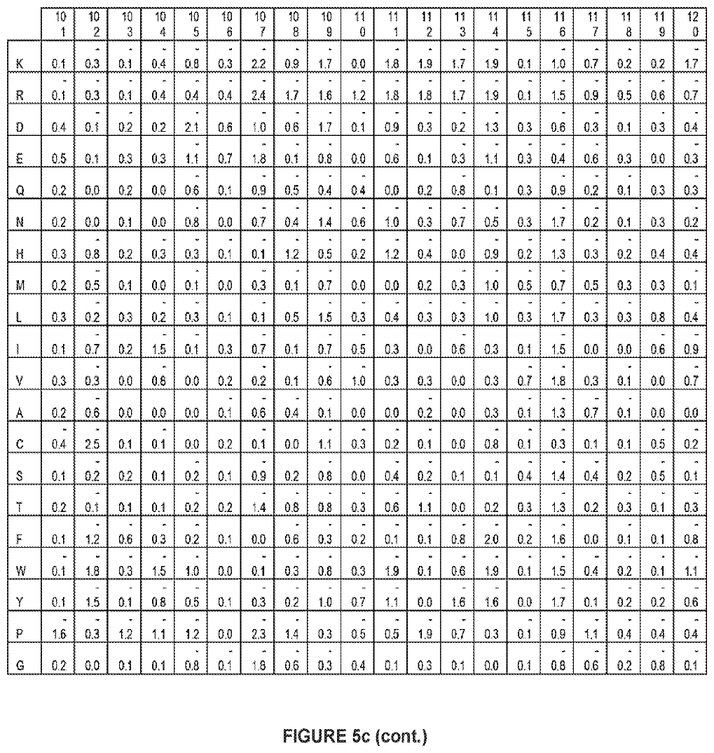

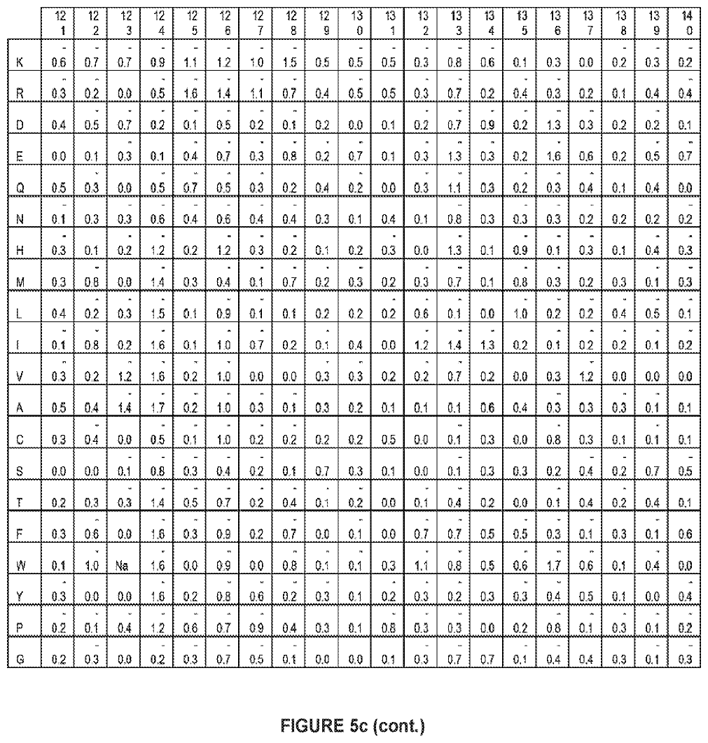

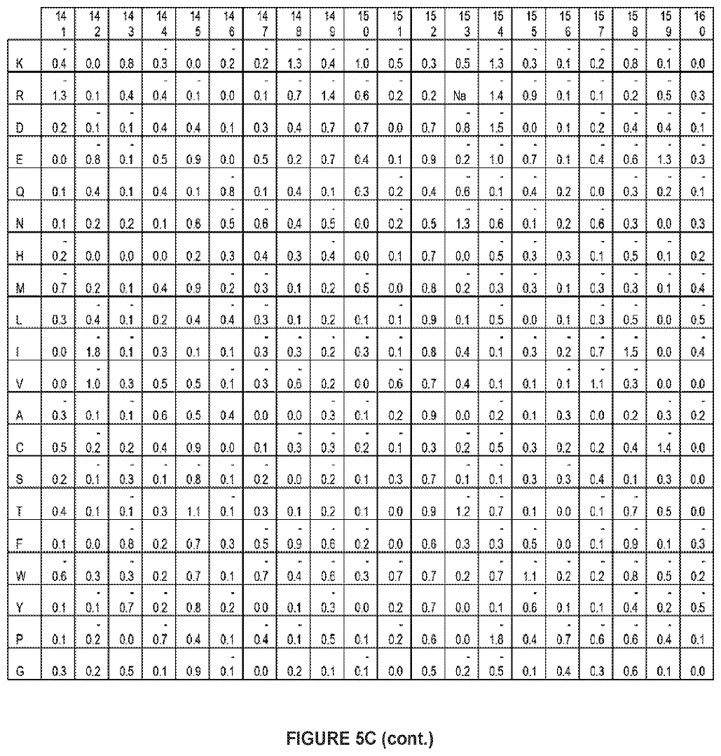

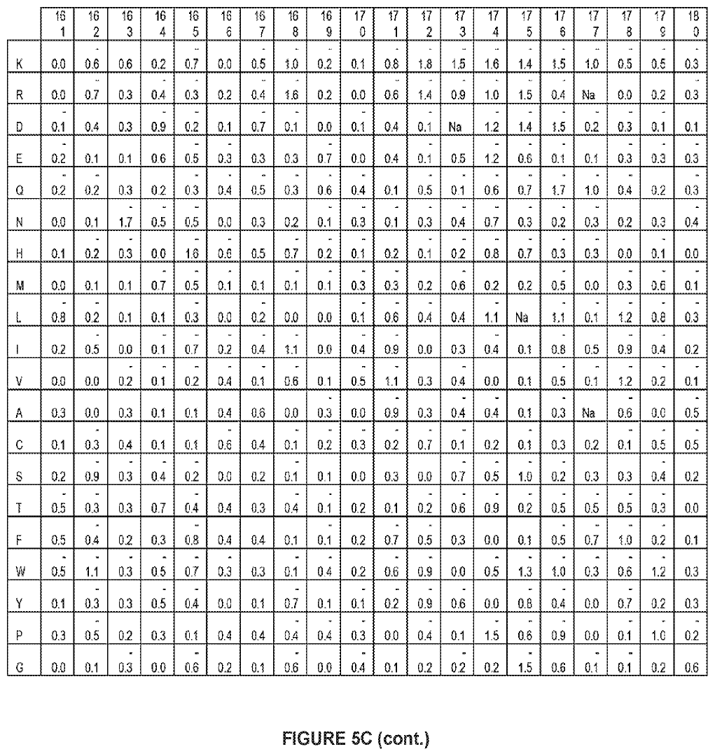

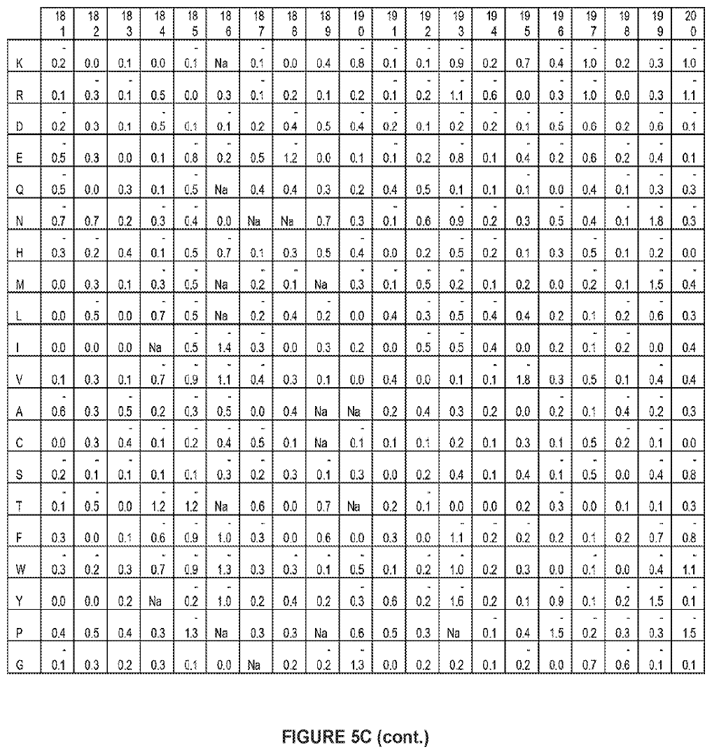

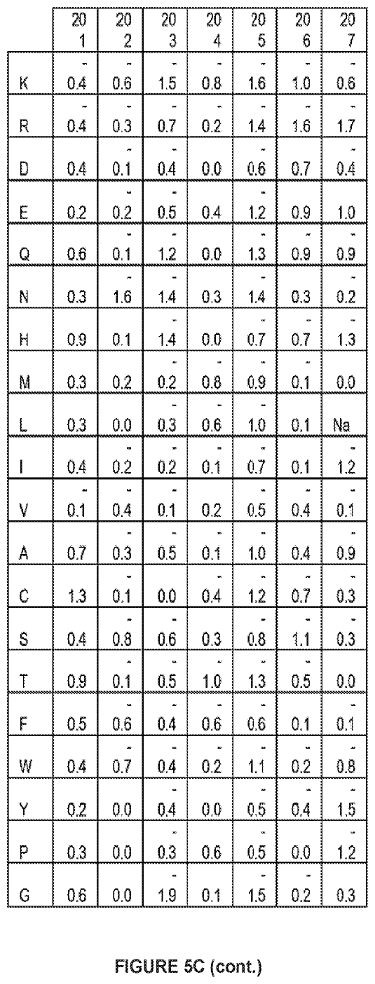

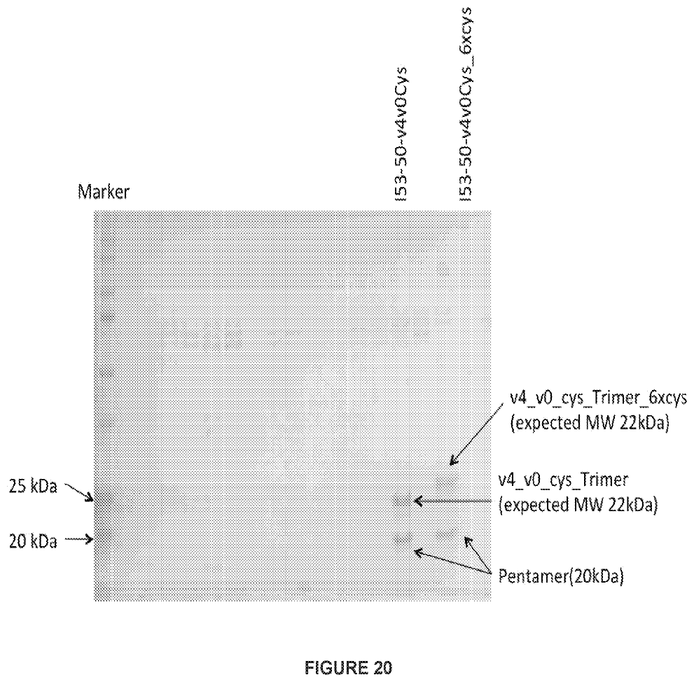

We performed three rounds of selection comprising expression, purification. RNase challenge, RNA recovery, and re-cloning (FIG. 2a). The RNA recovered from the selected population after each round was reverse-transcribed and sequenced on an Illumina MiSeq. The net interior charge of the evolved population converged to narrow distributions around 388±87 (mean±standard deviation of the population) in the absence of Btat and 662±91 (480 of which are from 60 copies of Btat) in the presence of Btat (FIG. 2b). 1170 different variants exhibited higher enrichment than I53-50-v1 (FIG. 2c); there are evidently many solutions to the genome packaging problem. The presence or absence of the positively charged Btat peptide influenced the identities of beneficial mutations—all except two of the lysine residues were beneficial in the absence of Btat (FIG. 2d), whereas most lysine residues were disfavored in the presence of Btat (FIG. 2e). We combined the substitutions from one of the most highly enriched variants from the library lacking Btat (FIG. 2c; trimeric subunit: K178N, K183N, E189K; pentameric subunit: K123N, H125K) with the most enriched substitution from a separate library of mutants in the trimer-pentamer interface (pentameric subunit: E24F; Table 2) to produce I53-50-v2, which exhibited improved genome packaging efficiency as assessed by RT-qPCR (FIG. 5). The net interior charge did not change between I53-50-v1 and I53-50-v2—the improved genome packaging and protection results from reconfiguration of the position of the charges (FIG. 20. I53-50-v2 outperformed the best variants from the I53-50-Btat library (FIG. 5A), so we focused on I53-50-v2 for subsequent evolution experiments.

The ability to evolve the nucleocapsids enabled comprehensive mapping of how each residue affects the fitness of a synthetic, 2.5 megadalton complex comprising 22,920 amino acids and 1,370 RNA bases. We produced a deep mutational scanning library of I53-50-v2 with every residue in each protein subunit substituted with each of the 20 amino acids, and performed two consecutive rounds of selection with two biological replicates. Selection in the first round was performed at room temperature with 10 μg/mL RNase A for 10 minutes to deplete non-assembling variants from the population, and selection in the second round was at 37° C. for 1 hour with either 10 mg/mL RNase A or heparinized whole murine blood. Each replicate of the naive, round 1, and round 2 populations was sequenced on an Illumina MiSeq, and enrichment values were calculated from the fraction of the population corresponding to each variant before and after selection; 7,156 out of the possible 7,240 single mutants were observed with at least 10 counts in the pre-selection population). The enrichments of individual mutations were correlated between the RNase A and whole murine blood selections), suggesting that similar mechanisms underlie the increased genome protection in both cases.

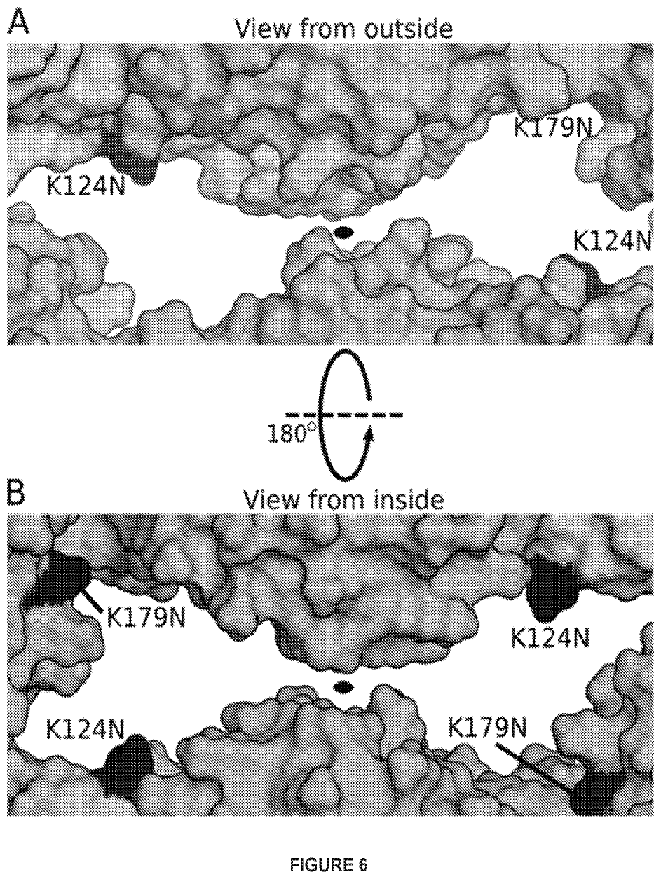

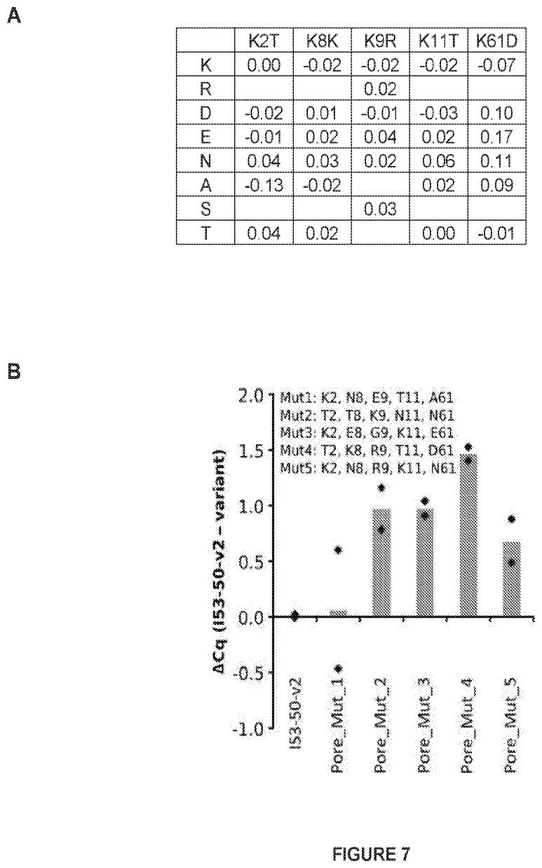

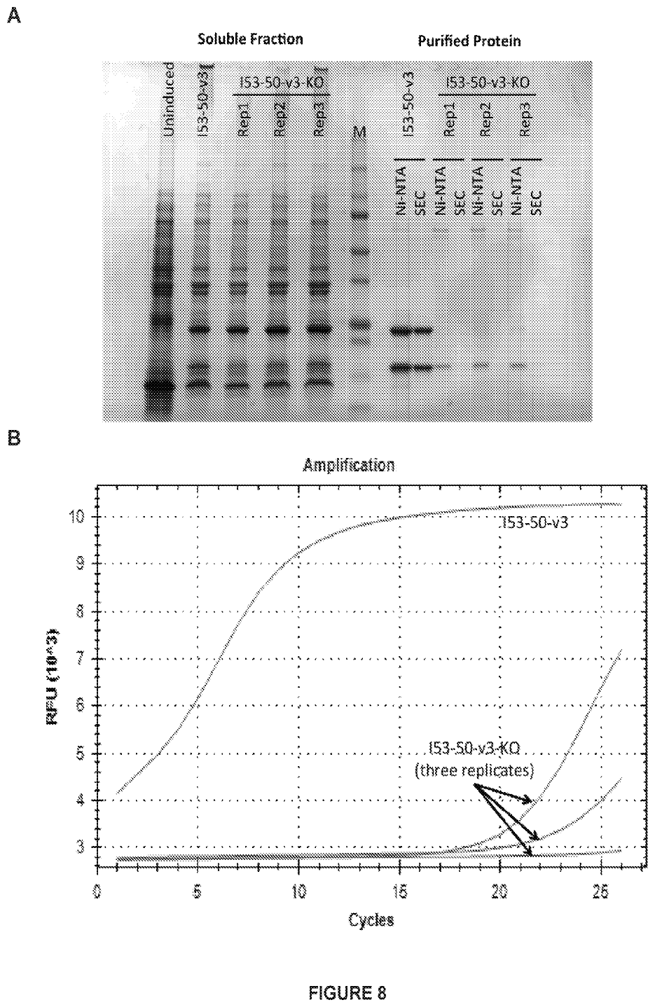

Evaluating the enrichment values in the context of the I53-50 design model provides insight into the features important for genome encapsulation and protection. I53-50 is composed of 20 trimers and 12 pentamers; the hydrophobic protein cores, intra-oligomer interfaces, and designed inter-oligomer interface were conserved—proteins bearing mutations that disrupt the stability of the assembly likely fail to protect their genomes and are removed from the population. Strong selective pressure also operated on the electrostatics of the surface lining the pore between trimeric subunits of I53-50-v2—all highly depleted residues were lysines or arginines, whereas the nearby glutamate (residue E4) was highly conserved ( ). Lysine removal around the pore also occurred in the earlier transition from I53-50-v1 to I53-50-v2—K179N in the trimer and K124N in the pentamer (FIG. 2d, FIG. 6). Positively charged residues near the pores may compromise genome protection either by promoting protrusion of the encapsulated RNA from the interior of the icosahedral assembly—thereby rendering it susceptible to RNases—or by destabilizing the assembly through electrostatic repulsion between trimeric subunits. To test whether several of the most enriched mutations could be combined to produce a synthetic nucleocapsid with superior fitness, a combinatorial library was constructed containing charged and uncharged polar residues at positions where positively charged residues were deleterious in the deep mutational scanning data (trimeric subunit: K2, K8, K9, K11, K61). After selection in 10 μg/mL RNase A at 37° C. for 1 hour, the six most enriched variants were tested individually to evaluate their improvements over I53-50-v2 (FIG. 7). The one best protected under these conditions was designated I53-50-v3 (trimeric subunit: K2T, K9R, K11T, K61D). The failure of an assembly-defective variant to protect its genome (I53-50-v3-KO; trimeric subunit: V29R, pentameric subunit: A38R; FIG. 8) confirmed that encapsulation was required for RNA protection.

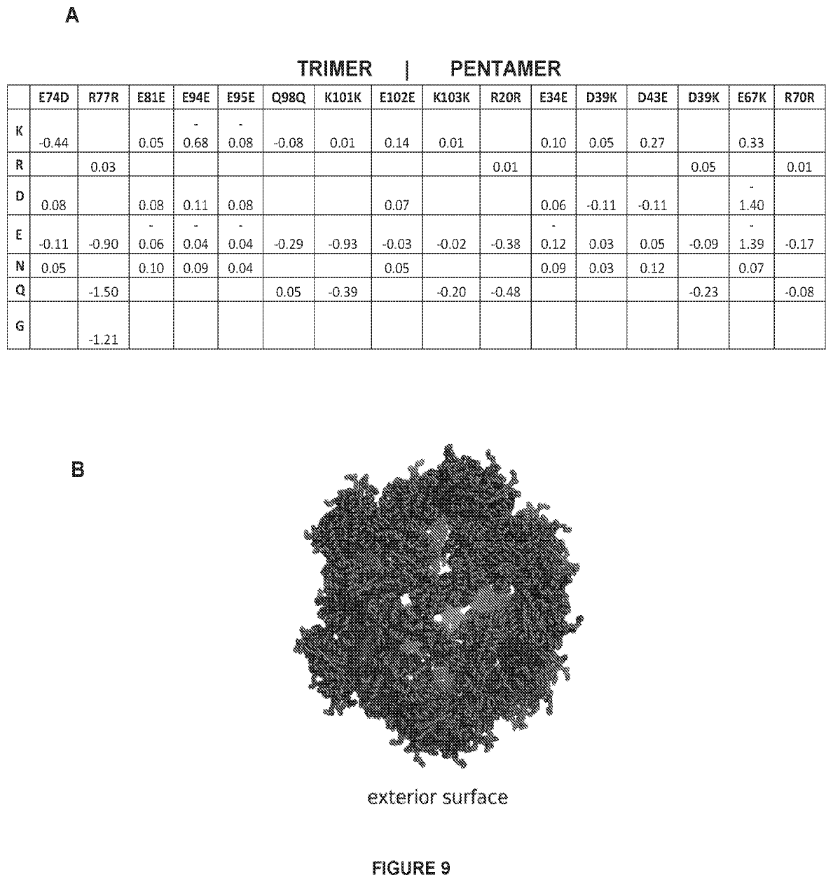

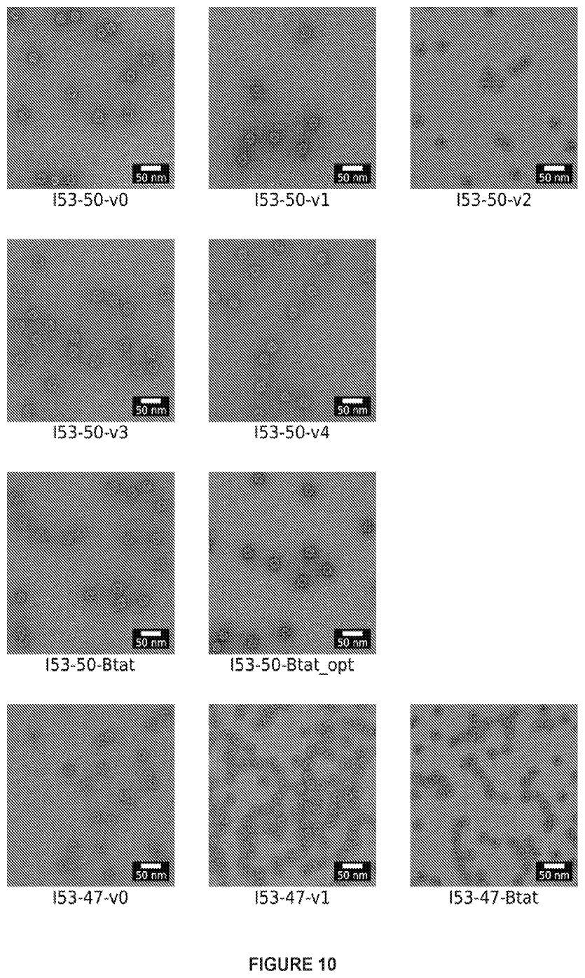

We next investigated whether synthetic nucleocapsids can evolve inside an animal. As long circulation times are desirable for in vivo applications such as drug delivery, we decided to focus on this property. We hypothesized that the hexahistidine tag might mediate undesired interactions in vivo, so we created cleavable versions that were used for all subsequent experiments (see supplementary methods). We produced two populations of synthetic nucleocapsids, one displaying hydrophilic 60-residue polypeptides of varying compositions intended to mimic viral glycosylation or PEGylation (SEQ ID NOS:58-518 (stabilization peptides) and another with 14 exterior surface positions combinatorially mutated to polar charged and uncharged amino acids (D, E, N, Q, K, R; Table 2). We administered each population to mice (n=5) by retro-orbital injection, and evaluated the survival of each member of the population in vivo by blood draws from the tail vein at successive time points. From both libraries, a number of distinct sequences drastically improved circulation times. An optimal amino acid composition emerged in the hydrophilic peptide library. Arbitrary polypeptides with similar amino acid composition (e.g., 4.5 repeats of PETSPASTEPEGS (SEQ ID NO:538) or 4 repeats of PESTGAPGETSPEGS (SEQ ID NO:539)) increased circulation time, whereas other polypeptides composed of different amino acids (e.g., 12 repeats of ESESG (SEQ ID NO:540)) did not ( ). From the exterior surface library, we isolated several variants exhibiting drastically enhanced circulation time compared to I53-50-v3 and found that the majority contained the E67K substitution in the pentameric subunit (FIG. 9). We generated I53-50-v4 by incorporating E67K along with a set of other consensus mutations (Table 1; as the hydrophilic polypeptides reduced nucleocapsid yield, they were not included) that were enriched in the selected population of synthetic nucleocapsids and may also contribute to increased expression and stability. Negative-stain electron micrographs of I53-50-v1, I53-50-v2, I53-50-v3, and I53-50-v4 showed that the functional improvements introduced by evolution did not compromise the designed icosahedral architecture (FIG. 10), and dynamic light scattering indicated uniform populations of nucleocapsids around the expected size (radius=13.5 nm).

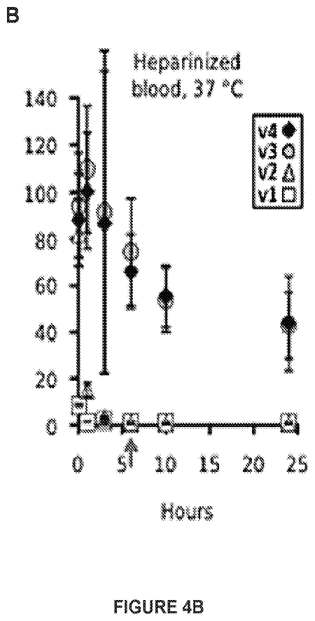

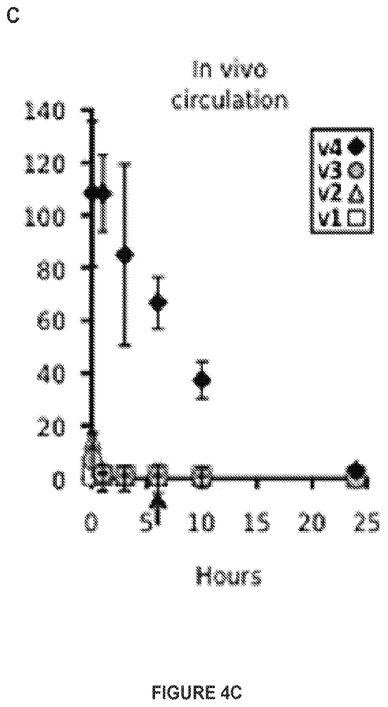

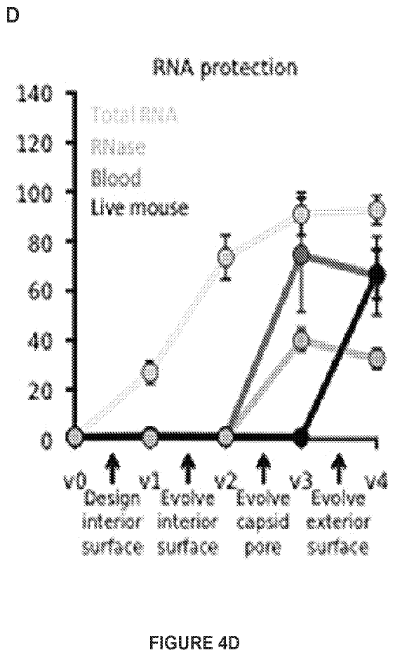

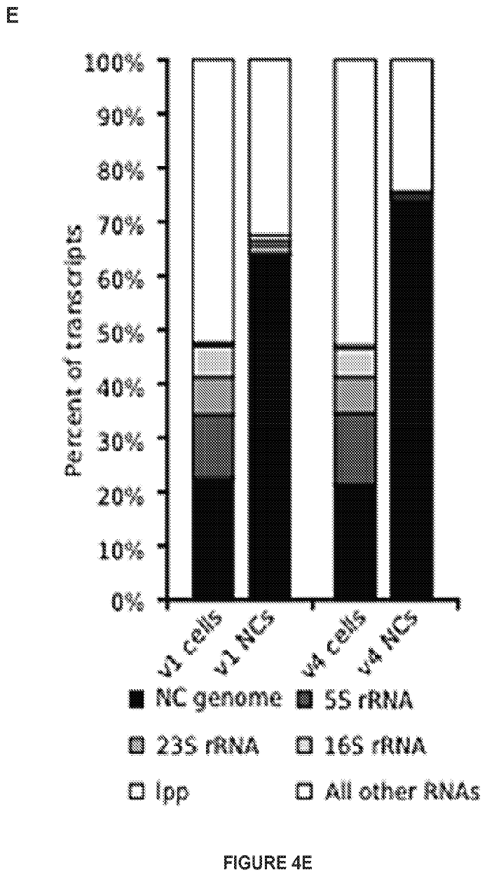

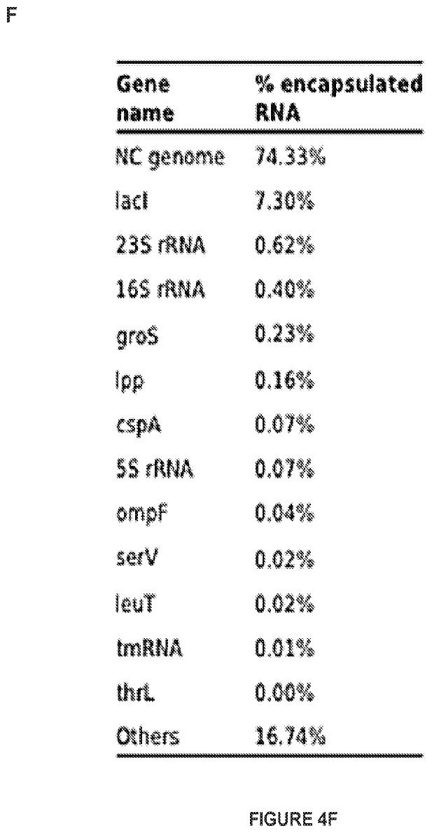

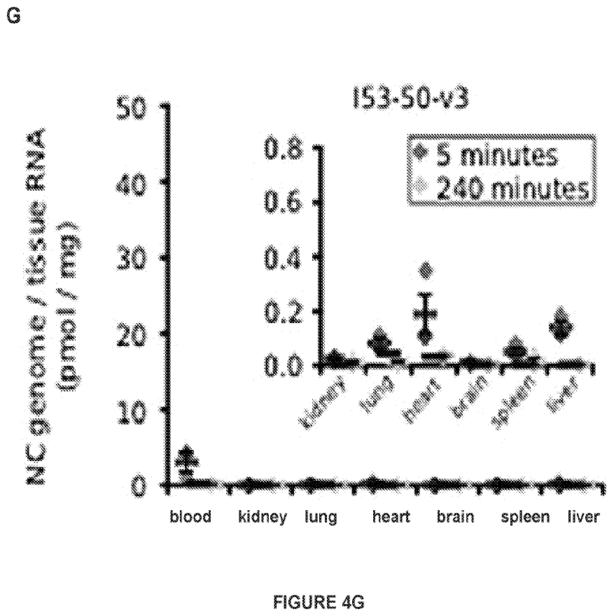

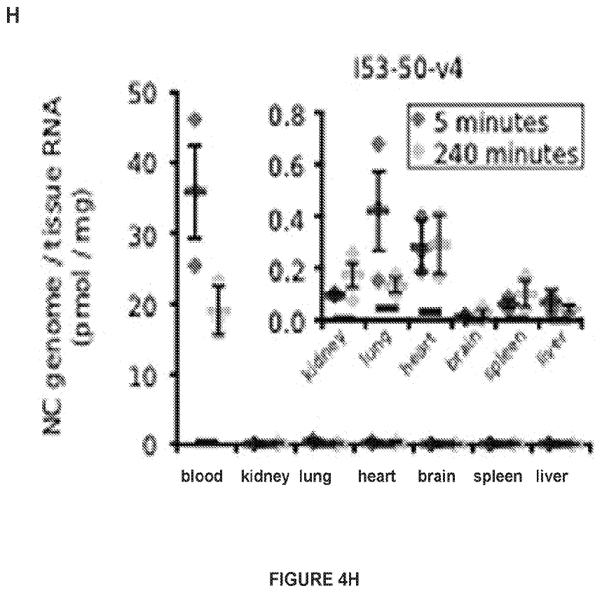

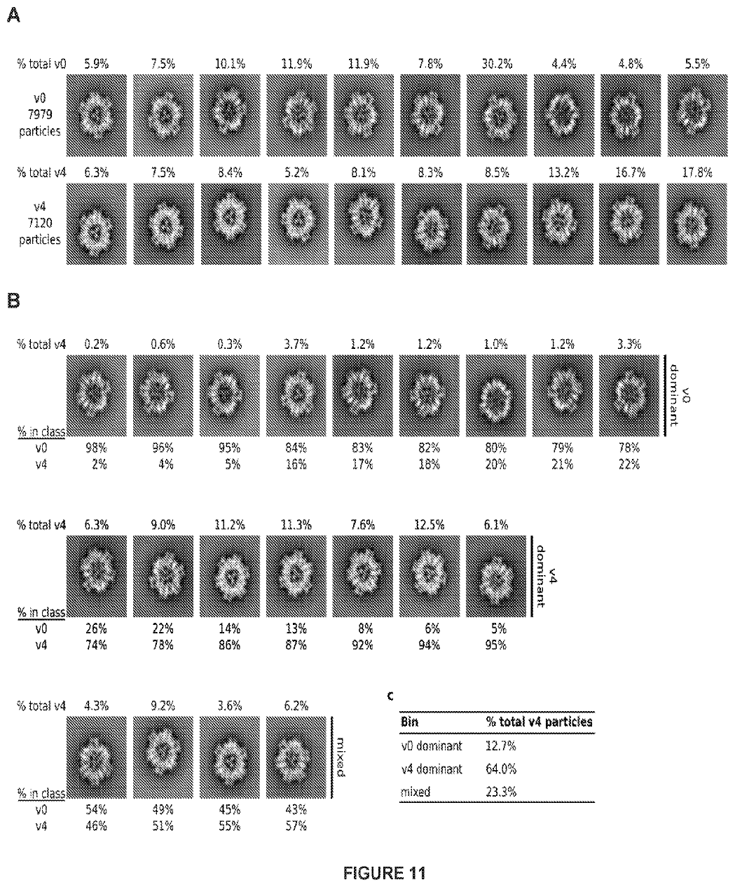

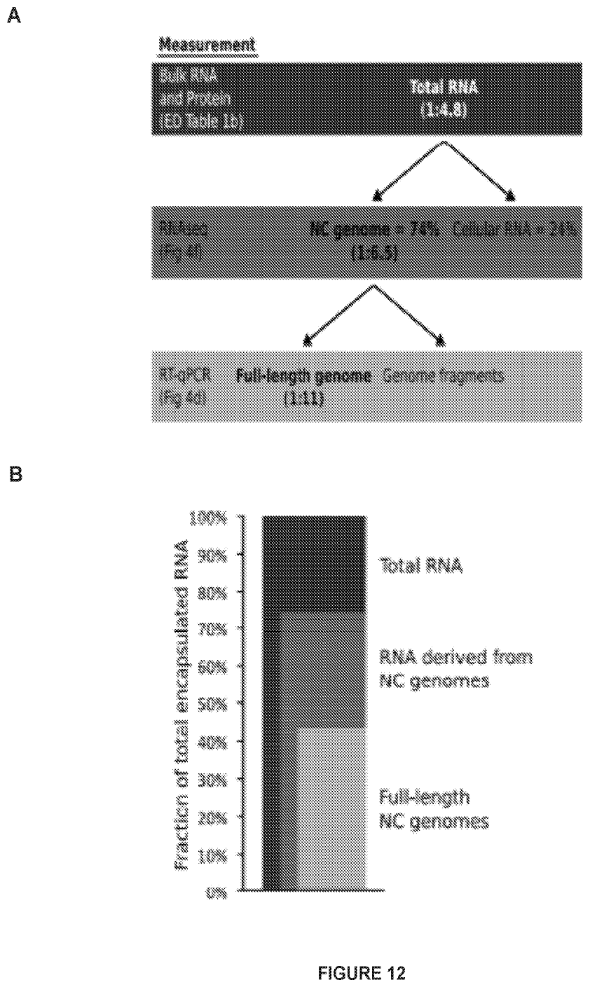

What fraction of the I53-50-v4 synthetic nucleocapsids are filled, and with which RNAs? Negative-stain electron microscopy analysis of 15,119 particles suggests that the majority of I53-50-v4 nucleocapsids are more electron-dense, likely due to encapsulated nucleic acid, than the unfilled I53-50-v0 assemblies (FIG. 11). Quantitation of bulk RNA and protein indicated that there is approximately one nucleocapsid genome-equivalent (1,433 nt) of total RNA encapsulated per 6.6 (I53-50-v1) and 4.8 (I53-50-v4) capsids (Table 3). Given that RNAseq showed that ˜74% of this total RNA was derived from the nucleocapsid genome (I53-50-v4, FIG. 4e-f) and may include genome fragments, these data are consistent with our RT-qPCR quantitation of one full-length genome per 11 capsids (FIG. 12). While capsid genomes are modestly enriched and ribosomal RNA is depleted in nucleocapsids relative to cells (FIG. 4e-f), I53-50-v4 does not exhibit increased specificity for its genome relative to I53-50-v1. Instead, packaging correlates strongly with expression level. The ability to package arbitrary RNA sequences combined with the ability to assemble in vitro from purified subunits could make synthetic nucleocapsids the basis of a highly flexible platform for RNA delivery.

TABLE 3
Genomes per nucleocapsid by bulk RNA and protein measurements

| | | Total | | | | | |
|---|---|---|---|---|---|---|---|
| | | encapsulated | | Total | Capsids/ | % RNA | |
| | Protein | RNA | Capsids | RNA | Genome | is NC | Capsids/ |
| Sample | (ug/mL) | (ng/uL) * | (M) † | (M) ‡ | equiv. § | genome ∥ | genome |
| I53-50-v0 | 184 | bd | 7.4E−08 | bd | bd | bd | bd |
| (rep 1) | | | | | | | |
| I53-50-v0 | 188 | bd | 7.6E−08 | bd | bd | bd | bd |
| (rep 2) | | | | | | | |
| I53-50-v1 | 436 | 14.0 | 1.7E−07 | 3.0E−08 | 5.7 | 64% | 8.9 |
| (rep 1) | | | | | | | |
| I53-50-v1 | 504 | 12.3 | 2.0E−07 | 2.6E−08 | 7.5 | 64% | 11.7 |
| (rep 2) | | | | | | | |
| I53-50-v4 | 217 | 8.0 | 8.5E−08 | 1.7E−08 | 5.0 | 74% | 6.7 |
| (rep 1) | | | | | | | |
| I53-50-v4 | 217 | 8.7 | 8.5E−08 | 1.9E−08 | 4.6 | 74% | 6.2 |
| (rep 2) | | | | | | | |

* bd = below detection
† Capsid MW: v0 = 2479.440 kDa, v1 = 2544.300 kDa, v4 = 2539.320 kDa
‡ Total RNA calculated by assigning nucleocapsid genome MW to total RNA: v0 = 443.618 kDa, v1 = 464.212 kDa, v4 = 463.971 kDa
§ Genome equivalents of total RNA (includes cellular RNA)
∥ Determined by RNAseq

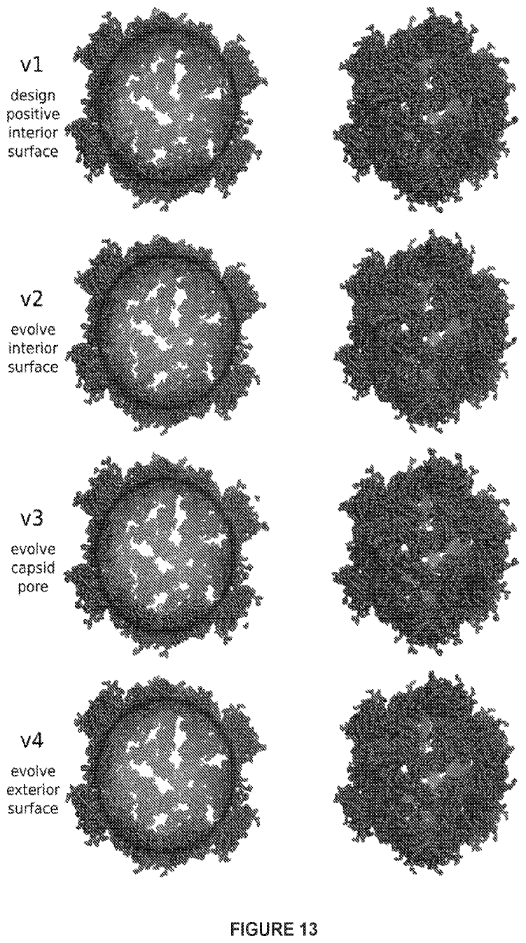

Like modern viruses, our evolved synthetic nucleocapsids exhibit genome packaging, nuclease protection, and sustained circulation in vivo. Each evolutionary step (Table 1; FIG. 13) improved the particular property under selection without compromising gains from previous steps (FIG. 4). The I53-50-v1 design provided a starting point for evolution, inefficiently packaging its own full-length genome. Evolving the interior surface produced I53-50-v2, which packages ˜1 RNA genome for every 14 capsids, rivaling the best recombinant AAVs^{8,9} (FIG. 4d). Subsequently, evolving the capsid pore for improved stability resulted in I53-50-v3, which protects 44% of its RNA when challenged by RNase A (10 μg/mL, 37° C., 6 hours) and 82% of its RNA when challenged by whole murine blood (37° C., 6 hours), whereas I53-50-v2 only protects 1.0% and 1.2%, respectively (FIG. 4a-b). Evolving the exterior surface of the capsid in circulation in live mice produced I53-50-v4, with a >54-fold increase in circulation half-life from less than 5 minutes for I53-50-v3 to 4.5 hours for I53-50-v4 (FIG. 4c). To further characterize the difference in behavior between these two nucleocapsids, we determined the relative biodistribution of intact nucleocapsids by RT-qPCR of full-length genomes at both 5 minutes and 4 hours. As expected, no obvious tissue tropism was observed for either nucleocapsid. Furthermore, there is no substantial intact I53-50-v3 remaining in any organs by 4 hours post-injection, consistent with the rapid elimination of I53-50-v3 compared to I53-50-v4 (FIG. 4g-h).

This work demonstrates that by acquiring positive charge on its interior, an otherwise inert self-assembling protein nanomaterial can package its own RNA genome and evolve under selective pressure. Starting from this “blank slate”, evolution uncovered multiple simple mechanisms to improve complex properties such as genome packaging, nuclease resistance, and in vivo circulation time. This suggests paths by which viruses could have arisen from protein assemblies that adopted simple mechanisms to package their own genetic information. Modern viruses are much more complex, having evolved under selective pressure to minimize genome size and to optimize multiple capsid functions required for a complete viral life cycle. However, this makes it difficult to change one property (e.g., alter tropism or remove epitopes for pre-existing antibodies^{19,20}) without compromising other functions. By contrast, the simplicity of our synthetic nucleocapsids should allow them to be further engineered more freely. Combining the evolvability of viruses with the accuracy and control of computational protein design, synthetic nucleocapsids can be custom-designed and then evolved to optimize function in complex biochemical environments.

### REFERENCES FOR EXAMPLE 1

1. Bale, J. B. et al. Accurate design of megadalton-scale two-component icosahedral protein complexes. Science 353, 389-394 (2016).
2. Gibson, D. G. et al. Enzymatic assembly of DNA molecules up to several hundred kilobases. Nat Methods 6, 343-345 (2009).
3. Kunkel, T. A. Rapid and efficient site-specific mutagenesis without phenotypic selection. Proc Natl Acad Sci USA 82, 488-492 (1985).
4. Rohland, N. & Reich, D. Cost-effective, high-throughput DNA sequencing libraries for multiplexed target capture. Genome Res 22, 939-946 (2012).
5. Alvarez, P., Buscaglia, C. A. & Campetella, O. Improving protein pharmacokinetics by genetic fusion to simple amino acid sequences. J Biol Chem 279, 3375-3381 (2004).
6. Schellenberger, V. et al. A recombinant polypeptide extends the in vivo half-life of peptides and proteins in a tunable manner. Nat Biotechnol 27, 1186-1190 (2009).
7. Benson, D. A. et al. GenBank. Nucleic Acids Res 41, D36-42 (2013).
8. Nannenga, B. L., Iadanza, M. G., Vollmar; B. S. & Gonen; T. Overview of electron crystallography of membrane proteins: crystallization and screening strategies using negative stain electron microscopy. Curr Protoc Protein Sci Chapter 17, Unit 17.15 (2013).
9. Subway, C. et al. Automated molecular microscopy: the new Leginon system. J Struct Biol 151, 41-60 (2005).
10. Tang, G. et al. EMAN2: an extensible image processing suite for electron microscopy. J Struct Biol 157, 38-46 (2007).
11. Fowler, D. M., Araya, C. L., Gerard, W. & Fields, S. Enrich: software for analysis of protein function by enrichment and depletion of variants. Bioinformatics 27, 3430-3431 (2011).
12. Hunter, J. D., Vol. 9 90-95 (Computing In Science \& Engineering: 2007).
13. Kim, D., Langmead, B. & Salzberg, S. L. HISAT: a fast spliced aligner with low memory requirements. Nat Methods 12, 357-360 (2015).
14. Li, H. et al. The Sequence Alignment/Map format and SAMtools. Bioinformatics 25, 2078-2079 (2009).
15. Pertea, M., Kim, D., Pertea, G. M., Leek, J. T. & Salzberg, S. L. Transcript-level expression analysis of RNA-seq experiments with HISAT, StringTie and Ballgown. Nat Protoc 11, 1650-1667 (2016).

### Materials and Methods

### Solutions and Buffers

Lysogeny Broth (LB): Autoclave 10 g tryptone, 5 g yeast extract, 5 g NaCl, 1 L dH₂O.

LB agar plates: Autoclave LB with 15 g/L bacto agar.

Terrific Broth (TB): Autoclave 12 g tryptone, 24 g yeast extract, 4 mL glycerol, 950 mL dH₂O separately from KPO₄ salts (23.14 g KH₂PO₄, 125.31 g K₂HPO₄, 1 L dH₂O); Mix 950 mL broth with 50 mL KPO₄ salts at room temperature.

Antibiotics: Kanamycin (50 μg/mL final).

Inducers: β-d-1-thiogalactopyranoside (IPTG, 500 μM final).

Tris-buffered saline with imidazole (TBSI): 250 mM NaCl, 20 mM Imidazole, 25 mM Tris-HCl, pH=8.

Lysis buffer: TBSI supplemented with 1 mg/mL Lysozyme (sigma, L6876, from chicken egg), 1 mg/mL DNase I (sigma, DN25, from bovine pancreas), and 1 mM Phenyl Methane Sulfonyl Fluoride (PMSF).

Elution buffer: 250 mM NaCl, 500 mM Imidazole, 25 mM Tris-HCl, pH=8.

Phosphate-buffered saline (PBS): 150 mM NaCl, 20 mM NaPO₄.

Lithium borate buffer: 10 mM lithium acetate, 10 mM Boric acid.

Tris-glycine buffer: 25 mM Tris, 192 mM glycine, 0.1% SDS, pH=8.3.

### DNA Cloning by PCR Mutagenesis and Isothermal Assembly

Synthetic genes encoding I53-50 and I53-47¹ were amplified using Kapa High Fidelity Polymerase according to manufacturer's protocols with primers incorporating the desired mutations or the Btat peptide. The resulting amplicons were isothermally assembled² with PCR-amplified or restriction digested (NdeI and XhoI) pET29b fragments and transformed into chemically competent E. coli XL1-Blue cells. Individual colonies were verified by Sanger sequencing. Plasmid DNA was purified using a Qiagen miniprep kit and transformed into chemically competent BL21(DE3)* cells for protein expression.

### Kunkel Mutagenesis

Kunkel mutagenesis was performed as previously described³. Briefly, E. coli 0236 was transformed with the desired pET vector and then infected with bacteriophage M13K07. Single-stranded DNA (ssDNA) was purified from PEG/NaCl-precipitated bacteriophage using a Qiaprep™ M13 kit. Oligonucleotides were phosphorylated for 1 hour with T4 polynucleotide kinase (NEB, M0201) and annealed to purified ssDNA plasmids. For routine cloning, annealing was performed using a temperature ramp from 95° C. to 25° C. over 30 minutes. For library generation, annealing mixtures were denatured at 95° C. for 2 minutes, followed by annealing for 5 minutes at either 55° C. (220 bp agilent oligonucleotides) or 50° C. (all other oligonucleotides). Oligonucleotides were extended using T7 DNA polymerase (NEB) for one hour at 20° C. and transformed into E. coli as described for either routine cloning or library generation.

### Transformation of DNA Libraries

Plasmid DNA generated as described above by isothermal assembly or kunkel mutagenesis was purified by SPRI purification⁴ and electrotransformed into E. coli DH10B (Invitrogen 18290-015) to produce libraries with at least 10× coverage. Transformed libraries were grown as lawns on LB agar plates containing 50 kanamycin. Additionally, a 10-fold dilution series of the transformed library was spotted onto an additional plate to assess library size. After 12-18 hours of growth, the resulting lawn of cells was scraped from the plate into 1 mL of LB and pelleted at 16,000 rcf for 30 seconds. Plasmid DNA was purified directly from this cell pellet using a Qiagen miniprep kit and electrotransformed into E. coli BL21(DE3)* with a minimum of 10× coverage of the library. The resulting bacterial lawns were then lifted from plates in 1 mL TB and inoculated directly into expression cultures.

### Deep Mutational Scanning Library Design, Amplification, and Purification

For the deep mutational scanning library, the DNA sequence encoding the two components of I53-50-v2 was divided into 7 windows of 159 bp. For each window, a pool of oligonucleotides was synthesized to mutate every residue of I53-50-v2 in the specified window (Agilent SurePrint™ Oligonucleotide Library Synthesis, OLS). Each oligonucleotide encoded a single amino acid change using the most common codon in E. coli for that amino acid. To disambiguate bona fide mutations from sequencing and reverse transcription errors, silent mutations were added on either side of the target being modified by the oligo to identify the position being mutated. Each of the 7 oligonucleotide pools was amplified from the OLS pool using primers annealing to constant regions flanking the mutagenic sequences. Reaction progress was monitored by SYBR green fluorescence on a Bio-Rad CFX96 to prevent over-amplification. The resulting amplicons were then PAGE purified and subjected to an additional round of amplification. Amplicons were then SPRI purified, and a final PCR reaction was set up with only the reverse primer to perform linear amplification of the desired primer sequence (50 cycles of temperature cycling were performed to generate a DNA sample highly enriched for the reverse strand). This sample was then purified using a Qiagen QIAquick™ PCR Purification Kit. The resulting pool of single stranded oligonucleotides was then used in a kunkel reaction as described above for library generation.

### Hydrophilic Polypeptide Library Design, Amplification, and Purification

The hydrophilic polypeptide library was generated by alternating sets of hydrophilic amino acids (DE, ST, QN, GE, EK, ES, EQ, EP, PAS) with a guest residue (A, S, T, E, D, Q, N, K, R, P, G, L, I) introduced between every 1, 2, or 5 occurrences to generate a final peptide of 59 amino acids in length. An additional 21 peptides were generated by splitting known hydrophilic peptides^{5,6} into 59 amino acid chunks or repeating one of their primary repeating units. All polypeptide sequences were reverse translated to DNA using codon frequencies found in E. coli K12⁷, and flanking sequences were added for amplification. These oligo sequences were synthesized using Agilent OLS technology. After amplification, flanking regions were removed using the AgeI and HindIII restriction enzymes, and cloned onto the C-terminus of the I53-50-v3 pentamer subunit by ligation (T4 ligase, NEB M0202, Final Concentration: 40 units; μL, 1×T4 ligase buffer with 1 mM ATP). The resulting DNA was SPRI purified and transformed as described above for library transformation.

### Protein Expression/Purification

E. coli BL21(DE3)* expression cultures were grown to an optical density of 0.6 in 500 mL TB supplemented with 50 μg/mL kanamycin at 37° C. with shaking at 225 rpm. Expression was induced by the addition of IPTG (500 μM final). Expression proceeded for 4 hours at 37° C. with shaking at 225 rpm. Cultures were harvested by centrifugation at 5,000 rcf for 10 minutes and stored at −80° C.

Cell pellets were resuspended in TBSI and lysed by sonication or homogenization using a Fastprep96 with lysing matrix B. Lysate was clarified by centrifugation at 24,000 rcf for 30 minutes and passed through 2 mL of Nickel-Nitrilotriacetic acid agarose (Ni-NTA) (Qiagen cat No. 30250), washed 3 times with 10 mL TBSI, and eluted in 3 mL of Elution buffer, of which only the second and third mL were kept. EDTA was immediately added to 5 mM final concentration to prevent Ni-mediated aggregation.

For in vitro evolution and all experiments involving hydrophilic tails, synthetic nucleocapsids were prepared with a C-terminal hexahistidine tag on the pentameric subunit. For these constructs, purification proceeded immediately from IMAC elution to size exclusion chromatography (SEC) using a Superose 6 Increase column (GE Healthcare, 29-0915-96) in TBSI.

For all in vivo evolution experiments, synthetic nucleocapsids were prepared with a N-terminal, thrombin cleavable hexahistidine tag on the pentameric subunit to allow scarless removal. This was done to allow removal of the affinity tag for in vivo use and to prevent the divalent cation-dependent aggregation observed in the C-terminal hexahistidine constructs. After elution from the IMAC column, these samples were dialyzed into PBS, treated with thrombin at a final concentration of 0.00264 units/4 for 90 minutes at 20° C. to remove the histidine tag. Thrombin was inactivated by addition of PMSF (1 mM final concentration), and nucleocapsids were purified by SEC using a Superose 6 Increase column in PBS.

Endotoxin was removed from all samples intended for animal studies. Endotoxin removal was performed after thrombin cleavage by addition of triton x-114 (1% final concentration volume/volume) followed by incubation at 4° C. for 5 minutes, incubation at 37° C. for 5 minutes, and centrifugation at 24,000 rcf at 37° C. for 2 minutes. The supernatant was then removed, incubated 4° C. for 5 minutes, incubated at 37° C. for 5 minutes, and centrifuged at 24,000 rcf at 37° C. for 2 minutes to ensure optimal endotoxin removal before continuing with SEC purification in PBS.

### Gel Electrophoresis

Native agarose gels: Agarose gels were prepared using 1% Ultrapure agarose (Invitrogen) in lithium borate buffer. For synthetic nucleocapsid samples, 20 μL purified synthetic nucleocapsids were treated with 10 μg/mL RNase A (20° C. for 10 minutes), mixed with 4 μL 6× loading dye (NEB B7025S, no SDS), and electrophoresed at 100 volts for 45 minutes. Gels were then stained with SYBR gold (Thermo-Fisher S11494) for RNA followed by Gelcode (Thermo-Fischer 24590) for protein.

DNA gels: 1% agarose gels were prepared containing SYBR Safe™ (Invitrogen) according to the manufacturer's protocols.

Protein SDS-PAGE: SDS-PAGE was performed using 4-20% polyacrylamide gels (Bio-Rad) in tris-glycine buffer.

### RNA Purification and Reverse Transcription

RNA was purified using (Thermo-Fisher Scientific, 15596018) and the Qiagen RNeasy kit (Qiagen, 74106) according to the manufacturers' instructions. Briefly, 100 μL synthetic nucleocapsid samples were mixed vigorously with 500 μL TRIzol. 100 μL chloroform was added and mixed vigorously, and then the solution was centrifuged for 10 min at 24,000 rcf. 150 μL of the aqueous phase was mixed with 150 μL, of 100% ethanol, transferred to a RNeasy spin column for purification according to manufacturer's instructions, and eluted in 50 μL nuclease-free dH₂O. For samples intended for absolute quantification (including standards) yeast tRNA was added to 100 ng/4 final concentration to ensure consistent sample complexity.

Reverse transcription was carried out using Thermoscript Reverse Transcriptase according to the manufacturer's instructions for one hour at 53° C., with the only modifications being that a gene-specific primer (skpp_reverse) was used. Thus, a 10 μL reaction contained: 1 μL dNTPs (10 mM each), 1 μL DTT (100 μM), 1 μL Thermoscript Reverse Transcriptase, 2 μL cDNA synthesis buffer, 1 μL RNase-Out, 1 μL skpp_reverse (10 μM), 2 μL, purified RNA template, and 1 μL nuclease-free dH₂O. Controls lacking reverse transcriptase were set up identically except with the substitution of nuclease-free dH₂O in place of Thermoscript™ Reverse Transcriptase.

### Quantitative PCR

Quantitative PCR was performed in a 10 μL reaction using a Kapa High Fidelity™ PCR kit (Kapa Biosystems, KK2502) according to the manufacturer's instructions with the addition of SYBR green at 1× concentration and 0.5 μM forward and reverse primers (skpp_fwd and skpp_Offset_Rev) for quantification of nucleocapsid RNA. Thermocycling and Cq calculations were performed on a Bio-Rad CFX96 with the following protocol: 5 min at 95° C., then 40 cycles of: 98° C. for 20 seconds, 64° C. for 15 seconds, 72° C. for 90 seconds.

Allele specific qPCR was performed using Kapa 2G Fast polymerase readymix along with 1×SYBR green, 3 μL of 100× diluted cDNA template, and 0.5 μM each of the forward and reverse allele specific primer specific for each construct. Thermocycling and Cq calculations were performed on a Bio-Rad CFX96 with the following protocol: 5 min at 95° C., then 40 cycles of: 95° C. for 15 seconds, 58° C. for 15 seconds, 72° C. for 90 seconds.

Absolute quantitation of full length RNA per protein capsid was calculated from Cq values using a linear fit (−log([RNA])=m*(Cq) b) of a standard curve comprised of in vitro transcribed nucleocapsid RNA. In vitro transcription was performed using a NEB HiScribe™ T7 high yield RNA synthesis kit (NEB, E2040S) according to the manufacturer's protocols. Excess DNA was degraded using RNase-free DNAse I (NEB, M0303), and RNA was purified using Agencourt™ RNAClean™ XP (Beckman Coulter, A63987) according to manufacturer protocols. The concentration of this standard was measured using a Qubit™ RNA HS Assay Kit (Life Technologies, Q32852), and a 10-fold dilution series was prepared in nuclease-free dH₂O supplemented with 100 ng/μL yeast tRNA. The dilution series samples were then processed in parallel with the synthetic nucleocapsid samples using the RNA purification and reverse transcription protocol above, and run on the same qPCR plate as the samples quantified.

In the pooled samples used to compare the fitness of I53-50-v1, I35-50-v2, I53-50-v3, and I53-50-v4, the total amount of full-length nucleocapsid genome was quantified by qPCR performed with skpp_fwd and skpp_rev using the Kapa™ High Fidelity PCR kit as described above. Subsequently, the relative fraction of RNA corresponding to each version was determined by allele specific PCR as described above using allele-specific primers (Table S6) unique to each version. Absolute quantitation was with respect to a standard curve for each version prepared as described above. The fractional RNA content from each version was then multiplied by total amount of full-length genomes.

### In Vitro Synthetic Nucleocapsid Selection Conditions

The total amount of RNA packaged in nucleocapsids was evaluated by treating 100 μL synthetic nucleocapsids with 10 μg/mL RNase A at 20° C. for 10 minutes (“Total RNA”) so as to degrade non-encapsulated RNA. Reaction buffer was PBS for N-terminal histidine tag constructs or TBSI for C-terminal histidine tag constructs. More stringent RNase protection assays were performed with 10 μg/mL RNase A at 37° C. for the specified duration (“RNase”). Protection from blood was assessed by diluting synthetic nucleocapsids 1:10 in heparinized whole murine blood (collected from the vena cava of mice sacrificed using a lethal dose of avertin and stabilized in 6 units/mL heparin) and incubating at 37° C. for the specified duration (“Blood”). Samples were then centrifuged at 24,000 rcf for 2 minutes before adding the supernatant to TRIzol. RNA was purified as described in the RNA Purification and RT-qPCR sections. All reactions were quenched by adding the sample directly to 500 μL TRIzol.

Within the work described here, a full length RNA is defined as the mRNA molecule encoding the polypeptide components of the nanostructure. However, in some embodiments, an RNA fragment encoding only a subset of the nanostructure, or an RNA payload unrelated to the nanostructure, is used in a particular application, the minimal RNA sequence capable of carrying out the intended function should be quantified for purposes of determining packaging efficiency. The packaging efficiency is defined as the number of moles of full length RNA or (by RT-qPCR) per molar equivalent of intact nanomaterial protein as measured by qubit assay. Further assay details are described in methods under In vitro synthetic nucleocapsid selection conditions.

### In Vivo Synthetic Nucleocapsid Selection Conditions

6-8 week old Balbc mice were retro-orbitally injected with 150 μL of synthetic nucleocapsids. Synthetic nucleocapsid libraries containing either hydrophilic polypeptides (104 μg/mL) or exterior surface mutations (570 μg/mL) were created and selected for circulation time in live mice. Five mice per library underwent retro-orbital injections and tail lancet blood draws at 5, 10, 15, and 30 minutes, with a final sacrifice and blood draw at 60 minutes. Following Illumina MiSeq™ sequencing of the selected nucleocapsid libraries, the circulation times of several selected variants (10 hydrophilic polypeptide variants, 4 surface mutation variants, I53-50-v1, I53-50-v2, and I53-50-v3 were pooled to 570n/mL, total protein) were compared in 5 mice with tail lancet blood draws at 5, 15, 30, 60, and 120 minutes, submental collection¹⁰ at 4 hours, and final sacrifice and blood draw at 6 hours. I53-50-v4 was created based on the consensus sequence of the most common residues in the library after in vivo selection.

### Synthetic Nucleocapsid Characterization for FIG. 4a-d

I53-50-v1; I53-50-v2, I53-50-v3, and I53-50-v4 were expressed in E. coli BL21(DE3)*, harvested, purified by IMAC, dialyzed into PBS, cleaved by thrombin, subjected to endotoxin removal, and purified by SEC. The protein concentrations for each sample were determined using a Qubit Protein Assay Kit (Thermofisher Scientific, Q33211) and samples were mixed to give a final concentration of 170 μg/mL nucleocapsid protein for each version (680 μg/mL total). This pool was split into four different samples that were each subjected to the Total RNA, RNase, Blood, and in vivo selection conditions described above. For in vivo selection, 150 μL of the pool was injected retro-orbitally, and tail lancet draws were performed at 5 minutes, 1 hour, 3 hours, and 6 hours, submental collection¹⁰ at 10 hours, and final sacrifice and blood draw at 24 hours.

### Synthetic Nucleocapsid Biodistribution

I53-50-v3 and I53-50-v4 were injected into 6 mice each. Animals were then sacrificed after either 5 minutes or 4 hours (3 animals per nucleocapsid version at each time point). Half of each bisected organ and 20 μL of whole blood were collected into tubes containing 500 μL TRIzol and homogenized. RNA was purified, total tissue RNA was measured by either A₂₆₀ (organs) or Qubit RNA HS Assay Kit (Blood, due to its lower total RNA) and full-length nucleocapsid genomes were quantitated by RT-qPCR as described above.

### Negative-Stain Electron Microscopy Specimen Preparation, Data Collection, and Data Processing

6 μl of purified protein (I53-50-v0, I53-50-v1, I53-50-v2, I53-50-v3, I53-50-v4, I53-50-Btat, I53-47-v0, I53-47-v1, I53-47-Btat) at 0.04-0.3 mg/mL were applied to glow discharged, carbon-coated 300-mesh copper grids (Ted Pella), washed with Milli-Q water and stained with 0.75% uranyl formate as described previously⁸. Screening and sample optimization was performed on a 100 kV Morgagni M268 transmission electron microscope (FEI) equipped with an Orius charge-coupled device (CCD) camera (Gatan). Data were collected with Leginon automatic data-collection software⁹ on a 120 kV Tecnai G2 Spirit™ transmission electron microscope (FEI) using a defocus of 1 μm with a total exposure of 30 e-/A². All final images were recorded using an Ultrascan™ 4000 4 k×4 k CCD camera (Gatan) at 52,000× magnification at the specimen level. For data collection used in two-dimensional class averaging, the dose of the electron beam was 80 e-/Å², and micrographs were collected with a defocus range between 1.0 and 2.0 μm. Coordinates for unique particles (7,979 for I53-50-v0 and 7,130 for I53-50-v4) were obtained for averaging using EMAN2¹⁰. Boxed particles were used to obtain two-dimensional class averages by refinement in EMAN2.

### Illumina Sequencing Sample Preparation Evolution Experiments

Evolution experiments were analyzed by performing targeted RNAseq on full-length nucleocapsid genomes surviving the specified selection condition (RT-qPCR using skpp_reverse as the RT primer and qPCR with skpp_fwd and skpp_Offset_Rev). The starting populations and selected populations were evaluated by sequencing nucleocapsid genomes extracted from producer cells or nucleocapsids, respectively. Following SPRI purification, two sequential Kapa HiFi qPCR reactions were performed using Kapa HiFi polymerase to add sequencing adapters and barcodes, respectively. qPCR reactions were monitored by SYBR green fluorescence and terminated prior to completion so as to prevent over-amplification. The resulting amplicons were purified using SPRI purification or a Qiagen QIAquick™ Gel Extraction Kit. The resulting amplicons were then denatured and loaded into a Miseq™ 600 cycle v3 (Illumina) kit and sequenced on an Illumina MiSeq™ according to the manufacturer's instructions.

### Illumina Sequencing Sample Preparation for Comprehensive RNAseq

The composition of encapsulated RNA was evaluated by performing comprehensive RNAseq on total RNA from producer cells (representing expression levels) and nucleocapsids (representing encapsulated RNA). RNA was extracted using TRIzol and purified using a Direct-zol™ RNA MiniPrep Plus kit (Zymo Research, R2072) with on-column DNAse digestion. The purified RNA was quantitated using a Qubit RNA HS Assay Kit, and 100 ng of RNA was used to prepare each RNAseq library with a NEBNext® Ultra™ RNA Library Prep Kit for Illumina® kit (NEB, E7530S). Each library was PCR amplified using Kapa HiFi™ polymerase to add sequencing barcodes before being pooled for sequencing. The resulting libraries were then denatured and loaded into an Illumina NextSeq™ 500/550 High Output Kit v2 (75 cycles) kit and sequenced on an Illumina NextSeq™ according to the manufacturer's instructions.

### Sequencing Analysis for Evolution Experiments

Raw sequencing reads were converted to fastq format and parsed into separate files for each sequencing barcode using the Generate Fastq workflow on the Illumina MiSeq™. Forward and reverse reads were combined using the read_fuser script from the enrich package¹¹.

For all libraries, enrichment values were calculated as the change in fraction of the library corresponding to each linked sequence (rank order of variants) or unlinked substitutions (heatmaps) that were observed at least 10 times in the naïve library. The base 10 logarithm of each value was then taken in order to give enrichment values that more symmetrically span enrichment and depletion.

For the charge optimization library, the total interior charge of each variant was calculated by summing the number of Lys and Arg residues, and subtracting the number of Asp and Glu residues in the regions of the sequence determined to be on the interior surface by visual inspection of the design model. In I53-50, the interior surface positions were determined to be: Trimer([136:152], [156:170], [179:205]) Pentamer ([81:89], [117:127]). This results in a net charge of +420 for I53-50-v1 and I53-50-v2. I53-50-v0 (SEQ ID 1 modified by R119N, R121D) and shown to package <0.69 genomes per 1000 capsids) has an interior net charge of 0. As ananother example; these positions would for I53-47: Trimer: [30:37], [65:73], [100:108] Pentamer: [82:89]; [117:128].

For the deep mutational scanning library, substitutions were only counted if they contained the expected silent mutation barcodes as described in oligonucleotide design. This greatly reduces the effect of both RT-PCR errors and sequencing errors because instead of a minimum of one error allowing a miscalled amino acid mutation, a minimum of three errors are required for a mutation to be miscalled.

Heatmaps were generated using a custom MatPlotLib¹² script by mapping the calculated log enrichment values onto a LinearSegmentedColormap (purple, white, orange; rgb=(0.75, 0, 0.75), (1, 1, 1), (1.0, 0.5, 0)) using the pcolormesh function. The minimum and maximum values of the colormesh were set as shown in each figure to fully utilize the dynamic range of the colormap. A pymol session colored by the average log enrichment of all 20 amino acids at each position was created by substituting average log enrichment values for B-factors in the pdb file and running the command: spectrum b, purple white white orange, minimum=−1.5, maximum=0.6. Note that this is rescaled relative to the coloring of individual residues because the averages span a smaller range than the individual values and thus a different color range is needed to clearly differentiate values.

### Sequencing Analysis for Comprehensive RNAseq

RNAseq data was converted from bcl format to fastQ format using Illumina's bcl2fastq script. Hisat2¹³ converted fastQ to sam, and samtools¹⁴ converted sam files to sorted barn files. Stringtie¹⁵ was used to calculate gene expression as TPM (Transcripts Per kilobase Million).

### Dynamic Light Scattering

Dynamic Light Scattering was performed on a DynaPro™ NanoStar™ (Wyatt) DLS setup. I53-50-v0, I53-50-v1, and I53-50-v4 were evaluated with 0.2 mg/mL of nucleocapsid protein in PBS at 25° C. Data analysis was performed using DYNAMICS™ v7 (Wyatt) with regularization fits.

### REFERENCES FOR EXAMPLE 1 MATERIALS AND METHODS

1. Deverman, B. E. et al. Cre-dependent selection yields AAV variants for widespread gene transfer to the adult brain. Nat Biotechnol 34, 204-209 (2016).
2. Chackerian, B., Caldeira Jdo, C., Peabody, J. & Peabody, D. S. Peptide epitope identification by affinity selection on bacteriophage MS2 virus-like particles. J Mol Biol 409, 225-237 (2011).
3. Smith, G. P. Filamentous fusion phage: novel expression vectors that display cloned antigens on the virion surface. Science 228, 1315-1317 (1985).
4. Soderlind, E., Simonsson, A. C. & Borrebaeck, C. A. Phage display technology in antibody engineering: design of phagemid vectors and in vitro maturation systems. Immunol Rev 130, 109-124 (1992).
5. Bale, J. B. et al. Accurate design of megadalton-scale two-component icosahedral protein complexes. Science 353, 389-394 (2016).
6. Hsia, Y. et al. Design of a hyperstable 60-subunit protein icosahedron. Nature 535, 136-139 (2016).
7. Drouin, L. M. et al. Cryo-electron Microscopy Reconstruction and Stability Studies of the Wild Type and the R432A Variant of Adeno-associated Virus Type 2 Reveal that Capsid Structural Stability Is a Major Factor in Genome Packaging. J Virol 90, 8542-8551 (2016).
8. Sommer, J. M. et al. Quantification of adeno-associated virus particles and empty capsids by optical density measurement. Mol Ther 7, 122-128 (2003).
9. Pascual, E. et al. Structural basis for the development of avian virus capsids that display influenza virus proteins and induce protective immunity. J Virol 89, 2563-2574 (2015).
10. Waehler, R., Russell, S. J. & Curiel, D. T. Engineering targeted viral vectors for gene therapy. Nat Rev Genet 8, 573-587 (2007).
11. Harrison, S. C., Olson, A. J., Schutt, C. E., Winkler, F. K. & Bricogne, G. Tomato bushy stunt virus at 2.9 A resolution. Nature 276, 368-373 (1978).
12. Lilavivat, S., Sardar, D., Jana. S., Thomas, G. C. & Woycechowsky, K. J. In vivo encapsulation of nucleic acids using an engineered nonviral protein capsid. J Am Chem Soc 134, 13152-13155 (2012).
13. Hernandez-Garcia, A. et al. Design and self-assembly of simple coat proteins for artificial viruses. Nat Nanotechnol 9, 698-702 (2014).
14. Worsdorfer, B., Woycechowsky, K. J. & Hilvert, D. Directed evolution of a protein container. Science 331, 589-592 (2011).
15. Puglisi, J. D., Chen, L., Blanchard, S. & Frankel, A. D. Solution structure of a bovine immunodeficiency virus Tat-TAR peptide-RNA complex. Science 270, 1200-1203 (1995).
16. Starita, L. M. & Fields, S. Deep Mutational Scanning: A Highly Parallel Method to Measure the Effects of Mutation on Protein Function. Cold Spring Harb Protoc 2015, 711-714 (2015).
17. Whitehead, T. A, et al. Optimization of affinity, specificity and function of designed influenza inhibitors using deep sequencing. Nat Biotechnol 30, 543-548 (2012).
18. Knop, K., Hoogenboom, R., Fischer, D. & Schubert, U.S. Poly(ethylene glycol) in drug delivery: pros and cons as well as potential alternatives. Angew Chem Int Ed Engl 49, 6288-6308 (2010).
19. Hui, D. J. et al. AAV capsid CD8+ T-cell epitopes are highly conserved across AAV serotypes. Mol Ther Methods Clin Dev 2, 15029- (2015).
20. Mingozzi, F. et al. CD8(+) T-cell responses to adeno-associated virus capsid in humans. Nat Med 13, 419-422 (2007).

### Example 2

We describe synthetic nucleocapsids and their protein assemblies that can be modified to package diverse cargos and linked to one or more targeting domains that target cell-specific cell surface markers/motifs. The ability to modularly modify the exterior and interior surfaces of synthetic nucleocapsids and their protein assemblies sets them apart from natural viruses, which are more difficult to engineer. The interior surface may be modified to display different cargo packaging domains, whereas the exterior surface may be modified to bind to specific cell types expressing target cell surface markers. In this way, synthetic nucleocapsids and their protein assemblies can function in two distinct modes: evolution mode and formulation mode. For example, genome-packaging versions of the synthetic nucleocapsids and their protein assemblies can be mutated and selected to evolve desired properties such as cell targeting, and then the interior surfaces of the resulting improved variants can be modified so that they no longer package their genome, but package a different useful cargo (e.g., cytotoxins, fluorophores, peptides, proteins, enzymes, ssDNA, dsDNA, mRNA, siRNA, etc.).

We have shown herein the modularly targeting of synthetic nucleocapsids to specific cell types by attaching one or more polypeptide targeting domains either by direct genetic fusion or by post-translational crosslinking (e.g., Spycatcher™/Spytag™). These polypeptide targeting domains can be derived from diverse classes of protein scaffolds, including, for example, affibodies, DARPins, adnectins/monobodies, and spycatcher.

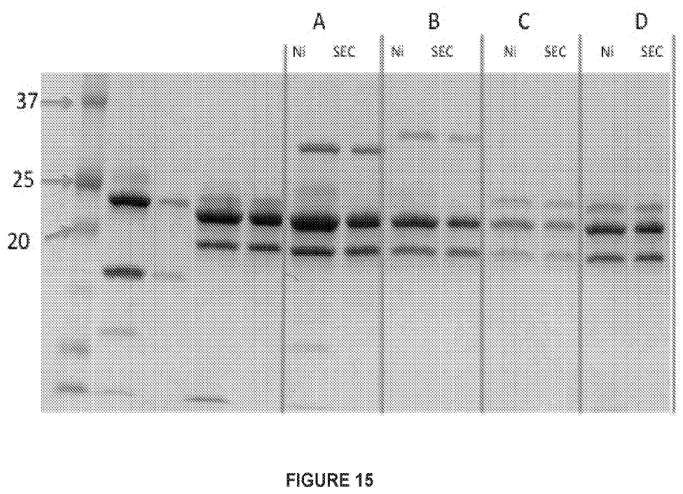

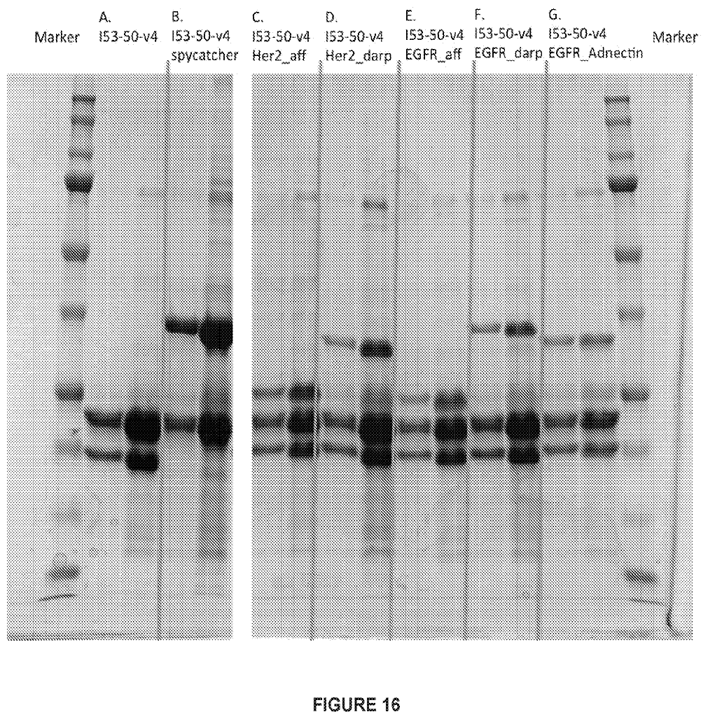

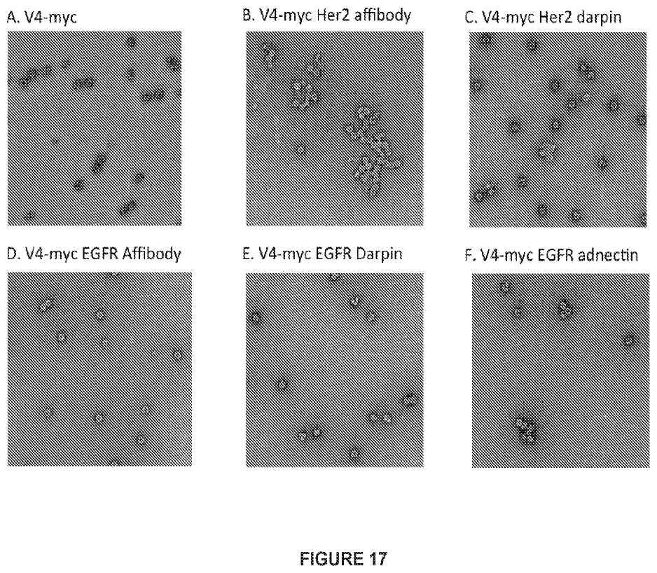

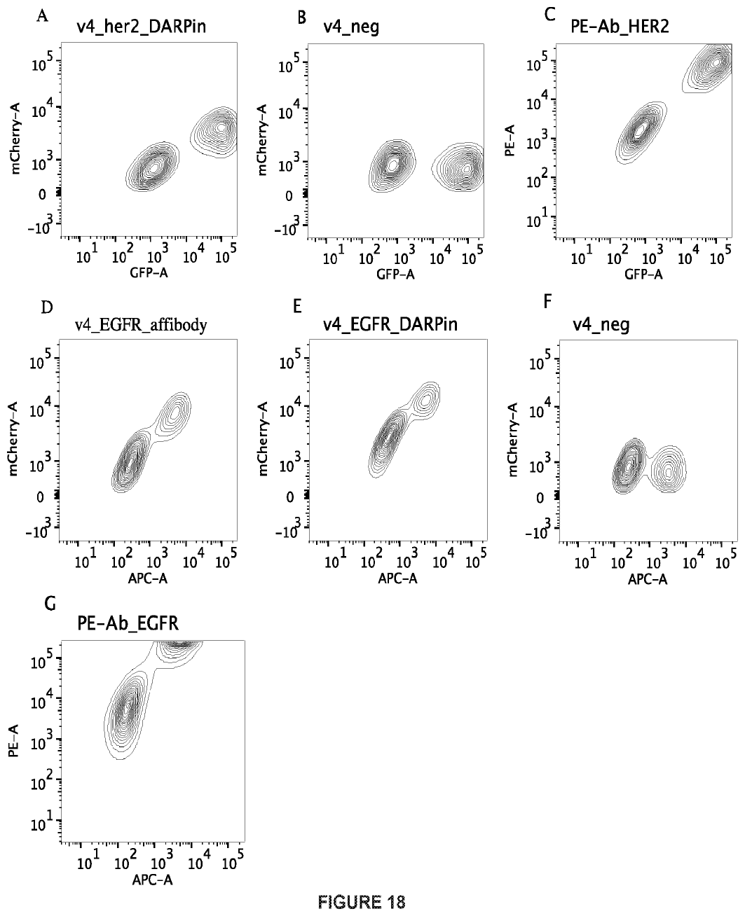

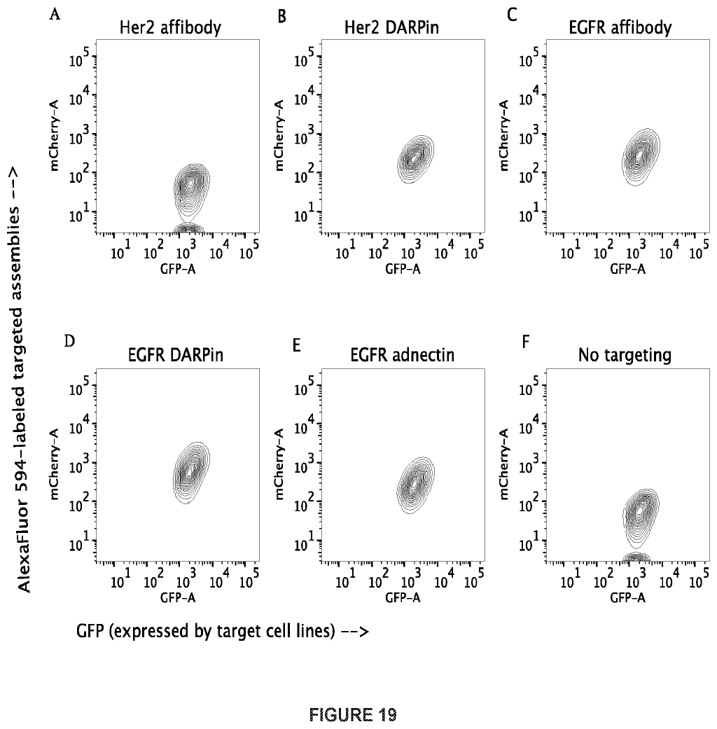

In FIGS. 15 and 16, we used SDS-PAGE to show that synthetic nucleocapsids displaying modular targeting domains may be soluble and can be purified by immobilized metal affinity chromatography. We could either display full valency targeting protein (60 copies; e.g., spycatcher, FIG. 16b) or partial valency targeting protein by using a GSprfB linker (e.g., DARPin, affibody, adnectin). In the case of full valency, two protein species are visualized by SDS-PAGE: the unmodified trimeric subunit and the Spycatcher™-displaying pentameric subunit. In the case of the partial valency, three protein species are visualized by SDS-PAGE: the unmodified trimeric subunit, the unmodified pentameric subunit, and the targeting-domain-displaying pentameric subunit. Based on densitometry, we estimate that approximately 30% of pentameric subunits display the targeting domain. We then used mass spectrometry to confirm the correct masses of these three protein species for the synthetic nucleocapsids displaying the anti-HER2 DARPin, anti-HER2 affibody, anti-EGFR affibody, and anti-EGFR DARPin (data not shown). We also used dynamic light scattering (data not shown) and negative-stain transmission electron microscopy (FIG. 17) to confirm that the resulting nucleocapsids are still well-formed, monodisperse icosahedral assemblies.

After biochemically characterizing the synthetic nucleocapsids, we used cell lines expressing either HER2 or EGFR to evaluate whether synthetic nucleocapsids displaying targeting domains could specifically bind to cells expressing their cognate cell surface markers. We used a mixed population of 293 Freestyle™ cells stably expressing no target, HER2, EGFR, or HER2/EGFR, and we used RAJI cells stably expressing both HER2 and EGFR. The following targeting domains showed specific binding to HER2-expressing cells: anti-HER2 DARPin. The following targeting domains showed specific binding to EGFR-expressing cells: anti-EGFR affibody, anti-EGFR DARPin, anti-EGFR adnectin. The anti-HER2 affibody did not bind to HER2-expressing cells, perhaps because it precipitated during storage at 4° C. The non-targeted negative control nucleocapsid exhibited minimal binding to target cells in a HER2- and EGFR-independent manner.

Some applications of synthetic nucleocapsids may require covalent attachment of a small molecule. In a subset of those cases, simultaneous packaging of RNA may be undesirable. In anticipation of such applications, we generated a set of nucleocapsids in which RNA packaging mutations were reverted to the amino acid in the original, non-RNA packaging versions. Further, cysteine residues were mutated such that each pair of trimeric and pentameric subunits contained a single cysteine residue (for 60 cysteines in an assembled nucleocapsid) at a favorable location for conjugation on the interior surface of the assembled particle. An additional version was made in which a flexible linker region containing 6 cysteines was appended to the trimeric subunit to allow conjugation of a higher number of small molecules. These particles were produced in E. coli and purified by IMAC. SDS-PAGE analysis (FIG. 20) of the resulting particles clearly showed successful production and stoichiometric assembly of the two components in the case of both the 60 and 360 cysteine nucleocapsid.

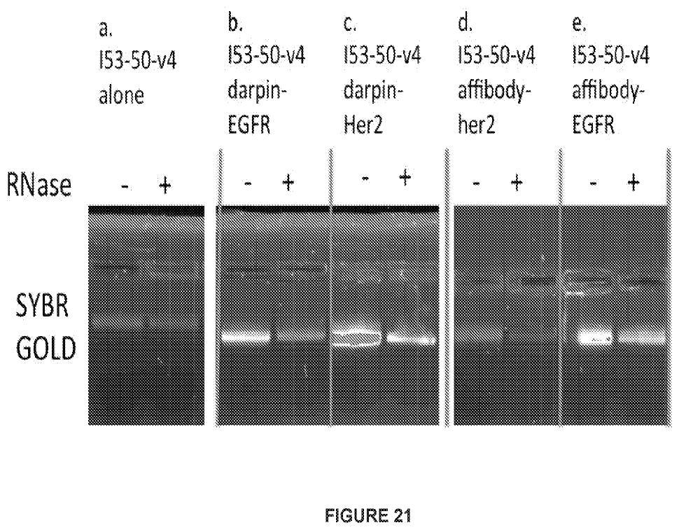

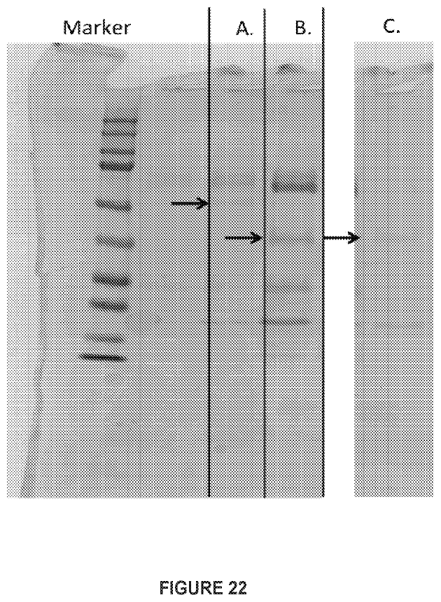

To show that the targeted nucleocapsids retained RNA packaging when modified with a targeting domain, we ran 4 nucleocapsids on a native agarose gel stained with SYBR gold(I53-50v-4, I53-50v-4-EGFR darpin, I53-50v-4-Her2 darpin, I53-50v-4-affibody-Her2, I53-50v-4-affibody-EGFR). These nucleocapsids all showed monodisperse, RNase resistant bands under SYBR gold staining indicative of RNA packaging (FIG. 21).

We tested several additional fusion domains on the trimeric subunit-scFV targeting CD3, adnectin targeting EGFR, and spycatcher. These domains also showed bands of the correct size on SDS-PAGE after IMAC purification, suggesting successful production of the targeted nucleocapsid.

As demonstrated herein, diverse protein scaffolds can be modularly displayed on synthetic nucleocapsids. Other targeting domains, such as for example, single chain variable fragments (scFvs), nanobodies, or other non-immunoglobulin-derived scaffolds, including those described by Skrlec et al. (Katja Skrlec, Borut Strukelj, and Ales Berlec Non-immunoglobulin scaffolds: a focus on their targets Trends in Biotechnology, July 2015, Vol. 33, No. 7), and the like, may be substituted for the protein scaffolds described herein. Furthermore, the Spycatcher™-displaying synthetic nucleocapsid provides an opportunity to post-translationally link targeting domains produced using other methods (e.g., mammalian protein expression).

### Methods for Example 2

### Solutions and Buffers

Lysogeny Broth (LB): Autoclave 10 g tryptone, 5 g yeast extract, 5 g NaCl, 1 L dH₂O. LB agar plates: Autoclave LB with 15 g/L bacto agar. Terrific Broth (TB): Autoclave 12 g tryptone, 24 g yeast extract, 4 mL glycerol, 950 mL dH₂O separately from KPO₄ salts (23.14 g KH₂PO₄, 125.31 g K₂HPO₄, 1 L dH₂O); Mix 950 mL broth with 50 mL KPO₄ salts at room temperature. Antibiotics: Kanamycin (50 μg/mL final). Inducers: β-d-1-thiogalactopyranoside (IPTG, 500 μM final). Tris-buffered saline with imidazole (TBSI): 250 mM NaCl, 20 mM imidazole, 25 mM Tris-HCl, pH 8.0.

Lysis buffer: TBSI supplemented with 1 mg/mL lysozyme (sigma, L6876, from chicken egg), 1 mg/mL DNase I (sigma, DN25, from bovine pancreas), and 1 mM phenyl methane sulfonyl fluoride (PMSF). Elution buffer: 250 mM NaCl, 500 mM imidazole, 25 mM Tris-HCl, pH 8.0. Phosphate-buffered saline (PBS): 150 mM NaCl, 20 mM NaPO₄. PBSF: PBS supplemented with 0.1% w/v bovine serum albumin (BSA) 20× lithium borate buffer (use at 1×): 1 L dH₂O, 8.3 g lithium hydroxide monohydrate, 36 g boric acid. Tris-glycine buffer: 25 mM Tris-HCl, 192 mM glycine, 0.1% SDS, pH 8.3.

### Generation of DNA Encoding Invention:

Synthetic genes encoding the Synthetic Nucleocapsid and desired targeting modifications were amplified using Kapa™ High Fidelity Polymerase according to manufacturer's protocols with primers incorporating the desired mutations. The resulting amplicons were isothermally assembled with PCR-amplified or restriction-digested (NdeI and)(hop pET29b fragments and transformed into chemically competent E. coli XL1-Blue cells. Monoclonal colonies were verified by Sanger sequencing. Plasmid DNA was purified using a Qiagen miniprep kit and transformed into chemically competent E. coli Lemo21 cells for protein expression.

### Protein Production

Expression cultures were grown to an optical density of 0.6 at 600 nm in 500 ml TB supplemented with 100 μg ml⁻¹ kanamycin at 37° C. with shaking at 225 r.p.m. Expression was induced by the addition of IPTG (500 μM final). Expression proceeded for 4 h at 37° C. with shaking at 225 r.p.m. Cultures were harvested by centrifugation at 5,000 r.c.f for 10 min and stored at −80° C.

Cell pellets were resuspended in TBSI and lysed by microfluidizing. Lysate was clarified by centrifugation at 24,000 r.c.f. for 30 min and passed through 2 ml of nickel-nitrilotriacetic acid agarose (Ni-NTA) (Qiagen, 30250), washed 3 times with 10 ml TBSI, and eluted in 3 ml of elution buffer, of which only the second and third milliliters were kept. EDTA was immediately added to 5 mM final concentration to prevent Ni-mediated aggregation.

Synthetic nucleocapsids were prepared with a N-terminal, thrombin cleavable histidine tag on the pentameric subunit to allow scarless removal. After elution from the IMAC column, these samples were dialysed into PBS, treated with thrombin at a final concentration of 0.00264 U μl⁻¹ for 14-18 hours at 4° C. to remove the histidine tag. Thrombin was inactivated by addition of PMSF (1 mM final concentration), and synthetic nucleocapsids were purified by SEC using a Superose™ 6 Increase column in HEPES buffer (25 mM HEPES, 150 mM NaCl, pH=7.4).

SDS-PAGE was performed on purified samples using 4-20% polyacrylamide gels (Bio-Rad) in Tris-glycine buffer.

### Dynamic Light Scattering

Dynamic light scattering was performed on a DynaPro™ NanoStar (Wyatt) DLS setup. 0.2-0.4 mg ml⁻¹ of synthetic nucleocapsid protein in PBS at 25° C. Data analysis was performed using DYNAMICS™ v7 (Wyatt) with regularization fits.

### Native Gels

Agarose gels were prepared using 1% ultrapure agarose (Invitrogen) in lithium borate buffer. For synthetic nucleocapsid samples, 20 μl purified synthetic nucleocapsids were treated with 10 μg ml⁻¹ RNase A (20° C. for 10 min), mixed with 4 μl 6× loading dye (NEB B7025S, no SDS), and electrophoresed at 100 V for 45 min. Gels were stained with SYBR™ gold (Thermo Fischer Scientific, S11494) for RNA.

### Negative-Stain Electron Microscopy Specimen Preparation, Data Collection, and Data Processing

6 μl of purified protein at 0.001-0.01 mg/mL were applied to glow discharged, carbon-coated 300-mesh copper grids (Ted Pella), washed with Milli-Q water and stained with 0.75% uranyl formate as described previously⁽¹⁾. Data were collected on a 100 kV Morgagni M268 transmission electron microscope (FEI) equipped with an Onus charge-coupled device (CCD) camera (Gatan).
1. Nannenga, B. L., Iadanza, M. G., Vollmar, B. S. &. Gonen, T. Overview of electron crystallography of membrane proteins: crystallization and screening strategies using negative stain electron microscopy. Curr Protoc Protein Sci Chapter 17, Unit 17.15 (2013).

### Additional Methods:

Mass Spectrometry Molecular weights of designs were confirmed using electrospray ionization mass spectrometry (ESI-MS) on a Thermo Scientific TSQ Quantum Access mass spectrometer. Raw data was deconvoluted using the ProMass™ software from Novatia. Samples were run at 0.2-0.4 mg/mL.

Cell culture: 293Freestyle cell lines were maintained in Freestyle 293 expression media, and Raji cell lines were maintained in RPMI complete media (RPMI supplemented with 10% fetal bovine serum, MEM non-essential amino acids, HEPES, and penicillin-streptomycin solution).

Flow cytometry: Prior to binding, cells were washed once and resuspended at a density of 2×10⁶ cells/mL in PBSF (150 mM NaCl, 20 mM NaPO₄, and 0.1% w/v BSA, pH 8.0). Individual binding reactions were composed of 100 μL of cells (2×10⁵ cells) supplemented with the specified concentration of AF680-labeled protein and incubated on ice for 30 minutes. The cells were washed once in 500 μL PBSF to remove unbound protein and then resuspended in 500 μL binding buffer. Flow cytometry was performed on an LSRII to analyze AlexaFluor™ 568 binding (561 nm laser, 610/20 detector), HER2-EGFP expression (488 nm laser, 530/30 detector), EGFR-iRED expression (637 nm laser, 670/30 detector), and PE binding (561 nm laser, 582115 detector).

1. An isolated polypeptide comprising
(a) an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length to the amino acid sequence of SEQ ID NO:1, wherein the polypeptide includes one or more amino acid change from SEQ ID NO:1 selected from the group consisting of K2T, K9R, K11T, K61D, E74D, T126D, E166K, S179K/N, T185K/N, E188K, A195K, and E198K; or
(b) an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence of SEQ ID NO:2, wherein the polypeptide includes one or more amino acid change from SEQ ID NO:2 selected from the group consisting of H6Q, Y9H/Q, E24F/M, A38R, D39K, D43E, E67K, S105D, R119N, R121D, D122K, D124K/N, and H126K; or
(c) comprising an amino acid sequence that is at least 50% identical to the full length of the amino acid sequence of SEQ ID NO:3, wherein the polypeptide includes one or more amino acid change from SEQ ID NO:3 selected from the group consisting of T13D, S71K, N101R, and D105K; or
(d) comprising an amino acid sequence that is at least 50% identical to the full length of the amino acid sequence of SEQ ID NO:4, wherein the polypeptide includes one or more amino acid change from SEQ ID NO:4 selected from the group consisting of S105D, R119N, R121D, D122K, A124K, and A150N.

2. The isolated polypeptide of claim 1, comprising an amino acid sequence that is at least 75% identical to the full length of the amino acid sequence of SEQ ID NO:1, 2, 3, or 4.

3. The isolated polypeptide of claim 1, comprising an amino acid sequence that is at least 90% identical to the full length of the amino acid sequence of SEQ ID NO:1, 2, 3, or 4.

4. The isolated polypeptide of claim 1, wherein the amino acid sequence of the polypeptide is identical to the amino acid sequence of SEQ ID NO:1 at least at 1, 2, 3, or all 4 identified interface position selected from the group consisting of residues 25, 29, 33, and 54, and wherein the polypeptide is optionally identical to the amino acid sequence of SEQ ID NO:1 at residue 57.

5.-8. (canceled)

9. The isolated polypeptide of claim 1, wherein the polypeptide includes each of the following amino acid changes from SEQ ID NO:1: E74D, C76A, C100A, T126D, C165A, C203A, and optionally includes the following additional amino acid change from SEQ ID NO:1: N160C.

10. The isolated polypeptide of claim 1, wherein the polypeptide includes 1, 2, 3, 4, or all 5 or more of the following amino acid changes from SEQ ID NO:1: C76A, C100A, N160C, C165A, and C203A.

11.-16. (canceled)

17. The isolated polypeptide of claim 1, wherein the amino acid sequence of the polypeptide is identical to the amino acid sequence of SEQ ID NO:2 at residue 132.

18. The isolated polypeptide of claim 1, wherein the amino acid sequence of the polypeptide is identical to the amino acid sequence of SEQ ID NO:2 at least at 1, 2, 3, 4, or all 5 identified interface position selected from the group consisting of residues 128, 131, 132, 133, and 135.

19. The isolated polypeptide of claim 1, wherein the polypeptide includes 7 or more amino acid changes from SEQ ID NO:2 selected from the group consisting of H6Q, Y9H/Q, E24F/M, A38R, D39K, D43E, E67K, S105D, R119N, R121D, D122K, D124K/N, and H126K.

20.-29. (canceled)

30. The isolated polypeptide of claim 1, wherein the amino acid sequence of the polypeptide is identical to the amino acid sequence of SEQ ID NO:3 at least at 1, 2, 3, 4, 5, 6, or all 7, identified interface position selected from the group consisting of residues 22, 25, 29, 72, 79, 86, and 87.

31. The isolated polypeptide of claim 1, wherein the polypeptide includes two or more amino acid changes from SEQ ID NO:3 selected from the group consisting of T13D, S71K, N101R, and D105K.

32.-37. (canceled)

38. The isolated polypeptide of claim 1, wherein the amino acid sequence of the polypeptide is identical to the amino acid sequence of SEQ ID NO:4 at least at 1, 2, 3, 4, 5, 6, 7, 8, 9, or all 10 identified interface position selected from the group consisting of residues 28, 31, 35, 36, 39, 131, 132, 135, 139, and 146.

39. The polypeptide of claim 1, wherein the polypeptide comprises an amino acid sequence at least 50%, 60%, 70%, 75%, 80%, 85%, 90%, 95%, 96%, 97%, 98%, 99%, or 100% identical to the full length of the amino acid sequence of SEQ ID NO: 23.

40. The polypeptide of claim 1, further comprising a targeting domain linked to the polypeptide.

41.-57. (canceled)

58. A nanostructure, comprising:
(I) (a) a plurality of first assemblies, each first assembly comprising a plurality of identical first polypeptides, wherein the first polypeptides comprise an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length to the amino acid sequence of SEQ ID NO:1, wherein the polypeptide includes one or more amino acid change from SEQ ID NO:1 selected from the group consisting of K2T, K9R, K11T, K61D, E74D, T126D, E166K, S179K/N, T185K/N, E188K, A195K, and E198K; and
(b) a plurality of second assemblies, each second assembly comprising a plurality of identical second polypeptides, wherein the second polypeptides
(i) comprise an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence of SEQ ID NO:2, wherein the polypeptide includes one or more amino acid change from SEQ ID NO:2 selected from the group consisting of H6Q, Y9H/Q, E24F/M, A38R, D39K, D43E, E67K, S105D, R119N, R121D, D122K, D124K/N, and H126K, or
(ii) are at least 75%, 80%, 85%, 90%, 91%, 92%, 93%, 94%, 95%, 96%, 97%, 98%, or 99% identical over the length of the amino acid sequence selected from the group consisting of SEQ IDS NOS: 2, and 519-522;
wherein the plurality of first assemblies non-covalently interact with the plurality of second assemblies to form a nanostructure; or
(II) (a) a plurality of first assemblies, each first assembly comprising a plurality of identical first polypeptides, wherein the first polypeptides
(i) comprise an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length to the amino acid sequence of SEQ ID NO:1, wherein the polypeptide includes one or more amino acid change from SEQ ID NO:1 selected from the group consisting of K2T, K9R, K11T, K61D, E74D, T126D, E166K, S179K/N, T185K/N, E188K, A195K, and E198K, or
(ii) are at least 75%, 80%, 85%, 90%, 91%, 92%, 93%, 94%, 95%, 96%, 97%, 98%, or 99% identical over the length of the amino acid sequence selected from the group consisting of SEQ IDS NO:1 and 523-526; and
(b) a plurality of second assemblies, each second assembly comprising a plurality of identical second polypeptides, wherein the second polypeptides comprise an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence of SEQ ID NO:2, wherein the polypeptide includes one or more amino acid change from SEQ ID NO:2 selected from the group consisting of H6Q, Y9H/Q, E24F/M, A38R, D39K, D43E, E67K, S105D, R119N, R121D, D122K, D124K/N, and H126K;
wherein the plurality of first assemblies non-covalently interact with the plurality of second assemblies to form a nanostructure; or
(III) (a) a plurality of first assemblies, each first assembly comprising a plurality of identical first polypeptides, wherein the first polypeptides comprise an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length to the amino acid sequence of SEQ ID NO:1, wherein the polypeptide includes one or more amino acid change from SEQ ID NO:1 selected from the group consisting of K2T, K9R, K11T, K61D, E74D, T126D, E166K, S179K/N, T185K/N, E188K, A195K, and E198K; and
(b) a plurality of second assemblies, each second assembly comprising a plurality of identical second polypeptides, wherein the second polypeptides comprise an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence of SEQ ID NO:2, wherein the polypeptide includes one or more amino acid change from SEQ ID NO:2 selected from the group consisting of H6Q, Y9H/Q, E24F/M, A38R, D39K, D43E, E67K, S105D, R119N, R121D, D122K, D124K/N, and H126K;
wherein the plurality of first assemblies non-covalently interact with the plurality of second assemblies to form a nanostructure; or
(IV) (a) a plurality of first assemblies, each first assembly comprising a plurality of identical first polypeptides, wherein the first polypeptides comprising an amino acid sequence that is at least 50% identical to the full length of the amino acid sequence of SEQ ID NO:3, wherein the polypeptide includes one or more amino acid change from SEQ ID NO:3 selected from the group consisting of T13D, S71K, N101R, and D105K; and
(b) a plurality of second assemblies, each second assembly comprising a plurality of identical second polypeptides, wherein the second polypeptides
(i) comprise the polypeptide comprising an amino acid sequence that is at least 50% identical to the full length of the amino acid sequence of SEQ ID NO:4, wherein the polypeptide includes one or more amino acid change from SEQ ID NO:4 selected from the group consisting of S105D, R119N, R121D, D122K, A124K, and A150N, or
(ii) are at least 75%, 80%, 85%, 90%, 91%, 92%, 93%, 94%, 95%, 96%, 97%, 98%, or 99% identical over the length of the amino acid sequence selected from the group consisting of SEQ IDS NOS: 4 and 527-529;
wherein the plurality of first assemblies non-covalently interact with the plurality of second assemblies to form a nanostructure; or
(V) (a) a plurality of first assemblies, each first assembly comprising a plurality of identical first polypeptides, wherein the first polypeptides
(i) comprise a polypeptide comprising an amino acid sequence that is at least 50% identical to the full length of the amino acid sequence of SEQ ID NO:3, wherein the polypeptide includes one or more amino acid change from SEQ ID NO:3 selected from the group consisting of T13D, S71K, N101R, and D105K, or
(ii) wherein the first polypeptides are at least 75%, 80%, 85%, 90%, 91%, 92%, 93%, 94%, 95%, 96%, 97%, 98%, or 99% identical over the length of the amino acid sequence selected from the group consisting of SEQ IDS NOS:3 and 530-532; and
(b) a plurality of second assemblies, each second assembly comprising a plurality of identical second polypeptides, wherein the second polypeptides comprise an amino acid sequence that is at least 50% identical to the full length of the amino acid sequence of SEQ ID NO:4, wherein the polypeptide includes one or more amino acid change from SEQ ID NO:4 selected from the group consisting of S105D, R119N, R121D, D122K, A124K, and A150N;
wherein the plurality of first assemblies non-covalently interact with the plurality of second assemblies to form a nanostructure; or
(VI) (a) a plurality of first assemblies, each first assembly comprising a plurality of identical first polypeptides, wherein the first polypeptides comprise an amino acid sequence that is at least 50% identical to the full length of the amino acid sequence of SEQ ID NO:3, wherein the polypeptide includes one or more amino acid change from SEQ ID NO:3 selected from the group consisting of T13D, S71K, N101R, and D105K; and
(b) a plurality of second assemblies, each second assembly comprising a plurality of identical second polypeptides, wherein the second polypeptides comprise an amino acid sequence that is at least 50% identical to the full length of the amino acid sequence of SEQ ID NO:4, wherein the polypeptide includes one or more amino acid change from SEQ ID NO:4 selected from the group consisting of S105D, R119N, R121D, D122K, A124K, and A150N;
wherein the plurality of first assemblies non-covalently interact with the plurality of second assemblies to form a nanostructure.

59.-92. (canceled)

93. A polynucleotide encoding the polypeptide of claim 1.

94.-95. (canceled)

96. A recombinant expression vector comprising the polynucleotide of claim 93 operably linked to a control sequence.

97. (canceled)

98. A recombinant host cell comprising the recombinant expression vector of claim 96.

99.-114. (canceled)

115. A method of generating polypeptides that self-assemble and package nucleic acid that encodes the polypeptides, comprising:
(a) symmetrically docking one or more polypeptides into an icosahedral geometry;
(b) redesigning the interior surfaces of the polypeptides to have a net charge between −200 and +1200, or between +100 and +900;
(c) encoding the polypeptides in a nucleic acid sequence;
(d) optionally introducing sequence variation in the nucleic acid sequence;
(e) introducing the nucleic acid(s) into a cell;
culturing the cell under conditions to cause expression of the nucleic acid to produce the polypeptide in the cell; and
(g) isolating polypeptides that self-assemble and package the nucleic acid that encodes the polypeptide.

116.-119. (canceled)

120. A synthetic nucleocapsid comprising:
(a) a plurality of first oligomeric polypeptides, each first oligomeric polypeptide comprising a plurality of identical first synthetic polypeptides;
a plurality of second oligomeric polypeptides, each second oligomeric polypeptide comprising a plurality of identical second synthetic polypeptides;
wherein the plurality of first oligomeric polypeptides and the plurality of second oligomeric polypeptides interact non-covalently and assemble into an icosahedral geometry with an interior cavity (a synthetic capsid) that contacts a nucleic acid encoding the polypeptide components of the synthetic nucleocapsid;
wherein the synthetic nucleocapsid does not require viral proteins or naturally-occurring non-viral container proteins, and the first oligomeric polypeptides and second oligomeric polypeptides are selected to provide a positive net charge on the interior surface; or
(b) a synthetic nucleocapsid composed of a computationally-designed capsid derived from proteins that are of non-viral and/or non-container origin and designed to contact each other, wherein the capsid contacts a nucleic acid encoding its own genetic information.

121.-177. (canceled)
